SEQUENCE LISTING (23 sequences)

SEQ ID NO: 1 - DNA, 3464 bp, Homo sapiens
  CDS (264)...(1796)
  misc_feature (1)...(3464): 191P4D12
ggccgtcgttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagagaaaaaaaaaattaaagaaagcctttagatttatccaatgtttactactgggattgcttaaagtgaggcccctccaacaccagggggttaattcctgtgattgtgaaaggggctacttccaaggcatcttcatgcaggcagccccttgggagggcacctgagagctggtagagtctgaaattagggatgtgagcctcgtggttactgagtaaggtaaaattgcatccaccattgtttgtgataccttagggaattgcttggacctggtgacaagggctcctgttcaatagtggtgttggggagagagagagcagtgattatagaccgagagagtaggagttgaggtgaggtgaaggaggtgctgggggtgagaatgtcgcctttccccctgggttttggatcactaattcaaggctcttctggatgtttctctgggttggggctggagttcaatgaggtttatttttagctggcccacccagatacactcagccagaatacctagatttagtacccaaactcttcttagtctgaaatctgctggatttctggcctaagggagaggctcccatccttcgttccccagccagcctaggacttcgaatgtggagcctgaagatctaagatcctaacatgtacattttatgtaaatatgtgcatatttgtacataaaatgatattctgtttttaaataaacagacaaaacttgaaaaa

SEQ ID NO: 2 - protein, 510 aa, Homo sapiens
  misc_feature (1)...(510): 191P4D12
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 1432 bp, Homo sapiens
  CDS (32)...(1432)
  misc_feature (1)...(1432): Ha22-2(2,4)6.1 heavy chain
ggtgatcagcactgaacacagaggactcaccatggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctataacatgaactgggtccgccaggctccagggaaggggctggagtgggtttcatacattagtagtagtagtagtaccatatactacgcagactctgtgaagggccgattcaccatctccagagacaatgccaagaactcactgtctctgcaaatgaacagcctgagagacgaggacacggctgtgtattactgtgcgagagcatactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 466 aa, Homo sapiens
  misc_feature (1)...(466): Ha22-2(2,4)6.1 heavy chain
MELGLCWVFLVAILEGVQCEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 735 bp, Homo sapiens
  CDS (25)...(735)
  misc_feature (1)...(735): Ha22-2(2,4)6.1 light chain
agtcagacccagtcaggacacagcatggacatgagggtccccgctcagctcctggggctcctgctgctctggttcccaggttccagatgcgacatccagatgacccagtctccatcttccgtgtctgcatctgttggagacagagtcaccatcacttgtcgggcgagtcagggtattagcggctggttagcctggtatcagcagaaaccagggaaagcccctaagttcctgatctatgctgcatccactttgcaaagtggggtcccatcaaggttcagcggcagtggatctgggacagatttcactctcaccatcagcagcctgcagcctgaagattttgcaacttactattgtcaacaggctaacagtttccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 236 aa, Homo sapiens
  misc_feature (1)...(236): Ha22-2(2,4)6.1 light chain
MDMRVPAQLLGLLLLWFPGSRCDIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 466 aa, Homo sapiens; identical to SEQ ID NO: 4
  misc_feature (1)...(466): Ha22-2(2,4)6.1 heavy chain

SEQ ID NO: 8 - protein, 236 aa, Homo sapiens; identical to SEQ ID NO: 6
  misc_feature (1)...(236): Ha22-2(2,4)6.1 light chain

SEQ ID NO: 9 - protein, 5 aa, Homo sapiens
  CDR1 of heavy chain
SYNMN

SEQ ID NO: 10 - protein, 17 aa, Homo sapiens
  CDR2 of heavy chain
YISSSSSTIYYADSVKG

SEQ ID NO: 11 - protein, 8 aa, Homo sapiens
  CDR3 of heavy chain
AYYYGMDV

SEQ ID NO: 12 - protein, 11 aa, Homo sapiens
  CDR1 of light chain
RASQGISGWLA

SEQ ID NO: 13 - protein, 7 aa, Homo sapiens
  CDR2 of light chain
AASTLQS

SEQ ID NO: 14 - protein, 9 aa, Homo sapiens
  CDR3 of light chain
QQANSFPPT

SEQ ID NO: 15 - protein, 4 aa, Artificial Sequence
  linker
GFLG

SEQ ID NO: 16 - protein, 8 aa, Artificial Sequence
  VH CDR1 according to IMGT
GFTFSSYN

SEQ ID NO: 17 - protein, 8 aa, Artificial Sequence
  VH CDR2 according to IMGT
ISSSSSTI

SEQ ID NO: 18 - protein, 10 aa, Artificial Sequence
  VH CDR3 according to IMGT
ARAYYYGMDV

SEQ ID NO: 19 - protein, 6 aa, Artificial Sequence
  VL CDR1 according to IMGT
QGISGW

SEQ ID NO: 20 - protein, 3 aa, Artificial Sequence
  VL CDR2 according to IMGT
AAS

SEQ ID NO: 21 - protein, 9 aa, Artificial Sequence
  VL CDR3 according to IMGT
QQANSFPPT

SEQ ID NO: 22 - protein, 117 aa, Artificial Sequence
  heavy chain variable region (VH), the 20th amino acid (glutamic acid) to the 136th amino acid (serine) of SEQ ID NO7
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 108 aa, Artificial Sequence
  light chain variable region (VL), the 23rd amino acid (aspartic acid) to the 130th amino acid (arginine) of SEQ ID NO8
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR

(19) United States
(12) Patent Application Publication
(10) Pub. No.: US 2023/0330251 A1
(43) Pub. Date: Oct. 19, 2023

(54) METHODS FOR TREATING CANCERS WITH ANTIBODY DRUG CONJUGATES (ADC) THAT BIND TO 191P4D12 PROTEINS

(71) Applicants: AGENSYS, INC., Northbrook, IL (US); SEAGEN INC., Bothell, WA (US)

(72) Inventor: Elaina Marie GARTNER, Lake Forest Park, WA (US)

(73) Assignees: AGENSYS, INC., Northbrook, IL (US); SEAGEN INC., Bothell, WA (US)

(21) Appl. No.: 18/025,349

(22) PCT Filed: Sep. 16, 2021
(86) PCT No.: PCT/US2021/050627

§ 371 Date: Mar. 8, 2023

Related U.S. Application Data
(60) Provisional application No. 63/240,794, filed on Sep. 3, 2021.
(60) Provisional application No. 63/196,641, filed on Jun. 3, 2021.
(60) Provisional application No. 63/080,013, filed on Sep. 17, 2020.

(51) Int. Cl. A61K 47/68; A61P 35/00; C07K 16/28

(52) U.S. Cl. CPC A61K 47/6849; A61P 35/00; C07K 16/2803; A61K 47/6817; A61K 47/6889; A61K 2039/505

(57) ABSTRACT

Provided herein are methods for the treatment of cancers with antibody drug conjugates (ADC) that bind to 191P4D12 proteins.

### CROSS-REFERENCE TO RELATED APPLICATIONS

This application claims the benefit of U.S. Application No. 63/080,013, filed Sep. 17, 2020, U.S. Application No. 63/196,641, filed Jun. 3, 2021, and U.S. Application No. 63/240,794, filed Sep. 3, 2021, the disclosure of each of which is incorporated by reference herein in its entirety.

### REFERENCE TO SEQUENCE LISTING SUBMITTED ELECTRONICALLY

This application contains a sequence listing, which is submitted electronically via EFS-Web as an ASCII formatted sequence listing with a file name of “14369-274-228_SEQ_LISTING.txt” and a creation date of Sep. 13, 2021 and having a size of 39,755 bytes. The sequence listing submitted via EFS-Web is part of the specification and is herein incorporated by reference in its entirety.

### 1. FIELD

Provided herein are methods for treating cancers with antibody drug conjugates (ADC) that bind to 191P4D12 protein (Nectin-4).

### 2. BACKGROUND

Cancer is the leading cause of death in the US for people 35 to 65 years of age and it is the second leading cause of death worldwide. It was estimated in 2019 that there would be approximately 1.7 million new cancer cases and approximately 610000 deaths from cancer in the US (National Cancer Institute. 2019. Cancer Stat Facts: Cancer of Any Site. seer.cancer.gov/statfacts/html/all.html. Accessed 5 Jun. 2019). Globally there were an estimated 18.1 million new cancer cases in 2018 and approximately 9.6 million deaths attributed to cancer in 2018 (World Health Organization. Press Release. September 2018. who.int/cancer/PRGlobocanFinal.pdf. Accessed 5 Jun. 2019). Most deaths now occur in patients with metastatic cancers. In fact, in the last 20 years, advances in treatment, including surgery, radiotherapy and adjuvant chemotherapy cured most patients with localized cancer. Patients whose cancer presented or recurred as metastatic disease obtained only modest benefit from conventional therapies in terms of overall survival (OS) and were rarely cured.

New therapeutic strategies for advanced and/or metastatic cancers include targeting molecular pathways important for cancer cell survival and novel cytotoxic compounds. The benefit of these novel drugs is reflected in prolonged survival; however, the outcome for most patients with distant metastases is still poor and novel therapies are needed.

191P4D12 (which is also known as Nectin-4) is a type I transmembrane protein and member of a family of related immunoglobulin-like adhesion molecules implicated in cell-to-cell adhesion. 191P4D12 belongs to the Nectin family of adhesion molecules. 191P4D12 is composed of an extracellular domain (ECD) containing 3 Ig-like subdomains, a transmembrane helix, and an intracellular region (Takai Y et al, Annu Rev Cell Dev Biol 2008; 24:309-42). Nectins are thought to mediate Ca²⁺-independent cell-cell adhesion via both homophilic and heterophilic trans interactions at adherens junctions where they can recruit cadherins and modulate cytoskeletal rearrangements (Rikitake & Takai, Cell Mol Life Sci. 2008; 65(2):253-63). Sequence identity of 191P4D12 to other Nectin family members is low and ranges between 25% to 30% in the ECD (Reymond N et al, J Biol Chem 2001; 43205-15). Nectin-facilitated adhesion supports several biological processes, such as immune modulation, host-pathogen interaction, and immune evasion (Sakisaka T et al, Current Opinion in Cell Biology 2007; 19:593-602).

Urothelial Cancer

According to the International Agency for Research on Cancer (IARC), urothelial cancer kills more than 165000 patients annually and is the ninth most common cancer overall worldwide. Approximately 151000 new cases of urothelial cancer are diagnosed annually in Europe, with 52000 deaths per year. Over 22000 new cases are diagnosed annually in Japan, with 7600 deaths per year. Cancer Fact Sheets: All cancers excluding Non-Melanoma Skin. International Agency for Research on Cancer 2017. Retrieved from gco.iarc.fr/today/fact-sheets-cancers?cancer=29&type=0&sex=0. Accessed 19 Dec. 2017. According to National Cancer Institute estimates, over 79000 new cases of urothelial cancer were diagnosed in 2017, and more than 16000 people died from the disease in the United States (US). SEER Cancer Stat Facts: Bladder Cancer. National Cancer Institute. Bethesda, MD, seer.cancer.govstatfacts/html/urinb.html. Accessed 19 Dec. 2017.

First-line therapy for metastatic urothelial cancer in patients with sufficient renal function consists of cisplatin-based combinations, like methotrexate, vinblastine, doxorubicin, and cisplatin (MVAC) or gemcitabine plus cisplatin, which demonstrate overall response rates up to 50%, including approximately 10-15% complete response (CRs). Bellmunt J, et al., N Engl. Med. 2017; 376:1015-26. DOI: 10.1056/NEJMoa1613683. Despite initial chemosensitivity, patients are not cured and the outcome of metastatic urothelial cancer after these regimens is poor: median time to progression is only 7 months and median overall survival (OS) is 14 months. Approximately 15% of patients survive at least 5 years and the prognosis is particularly poor among patients with visceral metastases for whom the 5-year OS rate is 7%. von der Maase H, et al., J Clin Oncol. 2005; 23:4602-8.

For second line treatment, the small-molecule tubulin inhibitor vinflunine (Javlor®) is approved only in Europe. The median OS is 6.9 months compared to a median OS of 4.6 months for best supportive care. Bellmunt J, et al., J Clin Oncol. 2009; 27:4454-61. For decades, there were no major changes to the treatment landscape with only cytotoxic chemotherapies available, until the recent approvals of immune check point inhibitors (CPI) targeting the programmed death 1/programmed death-ligand 1 (PD-1/PD-L1). As of May 2016, starting with the PD-L1 inhibitor atezolizumab, several CPIs have received FDA approval for urothelial cancer for platinum-pretreated patients in the United States. Most approvals have been based on single arm phase II data. See Tecentriq Prescribing Information, Genentech, April 2017, Opdivo Prescribing Information, Bristol-Myers Squibb, September 2017, Imfinzi Prescribing Information, AstraZeneca, May 2017 and Bavencio Prescribing Information, EMD Serono, March 2017. However, in 2017, results from the phase III trial KEYNOTE-045 demonstrated that patients treated with pembrolizumab had significantly longer survival when compared with the standard second-line chemotherapy. Bellmunt J, et al., N Engl J Med. 2017; 376:1015-26. DOI: 10.1056/NEJMoa1613683. This led to the regular approval of pembrolizumab as second line treatment for patients with locally advanced or metastatic urothelial cancer (mUC; Keytruda Prescribing Information, Merck, September 2017). The approval was based on a median OS of 10.3 months for pembrolizumab compared with 7.4 months with taxane chemotherapy or vinflunine. Bellmunt J, et al., N Engl J Med. 2017; 376:1015-26. DOI: 10.1056/NEJMoa1613683. Marketing approval of CPIs in Europe have followed and approvals in Asia are expected. Other PD-1 and PD-L1 inhibitors are currently being evaluated in clinical trials for urothelial cancer, as first and second line therapy. Mullane S A & Bellmunt J. Curr Opin 2016; 26:556-63.

Currently, no therapies are approved for patients with locally advanced or mUC previously treated with a CPI.

Bladder Cancer

Of all new cases of cancer in the United States, bladder cancer represents approximately 5 percent in men (fifth most common neoplasm) and 3 percent in women (eighth most common neoplasm). The incidence is increasing slowly, concurrent with an increasing older population. American Cancer Society (cancer.org) estimates that there are 81,400 new cases annually, including 62,100 in men and 19,300 in women, which accounts for 4.5% of all cancer cases. The age-adjusted incidence in the United States is 20 per 100,000 for men and women. There are an estimated 17,980 deaths from bladder cancer in annually (13,050 in men and 4,930 in women), which accounts for 3% of cancer related deaths. Bladder cancer incidence and mortality strongly increase with age and will be an increasing problem as the population becomes more elderly.

Most bladder cancers recur in the bladder. Bladder cancer is managed with a combination of transurethral resection of the bladder (TUR) and intravesical chemotherapy or immunotherapy. The multifocal and recurrent nature of bladder cancer points out the limitations of TUR. Most muscle-invasive cancers are not cured by TUR alone. Radical cystectomy and urinary diversion is the most effective means to eliminate the cancer but carry an undeniable impact on urinary and sexual function. There continues to be a significant need for treatment modalities that are beneficial for bladder cancer patients.

There is a significant need for additional therapeutic methods for urothelial and bladder cancers. These include the use of antibodies and antibody drug conjugates as treatment modalities.

### 3. SUMMARY

Provided herein are methods for the treatment of various cancers in subjects, including subjects with previously treated locally advanced or metastatic urothelial cancer, using an antibody drug conjugate (ADC) that binds 191P4D12.

In some embodiments, the previous treatment includes platinum-based chemotherapy. In certain embodiments, the previous treatment includes an immune checkpoint inhibitor (CPI). In other embodiments, the previous treatment includes both platinum-based chemotherapy and a CPI.

Embodiment 1. A method of treating urothelial or bladder cancer in a human subject having liver metastases, comprising administering to the subject having liver metastases an effective amount of an antibody drug conjugate,
wherein the subject has received an immune checkpoint inhibitor (CPI) therapy, and
wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

Embodiment 2. A method of treating urothelial or bladder cancer in a human subject having a primary site of tumor in the upper urinary tract, comprising administering to the subject having a primary site of tumor in the upper urinary tract an effective amount of an antibody drug conjugate,
wherein the subject has received an immune checkpoint inhibitor (CPI) therapy, and
wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

Embodiment 3. A method of treating urothelial or bladder cancer in a human subject, comprising administering to the subject an effective amount of an antibody drug conjugate,
wherein the subject has received an immune checkpoint inhibitor (CPI) therapy,
wherein the subject had progression or recurrence of the cancer during or following the CPI therapy, and
wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

Embodiment 4. The method of any one of embodiments 1 to 3, wherein the subject has a duration of response of at least or about 7 months following the treatment.

Embodiment 5. The method of any one of embodiments 1 to 3, wherein the subject has a duration of response ranging from 5 to 9 months following the treatment.

Embodiment 6. The method of embodiment 1, wherein the subject has a progression free survival of at least or about 4 months following the treatment.

Embodiment 7. The method of embodiment 2 or 3, wherein the subject has a progression free survival of at least or about 5 months following the treatment.

Embodiment 8. The method of embodiment 1, wherein the subject has a progression free survival ranging from 4 to 9 months following the treatment.

Embodiment 9. The method of embodiment 2 or 3, wherein the subject has a progression free survival ranging from 5 to 9 months following the treatment.

Embodiment 10. The method of embodiment 1, wherein the subject has an overall survival of at least or about 9 months following the treatment.

Embodiment 11. The method of embodiment 2, wherein the subject has an overall survival of at least or about 12 months following the treatment.

Embodiment 12. The method of embodiment 3, wherein the subject has an overall survival of at least or about 11 months following the treatment.

Embodiment 13. The method of any one of embodiments 1 to 3, wherein the subject has an overall survival ranging from 9 to 19 months following the treatment.

Embodiment 14. The method of any one of embodiments 1 to 3, wherein a population of the subjects is treated by the methods, and wherein the percentage of the subjects having complete response in the treated population is at least or about 4%.

Embodiment 15. The method of any one of embodiments 1 to 3, wherein a population of the subjects is treated by the methods, and wherein the percentage of the subjects having partial response in the treated population is at least or about 35%.

Embodiment 16. The method of embodiment 1, wherein a population of the subjects is treated by the methods, and wherein overall response rate in the treated population is at least or about 35%.

Embodiment 17. The method of embodiment 2, wherein a population of the subjects is treated by the methods, and wherein overall response rate in the treated population is at least or about 43%.

Embodiment 18. The method of embodiment 3, wherein a population of the subjects is treated by the methods, and wherein overall response rate in the treated population is at least or about 39%.

Embodiment 19. The method of any one of embodiments 1 to 3, wherein a population of the subjects is treated by the methods, and wherein the percentage of the subjects having stable disease in the treated population is at least or about 30%.

Embodiment 20. The method of any one of embodiments 1 to 3, wherein a population of the subjects is treated by the methods, and wherein median duration of response in the treated population is at least or about 7 months.

Embodiment 21. The method of any one of embodiments 1 to 3, wherein a population of the subjects is treated by the methods, and wherein duration of response in the treated population ranges from 5 to 9 months.

Embodiment 22. The method of embodiment 1, wherein a population of the subjects is treated by the methods, and wherein median progression free survival in the treated population is at least or about 4 months.

Embodiment 23. The method of embodiment 1, wherein a population of the subjects is treated by the methods, and wherein progression free survival in the treated population ranges from 4 to 9 months.

Embodiment 24. The method embodiment 2 or 3, wherein a population of the subjects is treated by the methods, and wherein median progression free survival in the treated population is at least or about 5 months.

Embodiment 25. The method of embodiment 2 or 3, wherein a population of the subjects is treated by the methods, and wherein progression free survival in the treated population ranges from 5 to 9 months.

Embodiment 26. The method of embodiment 1, wherein a population of the subjects is treated by the methods, and wherein median overall survival in the treated population is at least or about 9 months.

Embodiment 27. The method of embodiment 2, wherein a population of the subjects is treated by the methods, and wherein median overall survival in the treated population is at least or about 12 months.

Embodiment 28. The method of embodiment 3, wherein a population of the subjects is treated by the methods, and wherein median overall survival in the treated population is at least or about 11 months.

Embodiment 29. The method of any one of embodiments 1 to 3, wherein a population of the subjects is treated by the methods, and wherein overall survival in the treated population ranges from 9 to 19 months.

Embodiment 30. The method of any one of embodiments 1 to 3, wherein the complete response rate is at least or about 4% for a population of subjects treated with the method.

Embodiment 31. The method of any one of embodiments 1 to 3, wherein the partial response rate is at least or about 35% for a population of subjects treated with the method.

Embodiment 32. The method of embodiment 1, wherein overall response rate is at least or about 35% for a population of subjects treated with the method.

Embodiment 33. The method of embodiment 2, wherein overall response rate is at least or about 43% for a population of subjects treated with the method.

Embodiment 34. The method of embodiment 3, wherein overall response rate is at least or about 39% for a population of subjects treated with the method.

Embodiment 35. The method of any one of embodiments 1 to 3, wherein the median duration of response is at least or about 7 months for a population of subjects treated with the method.

Embodiment 36. The method of any one of embodiments 1 to 3, wherein the duration of response is from 5 to 9 months for a population of subjects treated with the method.

Embodiment 37. The method of embodiment 1, wherein the median progression free survival is at least or about 4 months for a population of subjects treated with the method.

Embodiment 38. The method of any one of embodiments 1 to 3, wherein the progression free survival is from 4 to 9 months for a population of subjects treated with the method.

Embodiment 39. The method of embodiment 2 or 3, wherein the median progression free survival is at least or about 5 months for a population of subjects treated with the method.

Embodiment 40. The method of embodiment 2 or 3, wherein the progression free survival is from 5 to 9 months for a population of subjects treated with the method.

Embodiment 41. The method of embodiment 1, wherein the median overall survival is at least or about 9 months for a population of subjects treated with the method.

Embodiment 42. The method of embodiment 2, wherein the median overall survival is at least or about 12 months for a population of subjects treated with the method.

Embodiment 43. The method of embodiment 3, wherein the median overall survival is at least or about 11 months for a population of subjects treated with the method.

Embodiment 44. The method of any one of embodiments 1 to 3, wherein the overall survival is from 9 to 19 months for a population of subjects treated with the method.

Embodiment 45. The method of any one of embodiments 1 to 44, wherein the subject is a subject that received platinum-based chemotherapy.

Embodiment 46. The method of any one of embodiments 1 to 45, wherein the cancer is urothelial cancer, and wherein the human subject has locally advanced or metastatic urothelial carcinoma.

Embodiment 47. The method of any one of embodiments 1 to 46, wherein the subject has one or more of the conditions selected from the group consisting of:
(i) absolute neutrophil count (ANC) no less than 1500/mm3;
(ii) platelet count no less than 100×109/L;
(iii) hemoglobin no less than 9 g/dL;
(iv) serum bilirubin no more than either of 1.5 times of upper limit of normal (ULN) or 3 times ULN for patients with Gilbert's disease;
(v) CrCl no less than 30 mL/min, and
(vi) alanine aminotransferase and aspartate aminotransferase no more than 3 fold of ULN.

Embodiment 48. The method of embodiment 47, wherein the subject has all of conditions (i) to (vi) of embodiment 47.

Embodiment 49. The method of embodiment 47 or 48, wherein the CrCl is measured by 24 hour urine collection or estimated by the Cockcroft-Gault criteria.

Embodiment 50. The method of any one of embodiments 1 to 49, wherein the subject has no more than Grade 2 sensory or motor neuropathy.

Embodiment 51. The method of any one of embodiments 1 to 50, wherein the subject has no active central nervous system metastases.

Embodiment 52. The method of any one of embodiments 1 to 51, wherein the subject has no uncontrolled diabetes.

Embodiment 53. The method of embodiment 52, wherein the uncontrolled diabetes is determined by hemoglobin A1c (HbA1c) no less than 8% or HbA1c between 7 and 8% with associated diabetes symptoms that are not otherwise explained.

Embodiment 54. The method of embodiment 53, wherein the associated diabetes symptoms comprise or consist of polyuria, polydipsia, or both polyuria and polydipsia.

Embodiment 55. The method of any one of embodiments 1 to 54, wherein the CPI therapy is a therapy of programmed death receptor-1 (PD-1) inhibitor.

Embodiment 56. The method of any one of embodiments 1 to 54, wherein the CPI therapy is a therapy of programmed death-ligand 1 (PD-L1) inhibitor.

Embodiment 57. The method of embodiment 55, wherein PD-1 inhibitor is nivolumab or pembrolizumab

Embodiment 58. The method of embodiment 56, wherein PD-L1 inhibitor is selected from a group consisting of atezolizumab, avelumab, and durvalumab.

Embodiment 59. The method of any one of embodiments 1 to 58, wherein the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or
wherein the antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

Embodiment 60. The method of any one of embodiments 1 to 58, wherein the antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
wherein the antibody or antigen binding fragment thereof comprises CDR-H1 consisting of the amino acid sequence of SEQ ID NO:16, CDR-H2 consisting of the amino acid sequence of SEQ ID NO:17, CDR-H3 consisting of the amino acid sequence of SEQ ID NO:18; CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.

Embodiment 61. The method of any one of embodiments 1 to 60, wherein the antibody or antigen binding fragment thereof comprises a heavy chain variable region comprising the amino acid sequence of SEQ ID NO:22 and a light chain variable region comprising the amino acid sequence of SEQ ID NO:23.

Embodiment 62. The method of any one of embodiments 1 to 61, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

Embodiment 63. The method of any one of embodiments 1 to 61, wherein the antigen binding fragment is an Fab, F(ab′)2, Fv or scFv.

Embodiment 64. The method of any one of embodiments 1 to 62, wherein the antibody is a fully human antibody.

Embodiment 65. The method of any one of embodiments 1 to 62 and 64, wherein the antibody is an IgG1 and light chain is a kappa light chain.

Embodiment 66. The method of any one of embodiments 1 to 65, wherein the antibody or antigen binding fragment thereof is recombinantly produced.

Embodiment 67. The method of any one of embodiments 1 to 66, wherein the antibody or antigen binding fragment is conjugated to each unit of MMAE via a linker.

Embodiment 68. The method of embodiment 67, wherein the linker is an enzyme-cleavable linker, and wherein the linker forms a bond with a sulfur atom of the antibody or antigen binding fragment thereof.

Embodiment 69. The method of embodiment 67 or 68, wherein the linker has a formula of: -Aa-Ww-Yy-; wherein -A- is a stretcher unit, a is 0 or 1; -W- is an amino acid unit, w is an integer ranging from 0 to 12; and -Y- is a spacer unit, y is 0, 1, or 2.

Embodiment 70. The method of embodiment 69, wherein the stretcher unit has the structure of Formula (1) below; the amino acid unit is valine-citrulline; and the spacer unit is a PAB group comprising the structure of Formula (2) below:

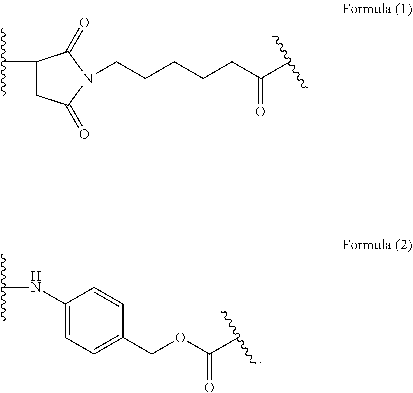 [SMILES: *C(=O)CCCCCN1C(=O)CC(*)C1=O ; *Nc1ccc(COC(*)=O)cc1]

Embodiment 71. The method of embodiment 69 or 70, wherein the stretcher unit forms a bond with a sulfur atom of the antibody or antigen binding fragment thereof; and wherein the spacer unit is linked to MMAE via a carbamate group.

Embodiment 72. The method of any one of embodiments 1 to 71, wherein the ADC comprises from 1 to 20 units of MMAE per antibody or antigen binding fragment thereof.

Embodiment 73. The method of any one of embodiments 1 to 72, wherein the ADC comprises from 1 to 10 units of MMAE per antibody or antigen binding fragment thereof.

Embodiment 74. The method of any one of embodiments 1 to 73, wherein the ADC comprises from 2 to 8 units of MMAE per antibody or antigen binding fragment thereof.

Embodiment 75. The method of any one of embodiments 1 to 74, wherein the ADC comprises from 3 to 5 units of MMAE per antibody or antigen binding fragment thereof.

Embodiment 76. The method of any one of embodiments 1 to 73, wherein the ADC has the following structure:

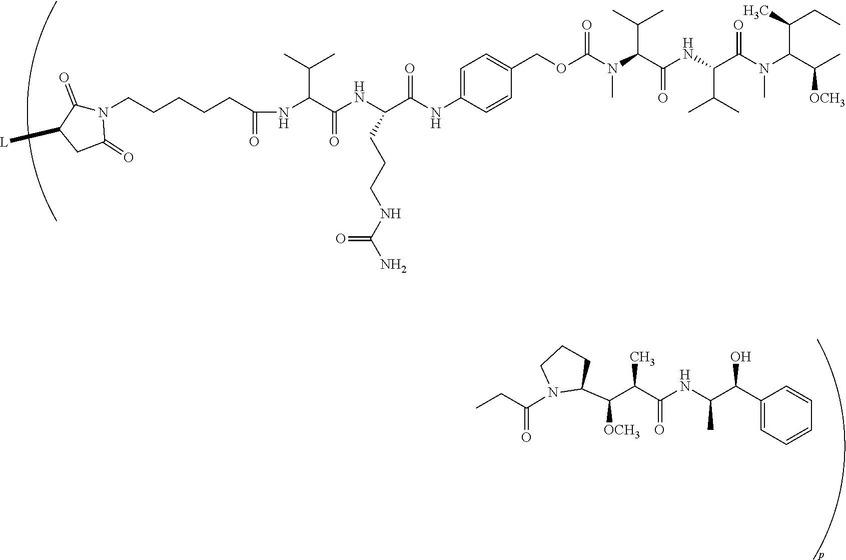

wherein L- represents the antibody or antigen binding fragment thereof and p is from 1 to 10.

Embodiment 77. The method of embodiment 76, wherein p is from 2 to 8.

Embodiment 78. The method of embodiment 76 or 77, wherein p is from 3 to 5.

Embodiment 79. The method of any one of embodiments 76 to 78, wherein p is from 3 to 4.

Embodiment 80. The method of any one of embodiments 77 to 79, wherein p is about 4.

Embodiment 81. The method of any one of embodiments 76 to 79, wherein the average p value of the effective amount of the antibody drug conjugates is about 3.8.

Embodiment 82. The method of any one of embodiments 1 to 81, wherein the ADC is administered at a dose of about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.

Embodiment 83. The method of any one of embodiments 1 to 82, wherein the ADC is administered at a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

Embodiment 84. The method of any one of embodiments 1 to 83, wherein the ADC is administered at a dose of about 1 mg/kg of the subject's body weight.

Embodiment 85. The method of any one of embodiments 1 to 83, wherein the ADC is administered at a dose of about 1.25 mg/kg of the subject's body weight.

Embodiment 86. The method of any one of embodiments 1 to 85, wherein the ADC is administered by an intravenous (IV) injection or infusion.

Embodiment 87. The method of any one of embodiments 1 to 86, wherein the ADC is administered by an IV injection or infusion three times every four-week cycle.

Embodiment 88. The method of any one of embodiments 1 to 87, wherein the ADC is administered by an IV injection or infusion on Days 1, 8 and 15 of every four-week cycle.

Embodiment 89. The method of any one of embodiments 1 to 88, wherein the ADC is administered by an IV injection or infusion over about 30 minutes three times every four-week cycle.

Embodiment 90. The method of any one of embodiments 1 to 89, wherein the ADC is administered by an IV injection or infusion over about 30 minutes on Days 1, 8 and 15 of every four-week cycle.

Embodiment 91. The method of any one of embodiments 1 to 90, wherein the ADC is formulated in a pharmaceutical composition comprising L-histidine, polysorbate-20 (TWEEN-20), and trehalose dehydrate.

Embodiment 92. The method of any one of embodiments 1 to 91, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

Embodiment 93. The method of any one of embodiments 1 to 91, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

Embodiment 94. The method of any one of embodiments 1 to 93, wherein the ADC is enfortumab vedotin (EV) or a biosimilar thereof, wherein the EV is administered at a dose of about 1.25 mg/kg of the subject's body weight, and wherein the dose is administered by an IV injection or infusion over about 30 minutes on Days 1, 8 and 15 of every four-week cycle.

Embodiment 95. The method of any one of embodiments 1 to 94, whereby a population of the subjects have a complete response following the treatment.

Embodiment 96. The method of any one of embodiments 1 to 94, wherein a population of the subjects have a partial response following the treatment.

Embodiment 97. The method of any one of embodiments 1 to 94, wherein a population of the subjects have a complete response or a partial response following the treatment.

Embodiment 98. The method of any one of embodiments 1 to 94, wherein a population of the subjects have a stable disease following the treatment.

### 4. BRIEF DESCRIPTION OF THE DRAWINGS

FIGS. 1A-1E depict the nucleotide and amino acid sequences of nectin-4 protein (FIG. 1A), the nucleotide and amino acid sequences of the heavy chain (FIG. 1B) and light chain (FIG. 1C) of Ha22-2(2.4)6.1, and the amino acid sequences of the heavy chain (FIG. 1D) and light chain of Ha22-2(2.4)6.1 (FIG. 1E).

FIG. 2A depicts the overall study design of the clinical study described in Section 6.1. FIG. 2B depicts the study scheme of the clinical study described in Section 6.1. FIG. 2C depicts the EuroQOL 5-dimensions (EQ-5D-5L) described in Section 6.1.

FIG. 3 depicts the analysis efficacy boundaries of the clinical study described in Section 6.1. #Note: The pre-defined efficacy boundary IDMC used for IA of OS (=0.00661) was adjusted using 299 deaths observed as of initial data snapshot per initial cutoff. Two more deaths were reported after the initial data snapshot and hence 301 deaths were displayed in the table of primary analysis of OS in TLR.

FIG. 4 depicts the Overall Survival, Kaplan Meier Plot—FAS of the clinical study described in Section 6.1.

FIG. 5 depicts the Overall Survival, Sub-groups Results of the clinical study described in Section 6.1.

FIG. 6 depicts the PFS, Kaplan Meier Plot—FAS of the clinical study described in Section 6.1.

FIG. 7 depicts the results of subgroup analyses, which show that a progression-free survival benefit with enfortumab vedotin (EV) was present across multiple subgroups.

FIG. 8 depicts the results of subgroup analyses of Overall Response Rate.

FIG. 9. depicts the Kaplan-Meier estimate for investigator-assessed duration of response based on treatment group in all patients with confirmed complete or partial response.

FIGS. 10A-10D depict Kaplan-Meier estimates of overall survival by subgroup. FIG. 10A depicts the age ≥65 years subgroup. FIG. 10B depicts the subgroup with presence of liver metastasis. FIG. 10C depicts the subgroup with primary upper tract disease. FIG. 10D depicts the nonresponsive to prior PD-1/L1 inhibitor subgroup. Abbreviations: CI, confidence interval; HR, hazard ratio; PD-1/L1, programmed cell death protein-1 or programmed death-ligand 1.

FIGS. 11A-11D depict Kaplan-Meier estimates of progression-free survival by subgroup. FIG. 11A depicts the age ≥65 years subgroup. FIG. 11B depicts the subgroup with presence of liver metastasis. FIG. 11C depicts the subgroup with primary upper tract disease. FIG. 11D depicts the nonresponsive to prior PD-1/L1 inhibitor subgroup. Abbreviations: CI, confidence interval; HR, hazard ratio; PD-1/L1, programmed cell death protein-1 or programmed death-ligand 1.

FIG. 12 depicts treatment-related adverse events (safety population). Abbreviations: EV, enfortumab vedotin; PD-1/L1, programmed cell death protein-1 or programmed death-ligand 1; SC, standard chemotherapy.

FIG. 13 depicts exposure-adjusted grade ≥3 treatment-related adverse events in hard-to-treat subgroups. Abbreviations: EV, enfortumab vedotin; PD-1/L1, programmed cell death protein-1 or programmed death-ligand 1; SC, standard chemotherapy.

### 5. DETAILED DESCRIPTION

Before the present disclosure is further described, it is to be understood that the disclosure is not limited to the particular embodiments set forth herein, and it is also to be understood that the terminology used herein is for describing particular embodiments only, and is not intended to be limiting.

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dithel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)2, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well-known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment thereof described herein terms such as “bind to,” “that specifically bind to,” and analogous terms are also used interchangeably herein and refer to binding molecules of antigen binding domains that specifically bind to an antigen, such as a polypeptide. An antibody or antigen binding fragment that binds to or specifically binds to an antigen may be cross-reactive with related antigens. In certain embodiments, an antibody or antigen binding fragment that binds to or specifically binds to an antigen does not cross-react with other antigens. An antibody or antigen binding fragment that binds to or specifically binds to an antigen can be identified, for example, by immunoassays, Octet®, Biacore®, or other techniques known to those of skill in the art. In some embodiments, an antibody or antigen binding fragment binds to or specifically binds to an antigen when it binds to an antigen with higher affinity than to any cross-reactive antigen as determined using experimental techniques, such as radioimmunoassays (MA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen binding fragment to a “non-target” protein is less than about 10% of the binding of the binding molecule or antigen binding domain to its particular target antigen, for example, as determined by fluorescence activated cell sorting (FACS) analysis or MA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding fragment that binds to an antigen includes one that is capable of binding the antigen with sufficient affinity such that the binding molecule is useful, for example, as a diagnostic agent in targeting the antigen. In certain embodiments, an antibody or antigen binding fragment that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding fragment binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, a Octet® QK384 system, or by Biacore®, using, for example, a Biacore® TM-2000 or a Biacore® TM-3000. An “on-rate” or “rate of association” or “association rate” or “kon” may also be determined with the same biolayer interferometry (BLI) or surface plasmon resonance (SPR) techniques described above using, for example, the Octet® QK384, the Biacore® TM-2000, or the Biacore® TM-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate comprising the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies comprising variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising nonhuman antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Bruggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well-known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well-known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dithel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MEW) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted below in Table 1.

TABLE 1

| | Kabat | AbM | Chothia | Contact | IMGT |
|---|---|---|---|---|---|
| CDR-L1 | L24--L34 | L24--L34 | L24--L34 | L30--L36 | L27--L38 |
| CDR-L2 | L50--L56 | L50--L56 | L50--L56 | L46--L55 | L56--L65 |
| CDR-L3 | L89--L97 | L89--L97 | L89--L97 | L89--L96 | L105-L117 |
| CDR-H1 | H31--H35B | H26--H35B | H26--H32 . . . 34 | H30--H35B | H27--H38 |
| | (Kabat | | | | |
| | Numbering) | | | | |
| CDR-H1 | H31--H35 | H26--H35 | H26--H32 | H30--H35 | |
| | (Chothia | | | | |
| | Numbering) | | | | |
| CDR-H2 | H50--H65 | H50--H58 | H52--H56 | H47--H58 | H56--H65 |
| CDR-H3 | H95--H102 | H95--H102 | H95--H102 | H93--H101 | H105-H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule comprising a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fe region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations comprising a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The abbreviation “MMAE” refers to monomethyl auristatin E.

Unless otherwise indicated by context, a hyphen (-) designates the point of attachment to the pendant molecule.

The term “Chemotherapeutic Agent” refers to all chemical compounds that are effective in inhibiting tumor growth. Non-limiting examples of chemotherapeutic agents include alkylating agents; for example, nitrogen mustards, ethyleneimine compounds and alkyl sulphonates; antimetabolites, for example, folic acid, purine or pyrimidine antagonists; mitotic inhibitors, for example, anti-tubulin agents such as vinca alkaloids, auristatins and derivatives of podophyllotoxin; cytotoxic antibiotics; compounds that damage or interfere with DNA expression or replication, for example, DNA minor groove binders; and growth factor receptor antagonists. In addition, chemotherapeutic agents include cytotoxic agents (as defined herein), antibodies, biological molecules and small molecules.

As used herein, the term “conservative substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)). Such exemplary substitutions are preferably made in accordance with those set forth in Table 2 and Table 3. For example, such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table 3 herein; pages 13-15 “Biochemistry” 2nd ED. Lubert Stryer ed (Stanford University); Henikoff et al., PNAS 1992 Vol 89 10915-10919; Lei et al., J Biol Chem 1995 May 19; 270(20):11882-11886). Other substitutions are also permissible and may be determined empirically or in accord with known conservative substitutions.

TABLE 2
Amino Acid Abbreviations

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE 3
Amino Acid Substitution or Similarity Matrix
Adapted from the GCG Software 9.0 BLOSUM62 amino acid
substitution matrix (block substitution matrix). The higher the
value, the more likely a substitution is found in related, natural proteins.

| A | C | D | E | F | G | H | I | K | L | M | N | P | Q | R | S | T | V | W | Y | . |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | 3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

The term “homology” or “homologous” is intended to mean a sequence similarity between two polynucleotides or between two polypeptides. Similarity can be determined by comparing a position in each sequence, which can be aligned for purposes of comparison. If a given position of two polypeptide sequences is not identical, the similarity or conservativeness of that position can be determined by assessing the similarity of the amino acid of the position, for example, according to Table 3. A degree of similarity between sequences is a function of the number of matching or homologous positions shared by the sequences. The alignment of two sequences to determine their percent sequence similarity can be done using software programs known in the art, such as, for example, those described in Ausubel et al., Current Protocols in Molecular Biology, John Wiley and Sons, Baltimore, MD (1999). Preferably, default parameters are used for the alignment, examples of which are set forth below. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

The term “homologs” of to a given amino acid sequence or a nucleic acid sequence is intended to indicate that the corresponding sequences of the “homologs” having substantial identity or homology to the given amino acid sequence or nucleic acid sequence.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the)(BLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of)(BLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, restrictocin, phenomycin, enomycin, curcin, crotin, calicheamicin, Sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Antibodies may also be conjugated to an anti-cancer pro-drug activating enzyme capable of converting the pro-drug to its active form.

The term “effective amount” or “therapeutically effective amount” as used herein refers to the amount of binding molecule (e.g., an antibody) or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

The term “cancer” or “cancer cell” is used herein to denote a tissue or cell found in a neoplasm which possesses characteristics which differentiate it from normal tissue or tissue cells. Among such characteristics include but are not limited to: degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, leukemia, lymphoma, polyp, and cirrus, transformation, neoplasm, and the like.

As used herein, a “locally advanced” cancer refers to a cancer that has spread from where it started to nearby tissue or lymph nodes.

As used herein, a “metastatic” cancer refers to a cancer that has spread from where it started to different part of the body.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

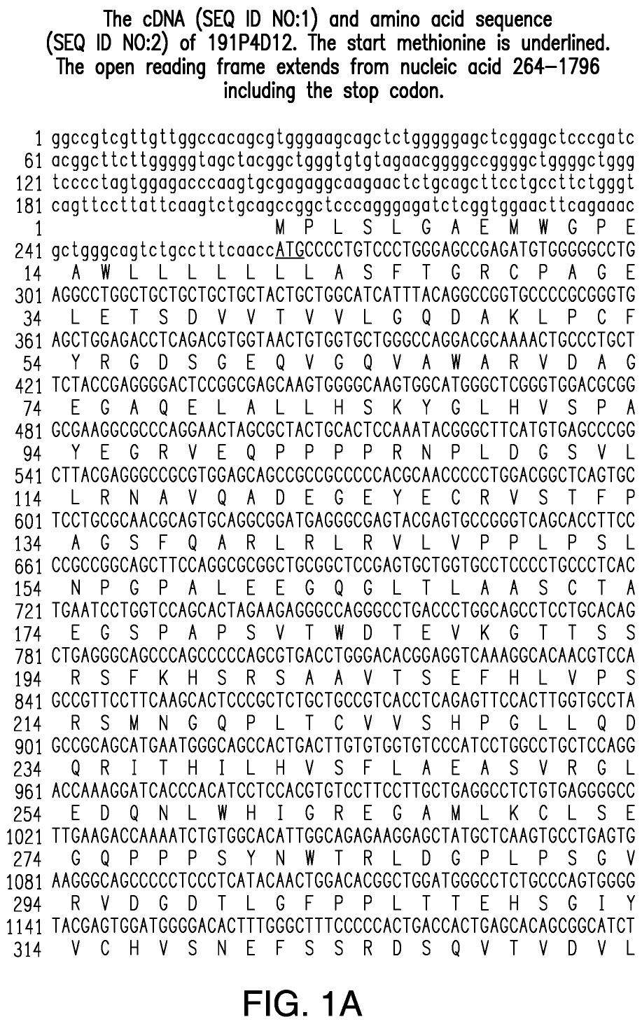

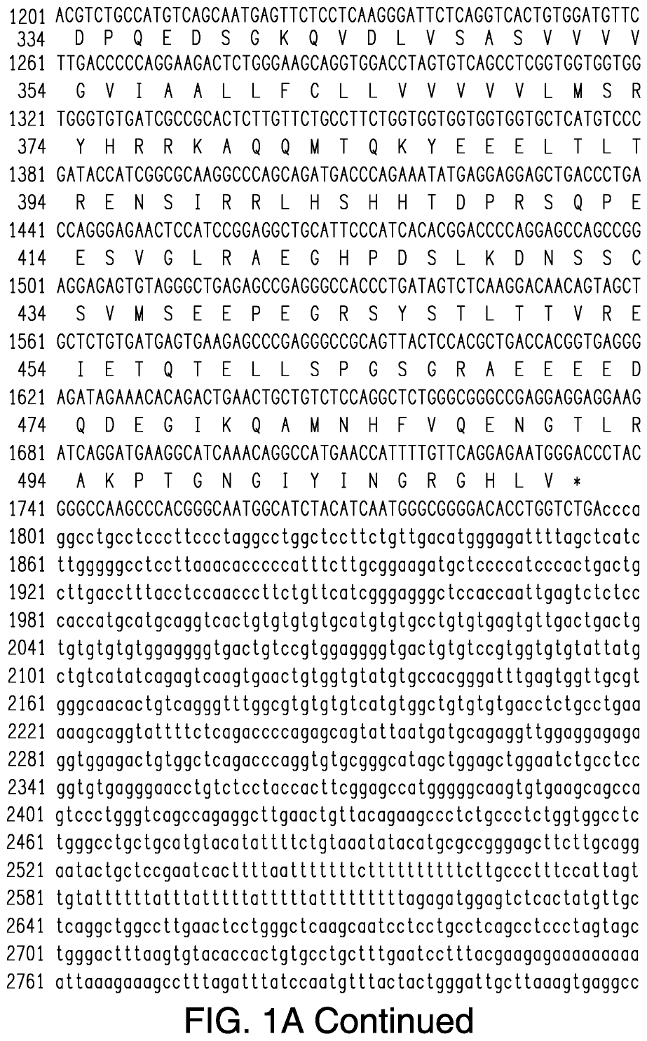

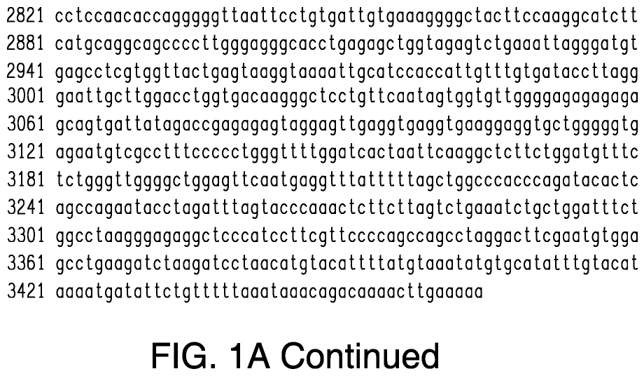

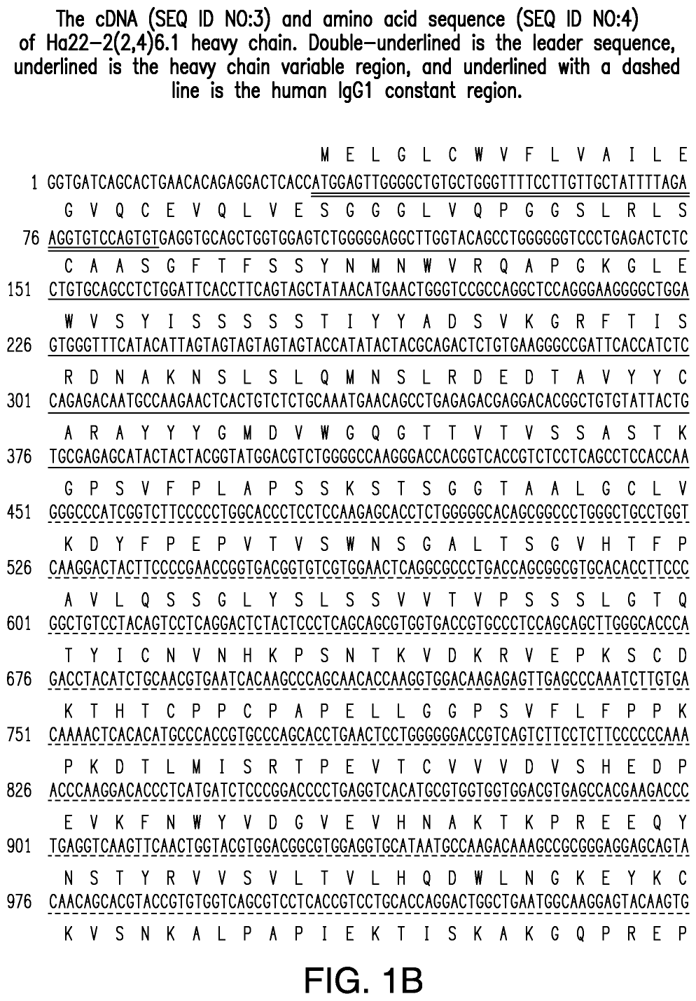

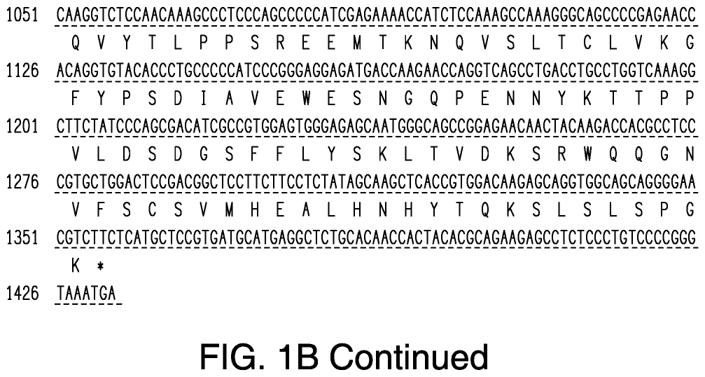

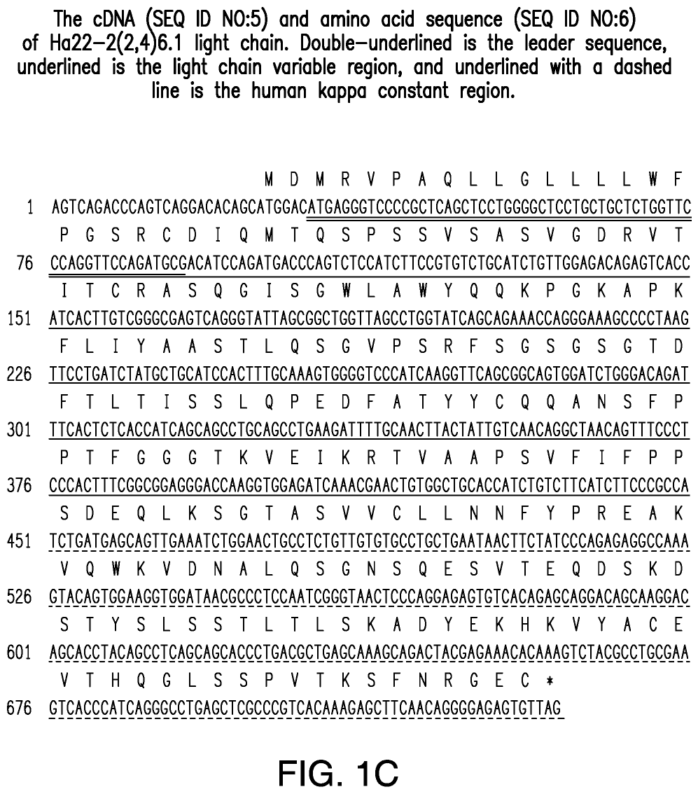

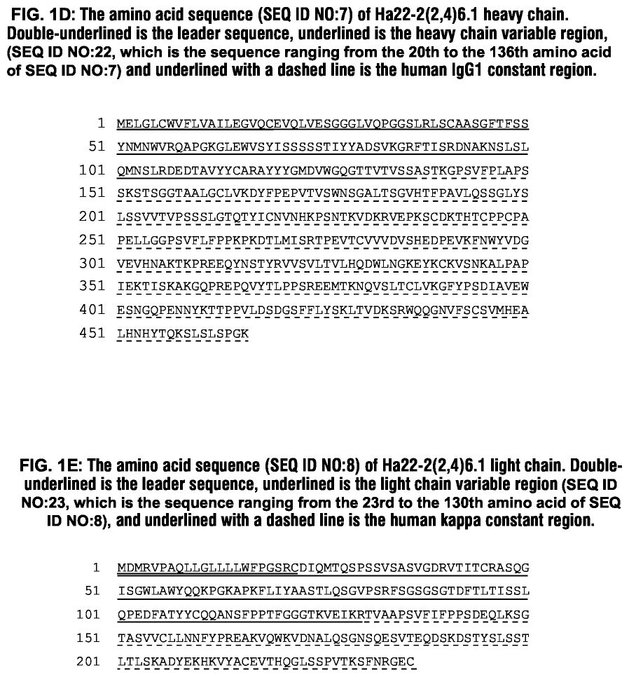

The term “variant” refers to a molecule that exhibits a variation from a described type or norm, such as a protein that has one or more different amino acid residues in the corresponding position(s) of a specifically described protein (e.g. the 191P4D12 protein shown in FIG. 1A.) An analog is an example of a variant protein. Splice isoforms and single nucleotides polymorphisms (SNPs) are further examples of variants.

The “191P4D12 proteins” and/or “191P4D12 related proteins” of the disclosure include those specifically identified herein (see, FIG. 1A), as well as allelic variants, conservative substitution variants, analogs and homologs that can be isolated/generated and characterized without undue experimentation following the methods outlined herein or readily available in the art. Fusion proteins that combine parts of different 191P4D12 proteins or fragments thereof, as well as fusion proteins of a 191P4D12 protein and a heterologous polypeptide are also included. Such 191P4D12 proteins are collectively referred to as the 191P4D12-related proteins, the proteins of the disclosure, or 191P4D12. The term “191P4D12-related protein” refers to a polypeptide fragment or a 191P4D12 protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 330, 335, 339 or more amino acids. The term “191P4D12” is used interchangeably with nectin-4.

### 5.2 Methods of Treating Cancer

### 5.2.1 Methods of Treating Cancer in General and for Patients Who have Received Prior Cancer Treatments

Provided herein are methods for the treatment of various cancers in subjects, including subjects with previously treated locally advanced or metastatic urothelial cancer, using an antibody drug conjugate (ADC) that binds 191P4D12.

In one aspect, provided herein are methods for the treatment of urothelial cancer in a subject using an antibody drug conjugate (ADC) that binds 191P4D12. In certain embodiments, the urothelial cancer has been previously treated with platinum-based chemotherapy and a CPI. In some embodiments, the urothelial cancer has been previously treated with platinum-based chemotherapy. In other embodiments, the urothelial cancer has been previously treated with a CPI. In one embodiment, the subject has been previously treated with platinum-based chemotherapy and a CPI. In another embodiment, the subject has been previously treated with platinum-based chemotherapy. In a further embodiment, the subject has been previously treated with a CPI.

In another aspect, provided herein are methods for the treatment of locally advanced urothelial cancer in a subject using an antibody drug conjugate (ADC) that binds 191P4D12. In certain embodiments, the locally advanced urothelial cancer has been previously treated with platinum-based chemotherapy and a CPI. In some embodiments, the locally advanced urothelial cancer has been previously treated with platinum-based chemotherapy. In other embodiments, the locally advanced urothelial cancer has been previously treated with a CPI. In one embodiment, the subject has been previously treated with platinum-based chemotherapy and a CPI. In another embodiment, the subject has been previously treated with platinum-based chemotherapy. In a further embodiment, the subject has been previously treated with a CPI.

In a further aspect, provided herein are methods for the treatment of metastatic urothelial cancer in a subject using an antibody drug conjugate (ADC) that binds 191P4D12. In certain embodiments, the metastatic urothelial cancer has been previously treated with platinum-based chemotherapy and a CPI. In some embodiments, the metastatic urothelial cancer has been previously treated with platinum-based chemotherapy. In other embodiments, the metastatic urothelial cancer has been previously treated with a CPI. In one embodiment, the subject has been previously treated with platinum-based chemotherapy and a CPI. In another embodiment, the subject has been previously treated with platinum-based chemotherapy. In a further embodiment, the subject has been previously treated with a CPI.

In one aspect, provided herein are methods for the treatment of urothelial cancer in a subject using an antibody drug conjugate (ADC) that binds 191P4D12. In certain embodiments, the urothelial cancer has been previously treated with platinum-based chemotherapy and a PD-1 inhibitor. In some embodiments, the urothelial cancer has been previously treated with platinum-based chemotherapy. In other embodiments, the urothelial cancer has been previously treated with a PD-1 inhibitor. In one embodiment, the subject has been previously treated with platinum-based chemotherapy and a PD-1 inhibitor. In another embodiment, the subject has been previously treated with platinum-based chemotherapy. In a further embodiment, the subject has been previously treated with a PD-1 inhibitor.

In another aspect, provided herein are methods for the treatment of locally advanced urothelial cancer in a subject using an antibody drug conjugate (ADC) that binds 191P4D12. In certain embodiments, the locally advanced urothelial cancer has been previously treated with platinum-based chemotherapy and a PD-1 inhibitor. In some embodiments, the locally advanced urothelial cancer has been previously treated with platinum-based chemotherapy. In other embodiments, the locally advanced urothelial cancer has been previously treated with a PD-1 inhibitor. In one embodiment, the subject has been previously treated with platinum-based chemotherapy and a PD-1 inhibitor. In another embodiment, the subject has been previously treated with platinum-based chemotherapy. In a further embodiment, the subject has been previously treated with a PD-1 inhibitor.

In a further aspect, provided herein are methods for the treatment of metastatic urothelial cancer in a subject using an antibody drug conjugate (ADC) that binds 191P4D12. In certain embodiments, the metastatic urothelial cancer has been previously treated with platinum-based chemotherapy and a PD-1 inhibitor. In some embodiments, the metastatic urothelial cancer has been previously treated with platinum-based chemotherapy. In other embodiments, the metastatic urothelial cancer has been previously treated with a PD-1 inhibitor. In one embodiment, the subject has been previously treated with platinum-based chemotherapy and a PD-1 inhibitor. In another embodiment, the subject has been previously treated with platinum-based chemotherapy. In a further embodiment, the subject has been previously treated with a PD-1 inhibitor.

In one aspect, provided herein are methods for the treatment of urothelial cancer in a subject using an antibody drug conjugate (ADC) that binds 191P4D12. In certain embodiments, the urothelial cancer has been previously treated with platinum-based chemotherapy and a PD-L1 inhibitor. In some embodiments, the urothelial cancer has been previously treated with platinum-based chemotherapy. In other embodiments, the urothelial cancer has been previously treated with a PD-L1 inhibitor. In one embodiment, the subject has been previously treated with platinum-based chemotherapy and a PD-L1 inhibitor. In another embodiment, the subject has been previously treated with platinum-based chemotherapy. In a further embodiment, the subject has been previously treated with a PD-L1 inhibitor.

In another aspect, provided herein are methods for the treatment of locally advanced urothelial cancer in a subject using an antibody drug conjugate (ADC) that binds 191P4D12. In certain embodiments, the locally advanced urothelial cancer has been previously treated with platinum-based chemotherapy and a PD-L1 inhibitor. In some embodiments, the locally advanced urothelial cancer has been previously treated with platinum-based chemotherapy. In other embodiments, the locally advanced urothelial cancer has been previously treated with a PD-L1 inhibitor. In one embodiment, the subject has been previously treated with platinum-based chemotherapy and a PD-L1 inhibitor. In another embodiment, the subject has been previously treated with platinum-based chemotherapy. In a further embodiment, the subject has been previously treated with a PD-L1 inhibitor.

In a further aspect, provided herein are methods for the treatment of metastatic urothelial cancer in a subject using an antibody drug conjugate (ADC) that binds 191P4D12. In certain embodiments, the metastatic urothelial cancer has been previously treated with platinum-based chemotherapy and a PD-L1 inhibitor. In some embodiments, the metastatic urothelial cancer has been previously treated with platinum-based chemotherapy. In other embodiments, the metastatic urothelial cancer has been previously treated with a PD-L1 inhibitor. In one embodiment, the subject has been previously treated with platinum-based chemotherapy and a PD-L1 inhibitor. In another embodiment, the subject has been previously treated with platinum-based chemotherapy. In a further embodiment, the subject has been previously treated with a PD-L1 inhibitor.

In some embodiments of the methods provided herein, the subject has been treated with one or more other cancer treatments. In certain embodiments of the methods provided herein, the urothelial cancer, including locally advanced or metastatic urothelial cancer, has been treated with one or more other cancer treatments.

In some embodiments, the CPI provided for the methods can comprise or consist of any CPI as described in this Section (Section 5.2.1).

In all the methods provided herein and specifically those described in the previous six paragraphs: the ADCs that can be used are described in Sections 3, 5.2, 5.3, 5.4, 5.5, and 6, selection of patients for treatment is described herein and exemplified in this Section (Section 5.2) and Sections 3 and 6, dosing regimens and pharmaceutical composition for administering the therapeutic agent are described in this Section (Section 5.2), Sections 5.6, 5.7 and 6 below, the biomarkers that can be used for identifying the therapeutic agents, selecting the patients, determining the outcome of these methods, and/or serving as criteria in any way for these methods are described herein and exemplified in this Section (Section 5.2, including 5.2.1 and 5.2.2) and Section 6, the biomarkers can be determined as described in Section 5.8 or as known in the art, therapeutic outcomes for the methods provided herein can be improvement of the biomarkers described herein, for example, those described and exemplified in this Section (Section 5.2 including 5.2.2) and Section 6. Therefore, a person skilled in the art would understand that the methods provided herein include all permutations and combinations of the patients, therapeutic agents, dosing regiments, biomarkers, and therapeutic outcomes as described above and below.

In certain embodiments, the methods provided herein are used for treating subjects having urothelial cancers that express 191P4D12 RNA, express 191P4D12 protein, or express both 191P4D12 RNA and 191P4D12 protein. In some embodiments, the methods provided herein are used for treating subjects who have urothelial cancers that express 191P4D12 RNA, express 191P4D12 protein, or express both 191P4D12 RNA and 191P4D12 protein and who have been previously treated with platinum-based chemotherapy and a CPI. In one embodiment, the methods provided herein are used for treating subjects who have urothelial cancers that express 191P4D12 RNA, express 191P4D12 protein, or express both 191P4D12 RNA and 191P4D12 protein and who have been previously treated with platinum-based chemotherapy. In another embodiment, the methods provided herein are used for treating subjects who have urothelial cancers that express 191P4D12 RNA, express 191P4D12 protein, or express both 191P4D12 RNA and 191P4D12 protein and who have been previously treated with a CPI.

In certain embodiments, the methods provided herein are used for treating subjects having locally advanced urothelial cancers that express 191P4D12 RNA, express 191P4D12 protein, or express both 191P4D12 RNA and 191P4D12 protein. In some embodiments, the methods provided herein are used for treating subjects who have locally advanced urothelial cancers that express 191P4D12 RNA, express 191P4D12 protein, or express both 191P4D12 RNA and 191P4D12 protein and who have been previously treated with platinum-based chemotherapy and a CPI. In one embodiment, the methods provided herein are used for treating subjects who have locally advanced urothelial cancers that express 191P4D12 RNA, express 191P4D12 protein, or express both 191P4D12 RNA and 191P4D12 protein and who have been previously treated with platinum-based chemotherapy. In another embodiment, the methods provided herein are used for treating subjects who have locally advanced urothelial cancers that express 191P4D12 RNA, express 191P4D12 protein, or express both 191P4D12 RNA and 191P4D12 protein and who have been previously treated with a CPI.

In certain embodiments, the methods provided herein are used for treating subjects having metastic cancers that express 191P4D12 RNA, express 191P4D12 protein, or express both 191P4D12 RNA and 191P4D12 protein. In some embodiments, the methods provided herein are used for treating subjects who have metastic urothelial cancers that express 191P4D12 RNA, express 191P4D12 protein, or express both 191P4D12 RNA and 191P4D12 protein and who have been previously treated with platinum-based chemotherapy and a CPI. In one embodiment, the methods provided herein are used for treating subjects who have metastic urothelial cancers that express 191P4D12 RNA, express 191P4D12 protein, or express both 191P4D12 RNA and 191P4D12 protein and who have been previously treated with platinum-based chemotherapy. In another embodiment, the methods provided herein are used for treating subjects who have metastic urothelial cancers that express 191P4D12 RNA, express 191P4D12 protein, or express both 191P4D12 RNA and 191P4D12 protein and who have been previously treated with a CPI.

In some embodiments, the 191P4D12 RNA expression in the cancers is determined by polynucleotide hybridization, sequencing (assessing the relative abundance of the sequences), and/or PCR (including RT-PCR). In some embodiments, the 191P4D12 protein expression in the cancers is determined by IHC, analysis in fluorescence-activated cell sorting (FACS), and/or western blotting. In some embodiments, the 191P4D12 protein expression in the cancers is determined by more than one method. In some embodiments, the 191P4D12 protein expression in the cancers is determined by two methods of IHC.

In some embodiments, the locally advanced or metastatic urothelial cancers are confirmed histologically, cytologically, or both histologically and cytologically. In some embodiments, the locally advanced or metastatic bladder cancers are confirmed histologically, cytologically, or both histologically and cytologically

In some embodiments, the subjects that can be treated in the methods provided herein include subjects who received one or more other treatments for cancer. In some embodiments, the subjects that can be treated in the methods provided herein include subjects who received one or more other treatments for cancer and whose cancer progressed or relapsed following the one or more treatments. Such one or more treatments include, for example, one or more lines of immune checkpoint inhibitor therapies, chemotherapies, and both immune checkpoint inhibitor therapies and chemotherapies. In some embodiments, the subjects that can be treated in the methods provided herein include subjects whose cancers progressed or relapsed following a therapy with a CPI. In some embodiments, the subjects that can be treated in the methods provided herein include subjects whose cancers progressed or relapsed following a platinum-containing chemotherapy. In some embodiments, the subjects that can be treated in the methods provided herein include subjects whose cancers progressed or relapsed following a platinum-containing chemotherapy in the neoadjuvant setting. In some embodiments, the subjects that can be treated in the methods provided herein include subjects whose cancers progressed or relapsed following a platinum-containing chemotherapy in the adjuvant setting. In some embodiments, the subjects that can be treated in the methods provided herein include subjects whose cancers progressed or relapsed following a platinum-containing chemotherapy in the neoadjuvant, locally advanced setting. In some embodiments, the subjects that can be treated in the methods provided herein include subjects whose cancers progressed or relapsed following a platinum-containing chemotherapy in the neoadjuvant, metastatic setting. In some embodiments, the subjects that can be treated in the methods provided herein include subjects whose cancers progressed or relapsed following a platinum-containing chemotherapy in the adjuvant, locally advanced setting. In some embodiments, the subjects that can be treated in the methods provided herein include subjects whose cancers progressed or relapsed following a platinum-containing chemotherapy in the adjuvant, metastatic setting. In some embodiments, the subjects that can be treated in the methods provided herein include subjects whose cancers progressed or relapsed following a platinum-containing chemotherapy in metastatic setting. In some embodiments, the subjects that can be treated in the methods provided herein include subjects whose cancers progressed or relapsed following a platinum-containing chemotherapy in the locally advanced setting.

In some specific embodiments, the subjects that can be treated in the methods provided herein include subjects whose cancers progressed or relapsed following a therapy with a CPI and a platinum-containing chemotherapy. In some specific embodiments, the subjects that can be treated in the methods provided herein include subjects whose cancer progressed or relapsed following a therapy with a CPI and a platinum-containing chemotherapy in the neoadjuvant setting. In some specific embodiments, the subjects that can be treated in the methods provided herein include subjects whose cancer progressed or relapsed following a therapy with a CPI and a platinum-containing chemotherapy in the adjuvant setting. In some specific embodiments, the subjects that can be treated in the methods provided herein include subjects whose cancer progressed or relapsed following a therapy with a CPI and a platinum-containing chemotherapy in the locally advanced setting. In some specific embodiments, the subjects that can be treated in the methods provided herein include subjects whose cancer progressed or relapsed following a therapy with a CPI and a platinum-containing chemotherapy in the metastatic setting. In some specific embodiments, the subjects that can be treated in the methods provided herein include subjects whose cancer progressed or relapsed following a therapy with a CPI and a platinum-containing chemotherapy in the neoadjuvant, locally advanced setting. In some specific embodiments, the subjects that can be treated in the methods provided herein include subjects whose cancer progressed or relapsed following a therapy with a CPI and a platinum-containing chemotherapy in the neoadjuvant, metastatic setting. In some specific embodiments, the subjects that can be treated in the methods provided herein include subjects whose cancer progressed or relapsed following a therapy with a CPI and a platinum-containing chemotherapy in the adjuvant, locally advanced setting. In some specific embodiments, the subjects that can be treated in the methods provided herein include subjects whose cancer progressed or relapsed following a therapy with a CPI and a platinum-containing chemotherapy in the adjuvant, metastatic setting.

In some specific embodiments, the subjects that can be treated in the methods provided herein include subjects whose cancers progressed or relapsed following a therapy with a CPI. In some specific embodiments, the subjects that can be treated in the methods provided herein include subjects whose cancer progressed or relapsed following a therapy with a CPI in the neoadjuvant setting. In some specific embodiments, the subjects that can be treated in the methods provided herein include subjects whose cancer progressed or relapsed following a therapy with a CPI in the adjuvant setting. In some specific embodiments, the subjects that can be treated in the methods provided herein include subjects whose cancer progressed or relapsed following a therapy with a CPI in the locally advanced setting. In some specific embodiments, the subjects that can be treated in the methods provided herein include subjects whose cancer progressed or relapsed following a therapy with a CPI in the metastatic setting. In some specific embodiments, the subjects that can be treated in the methods provided herein include subjects whose cancer progressed or relapsed following a therapy with a CPI in the neoadjuvant, locally advanced setting. In some specific embodiments, the subjects that can be treated in the methods provided herein include subjects whose cancer progressed or relapsed following a therapy with a CPI in the neoadjuvant, metastatic setting. In some specific embodiments, the subjects that can be treated in the methods provided herein include subjects whose cancer progressed or relapsed following a therapy with a CPI in the adjuvant, locally advanced setting. In some specific embodiments, the subjects that can be treated in the methods provided herein include subjects whose cancer progressed or relapsed following a therapy with a CPI in the adjuvant, metastatic setting.

In some specific embodiments, the subjects that can be treated in the methods provided herein include subjects whose cancers progressed or relapsed following a therapy with a PD-1 inhibitor and a platinum-containing chemotherapy. In some specific embodiments, the subjects that can be treated in the methods provided herein include subjects whose cancer progressed or relapsed following a therapy with a PD-1 inhibitor and a platinum-containing chemotherapy in the neoadjuvant setting. In some specific embodiments, the subjects that can be treated in the methods provided herein include subjects whose cancer progressed or relapsed following a therapy with a PD-1 inhibitor and a platinum-containing chemotherapy in the adjuvant setting. In some specific embodiments, the subjects that can be treated in the methods provided herein include subjects whose cancer progressed or relapsed following a therapy with a PD-1 inhibitor and a platinum-containing chemotherapy in the locally advanced setting. In some specific embodiments, the subjects that can be treated in the methods provided herein include subjects whose cancer progressed or relapsed following a therapy with a PD-1 inhibitor and a platinum-containing chemotherapy in the metastatic setting. In some specific embodiments, the subjects that can be treated in the methods provided herein include subjects whose cancer progressed or relapsed following a therapy with a PD-1 inhibitor and a platinum-containing chemotherapy in the neoadjuvant, locally advanced setting. In some specific embodiments, the subjects that can be treated in the methods provided herein include subjects whose cancer progressed or relapsed following a therapy with a PD-1 inhibitor and a platinum-containing chemotherapy in the neoadjuvant, metastatic setting. In some specific embodiments, the subjects that can be treated in the methods provided herein include subjects whose cancer progressed or relapsed following a therapy with a PD-1 inhibitor and a platinum-containing chemotherapy in the adjuvant, locally advanced setting. In some specific embodiments, the subjects that can be treated in the methods provided herein include subjects whose cancer progressed or relapsed following a therapy with a PD-1 inhibitor and a platinum-containing chemotherapy in the adjuvant, metastatic setting.

In some specific embodiments, the subjects that can be treated in the methods provided herein include subjects whose cancers progressed or relapsed following a therapy with a PD-1 inhibitor. In some specific embodiments, the subjects that can be treated in the methods provided herein include subjects whose cancer progressed or relapsed following a therapy with a PD-1 inhibitor in the neoadjuvant setting. In some specific embodiments, the subjects that can be treated in the methods provided herein include subjects whose cancer progressed or relapsed following a therapy with a PD-1 inhibitor in the adjuvant setting. In some specific embodiments, the subjects that can be treated in the methods provided herein include subjects whose cancer progressed or relapsed following a therapy with a PD-1 inhibitor in the locally advanced setting. In some specific embodiments, the subjects that can be treated in the methods provided herein include subjects whose cancer progressed or relapsed following a therapy with a PD-1 inhibitor in the metastatic setting. In some specific embodiments, the subjects that can be treated in the methods provided herein include subjects whose cancer progressed or relapsed following a therapy with a PD-1 inhibitor in the neoadjuvant, locally advanced setting. In some specific embodiments, the subjects that can be treated in the methods provided herein include subjects whose cancer progressed or relapsed following a therapy with a PD-1 inhibitor in the neoadjuvant, metastatic setting. In some specific embodiments, the subjects that can be treated in the methods provided herein include subjects whose cancer progressed or relapsed following a therapy with a PD-1 inhibitor in the adjuvant, locally advanced setting. In some specific embodiments, the subjects that can be treated in the methods provided herein include subjects whose cancer progressed or relapsed following a therapy with a PD-1 inhibitor in the adjuvant, metastatic setting.

In some specific embodiments, the subjects that can be treated in the methods provided herein include subjects whose cancers progressed or relapsed following a therapy with a PD-L1 inhibitor and a platinum-containing chemotherapy. In some specific embodiments, the subjects that can be treated in the methods provided herein include subjects whose cancer progressed or relapsed following a therapy with a PD-L1 inhibitor and a platinum-containing chemotherapy in the neoadjuvant setting. In some specific embodiments, the subjects that can be treated in the methods provided herein include subjects whose cancer progressed or relapsed following a therapy with a PD-L1 inhibitor and a platinum-containing chemotherapy in the adjuvant setting. In some specific embodiments, the subjects that can be treated in the methods provided herein include subjects whose cancer progressed or relapsed following a therapy with a PD-L1 inhibitor and a platinum-containing chemotherapy in the locally advanced setting. In some specific embodiments, the subjects that can be treated in the methods provided herein include subjects whose cancer progressed or relapsed following a therapy with a PD-L1 inhibitor and a platinum-containing chemotherapy in the metastatic setting. In some specific embodiments, the subjects that can be treated in the methods provided herein include subjects whose cancer progressed or relapsed following a therapy with a PD-L1 inhibitor and a platinum-containing chemotherapy in the neoadjuvant, locally advanced setting. In some specific embodiments, the subjects that can be treated in the methods provided herein include subjects whose cancer progressed or relapsed following a therapy with a PD-L1 inhibitor and a platinum-containing chemotherapy in the neoadjuvant, metastatic setting. In some specific embodiments, the subjects that can be treated in the methods provided herein include subjects whose cancer progressed or relapsed following a therapy with a PD-L1 inhibitor and a platinum-containing chemotherapy in the adjuvant, locally advanced setting. In some specific embodiments, the subjects that can be treated in the methods provided herein include subjects whose cancer progressed or relapsed following a therapy with a PD-L1 inhibitor and a platinum-containing chemotherapy in the adjuvant, metastatic setting.

In some specific embodiments, the subjects that can be treated in the methods provided herein include subjects whose cancers progressed or relapsed following a therapy with a PD-L1 inhibitor. In some specific embodiments, the subjects that can be treated in the methods provided herein include subjects whose cancer progressed or relapsed following a therapy with a PD-L1 inhibitor in the neoadjuvant setting. In some specific embodiments, the subjects that can be treated in the methods provided herein include subjects whose cancer progressed or relapsed following a therapy with a PD-L1 inhibitor in the adjuvant setting. In some specific embodiments, the subjects that can be treated in the methods provided herein include subjects whose cancer progressed or relapsed following a therapy with a PD-L1 inhibitor in the locally advanced setting. In some specific embodiments, the subjects that can be treated in the methods provided herein include subjects whose cancer progressed or relapsed following a therapy with a PD-L1 inhibitor in the metastatic setting. In some specific embodiments, the subjects that can be treated in the methods provided herein include subjects whose cancer progressed or relapsed following a therapy with a PD-L1 inhibitor in the neoadjuvant, locally advanced setting. In some specific embodiments, the subjects that can be treated in the methods provided herein include subjects whose cancer progressed or relapsed following a therapy with a PD-L1 inhibitor in the neoadjuvant, metastatic setting. In some specific embodiments, the subjects that can be treated in the methods provided herein include subjects whose cancer progressed or relapsed following a therapy with a PD-L1 inhibitor in the adjuvant, locally advanced setting. In some specific embodiments, the subjects that can be treated in the methods provided herein include subjects whose cancer progressed or relapsed following a therapy with a PD-L1 inhibitor in the adjuvant, metastatic setting.

In certain embodiments, the subjects that can be treated in the methods provided herein include those whose cancers have progressed or relapsed other treatments for cancers within 1, 2, 3, 4, 5, 6, 7, 8, 9, 10, 11, 12, 13, 14, 15, 16, 17, 18, 19, 20, 21, 22, 23, or 24 months after the other treatments, including for example and not by way of limitation, any or any combination of the treatments described in the preceding paragraphs. In some particular embodiment, the cancers in the subjects have progressed or relapsed within 6 months after the platinum-based therapy. In further embodiments, the cancers in the subjects have progressed or relapsed within 12 months after a platinum-based therapy.

In some embodiments, the subjects that can be treated in the methods provided herein have certain phenotypic or genotypic characteristics. In some embodiments, the subjects have any permutation and combination of the phenotypic or genotypic characteristics described herein.

In some embodiments, the phenotypic or genotypic characteristics are determined histologically, cytologically, or both histologically and cytologically. In some embodiments of methods provided herein, the histological and/or the cytological determination of the phenotypic and/or genotypic characteristics are performed as described in American Society of Clinical Oncology/College of American Pathologists (ASCO/CAP) guidelines based on the most recently analyzed tissue, which is incorporated herein in their entirety by reference. In some embodiments, the phenotypic or genotypic characteristics are determined by sequencing including the next generation sequencing (e.g. NGS from Illumina, Inc), DNA hybridization, and/or RNA hybridization.

In various aspects or embodiments of the methods provided herein, including the methods provided in this Section (Section 5.2) such as the methods provided in this and the preceding paragraphs, the methods involve a prior treatment with an immune checkpoint inhibitor as provided in the method. As used herein, the term “immune checkpoint inhibitor” or “checkpoint inhibitor” refers to molecules that totally or partially reduce, inhibit, interfere with or modulate one or more checkpoint proteins. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). Other exemplary checkpoint proteins include LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In certain embodiments, the checkpoint inhibitor for the methods provided herein can be an inhibitors or activators against a checkpoint protein that upregulated in cancer. In some specific embodiments, the checkpoint inhibitor for the methods provided herein can be an inhibitors or activators against a checkpoint protein including LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. In some embodiments, the checkpoint inhibitor for the methods provided herein can be an inhibitors or activators selected from the group consisting of a PD-1 inhibitor, a PD-L1 inhibitor, a PD-L2 inhibitor, a CTLA-4 inhibitor, a LAG-3 inhibitor, a B7 inhibitor, a TIM3 (HAVCR2) inhibitor, an OX40 (CD134) inhibitor, a GITR agonist, a CD137 agonist, or a CD40 agonist, a VTCN1 inhibitor, an IDO1 inhibitor, a CD276 inhibitor, a PVRIG inhibitor, a TIGIT inhibitor, a CD25 (IL2RA) inhibitor, an IFNAR2 inhibitor, an IFNAR1 inhibitor, a CSF1R inhibitor, a VSIR (VISTA) inhibitor, or a therapeutic agent targeting HLA. Such inhibitors, activators, or therapeutic agents are further provided below.

In some embodiments, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In certain embodiments, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In some embodiments, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity. In one embodiment, the PD-1 antibody is cemiplimab. In another embodiment, the PD-1 antibody is camrelizumab. In a further embodiment, the PD-1 antibody is sintilimab. In some embodiments, the PD-1 antibody is tislelizumab. In certain embodiments, the PD-1 antibody is TSR-042. In yet another embodiment, the PD-1 antibody is PDR001. In yet another embodiment, the PD-1 antibody is toripalimab.

In certain embodiments, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®). In a further embodiment, the PD-L1 inhibitor is avelumab.

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is a VTCN inhibitor. In one embodiment, the VTCN inhibitor is FPA150.

In one embodiment, the checkpoint inhibitor is an IDO inhibitor. In one embodiment, the IDO inhibitor is INCB024360. In another embodiment, the IDO inhibitor is indoximod. In one embodiment, the IDO inhibitor is epacadostat. In another embodiment, the IDO inhibitor is BMS986205. In yet another embodiment, the IDO inhibitor is Navoximod. In one embodiment, the IDO inhibitor is PF-06840003. In another embodiment, the IDO inhibitor is KHK2455. In yet another embodiment, the IDO inhibitor is RG70099. In one embodiment, the IDO inhibitor is IOM-E. In another embodiment, the IDO inhibitor is or IOM-D.

In some embodiments, the checkpoint inhibitor is a TIGIT inhibitor. In certain embodiments, the TIGIT inhibitor is an anti-TIGIT antibody. In one embodiment, the TIGIT inhibitor is MTIG7192A. In another embodiment, the TIGIT inhibitor is BMS-986207. In yet another embodiment, the TIGIT inhibitor is OMP-313M32. In one embodiment, the TIGIT inhibitor is MK-7684. In another embodiment, the TIGIT inhibitor is AB154. In yet another embodiment, the TIGIT inhibitor is CGEN-15137. In one embodiment, the TIGIT inhibitor is SEA-TIGIT. In another embodiment, the TIGIT inhibitor is ASP8374. In yet another embodiment, the TIGIT inhibitor is AJUD008.

In some embodiments, the checkpoint inhibitor is a VSIR inhibitor. In certain embodiments, the VSIR inhibitor is an anti-VSIR antibody. In one embodiment, the VSIR inhibitor is MTIG7192A. In another embodiment, the VSIR inhibitor is CA-170. In yet another embodiment, the VSIR inhibitor is JNJ 61610588. In one embodiment, the VSIR inhibitor is HMBD-002.

In some embodiments, the checkpoint inhibitor is a TIM3 inhibitor. In certain embodiments, the TIM3 inhibitor is an anti-TIM3 antibody. In one embodiment, the TIM3 inhibitor is AJUD009.

In some embodiments, the checkpoint inhibitor is a CD25 (IL2RA) inhibitor. In certain embodiments, the CD25 (IL2RA) inhibitor is an anti-CD25 (IL2RA) antibody. In one embodiment, the CD25 (IL2RA) inhibitor is daclizumab. In another embodiment, the CD25 (IL2RA) inhibitor is basiliximab.

In some embodiments, the checkpoint inhibitor is an IFNAR1 inhibitor. In certain embodiments, the IFNAR1 inhibitor is an anti-IFNAR1 antibody. In one embodiment, the IFNAR1 inhibitor is anifrolumab. In another embodiment, the IFNAR1 inhibitor is sifalimumab.

In some embodiments, the checkpoint inhibitor is a CSF1R inhibitor. In certain embodiments, the CSF1R inhibitor is an anti-CSF1R antibody. In one embodiment, the CSF1R inhibitor is pexidartinib. In another embodiment, the CSF1R inhibitor is emactuzumab. In yet another embodiment, the CSF1R inhibitor is cabiralizumab. In one embodiment, the CSF1R inhibitor is ARRY-382. In another embodiment, the CSF1R inhibitor is BLZ945. In yet another embodiment, the CSF1R inhibitor is AJUD010. In one embodiment, the CSF1R inhibitor is AMG820. In another embodiment, the CSF1R inhibitor is IMC-CS4. In yet another embodiment, the CSF1R inhibitor is JNJ-40346527. In one embodiment, the CSF1R inhibitor is PLX5622. In another embodiment, the CSF1R inhibitor is FPA008.

In some embodiments, the checkpoint inhibitor is a therapeutic agent targeting HLA. In certain embodiments, the therapeutic agent targeting HLA is an anti-HLA antibody. In one embodiment, the therapeutic agent targeting HLA is GSK01. In another embodiment, the therapeutic agent targeting HLA is IMC-C103C. In yet another embodiment, the therapeutic agent targeting HLA is IMC-F106C. In one embodiment, the therapeutic agent targeting HLA is IMC-G107C. In another embodiment, the therapeutic agent targeting HLA is ABBV-184.

In certain embodiments, the immune checkpoint inhibitors provided herein include two or more of the checkpoint inhibitors described herein (including checkpoint inhibitors of the same or different class). Moreover, the methods described herein can be used in combination with one or more second active agents as described herein where appropriate for treating diseases described herein and understood in the art.

In some embodiments, the checkpoint inhibitor is administered after the administration of the ADCs provided herein. In other embodiments, the checkpoint inhibitor is administered simultaneously (e.g., in the same dosing period) with the ADCs provided herein. In yet other embodiments, the checkpoint inhibitor is administered after the administration of the ADCs provided herein.

In some embodiments, the amount of the checkpoint inhibitor for the various methods provided herein can be determined by standard clinical techniques. In certain embodiments, the amount of the checkpoint inhibitor for the various methods are provided in Section 5.6.

In some embodiments, the subjects that can be treated in the methods provided herein is a mammal. In some embodiments, the subjects that can be treated in the methods provided herein is a human.

### 5.2.1.1 Additional Patient Demographics

Additionally, the human subjects for whom the methods provided herein can be used are human subjects having various other conditions. In one embodiment, the human subjects for whom the methods provided herein can have a primary site of tumor in the lower urinary tract. In some embodiments, the human subjects for whom the methods provided herein can have visceral metastases. In certain embodiments, the human subjects for whom the methods provided herein can have liver metastases. In other embodiments, the human subjects for whom the methods provided herein can have at least 1 Bellmunt risk factor. In yet other embodiments, the human subjects for whom the methods provided herein can have ECOG performance status score of 0. In one embodiment, the human subjects for whom the methods provided herein can have a primary site of tumor in the lower urinary tract and visceral metastases. In some embodiments, the human subjects for whom the methods provided herein can have a primary site of tumor in the lower urinary tract and liver metastases. In certain embodiments, the human subjects for whom the methods provided herein can have a primary site of tumor in the lower urinary tract and at least 1 Bellmunt risk factor. In other embodiments, the human subjects for whom the methods provided herein can have a primary site of tumor in the lower urinary tract and ECOG performance status score of 0. In further embodiments, the human subjects for whom the methods provided herein can have visceral metastases and liver metastases. In one embodiment, the human subjects for whom the methods provided herein can have visceral metastases and at least 1 Bellmunt risk factor. In some embodiments, the human subjects for whom the methods provided herein can have visceral metastases and ECOG performance status score of 0. In other embodiments, the human subjects for whom the methods provided herein can have liver metastases and at least 1 Bellmunt risk factor. In yet other embodiments, the human subjects for whom the methods provided herein can have liver metastases and ECOG performance status score of 0. In one embodiment, the human subjects for whom the methods provided herein can have at least 1 Bellmunt risk factor and ECOG performance status score of 0. In other embodiments, the human subjects for whom the methods provided herein can have a primary site of tumor in the lower urinary tract, visceral metastases, and liver metastases. In yet other embodiments, the human subjects for whom the methods provided herein can have a primary site of tumor in the lower urinary tract, visceral metastases and at least 1 Bellmunt risk factor. In further embodiments, the human subjects for whom the methods provided herein can have a primary site of tumor in the lower urinary tract, visceral metastases, and ECOG performance status score of 0. In some embodiments, the human subjects for whom the methods provided herein can have a primary site of tumor in the lower urinary tract, liver metastases and at least 1 Bellmunt risk factor. In certain embodiments, the human subjects for whom the methods provided herein can have a primary site of tumor in the lower urinary tract, liver metastases, and ECOG performance status score of 0. In yet other embodiments, the human subjects for whom the methods provided herein can have a primary site of tumor in the lower urinary tract, at least 1 Bellmunt risk factor and ECOG performance status score of 0. In some embodiments, the human subjects for whom the methods provided herein can have visceral metastases, liver metastases and at least 1 Bellmunt risk factor. In certain embodiments, the human subjects for whom the methods provided herein can have visceral metastases, liver metastases, and ECOG performance status score of 0. In yet other embodiments, the human subjects for whom the methods provided herein can have visceral metastases, at least 1 Bellmunt risk factor and ECOG performance status score of 0. In yet other embodiments, the human subjects for whom the methods provided herein can have liver metastases, at least 1 Bellmunt risk factor and ECOG performance status score of 0. In other embodiments, the human subjects for whom the methods provided herein can have a primary site of tumor in the lower urinary tract, visceral metastases, liver metastases, and at least 1 Bellmunt risk factor. In further embodiments, the human subjects for whom the methods provided herein can have a primary site of tumor in the lower urinary tract, visceral metastases, liver metastases, and ECOG performance status score of 0. In some embodiments, the human subjects for whom the methods provided herein can have a primary site of tumor in the lower urinary tract, visceral metastases, at least 1 Bellmunt risk factor, and ECOG performance status score of 0. In certain embodiments, the human subjects for whom the methods provided herein can have a primary site of tumor in the lower urinary tract, liver metastases, at least 1 Bellmunt risk factor, and ECOG performance status score of 0. In further embodiments, the human subjects for whom the methods provided herein can have visceral metastases, liver metastases, at least 1 Bellmunt risk factor, and ECOG performance status score of 0. In certain embodiments, the human subjects for whom the methods provided herein can have a primary site of tumor in the lower urinary tract, visceral metastases, liver metastases, at least 1 Bellmunt risk factor, and ECOG performance status score of 0. In some embodiments, the human subjects for whom the methods provided herein can have any one of a primary site of tumor in the lower urinary tract, visceral metastases, liver metastases, at least 1 Bellmunt risk factor, and ECOG performance status score of 0. In some embodiments, the human subjects for whom the methods provided herein can have any two of a primary site of tumor in the lower urinary tract, visceral metastases, liver metastases, at least 1 Bellmunt risk factor, and ECOG performance status score of 0, in any combination or permutation. In some embodiments, the human subjects for whom the methods provided herein can have any three of a primary site of tumor in the lower urinary tract, visceral metastases, liver metastases, at least 1 Bellmunt risk factor, and ECOG performance status score of 0, in any combination or permutation. In some embodiments, the human subjects for whom the methods provided herein can have any four of a primary site of tumor in the lower urinary tract, visceral metastases, liver metastases, at least 1 Bellmunt risk factor, and ECOG performance status score of 0, in any combination or permutation. In some embodiments, the human subjects for whom the methods provided herein can have all five of a primary site of tumor in the lower urinary tract, visceral metastases, liver metastases, at least 1 Bellmunt risk factor, and ECOG performance status score of 0.

In further embodiments, the human subjects for whom the methods provided herein can have a primary site of tumor in the upper urinary tract. In one embodiment, the human subjects for whom the methods provided herein can have a primary site of tumor in the upper urinary tract and visceral metastases. In some embodiments, the human subjects for whom the methods provided herein can have a primary site of tumor in the upper urinary tract and liver metastases. In certain embodiments, the human subjects for whom the methods provided herein can have a primary site of tumor in the upper urinary tract and at least 1 Bellmunt risk factor. In other embodiments, the human subjects for whom the methods provided herein can have a primary site of tumor in the upper urinary tract and ECOG performance status score of 0. In other embodiments, the human subjects for whom the methods provided herein can have a primary site of tumor in the upper urinary tract, visceral metastases, and liver metastases. In yet other embodiments, the human subjects for whom the methods provided herein can have a primary site of tumor in the upper urinary tract, visceral metastases and at least 1 Bellmunt risk factor. In further embodiments, the human subjects for whom the methods provided herein can have a primary site of tumor in the upper urinary tract, visceral metastases, and ECOG performance status score of 0. In some embodiments, the human subjects for whom the methods provided herein can have a primary site of tumor in the upper urinary tract, liver metastases and at least 1 Bellmunt risk factor. In certain embodiments, the human subjects for whom the methods provided herein can have a primary site of tumor in the upper urinary tract, liver metastases, and ECOG performance status score of 0. In yet other embodiments, the human subjects for whom the methods provided herein can have a primary site of tumor in the upper urinary tract, at least 1 Bellmunt risk factor and ECOG performance status score of 0. In other embodiments, the human subjects for whom the methods provided herein can have a primary site of tumor in the upper urinary tract, visceral metastases, liver metastases, and at least 1 Bellmunt risk factor. In further embodiments, the human subjects for whom the methods provided herein can have a primary site of tumor in the upper urinary tract, visceral metastases, liver metastases, and ECOG performance status score of 0. In some embodiments, the human subjects for whom the methods provided herein can have a primary site of tumor in the upper urinary tract, visceral metastases, at least 1 Bellmunt risk factor, and ECOG performance status score of 0. In certain embodiments, the human subjects for whom the methods provided herein can have a primary site of tumor in the upper urinary tract, liver metastases, at least 1 Bellmunt risk factor, and ECOG performance status score of 0. In certain embodiments, the human subjects for whom the methods provided herein can have a primary site of tumor in the upper urinary tract, visceral metastases, liver metastases, at least 1 Bellmunt risk factor, and ECOG performance status score of 0. In some embodiments, the human subjects for whom the methods provided herein can have any one of a primary site of tumor in the upper urinary tract, visceral metastases, liver metastases, at least 1 Bellmunt risk factor, and ECOG performance status score of 0. In some embodiments, the human subjects for whom the methods provided herein can have any two of a primary site of tumor in the upper urinary tract, visceral metastases, liver metastases, at least 1 Bellmunt risk factor, and ECOG performance status score of 0, in any combination or permutation. In some embodiments, the human subjects for whom the methods provided herein can have any three of a primary site of tumor in the upper urinary tract, visceral metastases, liver metastases, at least 1 Bellmunt risk factor, and ECOG performance status score of 0, in any combination or permutation. In some embodiments, the human subjects for whom the methods provided herein can have any four of a primary site of tumor in the upper urinary tract, visceral metastases, liver metastases, at least 1 Bellmunt risk factor, and ECOG performance status score of 0, in any combination or permutation. In some embodiments, the human subjects for whom the methods provided herein can have all five of a primary site of tumor in the upper urinary tract, visceral metastases, liver metastases, at least 1 Bellmunt risk factor, and ECOG performance status score of 0.

In further embodiments of the methods provided herein, including the methods of the preceding paragraphs, the human subjects for whom the methods provided herein can be used are human subjects having various other conditions. In one embodiment, the human subjects for whom the methods provided herein can be used also have the conditions of absolute neutrophil count no less than 1.0×10⁹/L. In some embodiments, the human subjects for whom the methods provided herein can be used also have the conditions of platelet count no less than 100×10⁹/L. In certain embodiments, the human subjects for whom the methods provided herein can be used also have the conditions of hemoglobin no less than 9 g/dL. In other embodiments, the human subjects for whom the methods provided herein can be used also have the conditions of serum bilirubin no more than either of 1.5 times of upper limit of normal (ULN) or 3 times ULN for patients with Gilbert's disease. In yet other embodiments, the human subjects for whom the methods provided herein can be used also have the conditions of CrCl no less than 30 mL/min. In another embodiment, the human subjects for whom the methods provided herein can be used also have the conditions of alanine aminotransferase (ALT) and aspartate aminotransferase (AST) no more than 3 fold of ULN. In one embodiment, the human subjects for whom the methods provided herein can be used also have the conditions of absolute neutrophil count no less than 1.0×10⁹/L and platelet count no less than 100×10⁹/L. In some embodiments, the human subjects for whom the methods provided herein can be used also have the conditions of absolute neutrophil count no less than 1.0×10⁹/L and hemoglobin no less than 9 g/dL. In certain embodiments, the human subjects for whom the methods provided herein can be used also have the conditions of absolute neutrophil count no less than 1.0×109/L and serum bilirubin no more than either of 1.5 times of ULN or 3 times ULN for patients with Gilbert's disease. In other embodiments, the human subjects for whom the methods provided herein can be used also have the conditions of absolute neutrophil count no less than 1.0×10⁹/L and CrCl no less than 30 mL/min. In some embodiments, the human subjects for whom the methods provided herein can be used also have the conditions of absolute neutrophil count no less than 1.0×10⁹/L and ALT and AST no more than 3 fold of ULN. In further embodiments, the human subjects for whom the methods provided herein can be used also have the conditions of platelet count no less than 100×10⁹/L and hemoglobin no less than 9 g/dL. In one embodiment, the human subjects for whom the methods provided herein can be used also have the conditions of platelet count no less than 100×10⁹/L and serum bilirubin no more than either of 1.5 times of ULN or 3 times ULN for patients with Gilbert's disease. In some embodiments, the human subjects for whom the methods provided herein can be used also have the conditions of platelet count no less than 100×10⁹/L and CrCl no less than 30 mL/min. In certain embodiments, the human subjects for whom the methods provided herein can be used also have the conditions of platelet count no less than 100×10⁹/L and ALT and AST no more than 3 fold of ULN. In other embodiments, the human subjects for whom the methods provided herein can be used also have the conditions of hemoglobin no less than 9 g/dL and serum bilirubin no more than either of 1.5 times of ULN or 3 times ULN for patients with Gilbert's disease. In yet other embodiments, the human subjects for whom the methods provided herein can be used also have the conditions of hemoglobin no less than 9 g/dL and CrCl no less than 30 mL/min. In some embodiments, the human subjects for whom the methods provided herein can be used also have the conditions of hemoglobin no less than 9 g/dL and ALT and AST no more than 3 fold of ULN. In one embodiment, the human subjects for whom the methods provided herein can be used also have the conditions of serum bilirubin no more than either of 1.5 times of ULN or 3 times ULN for patients with Gilbert's disease and CrCl no less than 30 mL/min. In another embodiment, the human subjects for whom the methods provided herein can be used also have the conditions of serum bilirubin no more than either of 1.5 times of ULN or 3 times ULN for patients with Gilbert's disease and ALT and AST no more than 3 fold of ULN. In another embodiment, the human subjects for whom the methods provided herein can be used also have the conditions of CrCl no less than 30 mL/min and ALT and AST no more than 3 fold of ULN. In other embodiments, the human subjects for whom the methods provided herein can be used also have the conditions of absolute neutrophil count no less than 1.0×10⁹/L, platelet count no less than 100×10⁹/L, and hemoglobin no less than 9 g/dL. In yet other embodiments, the human subjects for whom the methods provided herein can be used also have the conditions of absolute neutrophil count no less than 1.0×10⁹/L, platelet count no less than 100×10⁹/L and serum bilirubin no more than either of 1.5 times of ULN or 3 times ULN for patients with Gilbert's disease. In further embodiments, the human subjects for whom the methods provided herein can be used also have the conditions of absolute neutrophil count no less than 1.0×10⁹/L, platelet count no less than 100×109/L, and CrCl no less than 30 mL/min. In some embodiments, the human subjects for whom the methods provided herein can be used also have the conditions of absolute neutrophil count no less than 1.0×10⁹/L, platelet count no less than 100×10⁹/L, and ALT and AST no more than 3 fold of ULN. In some embodiments, the human subjects for whom the methods provided herein can be used also have the conditions of absolute neutrophil count no less than 1.0×10⁹/L, hemoglobin no less than 9 g/dL and serum bilirubin no more than either of 1.5 times of ULN or 3 times ULN for patients with Gilbert's disease. In certain embodiments, the human subjects for whom the methods provided herein can be used also have the conditions of absolute neutrophil count no less than 1.0×10⁹/L, hemoglobin no less than 9 g/dL, and CrCl no less than 30 mL/min. In some embodiments, the human subjects for whom the methods provided herein can be used also have the conditions of absolute neutrophil count no less than 1.0×10⁹/L, hemoglobin no less than 9 g/dL, and ALT and AST no more than 3 fold of ULN. In yet other embodiments, the human subjects for whom the methods provided herein can be used also have the conditions of absolute neutrophil count no less than 1.0×10⁹/L, serum bilirubin no more than either of 1.5 times of ULN or 3 times ULN for patients with Gilbert's disease and CrCl no less than 30 mL/min. In some embodiments, the human subjects for whom the methods provided herein can be used also have the conditions of absolute neutrophil count no less than 1.0×10⁹/L, serum bilirubin no more than either of 1.5 times of ULN or 3 times ULN for patients with Gilbert's disease and ALT and AST no more than 3 fold of ULN. In some embodiments, the human subjects for whom the methods provided herein can be used also have the conditions of absolute neutrophil count no less than 1.0×10⁹/L, CrCl no less than 30 mL/min and ALT and AST no more than 3 fold of ULN. In some embodiments, the human subjects for whom the methods provided herein can be used also have the conditions of platelet count no less than 100×10⁹/L, hemoglobin no less than 9 g/dL and serum bilirubin no more than either of 1.5 times of ULN or 3 times ULN for patients with Gilbert's disease. In certain embodiments, the human subjects for whom the methods provided herein can be used also have the conditions of platelet count no less than 100×10⁹/L, hemoglobin no less than 9 g/dL, and CrCl no less than 30 mL/min. In some embodiments, the human subjects for whom the methods provided herein can be used also have the conditions of platelet count no less than 100×10⁹/L, hemoglobin no less than 9 g/dL, and ALT and AST no more than 3 fold of ULN. In other embodiments, the human subjects for whom the methods provided herein can be used also have the conditions of platelet count no less than 100×10⁹/L, serum bilirubin no more than either of 1.5 times of ULN or 3 times ULN for patients with Gilbert's disease and CrCl no less than 30 mL/min. In yet other embodiments, the human subjects for whom the methods provided herein can be used also have the conditions of platelet count no less than 100×10⁹/L, serum bilirubin no more than either of 1.5 times of ULN or 3 times ULN for patients with Gilbert's disease and ALT and AST no more than 3 fold of ULN. In some embodiments, the human subjects for whom the methods provided herein can be used also have the conditions of platelet count no less than 100×10⁹/L, CrCl no less than 30 mL/min and ALT and AST no more than 3 fold of ULN. In yet other embodiments, the human subjects for whom the methods provided herein can be used also have the conditions of hemoglobin no less than 9 g/dL, serum bilirubin no more than either of 1.5 times of ULN or 3 times ULN for patients with Gilbert's disease and CrCl no less than 30 mL/min. In other embodiments, the human subjects for whom the methods provided herein can be used also have the conditions of hemoglobin no less than 9 g/dL, serum bilirubin no more than either of 1.5 times of ULN or 3 times ULN for patients with Gilbert's disease and ALT and AST no more than 3 fold of ULN. In certain embodiments, the human subjects for whom the methods provided herein can be used also have the conditions of hemoglobin no less than 9 g/dL, CrCl no less than 30 mL/min and ALT and AST no more than 3 fold of ULN. In some embodiments, the human subjects for whom the methods provided herein can be used also have the conditions of serum bilirubin no more than either of 1.5 times of ULN or 3 times ULN for patients with Gilbert's disease, CrCl no less than 30 mL/min and ALT and AST no more than 3 fold of ULN. In other embodiments, the human subjects for whom the methods provided herein can be used also have the conditions of absolute neutrophil count no less than 1.0×10⁹/L, platelet count no less than 100×10⁹/L, hemoglobin no less than 9 g/dL, and serum bilirubin no more than either of 1.5 times of ULN or 3 times ULN for patients with Gilbert's disease. In further embodiments, the human subjects for whom the methods provided herein can be used also have the conditions of absolute neutrophil count no less than 1.0×10⁹/L, platelet count no less than 100×10⁹/L, hemoglobin no less than 9 g/dL, and CrCl no less than 30 mL/min. In some embodiments, the human subjects for whom the methods provided herein can be used also have the conditions of absolute neutrophil count no less than 1.0×10⁹/L, platelet count no less than 100×10⁹/L, hemoglobin no less than 9 g/dL, and ALT and AST no more than 3 fold of ULN. In some embodiments, the human subjects for whom the methods provided herein can be used also have the conditions of absolute neutrophil count no less than 1.0×10⁹/L, platelet count no less than 100×10⁹/L, serum bilirubin no more than either of 1.5 times of ULN or 3 times ULN for patients with Gilbert's disease, and CrCl no less than 30 mL/min. In some embodiments, the human subjects for whom the methods provided herein can be used also have the conditions of absolute neutrophil count no less than 1.0×10⁹/L, platelet count no less than 100×10⁹/L, serum bilirubin no more than either of 1.5 times of ULN or 3 times ULN for patients with Gilbert's disease, and ALT and AST no more than 3 fold of ULN. In some embodiments, the human subjects for whom the methods provided herein can be used also have the conditions of absolute neutrophil count no less than 1.0×109/L, platelet count no less than 100×10⁹/L, CrCl no less than 30 mL/min, and ALT and AST no more than 3 fold of ULN. In certain embodiments, the human subjects for whom the methods provided herein can be used also have the conditions of absolute neutrophil count no less than 1.0×10⁹/L, hemoglobin no less than 9 g/dL, serum bilirubin no more than either of 1.5 times of ULN or 3 times ULN for patients with Gilbert's disease, and CrCl no less than 30 mL/min. In certain embodiments, the human subjects for whom the methods provided herein can be used also have the conditions of absolute neutrophil count no less than 1.0×10⁹/L, hemoglobin no less than 9 g/dL, serum bilirubin no more than either of 1.5 times of ULN or 3 times ULN for patients with Gilbert's disease, and ALT and AST no more than 3 fold of ULN. In certain embodiments, the human subjects for whom the methods provided herein can be used also have the conditions of absolute neutrophil count no less than 1.0×10⁹/L, hemoglobin no less than 9 g/dL, CrCl no less than 30 mL/min, and ALT and AST no more than 3 fold of ULN. In yet other embodiments, the human subjects for whom the methods provided herein can be used also have the conditions of absolute neutrophil count no less than 1.0×10⁹/L, serum bilirubin no more than either of 1.5 times of ULN or 3 times ULN for patients with Gilbert's disease, CrCl no less than 30 mL/min, and ALT and AST no more than 3 fold of ULN. In further embodiments, the human subjects for whom the methods provided herein can be used also have the conditions of platelet count no less than 100×10⁹/L, hemoglobin no less than 9 g/dL, serum bilirubin no more than either of 1.5 times of ULN or 3 times ULN for patients with Gilbert's disease, and CrCl no less than 30 mL/min. In some embodiments, the human subjects for whom the methods provided herein can be used also have the conditions of platelet count no less than 100×10⁹/L, hemoglobin no less than 9 g/dL, serum bilirubin no more than either of 1.5 times of ULN or 3 times ULN for patients with Gilbert's disease, and ALT and AST no more than 3 fold of ULN. In further embodiments, the human subjects for whom the methods provided herein can be used also have the conditions of platelet count no less than 100×10⁹/L, hemoglobin no less than 9 g/dL, CrCl no less than 30 mL/min, and ALT and AST no more than 3 fold of ULN. In some embodiments, the human subjects for whom the methods provided herein can be used also have the conditions of platelet count no less than 100×10⁹/L, serum bilirubin no more than either of 1.5 times of ULN or 3 times ULN for patients with Gilbert's disease, CrCl no less than 30 mL/min, and ALT and AST no more than 3 fold of ULN. In some embodiments, the human subjects for whom the methods provided herein can be used also have the conditions of hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN. In certain embodiments, the human subjects for whom the methods provided herein can be used also have the conditions of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, and CrCl no less than 30 mL/min. In some embodiments, the human subjects for whom the methods provided herein can be used also have the conditions of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, and ALT and AST no more than 3 fold of ULN. In some embodiments, the human subjects for whom the methods provided herein can be used also have the conditions of absolute neutrophil count no less than 1.0×10⁹/L, platelet count no less than 100×10⁹/L, hemoglobin no less than 9 g/dL, CrCl no less than 30 mL/min, and ALT and AST no more than 3 fold of ULN. In certain embodiments, the human subjects for whom the methods provided herein can be used also have the conditions of absolute neutrophil count no less than 1.0×10⁹/L, platelet count no less than 100×10⁹/L, serum bilirubin no more than either of 1.5 times of ULN or 3 times ULN for patients with Gilbert's disease, CrCl no less than 30 mL/min, and ALT and AST no more than 3 fold of ULN. In certain embodiments, the human subjects for whom the methods provided herein can be used also have the conditions of absolute neutrophil count no less than 1.0×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN. In some embodiments, the human subjects for whom the methods provided herein can be used also have the conditions of platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN. In some embodiments, the human subjects for whom the methods provided herein can be used also have the conditions of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN. In some embodiments, the human subjects for whom the methods provided herein can be used also have the conditions of any one of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN. In some embodiments, the human subjects for whom the methods provided herein can be used also have the conditions of any two of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN, in any combination or permutation. In some embodiments, the human subjects for whom the methods provided herein can be used also have the conditions of any three of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN, in any combination or permutation. In some embodiments, the human subjects for whom the methods provided herein can be used also have the conditions of any four of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN, in any combination or permutation. In some embodiments, the human subjects for whom the methods provided herein can be used also have the conditions of any five of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN, in any combination or permutation. In some embodiments, the human subjects for whom the methods provided herein can be used also have the conditions of all six of absolute neutrophil count no less than 1.0×10⁹/L, platelet count no less than 100×10⁹/L, hemoglobin no less than 9 g/dL, serum bilirubin no more than either of 1.5 times of ULN or 3 times ULN for patients with Gilbert's disease, CrCl no less than 30 mL/min, and ALT and AST no more than 3 fold of ULN.

In other embodiments of the methods provided herein, including the methods of the preceding paragraphs, the human subjects for whom the methods provided herein can be used are human subjects free from certain conditions. In one embodiment, the human subjects for whom the methods provided herein can have no more than Grade 2 sensory or motor neuropathy. In some embodiments, the human subjects for whom the methods provided herein can have no active central nervous system metastases. In certain embodiments, the human subjects for whom the methods provided herein can have no uncontrolled diabetes. In one embodiment, the human subjects for whom the methods provided herein can have no more than Grade 2 sensory or motor neuropathy and no active central nervous system metastases. In some embodiments, the human subjects for whom the methods provided herein can have no more than Grade 2 sensory or motor neuropathy and no uncontrolled diabetes. In further embodiments, the human subjects for whom the methods provided herein can have no active central nervous system metastases and no uncontrolled diabetes. In yet other embodiments, the human subjects for whom the methods provided herein can have no more than Grade 2 sensory or motor neuropathy, no active central nervous system metastases, and no uncontrolled diabetes. In some embodiments, the human subjects for whom the methods provided herein can have any one of no more than Grade 2 sensory or motor neuropathy, no active central nervous system metastases, and no uncontrolled diabetes. In some embodiments, the human subjects for whom the methods provided herein can have any two of no more than Grade 2 sensory or motor neuropathy, no active central nervous system metastases, and no uncontrolled diabetes, in any combination or permutation. In some embodiments, the human subjects for whom the methods provided herein can have all three of no more than Grade 2 sensory or motor neuropathy, no active central nervous system metastases, and no uncontrolled diabetes. In one embodiment of the methods provided in this paragraph, the uncontrolled diabetes is determined by hemoglobin A1c (HbA1c) no less than 8%. In some embodiments of the methods provided in this paragraph, the uncontrolled diabetes is determined by HbA1c between 7 and 8% with associated diabetes symptoms that are not otherwise explained. In further embodiments of the methods provided in this paragraph, the associated diabetes symptoms comprise or consist of polyuria. In some other embodiments of the methods provided in this paragraph, the associated diabetes symptoms comprise or consist of polydipsia. In yet other embodiments of the methods provided in this paragraph, the associated diabetes symptoms comprise or consist of both polyuria and polydipsia.

In some embodiments of the methods provided herein, the CrCl is measured by 24 hour urine collection. In other embodiments of the methods provided herein, the CrCl is estimated by the Cockcroft-Gault criteria.

In some embodiments of the methods provided herein, the subject has been treated with one or more other cancer treatments. In certain embodiments of the methods provided herein, the urothelial cancer, including locally advanced or metastatic urothelial cancer, has been treated with one or more other cancer treatments.

In some embodiments, the CPI provided for the methods can comprise or consist of any CPI as described in this Section (Section 5.2.1.1).

### 5.2.1.2 Therapeutic Outcome of the Methods Provided Herein

The methods provided herein, including in methods described in this Section (Section 5.2) and Sections 3 and 6, can provide beneficial therapeutic outcomes for these human subjects having cancer. In one embodiment, the human subject has a complete response following the treatment by a method provided herein. In another embodiment, the human subject has a partial response following the treatment by a method provided herein.

In some embodiments, the response (complete or partial response) is determined by evaluating the tumor or cancer site (lesions). The criteria for determining complete response (CR), partial response (PR), progressive disease (PD), and stable disease (SD) are described in Section 6 (e.g., at Section 6.1.6.3).

The therapeutic outcome of the methods provided herein thus can be evaluated based on any one or more of the response criteria described above. In one embodiment, the human subject has a partial response following the treatment by a method provided herein, wherein the partial response is defined by an at least or about 30% decrease in the sum of the diameters of target lesions, taking as reference the baseline sum diameters. In another embodiment, the human subject has a partial response following the treatment by a method provided herein, wherein the partial response is defined by an at least or about 35% decrease in the sum of the diameters of target lesions, taking as reference the baseline sum diameters. In a further embodiment, the human subject has a partial response following the treatment by a method provided herein, wherein the partial response is defined by an at least or about 40% decrease in the sum of the diameters of target lesions, taking as reference the baseline sum diameters. In yet another embodiment, the human subject has a partial response following the treatment by a method provided herein, wherein the partial response is defined by an at least or about 45% decrease in the sum of the diameters of target lesions, taking as reference the baseline sum diameters. In one embodiment, the human subject has a partial response following the treatment by a method provided herein, wherein the partial response is defined by an at least or about 50% decrease in the sum of the diameters of target lesions, taking as reference the baseline sum diameters. In another embodiment, the human subject has a partial response following the treatment by a method provided herein, wherein the partial response is defined by an at least or about 55% decrease in the sum of the diameters of target lesions, taking as reference the baseline sum diameters. In a further embodiment, the human subject has a partial response following the treatment by a method provided herein, wherein the partial response is defined by an at least or about 60% decrease in the sum of the diameters of target lesions, taking as reference the baseline sum diameters. In yet another embodiment, the human subject has a partial response following the treatment by a method provided herein, wherein the partial response is defined by an at least or about 65% decrease in the sum of the diameters of target lesions, taking as reference the baseline sum diameters. In one embodiment, the human subject has a partial response following the treatment by a method provided herein, wherein the partial response is defined by an at least or about 70% decrease in the sum of the diameters of target lesions, taking as reference the baseline sum diameters. In another embodiment, the human subject has a partial response following the treatment by a method provided herein, wherein the partial response is defined by an at least or about 75% decrease in the sum of the diameters of target lesions, taking as reference the baseline sum diameters. In a further embodiment, the human subject has a partial response following the treatment by a method provided herein, wherein the partial response is defined by an at least or about 80% decrease in the sum of the diameters of target lesions, taking as reference the baseline sum diameters. In yet another embodiment, the human subject has a partial response following the treatment by a method provided herein, wherein the partial response is defined by an at least or about 85% decrease in the sum of the diameters of target lesions, taking as reference the baseline sum diameters. In one embodiment, the human subject has a partial response following the treatment by a method provided herein, wherein the partial response is defined by an at least or about 90% decrease in the sum of the diameters of target lesions, taking as reference the baseline sum diameters. In another embodiment, the human subject has a partial response following the treatment by a method provided herein, wherein the partial response is defined by an at least or about 95% decrease in the sum of the diameters of target lesions, taking as reference the baseline sum diameters. In some embodiments, the diameter is determined according to the longest diameter of a lesion. In certain embodiments, the diameter is determined according to the longest diameter of a lesion in the plane of measurement. In some embodiments, the diameter is determined according to the longest diameter of a lesion in the plane of measurement with a minimal size of 10 mm by CT scan. In certain embodiments, the diameter is determined according to the longest diameter of a lesion in the plane of measurement with a minimal size of 10 mm by CT scan and CT slice thickness no greater than 5 mm.

The therapeutic outcomes of the methods provided herein can also be evaluated based on whether the disease is stable following the treatment. In one embodiment, the human subject has a stable disease following the treatment by a method provided herein. In another embodiment, the human subject does not have a progressive disease following the treatment by a method provided herein.

Alternatively, therapeutic outcomes based on the complete response, partial response, or stable disease can be evaluated with respect to a population of human subjects treated by a method provided herein by evaluating the percentage of the subjects having complete response, partial response, or stable disease in the treated population. As such, in some embodiments, the therapeutic outcome or efficacy measure applies to outcomes achieved by actually treating a population of subjects. In other embodiments, the therapeutic outcome or efficacy measure refers to the outcome or efficacy that is capable of being achieved if a population of human subjects was treated with a method as disclosed herein. While the following sections discuss the treatment of an actual population of human subjects, is should be understood that corresponding methods in which the outcome or efficacy measure is capable of being achieved in a patient population are also encompassed herein. In short, both scenarios described above apply to the following sections; only one scenario is described below in the interest of simplicity and to avoid redundancy.

In some embodiments of the methods provided herein, including in Sections 3, 5.3, and this Section (Section 5.2), the ADC is enfortumab vedotin. In certain 6 embodiments of the methods provided herein, including in Sections 3, 5.3, and 6 and this Section (Section 5.2), the ADC is a biosimilar of enfortumab vedotin.

In one embodiment, a population of the human subjects is treated by a method provided herein, wherein the percentage of the subjects having complete response in the treated population is at least or about 2%. In another embodiment, a population of the human subjects is treated by a method provided herein, wherein the percentage of the subjects having complete response in the treated population is at least or about 3%. In another embodiment, a population of the human subjects is treated by a method provided herein, wherein the percentage of the subjects having complete response in the treated population is at least or about 4%. In another embodiment, a population of the human subjects is treated by a method provided herein, wherein the percentage of the subjects having complete response in the treated population is at least or about 5%. In another embodiment, a population of the human subjects is treated by a method provided herein, wherein the percentage of the subjects having complete response in the treated population is at least or about 6%. In another embodiment, a population of the human subjects is treated by a method provided herein, wherein the percentage of the subjects having complete response in the treated population is at least or about 7%. In another embodiment, a population of the human subjects is treated by a method provided herein, wherein the percentage of the subjects having complete response in the treated population is at least or about 8%. In another embodiment, a population of the human subjects is treated by a method provided herein, wherein the percentage of the subjects having complete response in the treated population is at least or about 9%. In another embodiment, a population of the human subjects is treated by a method provided herein, wherein the percentage of the subjects having complete response in the treated population is at least or about 10%. In another embodiment, a population of the human subjects is treated by a method provided herein, wherein the percentage of the subjects having complete response in the treated population is at least or about 15%. In a further embodiment, a population of the human subjects is treated by a method provided herein, wherein the percentage of the subjects having complete response in the treated population is at least or about 20%. In yet another embodiment, a population of the human subjects is treated by a method provided herein, wherein the percentage of the subjects having complete response in the treated population is at least or about 20.2%. In one embodiment, a population of the human subjects is treated by a method provided herein, wherein the percentage of the subjects having complete response in the treated population is at least or about 22%. In yet another embodiment, a population of the human subjects is treated by a method provided herein, wherein the percentage of the subjects having complete response in the treated population is at least or about 22.5%. In another embodiment, a population of the human subjects is treated by a method provided herein, wherein the percentage of the subjects having complete response in the treated population is at least or about 23%. In one embodiment, a population of the human subjects is treated by a method provided herein, wherein the percentage of the subjects having complete response in the treated population is at least or about 25%. In another embodiment, a population of the human subjects is treated by a method provided herein, wherein the percentage of the subjects having complete response in the treated population is at least or about 30%. In a further embodiment, a population of the human subjects is treated by a method provided herein, wherein the percentage of the subjects having complete response in the treated population is at least or about 35%. In yet another embodiment, a population of the human subjects is treated by a method provided herein, wherein the percentage of the subjects having complete response in the treated population is at least or about 40%.

Similarly, using percentage of partial response as the criteria, in one embodiment, a population of the human subjects is treated by a method provided herein, wherein the percentage of the subjects having partial response in the treated population is at least or about 20%. In yet another embodiment, a population of the human subjects is treated by a method provided herein, wherein the percentage of the subjects having partial response in the treated population is at least or about 25%. In one embodiment, a population of the human subjects is treated by a method provided herein, wherein the percentage of the subjects having partial response in the treated population is at least or about 28%. In one embodiment, a population of the human subjects is treated by a method provided herein, wherein the percentage of the subjects having partial response in the treated population is at least or about 30%. In one embodiment, a population of the human subjects is treated by a method provided herein, wherein the percentage of the subjects having partial response in the treated population is at least or about 31%. In another embodiment, a population of the human subjects is treated by a method provided herein, wherein the percentage of the subjects having partial response in the treated population is at least or about 32%. In another embodiment, a population of the human subjects is treated by a method provided herein, wherein the percentage of the subjects having partial response in the treated population is at least or about 33%. In another embodiment, a population of the human subjects is treated by a method provided herein, wherein the percentage of the subjects having partial response in the treated population is at least or about 34%. In a further embodiment, a population of the human subjects is treated by a method provided herein, wherein the percentage of the subjects having partial response in the treated population is at least or about 35%. In yet another embodiment, a population of the human subjects is treated by a method provided herein, wherein the percentage of the subjects having partial response in the treated population is at least or about 36%. In one embodiment, a population of the human subjects is treated by a method provided herein, wherein the percentage of the subjects having partial response in the treated population is at least or about 37%. In another embodiment, a population of the human subjects is treated by a method provided herein, wherein the percentage of the subjects having partial response in the treated population is at least or about 38%. In another embodiment, a population of the human subjects is treated by a method provided herein, wherein the percentage of the subjects having partial response in the treated population is at least or about 39%. In another embodiment, a population of the human subjects is treated by a method provided herein, wherein the percentage of the subjects having partial response in the treated population is at least or about 40%. In another embodiment, a population of the human subjects is treated by a method provided herein, wherein the percentage of the subjects having partial response in the treated population is at least or about 45%. In another embodiment, a population of the human subjects is treated by a method provided herein, wherein the percentage of the subjects having partial response in the treated population is at least or about 50%.

Likewise, overall response rate, which is the sum of percentage of subjects having completed response and those having partial response, can be used as the evaluation criteria for the therapeutic outcome in the human subjects treated by a method provided herein. In one embodiment, a population of the human subjects is treated by a method provided herein, wherein overall response rate in the treated population is at least or about 20%. In one embodiment, a population of the human subjects is treated by a method provided herein, wherein overall response rate in the treated population is at least or about 25%. In another embodiment, a population of the human subjects is treated by a method provided herein, wherein overall response rate in the treated population is at least or about 30%. In a further embodiment, a population of the human subjects is treated by a method provided herein, wherein overall response rate in the treated population is at least or about 35%. In yet another embodiment, a population of the human subjects is treated by a method provided herein, wherein overall response rate in the treated population is at least or about 36%. In yet another embodiment, a population of the human subjects is treated by a method provided herein, wherein overall response rate in the treated population is at least or about 37%. In one embodiment, a population of the human subjects is treated by a method provided herein, wherein overall response rate in the treated population is at least or about 38%. In another embodiment, a population of the human subjects is treated by a method provided herein, wherein overall response rate in the treated population is at least or about 39%. In one embodiment, a population of the human subjects is treated by a method provided herein, wherein overall response rate in the treated population is at least or about 40%. In a further embodiment, a population of the human subjects is treated by a method provided herein, wherein overall response rate in the treated population is at least or about 41%. In yet another embodiment, a population of the human subjects is treated by a method provided herein, wherein overall response rate in the treated population is at least or about 42%. In one embodiment, a population of the human subjects is treated by a method provided herein, wherein overall response rate in the treated population is at least or about 43%. In another embodiment, a population of the human subjects is treated by a method provided herein, wherein overall response rate in the treated population is at least or about 44%. In a further embodiment, a population of the human subjects is treated by a method provided herein, wherein overall response rate in the treated population is at least or about 45%. In yet another embodiment, a population of the human subjects is treated by a method provided herein, wherein overall response rate in the treated population is at least or about 50%. In one embodiment, a population of the human subjects is treated by a method provided herein, wherein overall response rate in the treated population is at least or about 55%. In another embodiment, a population of the human subjects is treated by a method provided herein, wherein overall response rate in the treated population is at least or about 60%. In a further embodiment, a population of the human subjects is treated by a method provided herein, wherein overall response rate in the treated population is at least or about 65%. In yet another embodiment, a population of the human subjects is treated by a method provided herein, wherein overall response rate in the treated population is at least or about 70%. In yet another embodiment, a population of the human subjects is treated by a method provided herein, wherein overall response rate in the treated population is at least or about 75%. In yet another embodiment, a population of the human subjects is treated by a method provided herein, wherein overall response rate in the treated population is at least or about 80%.

In one embodiment, a population of the human subjects is treated by a method provided herein, wherein overall response rate in the treated population ranges from 40% to 65%. In one embodiment, a population of the human subjects is treated by a method provided herein, wherein overall response rate in the treated population ranges from 40% to 65%. In another embodiment, a population of the human subjects is treated by a method provided herein, wherein overall response rate in the treated population ranges from 40% to 60%. In a further embodiment, a population of the human subjects is treated by a method provided herein, wherein overall response rate in the treated population ranges from 40% to 55%. In yet another embodiment, a population of the human subjects is treated by a method provided herein, wherein overall response rate in the treated population ranges from 40% to 50%. In one embodiment, a population of the human subjects is treated by a method provided herein, wherein overall response rate in the treated population ranges from 40% to 45%. In one embodiment, a population of the human subjects is treated by a method provided herein, wherein overall response rate in the treated population ranges from 35% to 65%. In one embodiment, a population of the human subjects is treated by a method provided herein, wherein overall response rate in the treated population ranges from 35% to 65%. In another embodiment, a population of the human subjects is treated by a method provided herein, wherein overall response rate in the treated population ranges from 35% to 60%. In a further embodiment, a population of the human subjects is treated by a method provided herein, wherein overall response rate in the treated population ranges from 35% to 55%. In yet another embodiment, a population of the human subjects is treated by a method provided herein, wherein overall response rate in the treated population ranges from 35% to 50%. In one embodiment, a population of the human subjects is treated by a method provided herein, wherein overall response rate in the treated population ranges from 35% to 45%. In one embodiment, a population of the human subjects is treated by a method provided herein, wherein overall response rate in the treated population ranges from 35% to 40%. In one embodiment, a population of the human subjects is treated by a method provided herein, wherein overall response rate in the treated population ranges from 30% to 65%. In one embodiment, a population of the human subjects is treated by a method provided herein, wherein overall response rate in the treated population ranges from 30% to 65%. In another embodiment, a population of the human subjects is treated by a method provided herein, wherein overall response rate in the treated population ranges from 30% to 60%. In a further embodiment, a population of the human subjects is treated by a method provided herein, wherein overall response rate in the treated population ranges from 30% to 55%. In yet another embodiment, a population of the human subjects is treated by a method provided herein, wherein overall response rate in the treated population ranges from 30% to 50%. In one embodiment, a population of the human subjects is treated by a method provided herein, wherein overall response rate in the treated population ranges from 30% to 45%. In one embodiment, a population of the human subjects is treated by a method provided herein, wherein overall response rate in the treated population ranges from 30% to 40%. In one embodiment, a population of the human subjects is treated by a method provided herein, wherein overall response rate in the treated population ranges from 30% to 35%.

Moreover, percentage of the subjects having stable disease can be used as the evaluation criteria for the therapeutic outcome in the human subjects treated by a method provided herein. In one embodiment, a population of the human subjects is treated by a method provided herein, wherein the percentage of the subjects having stable disease in the treated population is at least or about 10%. In one embodiment, a population of the human subjects is treated by a method provided herein, wherein the percentage of the subjects having stable disease in the treated population is at least or about 15%. In one embodiment, a population of the human subjects is treated by a method provided herein, wherein the percentage of the subjects having stable disease in the treated population is at least or about 20%. In one embodiment, a population of the human subjects is treated by a method provided herein, wherein the percentage of the subjects having stable disease in the treated population is at least or about 25%. In one embodiment, a population of the human subjects is treated by a method provided herein, wherein the percentage of the subjects having stable disease in the treated population is at least or about 26%. In one embodiment, a population of the human subjects is treated by a method provided herein, wherein the percentage of the subjects having stable disease in the treated population is at least or about 27%. In one embodiment, a population of the human subjects is treated by a method provided herein, wherein the percentage of the subjects having stable disease in the treated population is at least or about 28%. In one embodiment, a population of the human subjects is treated by a method provided herein, wherein the percentage of the subjects having stable disease in the treated population is at least or about 29%. In one embodiment, a population of the human subjects is treated by a method provided herein, wherein the percentage of the subjects having stable disease in the treated population is at least or about 30%. In one embodiment, a population of the human subjects is treated by a method provided herein, wherein the percentage of the subjects having stable disease in the treated population is at least or about 31%. In one embodiment, a population of the human subjects is treated by a method provided herein, wherein the percentage of the subjects having stable disease in the treated population is at least or about 32%. In one embodiment, a population of the human subjects is treated by a method provided herein, wherein the percentage of the subjects having stable disease in the treated population is at least or about 33%. In one embodiment, a population of the human subjects is treated by a method provided herein, wherein the percentage of the subjects having stable disease in the treated population is at least or about 34%. In one embodiment, a population of the human subjects is treated by a method provided herein, wherein the percentage of the subjects having stable disease in the treated population is at least or about 35%. In one embodiment, a population of the human subjects is treated by a method provided herein, wherein the percentage of the subjects having stable disease in the treated population is at least or about 40%. In one embodiment, a population of the human subjects is treated by a method provided herein, wherein the percentage of the subjects having stable disease in the treated population is at least or about 45%. In one embodiment, a population of the human subjects is treated by a method provided herein, wherein the percentage of the subjects having stable disease in the treated population is at least or about 50%. In one embodiment, a population of the human subjects is treated by a method provided herein, wherein the percentage of the subjects having stable disease in the treated population is at least or about 55%. In one embodiment, a population of the human subjects is treated by a method provided herein, wherein the percentage of the subjects having stable disease in the treated population is at least or about 60%.

Additionally, the therapeutic outcome of the methods provided herein can be evaluated based on the duration of response as set forth in Section 6.1.8.4. In one embodiment, the human subject has a duration of response of at least or about 4 months following the treatment. In another embodiment, the human subject has a duration of response of at least or about 5 months following the treatment. In another embodiment, the human subject has a duration of response of at least or about 6 months following the treatment. In a further embodiment, the human subject has a duration of response of at least or about 7 months following the treatment. In yet another embodiment, the human subject has a duration of response of at least or about 8 months following the treatment. In one embodiment, the human subject has a duration of response of at least or about 9 months following the treatment. In another embodiment, the human subject has a duration of response of at least or about 10 months following the treatment. In yet another embodiment, the human subject has a duration of response of at least or about 11 months following the treatment. In one embodiment, the human subject has a duration of response of at least or about 12 months following the treatment. In another embodiment, the human subject has a duration of response of at least or about 13 months following the treatment. In a further embodiment, the human subject has a duration of response of at least or about 14 months following the treatment. In yet another embodiment, the human subject has a duration of response of at least or about 15 months following the treatment. In one embodiment, the human subject has a duration of response of at least or about 16 months following the treatment. In another embodiment, the human subject has a duration of response of at least or about 17 months following the treatment. In a further embodiment, the human subject has a duration of response of at least or about 18 months following the treatment. In yet another embodiment, the human subject has a duration of response of at least or about 19 months following the treatment. In a further embodiment, the human subject has a duration of response of at least or about 20 months following the treatment.

In certain embodiments, the human subject has a duration of response ranging from 4 to 22 months following the treatment. In certain embodiment, the human subject has a duration of response ranging from 5 to more than 22 months following the treatment. In another embodiment, the human subject has a duration of response ranging from 5 to 21 months following the treatment. In a further embodiment, the human subject has a duration of response ranging from 5 to 20 months following the treatment. In yet another embodiment, the human subject has a duration of response ranging from 5 to 19 months following the treatment. In one embodiment, the human subject has a duration of response ranging from 5 to 18 months following the treatment. In another embodiment, the human subject has a duration of response ranging from 5 to 17 months following the treatment. In a further embodiment, the human subject has a duration of response ranging from 5 to 16 months following the treatment. In yet another embodiment, the human subject has a duration of response ranging from 5 to 15 months following the treatment. In one embodiment, the human subject has a duration of response ranging from 5 to 14 months following the treatment. In another embodiment, the human subject has a duration of response ranging from 5 to 13 months following the treatment. In a further embodiment, the human subject has a duration of response ranging from 5 to 12 months following the treatment. In a further embodiment, the human subject has a duration of response ranging from 5 to 11 months following the treatment. In a further embodiment, the human subject has a duration of response ranging from 5 to 10 months following the treatment. In a further embodiment, the human subject has a duration of response ranging from 5 to 9 months following the treatment. In a further embodiment, the human subject has a duration of response ranging from 5 to 8 months following the treatment. In a further embodiment, the human subject has a duration of response ranging from 5 to 7 months following the treatment. In yet another embodiment, the human subject has a duration of response ranging from 6 to 22 months following the treatment. In another embodiment, the human subject has a duration of response ranging from 6 to 21 months following the treatment. In a further embodiment, the human subject has a duration of response ranging from 6 to 20 months following the treatment. In yet another embodiment, the human subject has a duration of response ranging from 6 to 19 months following the treatment. In one embodiment, the human subject has a duration of response ranging from 6 to 18 months following the treatment. In another embodiment, the human subject has a duration of response ranging from 6 to 17 months following the treatment. In a further embodiment, the human subject has a duration of response ranging from 6 to 16 months following the treatment. In yet another embodiment, the human subject has a duration of response ranging from 6 to 15 months following the treatment. In one embodiment, the human subject has a duration of response ranging from 6 to 14 months following the treatment. In another embodiment, the human subject has a duration of response ranging from 6 to 13 months following the treatment. In a further embodiment, the human subject has a duration of response ranging from 6 to 12 months following the treatment. In a further embodiment, the human subject has a duration of response ranging from 6 to 11 months following the treatment. In a further embodiment, the human subject has a duration of response ranging from 6 to 10 months following the treatment. In a further embodiment, the human subject has a duration of response ranging from 6 to 9 months following the treatment. In a further embodiment, the human subject has a duration of response ranging from 6 to 8 months following the treatment. In one embodiment, the human subject has a duration of response ranging from 7 to 22 months following the treatment. In another embodiment, the human subject has a duration of response ranging from 7 to 21 months following the treatment. In a further embodiment, the human subject has a duration of response ranging from 7 to 20 months following the treatment. In yet another embodiment, the human subject has a duration of response ranging from 7 to 19 months following the treatment. In one embodiment, the human subject has a duration of response ranging from 7 to 18 months following the treatment. In another embodiment, the human subject has a duration of response ranging from 7 to 17 months following the treatment. In a further embodiment, the human subject has a duration of response ranging from 7 to 16 months following the treatment. In yet another embodiment, the human subject has a duration of response ranging from 7 to 15 months following the treatment. In one embodiment, the human subject has a duration of response ranging from 7 to 14 months following the treatment. In another embodiment, the human subject has a duration of response ranging from 7 to 13 months following the treatment. In a further embodiment, the human subject has a duration of response ranging from 7 to 12 months following the treatment. In another embodiment, the human subject has a duration of response ranging from 8 to 22 months following the treatment. In a further embodiment, the human subject has a duration of response ranging from 9 to 22 months following the treatment. In yet another embodiment, the human subject has a duration of response ranging from 10 to 22 months following the treatment. In a further embodiment, the human subject has a duration of response ranging from 11 to 22 months following the treatment. In one embodiment, the human subject has a duration of response ranging from 12 to 22 months following the treatment. In another embodiment, the human subject has a duration of response ranging from 13 to 22 months following the treatment. In a further embodiment, the human subject has a duration of response ranging from 14 to 22 months following the treatment. In yet another embodiment, the human subject has a duration of response ranging from 15 to 22 months following the treatment. In a further embodiment, the human subject has a duration of response ranging from 16 to 22 months following the treatment. In one embodiment, the human subject has a duration of response ranging from 17 to 22 months following the treatment. In another embodiment, the human subject has a duration of response ranging from 18 to 22 months following the treatment. In a further embodiment, the human subject has a duration of response ranging from 6 to 21 months following the treatment. In yet another embodiment, the human subject has a duration of response ranging from 7 to 20 months following the treatment. In a further embodiment, the human subject has a duration of response ranging from 8 to 19 months following the treatment. In one embodiment, the human subject has a duration of response ranging from 9 to 18 months following the treatment. In another embodiment, the human subject has a duration of response ranging from 10 to 17 months following the treatment. In a further embodiment, the human subject has a duration of response ranging from 11 to 16 months following the treatment. In yet another embodiment, the human subject has a duration of response ranging from 12 to 15 months following the treatment. In a further embodiment, the human subject has a duration of response ranging from 13 to 14 months following the treatment.

In some embodiments, the duration of response is evaluated for a population of human subjects treated by a method provided herein by evaluating the median or mean duration of response in the treated population. In one embodiment, a population of the human subjects is treated by a method provided herein, wherein the median or mean duration of response of in the treated population is at least or about 5 months. In another embodiment, a population of the human subjects is treated by a method provided herein, wherein the median or mean duration of response of in the treated population is at least or about 6 months. In a further embodiment, a population of the human subjects is treated by a method provided herein, wherein the median or mean duration of response of in the treated population is at least or about 7 months. In yet another embodiment, a population of the human subjects is treated by a method provided herein, wherein the median or mean duration of response of in the treated population is at least or about 8 months. In one embodiment, a population of the human subjects is treated by a method provided herein, wherein the median or mean duration of response of in the treated population is at least or about 9 months. In another embodiment, a population of the human subjects is treated by a method provided herein, wherein the median or mean duration of response of in the treated population is at least or about 10 months. In yet another embodiment, a population of the human subjects is treated by a method provided herein, wherein the median or mean duration of response of in the treated population is at least or about 11 months. In one embodiment, a population of the human subjects is treated by a method provided herein, wherein the median or mean duration of response of in the treated population is at least or about 12 months. In another embodiment, a population of the human subjects is treated by a method provided herein, wherein the median or mean duration of response of in the treated population is at least or about 13 months. In a further embodiment, a population of the human subjects is treated by a method provided herein, wherein the median or mean duration of response of in the treated population is at least or about 14 months. In yet another embodiment, a population of the human subjects is treated by a method provided herein, wherein the median or mean duration of response of in the treated population is at least or about 15 months. In one embodiment, a population of the human subjects is treated by a method provided herein, wherein the median or mean duration of response of in the treated population is at least or about 16 months. In another embodiment, a population of the human subjects is treated by a method provided herein, wherein the median or mean duration of response of in the treated population is at least or about 17 months. In a further embodiment, a population of the human subjects is treated by a method provided herein, wherein the median or mean duration of response of in the treated population is at least or about 18 months. In yet another embodiment, a population of the human subjects is treated by a method provided herein, wherein the median or mean duration of response of in the treated population is at least or about 19 months. In one embodiment, a population of the human subjects is treated by a method provided herein, wherein the median or mean duration of response of in the treated population is at least or about 20 months.

In certain embodiments, a population of the human subjects is treated by a method provided herein, wherein the duration of response of in the treated population ranges from 5 to 22 months. In some embodiments, a population of the human subjects is treated by a method provided herein, wherein the duration of response of in the treated population ranges from 5 to 21 months. In another embodiment, a population of the human subjects is treated by a method provided herein, wherein the duration of response of in the treated population ranges from 5 to 20 months. In a further embodiment, a population of the human subjects is treated by a method provided herein, wherein the duration of response of in the treated population ranges from 5 to 19 months. In yet another embodiment, a population of the human subjects is treated by a method provided herein, wherein the duration of response of in the treated population ranges from 5 to 18 months. In one embodiment, a population of the human subjects is treated by a method provided herein, wherein the duration of response of in the treated population ranges from 5 to 17 months. In another embodiment, a population of the human subjects is treated by a method provided herein, wherein the duration of response of in the treated population ranges from 5 to 16 months. In a further embodiment, a population of the human subjects is treated by a method provided herein, wherein the duration of response of in the treated population ranges from 5 to 15 months. In yet another embodiment, a population of the human subjects is treated by a method provided herein, wherein the duration of response of in the treated population ranges from 5 to 14 months. In one embodiment, a population of the human subjects is treated by a method provided herein, wherein the duration of response of in the treated population ranges from 5 to 13 months. In another embodiment, a population of the human subjects is treated by a method provided herein, wherein the duration of response of in the treated population ranges from 5 to 12 months. In another embodiment, a population of the human subjects is treated by a method provided herein, wherein the duration of response of in the treated population ranges from 5 to 11 months. In another embodiment, a population of the human subjects is treated by a method provided herein, wherein the duration of response of in the treated population ranges from 5 to 10 months. In another embodiment, a population of the human subjects is treated by a method provided herein, wherein the duration of response of in the treated population ranges from 5 to 9 months. In another embodiment, a population of the human subjects is treated by a method provided herein, wherein the duration of response of in the treated population ranges from 5 to 8 months. In another embodiment, a population of the human subjects is treated by a method provided herein, wherein the duration of response of in the treated population ranges from 5 to 7 months. In a further embodiment, a population of the human subjects is treated by a method provided herein, wherein the duration of response of in the treated population ranges from 6 to 22 months. In some embodiments, a population of the human subjects is treated by a method provided herein, wherein the duration of response of in the treated population ranges from 6 to 21 months. In another embodiment, a population of the human subjects is treated by a method provided herein, wherein the duration of response of in the treated population ranges from 6 to 20 months. In a further embodiment, a population of the human subjects is treated by a method provided herein, wherein the duration of response of in the treated population ranges from 6 to 19 months. In yet another embodiment, a population of the human subjects is treated by a method provided herein, wherein the duration of response of in the treated population ranges from 6 to 18 months. In one embodiment, a population of the human subjects is treated by a method provided herein, wherein the duration of response of in the treated population ranges from 6 to 17 months. In another embodiment, a population of the human subjects is treated by a method provided herein, wherein the duration of response of in the treated population ranges from 6 to 16 months. In a further embodiment, a population of the human subjects is treated by a method provided herein, wherein the duration of response of in the treated population ranges from 6 to 15 months. In yet another embodiment, a population of the human subjects is treated by a method provided herein, wherein the duration of response of in the treated population ranges from 6 to 14 months. In one embodiment, a population of the human subjects is treated by a method provided herein, wherein the duration of response of in the treated population ranges from 6 to 13 months. In another embodiment, a population of the human subjects is treated by a method provided herein, wherein the duration of response of in the treated population ranges from 6 to 12 months. In another embodiment, a population of the human subjects is treated by a method provided herein, wherein the duration of response of in the treated population ranges from 6 to 11 months. In another embodiment, a population of the human subjects is treated by a method provided herein, wherein the duration of response of in the treated population ranges from 6 to 10 months. In another embodiment, a population of the human subjects is treated by a method provided herein, wherein the duration of response of in the treated population ranges from 6 to 9 months. In another embodiment, a population of the human subjects is treated by a method provided herein, wherein the duration of response of in the treated population ranges from 6 to 8 months. In yet another embodiment, a population of the human subjects is treated by a method provided herein, wherein the duration of response of in the treated population ranges from 7 to 22 months. In some embodiments, a population of the human subjects is treated by a method provided herein, wherein the duration of response of in the treated population ranges from 7 to 21 months. In another embodiment, a population of the human subjects is treated by a method provided herein, wherein the duration of response of in the treated population ranges from 7 to 20 months. In a further embodiment, a population of the human subjects is treated by a method provided herein, wherein the duration of response of in the treated population ranges from 7 to 19 months. In yet another embodiment, a population of the human subjects is treated by a method provided herein, wherein the duration of response of in the treated population ranges from 7 to 18 months. In one embodiment, a population of the human subjects is treated by a method provided herein, wherein the duration of response of in the treated population ranges from 7 to 17 months. In another embodiment, a population of the human subjects is treated by a method provided herein, wherein the duration of response of in the treated population ranges from 7 to 16 months. In a further embodiment, a population of the human subjects is treated by a method provided herein, wherein the duration of response of in the treated population ranges from 7 to 15 months. In yet another embodiment, a population of the human subjects is treated by a method provided herein, wherein the duration of response of in the treated population ranges from 7 to 14 months. In one embodiment, a population of the human subjects is treated by a method provided herein, wherein the duration of response of in the treated population ranges from 7 to 13 months. In another embodiment, a population of the human subjects is treated by a method provided herein, wherein the duration of response of in the treated population ranges from 7 to 12 months. In another embodiment, a population of the human subjects is treated by a method provided herein, wherein the duration of response of in the treated population ranges from 7 to 11 months. In another embodiment, a population of the human subjects is treated by a method provided herein, wherein the duration of response of in the treated population ranges from 7 to 10 months. In another embodiment, a population of the human subjects is treated by a method provided herein, wherein the duration of response of in the treated population ranges from 7 to 9 months. In another embodiment, a population of the human subjects is treated by a method provided herein, wherein the duration of response of in the treated population ranges from 8 to 22 months. In a further embodiment, a population of the human subjects is treated by a method provided herein, wherein the duration of response of in the treated population ranges from 9 to 22 months. In yet another embodiment, a population of the human subjects is treated by a method provided herein, wherein the duration of response of in the treated population ranges from 10 to 22 months. In one embodiment, a population of the human subjects is treated by a method provided herein, wherein the duration of response of in the treated population ranges from 11 to 22 months. In another embodiment, a population of the human subjects is treated by a method provided herein, wherein the duration of response of in the treated population ranges from 12 to 12 months. In a further embodiment, a population of the human subjects is treated by a method provided herein, wherein the duration of response of in the treated population ranges from 13 to 22 months. In yet another embodiment, a population of the human subjects is treated by a method provided herein, wherein the duration of response of in the treated population ranges from 14 to 22 months. In one embodiment, a population of the human subjects is treated by a method provided herein, wherein the duration of response of in the treated population ranges from 15 to 22 months. In one embodiment, a population of the human subjects is treated by a method provided herein, wherein the duration of response of in the treated population ranges from 6 to 21 months. In another embodiment, a population of the human subjects is treated by a method provided herein, wherein the duration of response of in the treated population ranges from 7 to 20 months. In a further embodiment, a population of the human subjects is treated by a method provided herein, wherein the duration of response of in the treated population ranges from 8 to 19 months. In yet another embodiment, a population of the human subjects is treated by a method provided herein, wherein the duration of response of in the treated population ranges from 9 to 18 months. In one embodiment, a population of the human subjects is treated by a method provided herein, wherein the duration of response of in the treated population ranges from 10 to 17 months. In another embodiment, a population of the human subjects is treated by a method provided herein, wherein the duration of response of in the treated population ranges from 11 to 16 months. In a further embodiment, a population of the human subjects is treated by a method provided herein, wherein the duration of response of in the treated population ranges from 12 to 15 months.

Alternatively, the therapeutic outcome of the methods provided herein can be evaluated based on the progression free survival as set forth in Section 6.1.8.4. In one embodiment, the human subject has a progression free survival of at least or about 2 months following the treatment. In another embodiment, the human subject has a progression free survival of at least or about 3 months following the treatment. In a further embodiment, the human subject has a progression free survival of at least or about 4 months following the treatment. In yet another embodiment, the human subject has a progression free survival of at least or about 5 months following the treatment. In another embodiment, the human subject has a progression free survival of at least or about 6 months following the treatment. In one embodiment, the human subject has a progression free survival of at least or about 6.7 months following the treatment. In a further embodiment, the human subject has a progression free survival of at least or about 7 months following the treatment. In yet another embodiment, the human subject has a progression free survival of at least or about 8 months following the treatment. In one embodiment, the human subject has a progression free survival of at least or about 9 months following the treatment. In another embodiment, the human subject has a progression free survival of at least or about 10 months following the treatment. In a further embodiment, the human subject has a progression free survival of at least or about 11 months following the treatment. In yet another embodiment, the human subject has a progression free survival of at least or about 12 months following the treatment. In one embodiment, the human subject has a progression free survival of at least or about 13 months following the treatment. In another embodiment, the human subject has a progression free survival of at least or about 14 months following the treatment. In a further embodiment, the human subject has a progression free survival of at least or about 15 months following the treatment. In yet another embodiment, the human subject has a progression free survival of at least or about 16 months following the treatment. In one embodiment, the human subject has a progression free survival of at least or about 17 months following the treatment. In another embodiment, the human subject has a progression free survival of at least or about 18 months following the treatment. In a further embodiment, the human subject has a progression free survival of at least or about 19 months following the treatment. In yet another embodiment, the human subject has a progression free survival of at least or about 20 months following the treatment.

In one embodiment, the human subject has a progression free survival ranging from 5 to 10 months following the treatment. In some embodiments, the human subject has a progression free survival ranging from 5 to 9 months following the treatment. In a further embodiment, the human subject has a progression free survival ranging from 5 to 8 months following the treatment. In yet another embodiment, the human subject has a progression free survival ranging from 5 to 7 months following the treatment. In one embodiment, the human subject has a progression free survival ranging from 5 to 6 months following the treatment. In another embodiment, the human subject has a progression free survival ranging from 6 to 10 months following the treatment. In a further embodiment, the human subject has a progression free survival ranging from 7 to 10 months following the treatment. In yet another embodiment, the human subject has a progression free survival ranging from 8 to 10 months following the treatment. In one embodiment, the human subject has a progression free survival ranging from 9 to 10 months following the treatment. In another embodiment, the human subject has a progression free survival of ranging from 4 to 11 months following the treatment. In a further embodiment, the human subject has a progression free survival ranging from 4 to 10 months following the treatment. In yet another embodiment, the human subject has a progression free survival ranging from 4 to 9 months following the treatment. In one embodiment, the human subject has a progression free survival ranging from 4 to 8 months following the treatment. In another embodiment, the human subject has a progression free survival ranging from 4 to 7 months following the treatment. In a further embodiment, the human subject has a progression free survival ranging from 5 to 11 months following the treatment. In yet another embodiment, the human subject has a progression free survival ranging from 6 to 11 months following the treatment. In one embodiment, the human subject has a progression free survival ranging from 7 to 11 months following the treatment. In another embodiment, the human subject has a progression free survival ranging from 8 to 11 months following the treatment. In a further embodiment, the human subject has a progression free survival ranging from 9 to 11 months following the treatment. In yet another embodiment, the human subject has a progression free survival ranging from 10 to 11 months following the treatment.

In addition, in some embodiments, the progression free survival is evaluated for a population of human subjects treated by a method provided herein by evaluating the median or mean progression free survival in the treated population. In one embodiment, a population of the human subjects is treated by a method provided herein, wherein the median or mean progression free survival in the treated population is at least or about 2 months. In another embodiment, a population of the human subjects is treated by a method provided herein, wherein the median or mean progression free survival in the treated population is at least or about 3 months. In a further embodiment, a population of the human subjects is treated by a method provided herein, wherein the median or mean progression free survival in the treated population is at least or about 4 months. In yet another embodiment, a population of the human subjects is treated by a method provided herein, wherein the median or mean progression free survival in the treated population is at least or about 5 months. In another embodiment, a population of the human subjects is treated by a method provided herein, wherein the median or mean progression free survival in the treated population is at least or about 6 months. In a further embodiment, a population of the human subjects is treated by a method provided herein, wherein the median or mean progression free survival in the treated population is at least or about 7 months. In yet another embodiment, a population of the human subjects is treated by a method provided herein, wherein the median or mean progression free survival in the treated population is at least or about 8 months. In one embodiment, a population of the human subjects is treated by a method provided herein, wherein the median or mean progression free survival in the treated population is at least or about 9 months. In another embodiment, a population of the human subjects is treated by a method provided herein, wherein the median or mean progression free survival in the treated population is at least or about 10 months. In a further embodiment, a population of the human subjects is treated by a method provided herein, wherein the median or mean progression free survival in the treated population is at least or about 11 months. In yet another embodiment, a population of the human subjects is treated by a method provided herein, wherein the median or mean progression free survival in the treated population is at least or about 12 months. In one embodiment, a population of the human subjects is treated by a method provided herein, wherein the median or mean progression free survival in the treated population is at least or about 13 months. In another embodiment, a population of the human subjects is treated by a method provided herein, wherein the median or mean progression free survival in the treated population is at least or about 14 months. In a further embodiment, a population of the human subjects is treated by a method provided herein, wherein the median or mean progression free survival in the treated population is at least or about 15 months. In yet another embodiment, a population of the human subjects is treated by a method provided herein, wherein the median or mean progression free survival in the treated population is at least or about 16 months. In one embodiment, a population of the human subjects is treated by a method provided herein, wherein the median or mean progression free survival in the treated population is at least or about 17 months. In another embodiment, a population of the human subjects is treated by a method provided herein, wherein the median or mean progression free survival in the treated population is at least or about 18 months. In a further embodiment, a population of the human subjects is treated by a method provided herein, wherein the median or mean progression free survival in the treated population is at least or about 19 months. In yet another embodiment, a population of the human subjects is treated by a method provided herein, wherein the median or mean progression free survival in the treated population is at least or about 20 months.

In one embodiment, a population of the human subjects is treated by a method provided herein, wherein the progression free survival in the treated population ranges from 5 to 9 months. In another embodiment, a population of the human subjects is treated by a method provided herein, wherein the progression free survival in the treated population ranges from 5 to 8 months. In a further embodiment, a population of the human subjects is treated by a method provided herein, wherein the progression free survival in the treated population ranges from 5 to 7 months. In yet another embodiment, a population of the human subjects is treated by a method provided herein, wherein the progression free survival in the treated population ranges from 5 to 6 months. In one embodiment, a population of the human subjects is treated by a method provided herein, wherein the progression free survival in the treated population ranges from 6 to 9 months. In another embodiment, a population of the human subjects is treated by a method provided herein, wherein the progression free survival in the treated population ranges from 7 to 9 months. In a further embodiment, a population of the human subjects is treated by a method provided herein, wherein the progression free survival in the treated population ranges from 8 to 9 months. In yet another embodiment, a population of the human subjects is treated by a method provided herein, wherein the progression free survival in the treated population ranges from 4 to 10 months. In one embodiment, a population of the human subjects is treated by a method provided herein, wherein the progression free survival in the treated population ranges from 5 to 10 months. In another embodiment, a population of the human subjects is treated by a method provided herein, wherein the progression free survival in the treated population ranges from 6 to 10 months. In a further embodiment, a population of the human subjects is treated by a method provided herein, wherein the progression free survival in the treated population ranges from 7 to 10 months. In yet another embodiment, a population of the human subjects is treated by a method provided herein, wherein the progression free survival in the treated population ranges from 8 to 10 months. In one embodiment, a population of the human subjects is treated by a method provided herein, wherein the progression free survival in the treated population ranges from 9 to 10 months. In another embodiment, a population of the human subjects is treated by a method provided herein, wherein the progression free survival in the treated population ranges from 4 to 10 months. In another embodiment, a population of the human subjects is treated by a method provided herein, wherein the progression free survival in the treated population ranges from 4 to 9 months. In a further embodiment, a population of the human subjects is treated by a method provided herein, wherein the progression free survival in the treated population ranges from 4 to 8 months. In yet another embodiment, a population of the human subjects is treated by a method provided herein, wherein the progression free survival in the treated population ranges from 4 to 7 months. In one embodiment, a population of the human subjects is treated by a method provided herein, wherein the progression free survival in the treated population ranges from 4 to 6 months. In another embodiment, a population of the human subjects is treated by a method provided herein, wherein the progression free survival in the treated population ranges from 4 to 5 months. In a further embodiment, a population of the human subjects is treated by a method provided herein, wherein the progression free survival in the treated population ranges from 4 to 11 months. In yet another embodiment, a population of the human subjects is treated by a method provided herein, wherein the progression free survival in the treated population ranges from 5 to 11 months. In another embodiment, a population of the human subjects is treated by a method provided herein, wherein the progression free survival in the treated population ranges from 6 to 11 months. In a further embodiment, a population of the human subjects is treated by a method provided herein, wherein the progression free survival in the treated population ranges from 7 to 11 months. In yet another embodiment, a population of the human subjects is treated by a method provided herein, wherein the progression free survival in the treated population ranges from 8 to 11 months. In a further embodiment, a population of the human subjects is treated by a method provided herein, wherein the progression free survival in the treated population ranges from 9 to 11 months. In yet another embodiment, a population of the human subjects is treated by a method provided herein, wherein the progression free survival in the treated population ranges from 10 to 11 months.

Alternatively, the therapeutic outcome of the methods provided herein can be evaluated based on the overall survival. In one embodiment, the human subject has an overall survival of at least or about 5 months following the treatment. In another embodiment, the human subject has an overall survival of at least or about 6 months following the treatment. In a further embodiment, the human subject has an overall survival of at least or about 7 months following the treatment. In yet another embodiment, the human subject has an overall survival of at least or about 8 months following the treatment. In one embodiment, the human subject has an overall survival of at least or about 9 months following the treatment. In another embodiment, the human subject has an overall survival of at least or about 10 months following the treatment. In a further embodiment, the human subject has an overall survival of at least or about 11 months following the treatment. In yet another embodiment, the human subject has an overall survival of at least or about 12 months following the treatment. In one embodiment, the human subject has an overall survival of at least or about 13 months following the treatment. In another embodiment, the human subject has an overall survival of at least or about 14 months following the treatment. In yet another embodiment, the human subject has an overall survival of at least or about 15 months following the treatment. In one embodiment, the human subject has an overall survival of at least or about 16 months following the treatment. In another embodiment, the human subject has an overall survival of at least or about 17 months following the treatment. In a further embodiment, the human subject has an overall survival of at least or about 18 months following the treatment. In yet another embodiment, the human subject has an overall survival of at least or about 19 months following the treatment. In one embodiment, the human subject has an overall survival of at least or about 20 months following the treatment. In another embodiment, the human subject has an overall survival of at least or about 21 months following the treatment. In a further embodiment, the human subject has an overall survival of at least or about 22 months following the treatment. In a further embodiment, the human subject has an overall survival of at least or about 23 months following the treatment. In yet another embodiment, the human subject has an overall survival of at least or about 24 months following the treatment. In one embodiment, the human subject has an overall survival of at least or about 25 months following the treatment. In another embodiment, the human subject has an overall survival of at least or about 26 months following the treatment. In a further embodiment, the human subject has an overall survival of at least or about 27 months following the treatment. In one embodiment, the human subject has an overall survival of at least or about 28 months following the treatment. In another embodiment, the human subject has an overall survival of at least or about 29 months following the treatment. In a further embodiment, the human subject has an overall survival of at least or about 30 months following the treatment.

In one embodiment, the human subject has an overall survival ranging from 10 to 19 months following the treatment. In another embodiment, the human subject has an overall survival ranging from 10 to 18 months following the treatment. In a further embodiment, the human subject has an overall survival ranging from 10 to 17 months following the treatment. In yet another embodiment, the human subject has an overall survival ranging from 10 to 16 months following the treatment. In one embodiment, the human subject has an overall survival ranging from 10 to 15 months following the treatment. In another embodiment, the human subject has an overall survival ranging from 10 to 14 months following the treatment. In a further embodiment, the human subject has an overall survival ranging from 10 to 13 months following the treatment. In yet another embodiment, the human subject has an overall survival ranging from 10 to 12 months following the treatment. In one embodiment, the human subject has an overall survival ranging from 10 to 11 months following the treatment. In another embodiment, the human subject has an overall survival ranging from 11 to 19 months following the treatment. In a further embodiment, the human subject has an overall survival ranging from 12 to 19 months following the treatment. In yet another embodiment, the human subject has an overall survival ranging from 13 to 19 months following the treatment. In one embodiment, the human subject has an overall survival ranging from 14 to 18 months following the treatment. In one embodiment, the human subject has an overall survival ranging from 14 to 19 months following the treatment. In one embodiment, the human subject has an overall survival ranging from 15 to 18 months following the treatment. In another embodiment, the human subject has an overall survival ranging from 15 to 19 months following the treatment. In a further embodiment, the human subject has an overall survival ranging from 16 to 19 months following the treatment. In yet another embodiment, the human subject has an overall survival ranging from 17 to 19 months following the treatment. In one embodiment, the human subject has an overall survival ranging from 18 to 19 months following the treatment. In another embodiment, the human subject has an overall survival ranging from 11 to 18 months following the treatment. In a further embodiment, the human subject has an overall survival ranging from 12 to 17 months following the treatment. In a further embodiment, the human subject has an overall survival ranging from 13 to 16 months following the treatment. In yet another embodiment, the human subject has an overall survival ranging from 14 to 15 months following the treatment. In one embodiment, the human subject has an overall survival ranging from 10 to 20 months following the treatment. In another embodiment, the human subject has an overall survival ranging from 11 to 20 months following the treatment. In one embodiment, the human subject has an overall survival ranging from 11 to 24 months following the treatment. In one embodiment, the human subject has an overall survival ranging from 11 to 25 months following the treatment In one embodiment, the human subject has an overall survival ranging from 12 to 24 months following the treatment. In one embodiment, the human subject has an overall survival ranging from 12 to 25 months following the treatment. In a further embodiment, the human subject has an overall survival ranging from 12 to 20 months following the treatment. In one embodiment, the human subject has an overall survival ranging from 13 to 20 months following the treatment. In another embodiment, the human subject has an overall survival ranging from 14 to 20 months following the treatment. In a further embodiment, the human subject has an overall survival ranging from 15 to 20 months following the treatment. In yet another embodiment, the human subject has an overall survival ranging from 16 to 20 months following the treatment. In one embodiment, the human subject has an overall survival ranging from 17 to 20 months following the treatment. In another embodiment, the human subject has an overall survival ranging from 18 to 20 months following the treatment. In a further embodiment, the human subject has an overall survival ranging from 19 to 20 months following the treatment. In one embodiment, the human subject has an overall survival ranging from 9 to 20 months following the treatment. In another embodiment, the human subject has an overall survival ranging from 9 to 19 months following the treatment. In a further embodiment, the human subject has an overall survival ranging from 9 to 18 months following the treatment. In one embodiment, the human subject has an overall survival ranging from 9 to 17 months following the treatment. In another embodiment, the human subject has an overall survival ranging from 9 to 16 months following the treatment. In a further embodiment, the human subject has an overall survival ranging from 9 to 15 months following the treatment. In one embodiment, the human subject has an overall survival ranging from 9 to 14 months following the treatment. In another embodiment, the human subject has an overall survival ranging from 9 to 13 months following the treatment. In a further embodiment, the human subject has an overall survival ranging from 9 to 12 months following the treatment. In one embodiment, the human subject has an overall survival ranging from 9 to 11 months following the treatment. In another embodiment, the human subject has an overall survival ranging from 9 to 10 months following the treatment.

Additionally, in some embodiments, the overall survival is evaluated for a population of human subjects treated by a method provided herein by evaluating the median or mean overall survival in the treated population. In one embodiment, a population of the human subjects is treated by a method provided herein, wherein the median or mean overall survival in the treated population is at least or about 5 months. In another embodiment, a population of the human subjects is treated by a method provided herein, wherein the median or mean overall survival in the treated population is at least or about 6 months. In a further embodiment, a population of the human subjects is treated by a method provided herein, wherein the median or mean overall survival in the treated population is at least or about 7 months. In yet another embodiment, a population of the human subjects is treated by a method provided herein, wherein the median or mean overall survival in the treated population is at least or about 8 months. In one embodiment, a population of the human subjects is treated by a method provided herein, wherein the median or mean overall survival in the treated population is at least or about 9 months. In another embodiment, a population of the human subjects is treated by a method provided herein, wherein the median or mean overall survival in the treated population is at least or about 10 months. In a further embodiment, a population of the human subjects is treated by a method provided herein, wherein the median or mean overall survival in the treated population is at least or about 11 months. In yet another embodiment, a population of the human subjects is treated by a method provided herein, wherein the median or mean overall survival in the treated population is at least or about 12 months. In one embodiment, a population of the human subjects is treated by a method provided herein, wherein the median or mean overall survival in the treated population is at least or about 13 months. In another embodiment, a population of the human subjects is treated by a method provided herein, wherein the median or mean overall survival in the treated population is at least or about 14 months. In yet another embodiment, a population of the human subjects is treated by a method provided herein, wherein the median or mean overall survival in the treated population is at least or about 15 months. In one embodiment, a population of the human subjects is treated by a method provided herein, wherein the median or mean overall survival in the treated population is at least or about 16 months. In another embodiment, a population of the human subjects is treated by a method provided herein, wherein the median or mean overall survival in the treated population is at least or about 17 months. In a further embodiment, a population of the human subjects is treated by a method provided herein, wherein the median or mean overall survival in the treated population is at least or about 18 months. In yet another embodiment, a population of the human subjects is treated by a method provided herein, wherein the median or mean overall survival in the treated population is at least or about 19 months. In one embodiment, a population of the human subjects is treated by a method provided herein, wherein the median or mean overall survival in the treated population is at least or about 20 months. In another embodiment, a population of the human subjects is treated by a method provided herein, wherein the median or mean overall survival in the treated population is at least or about 21 months. In a further embodiment, a population of the human subjects is treated by a method provided herein, wherein the median or mean overall survival in the treated population is at least or about 22 months. In yet another embodiment, a population of the human subjects is treated by a method provided herein, wherein the median or mean overall survival in the treated population is at least or about 23 months. In one embodiment, a population of the human subjects is treated by a method provided herein, wherein the median or mean overall survival in the treated population is at least or about 24 months. In another embodiment, a population of the human subjects is treated by a method provided herein, wherein the median or mean overall survival in the treated population is at least or about 25 months. In a further embodiment, a population of the human subjects is treated by a method provided herein, wherein the median or mean overall survival in the treated population is at least or about 26 months. In yet another embodiment, a population of the human subjects is treated by a method provided herein, wherein the median or mean overall survival in the treated population is at least or about 27 months. In one embodiment, a population of the human subjects is treated by a method provided herein, wherein the median or mean overall survival in the treated population is at least or about 28 months. In another embodiment, a population of the human subjects is treated by a method provided herein, wherein the median or mean overall survival in the treated population is at least or about 29 months. In a further embodiment, a population of the human subjects is treated by a method provided herein, wherein the median or mean overall survival in the treated population is at least or about 30 months.

In one embodiment, a population of the human subjects is treated by a method provided herein, wherein the overall survival in the treated population ranges from 10 to 19 months. In another embodiment, a population of the human subjects is treated by a method provided herein, wherein the overall survival in the treated population ranges from 10 to 18 months. In a further embodiment, a population of the human subjects is treated by a method provided herein, wherein the overall survival in the treated population ranges from 10 to 17 months. In yet another embodiment, a population of the human subjects is treated by a method provided herein, wherein the overall survival in the treated population ranges from 10 to 16 months. In one embodiment, a population of the human subjects is treated by a method provided herein, wherein the overall survival in the treated population ranges from 10 to 15 months. In another embodiment, a population of the human subjects is treated by a method provided herein, wherein the overall survival in the treated population ranges from 10 to 14 months. In a further embodiment, a population of the human subjects is treated by a method provided herein, wherein the overall survival in the treated population ranges from 10 to 13 months. In yet another embodiment, a population of the human subjects is treated by a method provided herein, wherein the overall survival in the treated population ranges from 10 to 12 months. In one embodiment, a population of the human subjects is treated by a method provided herein, wherein the overall survival in the treated population ranges from 10 to 11 months. In another embodiment, a population of the human subjects is treated by a method provided herein, wherein the overall survival in the treated population ranges from 10 to 19 months. In a further embodiment, a population of the human subjects is treated by a method provided herein, wherein the overall survival in the treated population ranges from 11 to 19 months. In one embodiment, a population of the human subjects is treated by a method provided herein, wherein the overall survival in the treated population ranges from 11 to 24 months. In a further embodiment, a population of the human subjects is treated by a method provided herein, wherein the overall survival in the treated population ranges from 11 to 25 months. In a further embodiment, a population of the human subjects is treated by a method provided herein, wherein the overall survival in the treated population ranges from 12 to 24 months. In one embodiment, a population of the human subjects is treated by a method provided herein, wherein the overall survival in the treated population ranges from 12 to 25 months. In yet another embodiment, a population of the human subjects is treated by a method provided herein, wherein the overall survival in the treated population ranges from 12 to 19 months. In one embodiment, a population of the human subjects is treated by a method provided herein, wherein the overall survival in the treated population ranges from 13 to 19 months. In another embodiment, a population of the human subjects is treated by a method provided herein, wherein the overall survival in the treated population ranges from 14 to 19 months. In a further embodiment, a population of the human subjects is treated by a method provided herein, wherein the overall survival in the treated population ranges from 15 to 19 months. In yet another embodiment, a population of the human subjects is treated by a method provided herein, wherein the overall survival in the treated population ranges from 16 to 19 months. In one embodiment, a population of the human subjects is treated by a method provided herein, wherein the overall survival in the treated population ranges from 17 to 19 months. In another embodiment, a population of the human subjects is treated by a method provided herein, wherein the overall survival in the treated population ranges from 18 to 19 months. In a further embodiment, a population of the human subjects is treated by a method provided herein, wherein the overall survival in the treated population ranges from 11 to 18 months. In yet another embodiment, a population of the human subjects is treated by a method provided herein, wherein the overall survival in the treated population ranges from 12 to 17 months. In one embodiment, a population of the human subjects is treated by a method provided herein, wherein the overall survival in the treated population ranges from 13 to 16 months. In another embodiment, a population of the human subjects is treated by a method provided herein, wherein the overall survival in the treated population ranges from 14 to 15 months. In a further embodiment, a population of the human subjects is treated by a method provided herein, wherein the overall survival in the treated population ranges from 10 to 20 months. In yet another embodiment, a population of the human subjects is treated by a method provided herein, wherein the overall survival in the treated population ranges from 11 to 20 months. In one embodiment, a population of the human subjects is treated by a method provided herein, wherein the overall survival in the treated population ranges from 12 to 20 months. In another embodiment, a population of the human subjects is treated by a method provided herein, wherein the overall survival in the treated population ranges from 13 to 20 months. In a further embodiment, a population of the human subjects is treated by a method provided herein, wherein the overall survival in the treated population ranges from 14 to 20 months. In yet another embodiment, a population of the human subjects is treated by a method provided herein, wherein the overall survival in the treated population ranges from 15 to 20 months. In one embodiment, a population of the human subjects is treated by a method provided herein, wherein the overall survival in the treated population ranges from 16 to 20 months. In another embodiment, a population of the human subjects is treated by a method provided herein, wherein the overall survival in the treated population ranges from 17 to 20 months. In a further embodiment, a population of the human subjects is treated by a method provided herein, wherein the overall survival in the treated population ranges from 18 to 20 months. In yet another embodiment, a population of the human subjects is treated by a method provided herein, wherein the overall survival in the treated population ranges from 19 to 20 months. In one embodiment, a population of the human subjects is treated by a method provided herein, wherein the overall survival in the treated population ranges from 9 to 20 months. In another embodiment, a population of the human subjects is treated by a method provided herein, wherein the overall survival in the treated population ranges from 9 to 19 months. In a further embodiment, a population of the human subjects is treated by a method provided herein, wherein the overall survival in the treated population ranges from 9 to 18 months. In yet another embodiment, a population of the human subjects is treated by a method provided herein, wherein the overall survival in the treated population ranges from 9 to 17 months. In one embodiment, a population of the human subjects is treated by a method provided herein, wherein the overall survival in the treated population ranges from 9 to 16 months. In another embodiment, a population of the human subjects is treated by a method provided herein, wherein the overall survival in the treated population ranges from 9 to 15 months. In a further embodiment, a population of the human subjects is treated by a method provided herein, wherein the overall survival in the treated population ranges from 9 to 14 months. In yet another embodiment, a population of the human subjects is treated by a method provided herein, wherein the overall survival in the treated population ranges from 9 to 13 months. In one embodiment, a population of the human subjects is treated by a method provided herein, wherein the overall survival in the treated population ranges from 9 to 12 months. In another embodiment, a population of the human subjects is treated by a method provided herein, wherein the overall survival in the treated population ranges from 9 to 11 months. In a further embodiment, a population of the human subjects is treated by a method provided herein, wherein the overall survival in the treated population ranges from 9 to 10 months.

### 5.2.2 Methods of Treating Cancer in Selected Patient Populations

Provided herein are methods for the treatment of various cancers in subjects, wherein the cancers have any of the suitable markers and/or characteristics as provided in Section 6. Also provided herein are methods for the treatment of various cancers in subjects, wherein the subjects have any of the suitable characteristics as provided in Section 6.

In one aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has any of the suitable characteristics as provided in Section 6.

In some aspects, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the cancer has any of the suitable markers and/or characteristics as provided in Section 6.

In some aspects, provided herein is a method of treating urothelial or bladder cancer in a human subject having liver metastases, comprising administering to the subject having liver metastases an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the subject has received an immune checkpoint inhibitor (CPI) therapy, and wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has any of the suitable characteristics as provided in Section 6.

In some aspects, provided herein is a method of treating urothelial or bladder cancer in a human subject having liver metastases, comprising administering to the subject having liver metastases an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the subject has received an immune checkpoint inhibitor (CPI) therapy, and wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the urothelial or bladder cancer has any of the suitable markers and/or characteristics as provided in Section 6.

In some aspects, provided herein is a method of treating urothelial or bladder cancer in a human subject having a primary site of tumor in the upper urinary tract, comprising administering to the subject having a primary site of tumor in the upper urinary tract an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the subject has received an immune checkpoint inhibitor (CPI) therapy, and wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has any of the suitable characteristics as provided in Section 6.

In some aspects, provided herein is a method of treating urothelial or bladder cancer in a human subject having a primary site of tumor in the upper urinary tract, comprising administering to the subject having a primary site of tumor in the upper urinary tract an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the subject has received an immune checkpoint inhibitor (CPI) therapy, and wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the urothelial or bladder cancer has any of the suitable markers and/or characteristics as provided in Section 6.

In some aspects, provided herein is a method of treating urothelial or bladder cancer in a human subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the subject has received an immune checkpoint inhibitor (CPI) therapy, wherein the subject had progression or recurrence of the cancer during or following the CPI therapy, and wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has any of the suitable characteristics as provided in Section 6.

In some aspects, provided herein is a method of treating urothelial or bladder cancer in a human subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the subject has received an immune checkpoint inhibitor (CPI) therapy, wherein the subject had progression or recurrence of the cancer during or following the CPI therapy, and wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the urothelial or bladder cancer has any of the suitable markers and/or characteristics as provided in Section 6.

In all the methods provided herein and specifically those described in the Sections 5.2.1 and 5.2.2: the therapeutic agents that can be used are described in Section 5.3 and Section 6, selection of patients for treatment is described herein and exemplified in Section 5.2 including Section 5.2.2 and Section 6, dosing regimens and pharmaceutical composition for administering the therapeutic agent are described in Section 5.4, 5.6, 5.7, and Section 6 below, the biomarkers that can be used for identifying the therapeutic agents, selecting the patients, determining the outcome of these methods, and/or serving as criteria in any way for these methods are described herein and exemplified in Section 5.2 including Section 5.2.2 and Section 6, therapeutic outcomes for the methods provided herein can be improvement of the biomarkers described herein, for example, those described and exemplified in in Section 5.2 including Section 5.2.2 and Section 6. Therefore, a person skilled in the art would understand that the methods provided herein include all permutations and combinations of the patients, therapeutic agents, dosing regiments, biomarkers, and therapeutic outcomes as described above and below.

### 5.3 Antibody Drug Conjugates for the Methods

In various embodiments of the methods provided herein, including the methods provided in Section 5.2, the ADC used in the methods comprises or is an anti-191P4D12 ADC described herein and/or in U.S. Pat. No. 8,637,642, which is herein incorporated in its entirety by reference. In some embodiments, the anti-191P4D12 antibody drug conjugate provided for the methods herein comprises an antibody or antigen binding fragment thereof that binds to 191P4D12 as provided herein, including in Subsection 5.3.1, conjugated to one or more units of cytotoxic agents (drug units, or D) as provided herein, including in this Section (Section 5.3) with further disclosures in Subsection 5.3.2. In certain embodiments, the cytotoxic agents (drug units, or D) can be covalently linked directly or via a linker unit (LU).

In some embodiments, the antibody drug conjugate compound has the following formula:

L-(LU-D)ₚ (I)
or a pharmaceutically acceptable salt or solvate thereof; wherein:
L is the antibody unit, e.g., the anti-nectin-4 antibody or an antigen binding fragment thereof as provided in Subsection 5.3.1 below, and
(LU-D) is a linker unit-drug unit moiety, wherein:
LU- is a linker unit, and
D is a drug unit having cytostatic or cytotoxic activity against a target cell; and
p is an integer from 1 to 20.

In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20.

In some embodiments, the antibody drug conjugate compound has the following formula:

L-(Aₐ-W_{w}-Y_{y}-D)ₚ (II)
or a pharmaceutically acceptable salt or solvate thereof, wherein:
L is the Antibody unit, e.g., the anti-nectin-4 antibody or an antigen binding fragment thereof as provided in Subsection 5.3.1 below; and
-Aₐ-W_{w}-Y_{y}- is a linker unit (LU), wherein:
-A- is a stretcher unit,
a is 0 or 1,
each -W- is independently an amino acid unit,
w is an integer ranging from 0 to 12,
-Y- is a self-immolative spacer unit,
y is 0, 1 or 2;
D is a drug units having cytostatic or cytotoxic activity against the target cell; and
p is an integer from 1 to 20.

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

In some specific embodiments of the methods provided herein, including the methods provided in Section 5.2, the cytotoxic agent as part of any of the ADCs provided herein for the methods comprises, consists of, or is MMAE.

For compositions comprising a plurality antibodies or antigen binding fragments thereof, the drug loading is represented by p, the average number of drug molecules per antibody unit. Drug loading can range from 1 to 20 drugs (D) per antibody. The average number of drugs per antibody in preparation of conjugation reactions can be characterized by conventional means such as mass spectroscopy, ELISA assay, and HPLC. The quantitative distribution of antibody drug conjugates in terms of p can also be determined. In some instances, separation, purification, and characterization of homogeneous antibody drug conjugates where p is a certain value from antibody drug conjugates with other drug loadings can be achieved by means such as reverse phase HPLC or electrophoresis. In exemplary embodiments, p is from 2 to 8.

Additional embodiments of the ADC for the methods provided herein have been described in U.S. Pat. No. 8,637,642 and International Application No. PCT/US2019/056214 (Publication No. WO2020/117373), both of which are hereby incorporated in their entireties by reference.

### 5.3.1 Anti-191P4D12 Antibodies or Antigen Binding Fragments

In one embodiment, the antibody or antigen binding fragment thereof that binds to nectin-4-related proteins is an antibody or antigen binding fragment that specifically binds to nectin-4 protein comprising amino acid sequence of SEQ ID NO:2 (see FIG. 1A). The corresponding cDNA encoding the 191P4D12 protein has a sequence of SEQ ID NO:1 (see FIG. 1A).

The antibody that specifically binds to nectin-4 protein comprising amino acid sequence of SEQ ID NO:2 includes antibodies that can bind to other nectin-4-related proteins. For example, antibodies that bind nectin-4 protein comprising amino acid sequence of SEQ ID NO:2 can bind nectin-4-related proteins such as nectin-4 variants and the homologs or analogs thereof.

In some embodiments, the anti-nectin-4 antibody provided herein is a monoclonal antibody.

In some embodiments, the antibody comprises a heavy chain comprising an amino acid sequence of SEQ ID NO:4 (cDNA sequence of SEQ ID NO:3), and/or a light chain comprising an amino acid sequence of SEQ ID NO: 6 (cDNA sequence of SEQ ID NO:5), as shown in FIGS. 1B and 1C.

In some embodiments, the anti-nectin-4 antibody or antigen binding fragment thereof comprises a heavy chain variable region comprising complementarity determining regions (CDRs) comprising the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs comprising the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). In some embodiments, the anti-nectin-4 antibody or antigen binding fragment thereof comprises a heavy chain variable region comprising complementarity determining regions (CDRs) consisting of the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs consisting of the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 consisting of the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 consisting of the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). SEQ ID NO: 22, SEQ ID NO:23, SEQ ID NO:7 and SEQ ID NO:8 are as shown in FIGS. 1D and 1E and listed below:

SEQ ID NO: 22
EVQLVESGGGLVQPGGSLRLSCAASGFTFSSYNMNWVRQAPGKGLEWVS
YISSSSSTIYYADSVKGRFTISRDNAKNSLSLQMNSLRDEDTAVYYCAR
AYYYGMDVWGQGTTVTVSS
SEQ ID NO: 23
DIQMTQSPSSVSASVGDRVTITCRASQGISGWLAWYQQKPGKAPKFLIY
AASTLQSGVPSRFSGSGSGTDFTLTISSLQPEDFATYYCQQANSFPPTF
GGGTKVEIKR
SEQ ID NO: 7
MELGLCWVFLVAILEGVQCEVQLVESGGGLVQPGGSLRLSCAASGFTFS
SYNMNWVRQAPGKGLEWVSYISSSSSTIYYADSVKGRFTISRDNAKNSL
SLQMNSLRDEDTAVYYCARAYYYGMDVWGQGTTVTVSSASTKGPSVFPL
APSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGK
SEQ ID NO: 8
MDMRVPAQLLGLLLLWFPGSRCDIQMTQSPSSVSASVGDRVTITCRASQ
GISGWLAWYQQKPGKAPKFLIYAASTLQSGVPSRFSGSGSGTDFTLTIS
SLQPEDFATYYCQQANSFPPTFGGGTKVEIKRTVAAPSVFIFPPSDEQL
KSGTASVVCLLNNFYPREAKVQWKVDNALQSGNSQESVTEQDSKDSTYS
LSSTLTLSKADYEKHKVYACEVTHQGLSSPVTKSFNRGEC

CDR sequences can be determined according to well-known numbering systems. As described above, CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted in Table 1 above.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Kabat numbering and a light chain variable region comprising CDRs comprising the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to AbM numbering and a light chain variable region comprising CDRs comprising the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Chothia numbering and a light chain variable region comprising CDRs comprising the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Contact numbering and a light chain variable region comprising CDRs comprising the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to IMGT numbering and a light chain variable region comprising CDRs comprising the amino acid sequences of the CDRs of the light chain variable region set forth in SEQ ID NO:23 according to IMGT numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Kabat numbering and a light chain variable region comprising CDRs consisting of the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to AbM numbering and a light chain variable region comprising CDRs consisting of the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Chothia numbering and a light chain variable region comprising CDRs consisting of the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Contact numbering and a light chain variable region comprising CDRs consisting of the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to IMGT numbering and a light chain variable region comprising CDRs consisting of the amino acid sequences of the CDRs of the light chain variable region set forth in SEQ ID NO:23 according to IMGT numbering.

As described above, the CDR sequences according to different numbering systems can be readily determined, e.g., using online tools such as the one provided by Antigen receptor Numbering And Receptor ClassificatIon (ANARCI). For example, the heavy chain CDR sequences within SEQ ID NO:22, and the light chain CDR sequences within SEQ ID NO:23 according to Kabat numbering as determined by ANARCI are listed in Table 4 below.

TABLE 4

| | VH of | VL of |
|---|---|---|
| | SEQ ID NO: 22 | SEQ ID NO: 23 |
| CDR1 | SYNMN | RASQGISGWLA |
| | (SEQ ID NO: 9) | (SEQ ID NO: 12) |
| CDR2 | YISSSSSTIYYADSVKG | AASTLQS |
| | (SEQ ID NO: 10) | (SEQ ID NO: 13) |
| CDR3 | AYYYGMDV | QQANSFPPT |
| | (SEQ ID NO: 11) | (SEQ ID NO: 14) |

For another example, the heavy chain CDR sequences within SEQ ID NO:22, and the light chain CDR sequences within SEQ ID NO:23 according to IMGT numbering as determined by ANARCI are listed in Table 5 below.

TABLE 5

| | VH of | VL of |
|---|---|---|
| | SEQ ID NO: 22 | SEQ ID NO: 23 |
| CDR1 | GFTFSSYN | QGISGW |
| | (SEQ ID NO: 16) | (SEQ ID NO: 19) |
| CDR2 | ISSSSSTI | AAS |
| | (SEQ ID NO: 17) | (SEQ ID NO: 20) |
| CDR3 | ARAYYYGMDV | QQANSFPPT |
| | (SEQ ID NO: 18) | (SEQ ID NO: 21) |

In some embodiments, the antibody or antigen binding fragment thereof comprises CDR-H1 comprising an amino acid sequence of SEQ ID NO:9, CDR-H2 comprising an amino acid sequence of SEQ ID NO:10, CDR-H3 comprising an amino acid sequence of SEQ ID NO:11, CDR-L1 comprising an amino acid sequence of SEQ ID NO:NO:12, CDR-L2 comprising an amino acid sequence of SEQ ID NO:NO:13, and CDR-L3 comprising an amino acid sequence of SEQ ID NO:NO:14.

In some embodiments, the antibody or antigen binding fragment thereof comprises CDR-H1 comprising an amino acid sequence of SEQ ID NO:16, CDR-H2 comprising an amino acid sequence of SEQ ID NO:17, CDR-H3 comprising an amino acid sequence of SEQ ID NO:18, CDR-L1 comprising an amino acid sequence of SEQ ID NO:NO:19, CDR-L2 comprising an amino acid sequence of SEQ ID NO:NO:20, and CDR-L3 comprising an amino acid sequence of SEQ ID NO:NO:21.

In some embodiments, the antibody or antigen binding fragment thereof comprises CDR-H1 consisting of an amino acid sequence of SEQ ID NO:9, CDR-H2 consisting of an amino acid sequence of SEQ ID NO:10, CDR-H3 consisting of an amino acid sequence of SEQ ID NO:11, CDR-L1 consisting of an amino acid sequence of SEQ ID NO:NO:12, CDR-L2 consisting of an amino acid sequence of SEQ ID NO:NO:13, and CDR-L3 consisting of an amino acid sequence of SEQ ID NO:NO:14.

In some embodiments, the antibody or antigen binding fragment thereof comprises CDR-H1 consisting of an amino acid sequence of SEQ ID NO:16, CDR-H2 consisting of an amino acid sequence of SEQ ID NO:17, CDR-H3 consisting of an amino acid sequence of SEQ ID NO:18, CDR-L1 consisting of an amino acid sequence of SEQ ID NO:NO:19, CDR-L2 consisting of an amino acid sequence of SEQ ID NO:NO:20, and CDR-L3 consisting of an amino acid sequence of SEQ ID NO:NO:21.

In some embodiments, the antibody or antigen binding fragment thereof comprises a heavy chain variable region comprising the amino acid sequence of SEQ ID NO:22 and a light chain variable region comprising the amino acid sequence of SEQ ID NO:23.

In some embodiments, the antibody or antigen binding fragment thereof comprises a heavy chain variable region consisting of the amino acid sequence of SEQ ID NO:22 and a light chain variable region consisting of the amino acid sequence of SEQ ID NO:23.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, the antibody comprises a heavy chain consisting of the amino acid sequence ranging from the 20th amino acid (glutamic acid) to the 466th amino acid (lysine) of SEQ ID NO:7 and a light chain consisting of the amino acid sequence ranging from the 23rd amino acid (aspartic acid) to the 236th amino acid (cysteine) of SEQ ID NO:8.

In some embodiments, amino acid sequence modification(s) of antibodies described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes can alter post-translational processes of the antibody, such as changing the number or position of glycosylation sites or altering the membrane anchoring characteristics.

In some embodiments, the antibodies provided herein are chemically modified, for example, by the covalent attachment of any type of molecule to the antibody. The antibody derivatives can include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formulation, metabolic synthesis of tunicamycin, etc. Additionally, the antibody can contain one or more non-classical amino acids.

Variations can be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid comprising similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions can optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed can be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue comprising a side chain with a similar charge. As described above, families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined conservative (e.g., within an amino acid group with similar properties and/or side chains) substitutions can be made, so as to maintain or not significantly change the properties.

Amino acids can be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also can be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al, 1982, Nucl. Acids Res. 10:6487-500), cassette mutagenesis (see, e.g., Wells et al., 1985, Gene 34:315-23), or other known techniques can be performed on the cloned DNA to produce the anti-anti-MSLN antibody variant DNA.

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In certain embodiments, the antibody or antigen binding fragment provided herein comprises a heavy chain having certain homology or identity to the heavy chain as set forth in SEQ ID NO:7 and a light chain having certain homology or identity to the light chain as set forth in SEQ ID NO:8. Such embodiments of heavy/light chains with homology or identity are further provided as follows. In some embodiments, the antibody or antigen binding fragment provided herein comprises a heavy chain having more than 70% homology or identity to the heavy chain as set forth in SEQ ID NO:7. In some embodiments, the antibody or antigen binding fragment provided herein comprises a heavy chain having more than 75% homology or identity to the heavy chain as set forth in SEQ ID NO:7. In some embodiments, the antibody or antigen binding fragment provided herein comprises a heavy chain having more than 80% homology or identity to the heavy chain as set forth in SEQ ID NO:7. In some embodiments, the antibody or antigen binding fragment provided herein comprises a heavy chain having more than 85% homology or identity to the heavy chain as set forth in SEQ ID NO:7. In some embodiments, the antibody or antigen binding fragment provided herein comprises a heavy chain having more than 90% homology or identity to the heavy chain as set forth in SEQ ID NO:7. In some embodiments, the antibody or antigen binding fragment provided herein comprises a heavy chain having more than 95% homology or identity to the heavy chain as set forth in SEQ ID NO:7. In certain embodiments, the antibody or antigen binding fragment provided herein comprises a heavy chain having any of the provided homology or identity to the heavy chain as set forth in SEQ ID NO:7, wherein the CDRs (CDR-H1, CDR-H2, and CDR-H3) are identical to the CDRs in the heavy chain as set forth in SEQ ID NO:7. In some embodiments, the antibody or antigen binding fragment provided herein comprises a light chain having more than 70% homology or identity to the light chain as set forth in SEQ ID NO:8. In some embodiments, the antibody or antigen binding fragment provided herein comprises a light chain having more than 75% homology or identity to the light chain as set forth in SEQ ID NO:8. In some embodiments, the antibody or antigen binding fragment provided herein comprises a light chain having more than 80% homology or identity to the light chain as set forth in SEQ ID NO:8. In some embodiments, the antibody or antigen binding fragment provided herein comprises a light chain having more than 85% homology or identity to the light chain as set forth in SEQ ID NO:8. In some embodiments, the antibody or antigen binding fragment provided herein comprises a light chain having more than 90% homology or identity to the light chain as set forth in SEQ ID NO:8. In some embodiments, the antibody or antigen binding fragment provided herein comprises a light chain having more than 95% homology or identity to the light chain as set forth in SEQ ID NO:8. In certain embodiments, the antibody or antigen binding fragment provided herein comprises a light chain having any of the provided homology or identity to the light chain as set forth in SEQ ID NO:8, wherein the CDRs (CDR-L1, CDR-L2, and CDR-L3) are identical to the CDRs in the light chain as set forth in SEQ ID NO:8. In certain embodiments, the antibody or antigen binding fragment provided herein comprises any homologous light chain and any homologous heavy chain as provided in this paragraph in any combination or permutation.

In certain embodiments, the antibody or antigen binding fragment provided herein comprises a heavy chain variable region having certain homology or identity to the heavy chain variable region as set forth in SEQ ID NO:22 and a light chain variable region having certain homology or identity to the light chain variable region as set forth in SEQ ID NO:23. Such embodiments of heavy chain variable regions and light chain variable regions with homology or identity are further provided as follows. In some embodiments, the antibody or antigen binding fragment provided herein comprises a heavy chain variable region having more than 70% homology or identity to the heavy chain variable region as set forth in SEQ ID NO:22. In some embodiments, the antibody or antigen binding fragment provided herein comprises a heavy chain variable region having more than 75% homology or identity to the heavy chain variable region as set forth in SEQ ID NO:22. In some embodiments, the antibody or antigen binding fragment provided herein comprises a heavy chain variable region having more than 80% homology or identity to the heavy chain variable region as set forth in SEQ ID NO:22. In some embodiments, the antibody or antigen binding fragment provided herein comprises a heavy chain variable region having more than 85% homology or identity to the heavy chain variable region as set forth in SEQ ID NO:22. In some embodiments, the antibody or antigen binding fragment provided herein comprises a heavy chain variable region having more than 90% homology or identity to the heavy chain variable region as set forth in SEQ ID NO:22. In some embodiments, the antibody or antigen binding fragment provided herein comprises a heavy chain variable region having more than 95% homology or identity to the heavy chain variable region as set forth in SEQ ID NO:22. In certain embodiments, the antibody or antigen binding fragment provided herein comprises a heavy chain variable region having any of the provided homology or identity to the heavy chain variable region as set forth in SEQ ID NO:22, wherein the CDRs (CDR-H1, CDR-H2, and CDR-H3) are identical to the CDRs in the heavy chain variable region as set forth in SEQ ID NO:22. In some embodiments, the antibody or antigen binding fragment provided herein comprises a light chain variable region having more than 70% homology or identity to the light chain variable region as set forth in SEQ ID NO:23. In some embodiments, the antibody or antigen binding fragment provided herein comprises a light chain variable region having more than 75% homology or identity to the light chain variable region as set forth in SEQ ID NO:23. In some embodiments, the antibody or antigen binding fragment provided herein comprises a light chain variable region having more than 80% homology or identity to the light chain variable region as set forth in SEQ ID NO:23. In some embodiments, the antibody or antigen binding fragment provided herein comprises a light chain variable region having more than 85% homology or identity to the light chain variable region as set forth in SEQ ID NO:23. In some embodiments, the antibody or antigen binding fragment provided herein comprises a light chain variable region having more than 90% homology or identity to the light chain variable region as set forth in SEQ ID NO:23. In some embodiments, the antibody or antigen binding fragment provided herein comprises a light chain variable region having more than 95% homology or identity to the light chain variable region as set forth in SEQ ID NO:23. In certain embodiments, the antibody or antigen binding fragment provided herein comprises a light chain variable region having any of the provided homology or identity to the light chain variable region as set forth in SEQ ID NO:23, wherein the CDRs (CDR-L1, CDR-L2, and CDR-L3) are identical to the CDRs in the light chain variable region as set forth in SEQ ID NO:23. In certain embodiments, the antibody or antigen binding fragment provided herein comprises any homologous light chain variable region and any homologous heavy chain variable region as provided in this paragraph in any combination or permutation.

In some embodiments, the anti-nectin-4 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions comprising amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the anti-nectin-4 antibody provided herein comprises heavy and light chain CDR regions (CDR-H1, CDR-H2, CDR-H3, CDR-L1, CDR-L2, and CDR-L3) of an antibody designated Ha22-2(2,4)6.1 produced by a hybridoma deposited under the American Type Culture Collection (ATCC) Accession NO: PTA-11267, or heavy and light chain CDR regions consisting of amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) comprising the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) comprising the amino acid sequences of the light chain variable region CDRs set forth in the antibody produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) consisting of the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) consisting of the amino acid sequences of the light chain variable region CDRs set forth in the antibody produced by a hybridoma deposited under the American Type Culture Collection (ATCC) Accession NO: PTA-11267.

In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions comprising amino acid sequences that are homologous to the amino acid sequences of the heavy and light chain variable regions of Ha22-2(2,4)6.1, and wherein the antibodies retain the desired functional properties of the anti-nectin-4 antibody provided herein. In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions consisting of amino acid sequences that are homologous to the amino acid sequences of the heavy and light chain variable regions of Ha22-2(2,4)6.1, and wherein the antibodies retain the desired functional properties of the anti-nectin-4 antibody provided herein. As the constant region of the antibody of the disclosure, any subclass of constant region can be chosen. In one embodiment, human IgG1 constant region as the heavy chain constant region and human Ig kappa constant region as the light chain constant region can be used.

In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains comprising amino acid sequences that are homologous to the amino acid sequences of the heavy and light chains of Ha22-2(2,4)6.1, and wherein the antibodies retain the desired functional properties of the anti-nectin-4 antibody provided herein. In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains consisting of amino acid sequences that are homologous to the amino acid sequences of the heavy and light chains of Ha22-2(2,4)6.1, and wherein the antibodies retain the desired functional properties of the anti-nectin-4 antibody provided herein.

In some embodiments, the antibody or antigen binding fragment thereof provided herein comprises a heavy chain variable region and a light chain variable region, wherein:
(a) the heavy chain variable region comprises an amino acid sequence that is at least 80% homologous or identical to the heavy chain variable region amino acid sequence of the antibody produced by a hybridoma deposited under the American Type Culture Collection (ATCC) Accession NO: PTA-11267; and
(b) the light chain variable region comprises an amino acid sequence that is at least 80% homologous or identical to the light chain variable region amino acid sequence of the antibody produced by a hybridoma deposited under the American Type Culture Collection (ATCC) Accession NO: PTA-11267.

In certain embodiments, the antibody or antigen binding fragment provided herein comprises a heavy chain variable region having certain homology or identity to the heavy chain variable region amino acid sequence of the antibody produced by a hybridoma deposited under the American Type Culture Collection (ATCC) Accession NO: PTA-11267 and a light chain variable region having certain homology or identity to the light chain variable region amino acid sequence of the antibody produced by a hybridoma deposited under the American Type Culture Collection (ATCC) Accession NO: PTA-11267. Such embodiments of heavy chain variable regions and light chain variable regions with homology or identity are further provided as follows. In some embodiments, the heavy chain variable region comprises an amino acid sequence that is at least 85% homologous or identical to the heavy chain variable region amino acid sequence of the antibody produced by a hybridoma deposited under the American Type Culture Collection (ATCC) Accession NO: PTA-11267. In other embodiments, the heavy chain variable region comprises an amino acid sequence that is at least 90% homologous or identical to the heavy chain variable region amino acid sequence of the antibody produced by a hybridoma deposited under the American Type Culture Collection (ATCC) Accession NO: PTA-11267. In yet other embodiments, the heavy chain variable region comprises an amino acid sequence that is at least 95% homologous or identical to the heavy chain variable region amino acid sequence of the antibody produced by a hybridoma deposited under the American Type Culture Collection (ATCC) Accession NO: PTA-11267. In other embodiments, the heavy chain variable region can be 85%, 86%, 87%, 88%, 89%, 90%, 91%, 92%, 93%, 94%, 95%, 96%, 97%, 98% or 99% homologous or identical to the heavy chain variable region amino acid sequence of the antibody produced by a hybridoma deposited under the American Type Culture Collection (ATCC) Accession NO: PTA-11267. In some embodiments, the light chain variable region comprises an amino acid sequence that is at least 85% homologous or identical to the light chain variable region amino acid sequence of the antibody produced by a hybridoma deposited under the American Type Culture Collection (ATCC) Accession NO: PTA-11267. In other embodiments, the light chain variable region comprises an amino acid sequence that is at least 90% homologous or identical to the light chain variable region amino acid sequence of the antibody produced by a hybridoma deposited under the American Type Culture Collection (ATCC) Accession NO: PTA-11267. In yet other embodiments, the light chain variable region comprises an amino acid sequence that is at least 95% homologous or identical to the light chain variable region amino acid sequence of the antibody produced by a hybridoma deposited under the American Type Culture Collection (ATCC) Accession NO: PTA-11267. In other embodiments, the light chain variable region can be 85%, 86%, 87%, 88%, 89%, 90%, 91%, 92%, 93%, 94%, 95%, 96%, 97%, 98% or 99% homologous or identical to the light chain variable region amino acid sequence of the antibody produced by a hybridoma deposited under the American Type Culture Collection (ATCC) Accession NO: PTA-11267. In certain embodiments, the antibody or antigen binding fragment provided herein comprises any homologous light chain variable region and any homologous heavy chain variable region as provided in this paragraph in any combination or permutation.

In other embodiments, the antibody or antigen binding fragment thereof provided herein comprises a heavy chain and a light chain, wherein:
(a) the heavy chain comprises an amino acid sequence that is at least 80% homologous or identical to the heavy chain amino acid sequence of the antibody produced by a hybridoma deposited under the American Type Culture Collection (ATCC) Accession NO: PTA-11267; and
(b) the light chain comprises an amino acid sequence that is at least 80% homologous or identical to the light chain amino acid sequence of the antibody produced by a hybridoma deposited under the American Type Culture Collection (ATCC) Accession NO: PTA-11267.

In certain embodiments, the antibody or antigen binding fragment provided herein comprises a heavy chain having certain homology or identity to the heavy chain amino acid sequence of the antibody produced by a hybridoma deposited under the American Type Culture Collection (ATCC) Accession NO: PTA-11267 and a light chain having certain homology or identity to the light chain amino acid sequence of the antibody produced by a hybridoma deposited under the American Type Culture Collection (ATCC) Accession NO: PTA-11267. Such embodiments of heavy chains and light chains with homology or identity are further provided as follows. In some embodiments, the heavy chain comprises an amino acid sequence that is at least 85% homologous or identical to the heavy chain amino acid sequence of the antibody produced by a hybridoma deposited under the American Type Culture Collection (ATCC) Accession NO: PTA-11267. In other embodiments, the heavy chain comprises an amino acid sequence that is at least 90% homologous or identical to the heavy chain amino acid sequence of the antibody produced by a hybridoma deposited under the American Type Culture Collection (ATCC) Accession NO: PTA-11267. In yet other embodiments, the heavy chain comprises an amino acid sequence that is at least 95% homologous or identical to the heavy chain amino acid sequence of the antibody produced by a hybridoma deposited under the American Type Culture Collection (ATCC) Accession NO: PTA-11267. In other embodiments, the heavy chain can be 85%, 86%, 87%, 88%, 89%, 90%, 91%, 92%, 93%, 94%, 95%, 96%, 97%, 98% or 99% homologous or identical to the heavy chain amino acid sequence of the antibody produced by a hybridoma deposited under the American Type Culture Collection (ATCC) Accession NO: PTA-11267. In some embodiments, the light chain comprises an amino acid sequence that is at least 85% homologous or identical to the light chain amino acid sequence of the antibody produced by a hybridoma deposited under the American Type Culture Collection (ATCC) Accession NO: PTA-11267. In other embodiments, the light chain comprises an amino acid sequence that is at least 90% homologous or identical to the light chain amino acid sequence of the antibody produced by a hybridoma deposited under the American Type Culture Collection (ATCC) Accession NO: PTA-11267. In yet other embodiments, the light chain comprises an amino acid sequence that is at least 95% homologous or identical to the light chain amino acid sequence of the antibody produced by a hybridoma deposited under the American Type Culture Collection (ATCC) Accession NO: PTA-11267. In other embodiments, the light chain can be 85%, 86%, 87%, 88%, 89%, 90%, 91%, 92%, 93%, 94%, 95%, 96%, 97%, 98% or 99% homologous or identical to the light chain amino acid sequence of the antibody produced by a hybridoma deposited under the American Type Culture Collection (ATCC) Accession NO: PTA-11267. In certain embodiments, the antibody or antigen binding fragment provided herein comprises any homologous light chain and any homologous heavy chain as provided in this paragraph in any combination or permutation.

In some embodiments, the antibody or antigen binding fragment thereof provided herein binds to a specific epitope in 191P4D12. In some embodiments, the antibody or antigen binding fragment thereof provided herein binds to VC1 domain of 191P4D12. In some embodiments, the antibody or antigen binding fragment thereof provided herein binds to VC1 domain but not to C1C2 domain of 191P4D12. In some embodiments, the antibody or antigen binding fragment thereof provided herein binds to the 1st to 147th amino acid residues of 191P4D12. In some embodiments, the antibody or antigen binding fragment thereof provided herein binds to an epitope located in the 1st to 147th amino acid residues of 191P4D12. In some embodiments, the antibody or antigen binding fragment thereof provided herein binds to the 1st to 10th amino acid residues of 191P4D12. In some embodiments, the antibody or antigen binding fragment thereof provided herein binds to the 11th to 20th amino acid residues of 191P4D12. In some embodiments, the antibody or antigen binding fragment thereof provided herein binds to the 21st to 30th amino acid residues of 191P4D12. In some embodiments, the antibody or antigen binding fragment thereof provided herein binds to the 31st to 40th amino acid residues of 191P4D12. In some embodiments, the antibody or antigen binding fragment thereof provided herein binds to the 41st to 50th amino acid residues of 191P4D12. In some embodiments, the antibody or antigen binding fragment thereof provided herein binds to the 51st to 60th amino acid residues of 191P4D12. In some embodiments, the antibody or antigen binding fragment thereof provided herein binds to the 61st to 70th amino acid residues of 191P4D12. In some embodiments, the antibody or antigen binding fragment thereof provided herein binds to the 71st to 80th amino acid residues of 191P4D12. In some embodiments, the antibody or antigen binding fragment thereof provided herein binds to the 81st to 90th amino acid residues of 191P4D12. In some embodiments, the antibody or antigen binding fragment thereof provided herein binds to the 91st to 100th amino acid residues of 191P4D12. In some embodiments, the antibody or antigen binding fragment thereof provided herein binds to the 101st to 110th amino acid residues of 191P4D12. In some embodiments, the antibody or antigen binding fragment thereof provided herein binds to the 111th to 120th amino acid residues of 191P4D12. In some embodiments, the antibody or antigen binding fragment thereof provided herein binds to the 121st to 130th amino acid residues of 191P4D12. In some embodiments, the antibody or antigen binding fragment thereof provided herein binds to the 131st to 140th amino acid residues of 191P4D12. In some embodiments, the antibody or antigen binding fragment thereof provided herein binds to the 141st to 147th amino acid residues of 191P4D12. The binding epitopes of certain embodiments the antibodies or antigen binding fragments thereof provided herein have been determined and described in WO 2012/047724, which is incorporated herein in its entirety by reference.

In some embodiments, the antibody or antigen binding fragment thereof provided herein binds to epitopes in 191P4D12 that are common between the 191P4D12 variants observed in human. In some embodiments, the antibody or antigen binding fragment thereof provided herein binds to epitopes in 191P4D12 that are common between the 191P4D12 polymorphism observed in human. In some embodiments, the antibody or antigen binding fragment thereof provided herein binds to epitopes in 191P4D12 that are common between the 191P4D12 polymorphism observed in human cancers. In some embodiments, the antibody or antigen binding fragment thereof provided herein binds to epitopes in 191P4D12 that would bind, internalize, disrupt or modulate the biological function of 191P4D12 or 191P4D12 variants. In some embodiments, the antibody or antigen binding fragment thereof provided herein binds to epitopes in 191P4D12 that would disrupt the interaction between 191P4D12 with ligands, substrates, and binding partners.

Engineered antibodies provided herein include those in which modifications have been made to framework residues within VH and/or VL (e.g. to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the disclosure.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an anti-191P4D12 antibody provided herein can be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the anti-191P4D12 antibody.

In another embodiment, the Fc hinge region of an antibody is mutated to decrease the biological half-life of the anti-191P4D12 antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the anti-191P4D12 antibody is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

Reactivity of the anti-191P4D12 antibodies with a 191P4D12-related protein can be established by a number of well-known means, including Western blot, immunoprecipitation, ELISA, and FACS analyses using, as appropriate, 191P4D12-related proteins, 191P4D12-expressing cells or extracts thereof. A 191P4D12 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 191P4D12 epitopes are generated using methods generally known in the art. Homodimeric antibodies can also be generated by cross-linking techniques known in the art (e.g., Wolff et al., Cancer Res. 53: 2560-2565).

In yet another specific embodiment, the anti-191P4D12 antibody provided herein is an antibody comprising heavy and light chain of an antibody designated Ha22-2(2,4)6.1. The heavy chain of Ha22-2(2,4)6.1 consists of the amino acid sequence ranging from 20^{th} E residue to the 466^{th} K residue of SEQ ID NO:7 and the light chain of Ha22-2(2,4)6.1 consists of amino acid sequence ranging from 23^{rd} D residue to the 23^{6th} C residue of SEQ ID NO:8 sequence.

The hybridoma producing the antibody designated Ha22-2(2,4)6.1 was sent (via Federal Express) to the American Type Culture Collection (ATCC), P.O. Box 1549, Manassas, VA 20108 on 18 Aug. 2010 and assigned Accession number PTA-11267.

Additional embodiments of anti-nectin-4 antibody have been described in U.S. Pat. No. 8,637,642 and International Application No. PCT/US2019/056214 (Publication No. WO2020/117373), both of which are hereby incorporated in their entireties by reference.

### 5.3.2 Cytotoxic Agents (Drug Units)

As the ADC used in the methods provided herein comprises an antibody or antigen binding fragment thereof conjugated to a cytotoxic agent, the disclosure further provides various embodiments for the cytotoxic agent as part of the ADC for use in the methods. In various embodiments of the methods provided herein, including the methods provided in Section 5.2, the cytotoxic agent as part of any of the ADCs provided herein for the methods comprises, consists of, or is a tubulin disrupting agent. In one embodiment, the cytotoxic agent is a tubulindisrupting agent. In some embodiments, the tubulin disrupting agent is selected from the group consisting of a dolastatin, an auristatin, a hemiasterlin, a vinca alkaloid, a maytansinoid, an eribulin, a colchicine, a plocabulin, a phomopsin, an epothilone, a cryptophycin, and a taxane. In one specific embodiment, the tubulin disrupting agent is an auristatin. In a further specific embodiment, the auristatin is monomethyl auristatin E (MMAE), monomethyl auristatin F (MMAF), AFP, or auristain T. In yet another specific embodiment, the auristatin is monomethyl auristatin E (MMAE).

In various embodiments of the methods provided herein, including the methods provided in Section 5.2, the cytotoxic agent as part of any of the ADCs provided herein for the methods comprises, consists of, or is any agent selected from the cytotoxic agents described in U.S. Pat. No. 8,637,642 and International Application No. PCT/US2019/056214 (Publication No. WO2020/117373), both of which are hereby incorporated in their entireties by reference

In some embodiments, the auristatin is MMAE (wherein the wavy line indicates the covalent attachment to a linker of an antibody drug conjugate).

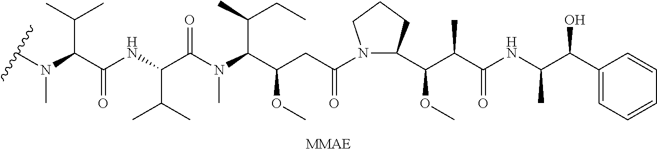 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, an exemplary embodiment comprising MMAE and a linker component (described further herein) has the following structure (wherein L presents the antibody (e.g. anti-nectin-4 antibody or antigen binding fragment thereof) and p ranges from 1 to 12):

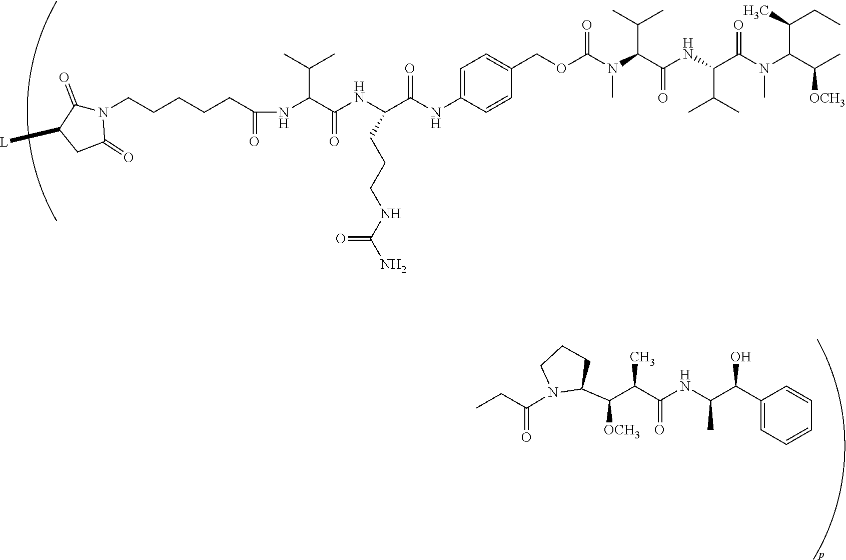

In some embodiments of the formula described in the preceding paragraph, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments of the formula described in the preceding paragraph, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments of the formula described in the preceding paragraph, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments of the formula described in the preceding paragraph, p is about 1. In some embodiments of the formula described in the preceding paragraph, p is about 2. In some embodiments of the formula described in the preceding paragraph, p is about 3. In some embodiments of the formula described in the preceding paragraph, p is about 4. In some embodiments of the formula described in the preceding paragraph, p is about 3.8. In some embodiments of the formula described in the preceding paragraph, p is about 5. In some embodiments of the formula described in the preceding paragraph, p is about 6. In some embodiments of the formula described in the preceding paragraph, p is about 7. In some embodiments of the formula described in the preceding paragraph, p is about 8. In some embodiments of the formula described in the preceding paragraph, p is about 9. In some embodiments of the formula described in the preceding paragraph, p is about 10. In some embodiments of the formula described in the preceding paragraph, p is about 11. In some embodiments of the formula described in the preceding paragraph, p is about 12. In some embodiments of the formula described in the preceding paragraph, p is about 13. In some embodiments of the formula described in the preceding paragraph, p is about 14. In some embodiments of the formula described in the preceding paragraph, p is about 15. In some embodiments of the formula described in the preceding paragraph, p is about 16. In some embodiments of the formula described in the preceding paragraph, p is about 17. In some embodiments of the formula described in the preceding paragraph, p is about 18. In some embodiments of the formula described in the preceding paragraph, p is about 19. In some embodiments of the formula described in the preceding paragraph, p is about 20.

Typically, peptide-based drug units can be prepared by forming a peptide bond between two or more amino acids and/or peptide fragments. Such peptide bonds can be prepared, for example, according to the liquid phase synthesis method (see E. Schröder and K. Lübke, “The Peptides”, volume 1, pp 76-136, 1965, Academic Press) that is well-known in the field of peptide chemistry. The auristatin/dolastatin drug units can be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat Biotechnol 21(7):778-784.

Additional embodiments of cytotoxic agent have been described in U.S. Pat. No. 8,637,642 and International Application No. PCT/US2019/056214 (Publication No. WO2020/117373), both of which are hereby incorporated in their entireties by reference.

### 5.3.3 Linkers

Typically, the antibody drug conjugates comprise a linker unit between the drug unit (e.g., MMAE) and the antibody unit (e.g., the anti-191P4D12 antibody or antigen binding fragment thereof). In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO:15)). In some embodiments, the peptidyl linker is at least two amino acids long or at least three amino acids long. In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT.

A “linker unit” (LU) is a bifunctional compound that can be used to link a drug unit and an antibody unit to form an antibody drug conjugate. In some embodiments, the linker unit has the formula:

-Aₐ-W_{w}-Y_{y}-
wherein: -A- is a stretcher unit,
a is 0 or 1,
each -W- is independently an amino acid unit,
w is an integer ranging from 0 to 12,
-Y- is a self-immolative spacer unit, and
y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0. The linker and each of the stretcher unit, the amino acid unit, and the spacer unit have been described in U.S. Pat. No. 8,637,642 and International Application No. PCT/US2019/056214 (Publication No. WO2020/117373), both of which are hereby incorporated in their entireties by reference.

Embodiments of the antibody-drug conjugates can include:

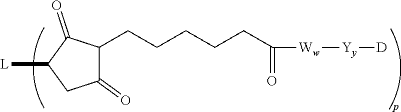

wherein w and y are each 0, 1 or 2, and,

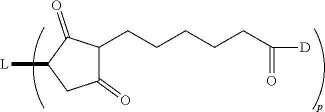

wherein w and y are each 0,

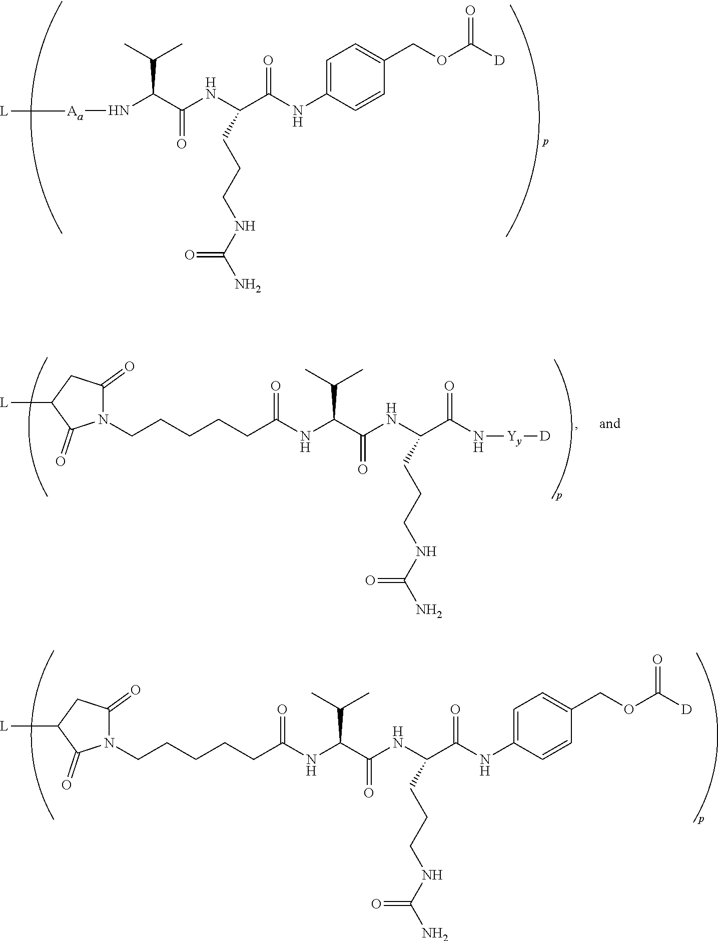

### 5.3.4 Drug Loading

Drug loading is represented by p and is the average number of drug units per antibody in a molecule. Drug loading can range from 1 to 20 drug units (D) per antibody. The ADCs provided herein include collections of antibodies or antigen binding fragments conjugated with a range of drug units, e.g., from 1 to 20. The average number of drug units per antibody in preparations of ADC from conjugation reactions can be characterized by conventional means such as mass spectroscopy and, ELISA assay. The quantitative distribution of ADC in terms of p can also be determined. In some instances, separation, purification, and characterization of homogeneous ADC where p is a certain value from ADC with other drug loadings can be achieved by means such as electrophoresis.

In certain embodiments, the drug loading for an ADC provided herein ranges from 1 to 20. In certain embodiments, the drug loading for an ADC provided herein ranges from 1 to 18. In certain embodiments, the drug loading for an ADC provided herein ranges from 1 to 15. In certain embodiments, the drug loading for an ADC provided herein ranges from 1 to 12. In certain embodiments, the drug loading for an ADC provided herein ranges from 1 to 10. In certain embodiments, the drug loading for an ADC provided herein ranges from 1 to 9. In certain embodiments, the drug loading for an ADC provided herein ranges from 1 to 8. In certain embodiments, the drug loading for an ADC provided herein ranges from 1 to 7. In certain embodiments, the drug loading for an ADC provided herein ranges from 1 to 6. In certain embodiments, the drug loading for an ADC provided herein ranges from 1 to 5. In certain embodiments, the drug loading for an ADC provided herein ranges from 1 to 4. In certain embodiments, the drug loading for an ADC provided herein ranges from 1 to 3. In certain embodiments, the drug loading for an ADC provided herein ranges from 2 to 12. In certain embodiments, the drug loading for an ADC provided herein ranges from 2 to 10. In certain embodiments, the drug loading for an ADC provided herein ranges from 2 to 9. In certain embodiments, the drug loading for an ADC provided herein ranges from 2 to 8. In certain embodiments, the drug loading for an ADC provided herein ranges from 2 to 7. In certain embodiments, the drug loading for an ADC provided herein ranges from 2 to 6. In certain embodiments, the drug loading for an ADC provided herein ranges from 2 to 5. In certain embodiments, the drug loading for an ADC provided herein ranges from 2 to 4. In certain embodiments, the drug loading for an ADC provided herein ranges from 3 to 12. In certain embodiments, the drug loading for an ADC provided herein ranges from 3 to 10. In certain embodiments, the drug loading for an ADC provided herein ranges from 3 to 9. In certain embodiments, the drug loading for an ADC provided herein ranges from 3 to 8. In certain embodiments, the drug loading for an ADC provided herein ranges from 3 to 7. In certain embodiments, the drug loading for an ADC provided herein ranges from 3 to 6. In certain embodiments, the drug loading for an ADC provided herein ranges from 3 to 5. In certain embodiments, the drug loading for an ADC provided herein ranges from 3 to 4.

In certain embodiments, the drug loading for an ADC provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the drug loading for an ADC provided herein is about 1, about 2, about 3, about 4, about 5, about 6, about 7, about 8, about 9, about 10, about 11, about 12, or more. In some embodiments, the drug loading for an ADC provided herein is about 3.1, about 3.2, about 3.3, about 3.4, about 3.5, about 3.6, about 3.7, about 3.8, or about 3.9.

In some embodiments, the drug loading for an ADC provided herein ranges from 2 to 20, 2 to 19, 2 to 18, 2 to 17, 2 to 16, 2 to 15, 2 to 14, or 2 to 13. In some embodiments, the drug loading for an ADC provided herein ranges from 3 to 20, 3 to 19, 3 to 18, 3 to 17, 3 to 16, 3 to 15, 3 to 14, or 3 to 13. In some embodiments, the drug loading for an ADC provided herein is about 1. In some embodiments, the drug loading for an ADC provided herein is about 2. In some embodiments, the drug loading for an ADC provided herein is about 3. In some embodiments, the drug loading for an ADC provided herein is about 4. In some embodiments, the drug loading for an ADC provided herein is about 3.8. In some embodiments, the drug loading for an ADC provided herein is about 5. In some embodiments, the drug loading for an ADC provided herein is about 6. In some embodiments, the drug loading for an ADC provided herein is about 7. In some embodiments, the drug loading for an ADC provided herein is about 8. In some embodiments, the drug loading for an ADC provided herein is about 9. In some embodiments, the drug loading for an ADC provided herein is about 10. In some embodiments, the drug loading for an ADC provided herein is about 11. In some embodiments, the drug loading for an ADC provided herein is about 12. In some embodiments, the drug loading for an ADC provided herein is about 13. In some embodiments, the drug loading for an ADC provided herein is about 14. In some embodiments, the drug loading for an ADC provided herein is about 15. In some embodiments, the drug loading for an ADC provided herein is about 16. In some embodiments, the drug loading for an ADC provided herein is about 17. In some embodiments, the drug loading for an ADC provided herein is about 18. In some embodiments, the drug loading for an ADC provided herein is about 19. In some embodiments, the drug loading for an ADC provided herein is about 20.

In certain embodiments, fewer than the theoretical maximum of drug units are conjugated to an antibody during a conjugation reaction. An antibody can contain, for example, lysine residues that do not react with the drug-linker intermediate or linker reagent. Generally, antibodies do not contain many free and reactive cysteine thiol groups which can be linked to a drug unit; indeed most cysteine thiol residues in antibodies exist as disulfide bridges. In certain embodiments, an antibody can be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the linker unit or a drug unit is conjugated via a lysine residue on the antibody unit. In some embodiments, the linker unit or a drug unit is conjugated via a cysteine residue on the antibody unit.

In some embodiments, the amino acid that attaches to a linker unit or a drug unit is in the heavy chain of an antibody or antigen binding fragment thereof. In some embodiments, the amino acid that attaches to a linker unit or a drug unit is in the light chain of an antibody or antigen binding fragment thereof. In some embodiments, the amino acid that attaches to a linker unit or a drug unit is in the hinge region of an antibody or antigen binding fragment thereof. In some embodiments, the amino acid that attaches to a linker unit or a drug unit is in the Fc region of an antibody or antigen binding fragment thereof. In other embodiments, the amino acid that attaches to a linker unit or a drug unit is in the constant region (e.g., CH1, CH2, or CH3 of a heavy chain, or CH1 of a light chain) of an antibody or antigen binding fragment thereof. In yet other embodiments, the amino acid that attaches to a linker unit or a drug unit is in the VH framework regions of an antibody or antigen binding fragment thereof. In yet other embodiments, the amino acid that attaches to a linker unit or a drug unit is in the VL framework regions of an antibody or antigen binding fragment thereof.

The loading (drug/antibody ratio) of an ADC can be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug unit reagent, then the resulting product is a mixture of ADC compounds with a distribution of one or more drug unit attached to an antibody unit. The average number of drugs per antibody can be calculated from the mixture by a dual ELISA antibody assay, which is specific for antibody and specific for the drug. Individual ADC molecules can be identified in the mixture by mass spectroscopy and separated by HPLC, e.g. hydrophobic interaction chromatography (see, e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value can be isolated from the conjugation mixture by electrophoresis or chromatography.

Methods for preparing, screening, and characterizing the antibody drug conjugates are known to a person of ordinary skill in the art, for example, as described in U.S. Pat. No. 8,637,642, which is herein incorporated in its entirety by reference.

In some embodiments, the antibody drug conjugate for the methods provided herein is AGS-22M6E, which is prepared according to the methods described in U.S. Pat. No. 8,637,642 and has the following formula:

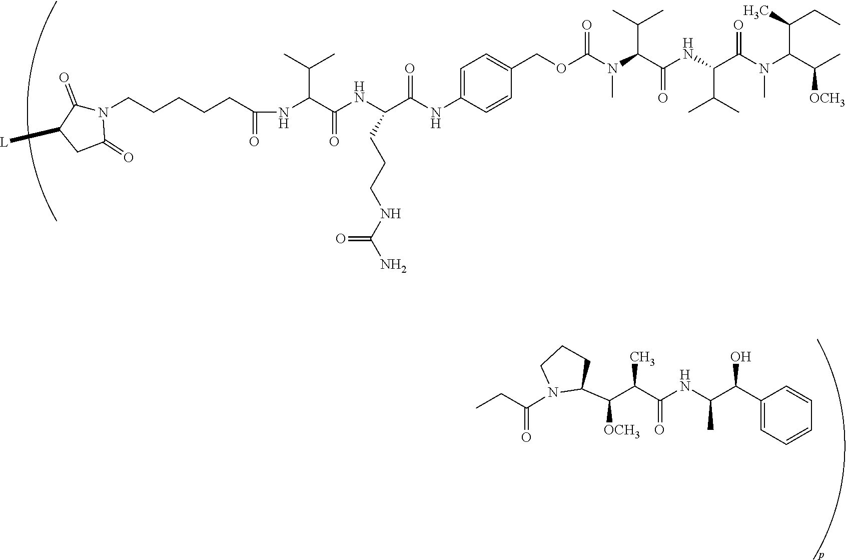

wherein L is Ha22-2(2,4)6.1 and p is from 1 to 20.

In some embodiments, p ranges from 1 to 20, 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10. In some embodiments, p is about 3.1. In some embodiments, p is about 3.2. In some embodiments, p is about 3.3. In some embodiments, p is about 3.4. In some embodiments, p is about 3.5. In other embodiments, p is about 3.6. In some embodiments, p is about 3.7. In some embodiments, p is about 3.8. In some embodiments, p is about 3.9. In some embodiments, p is about 4.0. In some embodiments, p is about 4.1. In some embodiments, p is about 4.2. In some embodiments, p is about 4.3. In some embodiments, p is about 4.4. In some embodiments, p is about 4.5. In other embodiments, p is about 4.6. In some embodiments, p is about 4.7. In some embodiments, p is about 4.8. In some embodiments, p is about 4.9. In some embodiments, p is about 5.0.

In some embodiments, the ADC used in the methods provided herein is enfortumab vedotin. Enfortumab vedotin is an ADC comprised of a fully human immunoglobulin G1 kappa (IgG1_{K}) antibody conjugated to the microtubule-disrupting agent (MMAE) via a protease-cleavable linker (Challita-Eid P M et al, Cancer Res. 2016; 76(10):3003-13]. Enfortumab vedotin induces antitumor activity by binding to 191P4D12 protein on the cell surface leading to internalization of the ADC-191P4D12 complex, which then traffics to the lysosomal compartment where MMAE is released via proteolytic cleavage of the linker. Intracellular release of MMAE subsequently disrupts tubulin polymerization resulting in G2/M phase cell cycle arrest and apoptotic cell death (Francisco J A et al, Blood. 2003 Aug. 15; 102(4):1458-65).

As described above and in in U.S. Pat. No. 8,637,642, AGS-22M6E is an ADC derived from a murine hybridoma cell line. Enfortumab vedotin is the a Chinese hamster ovary (CHO) cell line-derived equivalent of AGS-22M6E ADC and is an exemplary product used for human treatment. Enfortumab vedotin has the same amino acid sequence, linker and cytotoxic drug as AGS-22M6E. The comparability between enfortumab vedotin and AGS-22M6E was confirmed through extensive analytical and biological characterization studies, such as binding affinity to 191P4D12, in vitro cytotoxicity, and in vivo antitumor activity.

In one embodiment, the ADC provided herein is enfortumab vedotin, also known as EV, PADCEV, AGS-22M6E, AGS-22C3E, ASG-22C3E. The enfortumab vedotin includes an anti-191P4D12 antibody, wherein the antibody or antigen binding fragment thereof comprises a heavy chain comprising amino acid residue 20 to amino acid residue 466 of SEQ ID NO: 7 and a light chain comprising amino acid residue 23 to amino acid residue 236 of SEQ ID NO:8.

Enfortumab vedotin is a Nectin-4 directed antibody-drug conjugate (ADC) comprised of a fully human anti-nectin-4 IgG1 kappa monoclonal antibody (AGS-22C3) conjugated to the small molecule microtubule disrupting agent, monomethyl auristatin E (MMAE) via a protease-cleavable maleimidocaproyl valine-citrulline (vc) linker (SGD-1006). Conjugation takes place on cysteine residues that comprise the interchain disulfide bonds of the antibody to yield a product with a drug-to-antibody ratio of approximately 3.8:1. The molecular weight is approximately 152 kDa.

Enfortumab vedotin has the following structural formula:

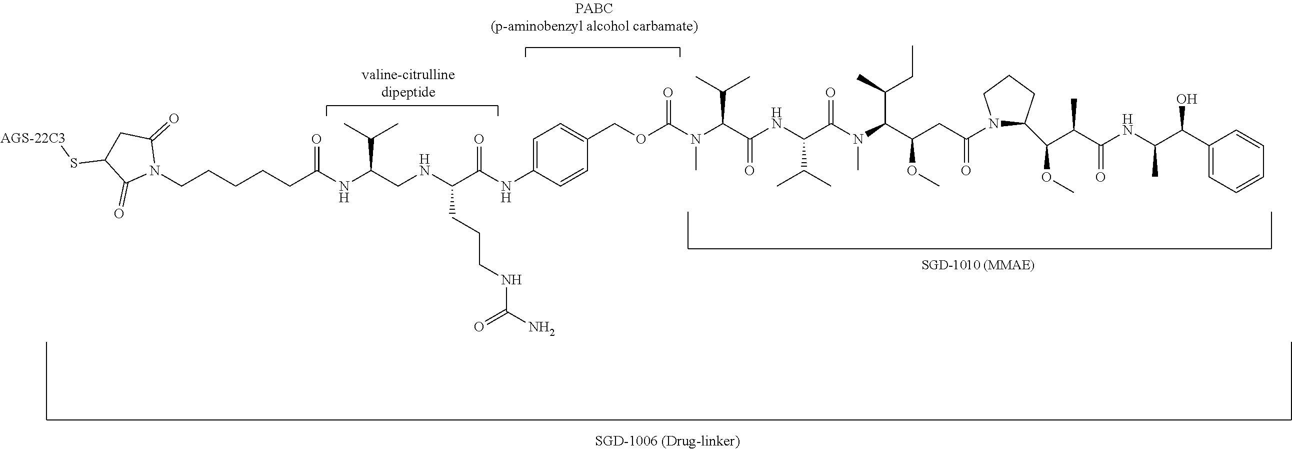

Approximately 4 molecules of MMAE are attached to each antibody molecule. Enfortumab vedotin is produced by chemical conjugation of the antibody and small molecule components. The antibody is produced by mammalian (Chinese hamster ovary) cells and the small molecule components are produced by chemical synthesis.

Enfortumab vedotin injection is provided as a sterile, preservative-free, white to off-white lyophilized powder in single-dose vials for intravenous use. Enfortumab vedotin is supplied as a 20 mg per vial and a 30 mg per vial and requires reconstitution with Sterile Water for Injection, USP, (2.3 mL and 3.3 mL, respectively) resulting in a clear to slightly opalescent, colorless to slightly yellow solution with a final concentration of 10 mg/mL. After reconstitution, each vial allows the withdrawal of 2 mL (20 mg) and 3 mL (30 mg). Each mL of reconstituted solution contains 10 mg of enfortumab vedotin, histidine (1.4 mg), histidine hydrochloride monohydrate (2.31 mg), polysorbate 20 (0.2 mg) and trehalose dihydrate (55 mg) with a pH of 6.0.

### 5.4 Pharmaceutical Compositions

In certain embodiments of the methods provided herein, the ADC used in the methods is provided in “pharmaceutical compositions.” Such pharmaceutical compositions include an antibody drug conjugate provided herein, and one or more pharmaceutically acceptable or physiologically acceptable excipients. In certain embodiments, the antibody drug conjugate are provided in combination with, or separate from, one or more additional agents. Also provided is a composition comprising such one or more additional agents and one or more pharmaceutically acceptable or physiologically acceptable excipients. In particular embodiments, the antibody drug conjugate and an additional agent(s) are present in a therapeutically acceptable amount. The pharmaceutical compositions can be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some embodiments, provided are pharmaceutical compositions of antibody drug conjugates that modulate a cancer or tumor.

In certain embodiments of the methods provided herein, the pharmaceutical compositions comprising the ADCs can further comprise other therapeutically active agents or compounds disclosed herein or known to the skilled artisan which can be used in the treatment or prevention of various diseases and disorders as set forth herein (e.g., a cancer). As set forth above, the additional therapeutically active agents or compounds can be present in a separate pharmaceutical composition(s).

Pharmaceutical compositions typically comprise a therapeutically effective amount of at least one of the antibody drug conjugates provided herein and one or more pharmaceutically acceptable formulation agents. In certain embodiments, the pharmaceutical composition further comprises one or more additional agents described herein.

In one embodiment, a pharmaceutical composition comprises an antibody drug conjugate provided herein. In some embodiments, a pharmaceutical composition comprises a therapeutically effective amount of an antibody drug conjugate provided herein. In certain embodiments, the pharmaceutical composition comprises a pharmaceutically acceptable excipient.

In some embodiments, the antibody drug conjugate in the pharmaceutical composition provided herein is selected from the antibody drug conjugates described in Section 5.3 above.

In certain embodiments, the pharmaceutical composition comprises the antibody drug conjugate at a concentration of from 0.1-100 mg/mL. In some embodiments, the pharmaceutical composition comprises the antibody drug conjugate at a concentration of from 1 to 20 mg/mL. In other embodiments, the pharmaceutical composition comprises the antibody drug conjugate at a concentration of from 5 to 15 mg/mL. In other embodiments, the pharmaceutical composition comprises the antibody drug conjugate at a concentration of from 8 to 12 mg/mL. In other embodiments, the pharmaceutical composition comprises the antibody drug conjugate at a concentration of from 9 to 11 mg/mL. In some embodiments, the pharmaceutical composition comprises the antibody drug conjugate at a concentration of about 9.5 mg/mL. In some embodiments, the pharmaceutical composition comprises the antibody drug conjugate at a concentration of about 9.6 mg/mL. In some embodiments, the pharmaceutical composition comprises the antibody drug conjugate at a concentration of about 9.7 mg/mL. In some embodiments, the pharmaceutical composition comprises the antibody drug conjugate at a concentration of about 9.8 mg/mL. In some embodiments, the pharmaceutical composition comprises the antibody drug conjugate at a concentration of about 9.9 mg/mL. In yet other embodiments, the pharmaceutical composition comprises the antibody drug conjugate at a concentration of about 10 mg/mL. In yet other embodiments, the pharmaceutical composition comprises the antibody drug conjugate at a concentration of about 10.1 mg/mL. In some embodiments, the pharmaceutical composition comprises the antibody drug conjugate at a concentration of about 10.2 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.4 mg/mL. In some embodiments, the pharmaceutical composition comprises the antibody drug conjugate at a concentration of about 10.5 mg/mL.

In some embodiments, the pharmaceutical composition provided herein comprises

L-histidine, TWEEN-20, and at least one of trehalose dihydrate or sucrose. In some embodiments, the pharmaceutical composition provided herein further comprises hydrochloric acid (HCl) or succinic acid.

In some embodiments, the concentration of L-histidine useful in the pharmaceutical compositions provided herein is in the range of between 5 and 50 mM. In some embodiments, the concentration of L-histidine in the pharmaceutical compositions provided herein is in the range of between 10 and 40 mM. In some embodiments, the concentration of L-histidine in the pharmaceutical compositions provided herein is in the range of between 15 and 35 mM.

In some embodiments, the concentration of L-histidine in the pharmaceutical compositions provided herein is in the range of between 15 and 30 mM. In some embodiments, the concentration of L-histidine in the pharmaceutical compositions provided herein is in the range of between 15 and 25 mM. In some embodiments, the concentration of L-histidine in the pharmaceutical compositions provided herein is in the range of between 15 and 35 mM. In some embodiments, the concentration of L-histidine in the pharmaceutical compositions provided herein is about 16 mM. In some embodiments, the concentration of L-histidine in the pharmaceutical compositions provided herein is about 17 mM. In some embodiments, the concentration of L-histidine in the pharmaceutical compositions provided herein is about 18 mM. In some embodiments, the concentration of L-histidine in the pharmaceutical compositions provided herein is about 19 mM. In some embodiments, the concentration of L-histidine in the pharmaceutical compositions provided herein is about 20 mM. In some embodiments, the concentration of L-histidine in the pharmaceutical compositions provided herein is about 21 mM. In some embodiments, the concentration of L-histidine in the pharmaceutical compositions provided herein is about 22 mM. In some embodiments, the concentration of L-histidine in the pharmaceutical compositions provided herein is about 23 mM. In some embodiments, the concentration of L-histidine in the pharmaceutical compositions provided herein is about 24 mM. In some embodiments, the concentration of L-histidine in the pharmaceutical compositions provided herein is about 25 mM.

In some embodiments, the concentration of TWEEN-20 useful in the pharmaceutical compositions provided herein is in the range of from 0.001 to 0.1% (v/v). In another embodiment, the concentration of TWEEN-20 is in the range of from 0.0025 to 0.075% (v/v). In one embodiment, the concentration of TWEEN-20 is in the range of from 0.005 to 0.05% (v/v). In another embodiment, the concentration of TWEEN-20 is in the range of from 0.0075 to 0.025% (v/v). In another embodiment, the concentration of TWEEN-20 is in the range of from 0.0075 to 0.05% (v/v). In another embodiment, the concentration of TWEEN-20 is in the range of from 0.01 to 0.03% (v/v). In one particular embodiment, the concentration of TWEEN-20 is about 0.01% (v/v). In one particular embodiment, the concentration of TWEEN-20 is about 0.015% (v/v). In one particular embodiment, the concentration of TWEEN-20 is about 0.016% (v/v). In one particular embodiment, the concentration of TWEEN-20 is about 0.017% (v/v). In one particular embodiment, the concentration of TWEEN-20 is about 0.018% (v/v). In one particular embodiment, the concentration of TWEEN-20 is about 0.019% (v/v). In one particular embodiment, the concentration of TWEEN-20 is about 0.02% (v/v). In one particular embodiment, the concentration of TWEEN-20 is about 0.021% (v/v). In one particular embodiment, the concentration of TWEEN-20 is about 0.022% (v/v). In one particular embodiment, the concentration of TWEEN-20 is about 0.023% (v/v). In one particular embodiment, the concentration of TWEEN-20 is about 0.024% (v/v). In one particular embodiment, the concentration of TWEEN-20 is about 0.025% (v/v).

In one embodiment, the concentration of trehalose dihydrate useful in the pharmaceutical compositions provided herein is in the range of between 1% and 20% (w/v). In another embodiment, the concentration of trehalose dihydrate is in the range of 2% and 15% (w/v). In one embodiment, the concentration of trehalose dihydrate is in the range of 3% and 10% (w/v). In another embodiment, the concentration of trehalose dihydrate is in the range of 4% and 9% (w/v). In another embodiment, the concentration of trehalose dihydrate is in the range of 4% and 8% (w/v). In another embodiment, the concentration of trehalose dihydrate is in the range of 4% and 7% (w/v). In another embodiment, the concentration of trehalose dihydrate is in the range of 4% and 6% (w/v). In another embodiment, the concentration of trehalose dihydrate is in the range of 4.5% and 6% (w/v). In another embodiment, the concentration of trehalose dihydrate is about 4.6% (w/v). In another embodiment, the concentration of trehalose dihydrate is about 4.7% (w/v). In another embodiment, the concentration of trehalose dihydrate is about 4.8% (w/v). In another embodiment, the concentration of trehalose dihydrate is about 4.9% (w/v). In another embodiment, the concentration of trehalose dihydrate is about 5.0% (w/v). In another embodiment, the concentration of trehalose dihydrate is about 5.1% (w/v). In another embodiment, the concentration of trehalose dihydrate is about 5.2% (w/v). In another embodiment, the concentration of trehalose dihydrate is about 5.3% (w/v). In another embodiment, the concentration of trehalose dihydrate is about 5.4% (w/v). In another embodiment, the concentration of trehalose dihydrate is about 5.5% (w/v). In another embodiment, the concentration of trehalose dihydrate is about 5.6% (w/v). In another embodiment, the concentration of trehalose dihydrate is about 5.7% (w/v). In another embodiment, the concentration of trehalose dihydrate is about 5.8% (w/v). In another embodiment, the concentration of trehalose dihydrate is about 5.9% (w/v). In another embodiment, the concentration of trehalose dihydrate is about 6.0% (w/v). In another embodiment, the concentration of trehalose dihydrate is about 6.1% (w/v). In another embodiment, the concentration of trehalose dihydrate is about 6.2% (w/v). In another embodiment, the concentration of trehalose dihydrate is about 6.3% (w/v). In another embodiment, the concentration of trehalose dihydrate is about 6.4% (w/v). In another embodiment, the concentration of trehalose dihydrate is about 6.5% (w/v).

In certain embodiments, the molarity of the trehalose dihydrate is from 50 to 300 mM. In other embodiments, the molarity of the trehalose dihydrate is from 75 to 250 mM. In some embodiments, the molarity of the trehalose dihydrate is from 100 to 200 mM. In other embodiments, the molarity of the trehalose dihydrate is from 130 to 150 mM. In some embodiments, the molarity of the trehalose dihydrate is from 135 to 150 mM. In certain embodiments, the molarity of the trehalose dihydrate is about 135 mM. In certain embodiments, the molarity of the trehalose dihydrate is about 136 mM. In certain embodiments, the molarity of the trehalose dihydrate is about 137 mM. In certain embodiments, the molarity of the trehalose dihydrate is about 138 mM. In certain embodiments, the molarity of the trehalose dihydrate is about 139 mM. In certain embodiments, the molarity of the trehalose dihydrate is about 140 mM. In certain embodiments, the molarity of the trehalose dihydrate is about 141 mM. In certain embodiments, the molarity of the trehalose dihydrate is about 142 mM. In certain embodiments, the molarity of the trehalose dihydrate is about 143 mM. In certain embodiments, the molarity of the trehalose dihydrate is about 144 mM. In certain embodiments, the molarity of the trehalose dihydrate is about 145 mM. In certain embodiments, the molarity of the trehalose dihydrate is about 146 mM. In certain embodiments, the molarity of the trehalose dihydrate is about 150 mM. In certain embodiments, the molarity of the trehalose dihydrate is about 151 mM. In certain embodiments, the molarity of the trehalose dihydrate is about 151 mM. In certain embodiments, the molarity of the trehalose dihydrate is about 152 mM. In certain embodiments, the molarity of the trehalose dihydrate is about 153 mM. In certain embodiments, the molarity of the trehalose dihydrate is about 154 mM. In certain embodiments, the molarity of the trehalose dihydrate is about 155 mM.

In one embodiment, the concentration of sucrose useful in the pharmaceutical compositions provided herein is in the range of between 1% and 20% (w/v). In another embodiment, the concentration of sucrose is in the range of 2% and 15% (w/v). In one embodiment, the concentration of sucrose is in the range of 3% and 10% (w/v). In another embodiment, the concentration of sucrose is in the range of 4% and 9% (w/v). In another embodiment, the concentration of sucrose is in the range of 4% and 8% (w/v). In another embodiment, the concentration of sucrose is in the range of 4% and 7% (w/v). In another embodiment, the concentration of sucrose is in the range of 4% and 6% (w/v). In another embodiment, the concentration of sucrose is in the range of 4.5% and 6% (w/v). In another embodiment, the concentration of sucrose is about 4.6% (w/v). In another embodiment, the concentration of sucrose is about 4.7% (w/v). In another embodiment, the concentration of sucrose is about 4.8% (w/v). In another embodiment, the concentration of sucrose is about 4.9% (w/v). In another embodiment, the concentration of sucrose is about 5.0% (w/v). In another embodiment, the concentration of sucrose is about 5.1% (w/v). In another embodiment, the concentration of sucrose is about 5.2% (w/v). In another embodiment, the concentration of sucrose is about 5.3% (w/v). In another embodiment, the concentration of sucrose is about 5.4% (w/v). In another embodiment, the concentration of sucrose is about 5.5% (w/v). In another embodiment, the concentration of sucrose is about 5.6% (w/v). In another embodiment, the concentration of sucrose is about 5.7% (w/v). In another embodiment, the concentration of sucrose is about 5.8% (w/v). In another embodiment, the concentration of sucrose is about 5.9% (w/v). In another embodiment, the concentration of sucrose is about 6.0% (w/v). In another embodiment, the concentration of sucrose is about 6.1% (w/v). In another embodiment, the concentration of sucrose is about 6.2% (w/v). In another embodiment, the concentration of sucrose is about 6.3% (w/v). In another embodiment, the concentration of sucrose is about 6.4% (w/v). In another embodiment, the concentration of sucrose is about 6.5% (w/v).

In certain embodiments, the molarity of the sucrose is from 50 to 300 mM. In other embodiments, the molarity of the sucrose is from 75 to 250 mM. In some embodiments, the molarity of the sucrose is from 100 to 200 mM. In other embodiments, the molarity of the sucrose is from 130 to 150 mM. In some embodiments, the molarity of the sucrose is from 135 to 150 mM. In certain embodiments, the molarity of the sucrose is about 135 mM. In certain embodiments, the molarity of the sucrose is about 136 mM. In certain embodiments, the molarity of the sucrose is about 137 mM. In certain embodiments, the molarity of the sucrose is about 138 mM. In certain embodiments, the molarity of the sucrose is about 139 mM. In certain embodiments, the molarity of the sucrose is about 140 mM. In certain embodiments, the molarity of the sucrose is about 141 mM. In certain embodiments, the molarity of the sucrose is about 142 mM. In certain embodiments, the molarity of the sucrose is about 143 mM. In certain embodiments, the molarity of the sucrose is about 144 mM. In certain embodiments, the molarity of the sucrose is about 145 mM. In certain embodiments, the molarity of the sucrose is about 146 mM. In certain embodiments, the molarity of the sucrose is about 150 mM. In certain embodiments, the molarity of the sucrose is about 151 mM. In certain embodiments, the molarity of the sucrose is about 151 mM. In certain embodiments, the molarity of the sucrose is about 152 mM. In certain embodiments, the molarity of the sucrose is about 153 mM. In certain embodiments, the molarity of the sucrose is about 154 mM. In certain embodiments, the molarity of the sucrose is about 155 mM.

In some embodiments, the pharmaceutical composition provided herein comprises HCl. In other embodiments, the pharmaceutical composition provided herein comprises succinic acid.

In some embodiments, the pharmaceutical composition provided herein

has a pH in a range of 5.5 to 6.5. In other embodiments, the pharmaceutical composition provided herein has a pH in a range of 5.7 to 6.3. In some embodiments, the pharmaceutical composition provided herein has a pH of about 5.7. In some embodiments, the pharmaceutical composition provided herein has a pH of about 5.8. In some embodiments, the pharmaceutical composition provided herein has a pH of about 5.9. In some embodiments, the pharmaceutical composition provided herein has a pH of about 6.0. In some embodiments, the pharmaceutical composition provided herein has a pH of about 6.1. In some embodiments, the pharmaceutical composition provided herein has a pH of about 6.2. In some embodiments, the pharmaceutical composition provided herein has a pH of about 6.3.

In some embodiments, the pH is taken at room temperature. In other embodiments,

the pH is taken at 15° C. to 27° C. In yet other embodiments, the pH is taken at 4° C. In yet other embodiments, the pH is taken at 25° C.

In some embodiments, the pH is adjusted by HCl. In some embodiments, the pharmaceutical composition comprises HCl, and the pharmaceutical composition has a pH in a range of 5.5 to 6.5 at room temperature. In some embodiments, the pharmaceutical composition comprises HCl, and the pharmaceutical composition has a pH in a range of 5.7 to 6.3 at room temperature. In some more specific embodiments, the pharmaceutical composition comprises HCl, and the pharmaceutical composition has a pH of about of 5.7 at room temperature. In some more specific embodiments, the pharmaceutical composition comprises HCl, and the pharmaceutical composition has a pH of about of 5.8 at room temperature. In some more specific embodiments, the pharmaceutical composition comprises HCl, and the pharmaceutical composition has a pH of about of 5.9 at room temperature. In some more specific embodiments, the pharmaceutical composition comprises HCl, and the pharmaceutical composition has a pH of about of 6.0 at room temperature. In some more specific embodiments, the pharmaceutical composition comprises HCl, and the pharmaceutical composition has a pH of about of 6.1 at room temperature. In some more specific embodiments, the pharmaceutical composition comprises HCl, and the pharmaceutical composition has a pH of about of 6.2 at room temperature. In some more specific embodiments, the pharmaceutical composition comprises HCl, and the pharmaceutical composition has a pH of about of 6.3 at room temperature.

In some embodiments, the pharmaceutical composition comprises HCl, and the pharmaceutical composition has a pH in a range of 5.5 to 6.5 at 15° C. to 27° C. In some embodiments, the pharmaceutical composition comprises HCl, and the pharmaceutical composition has a pH in a range of 5.7 to 6.3 at 15° C. to 27° C. In some more specific embodiments, the pharmaceutical composition comprises HCl, and the pharmaceutical composition has a pH of about of 5.7 at 15° C. to 27° C. In some more specific embodiments, the pharmaceutical composition comprises HCl, and the pharmaceutical composition has a pH of about of 5.8 at 15° C. to 27° C. In some more specific embodiments, the pharmaceutical composition comprises HCl, and the pharmaceutical composition has a pH of about of 5.9 at 15° C. to 27° C. In some more specific embodiments, the pharmaceutical composition comprises HCl, and the pharmaceutical composition has a pH of about of 6.0 at 15° C. to 27° C. In some more specific embodiments, the pharmaceutical composition comprises HCl, and the pharmaceutical composition has a pH of about of 6.1 at 15° C. to 27° C. In some more specific embodiments, the pharmaceutical composition comprises HCl, and the pharmaceutical composition has a pH of about of 6.2 at 15° C. to 27° C. In some more specific embodiments, the pharmaceutical composition comprises HCl, and the pharmaceutical composition has a pH of about of 6.3 at 15° C. to 27° C.

In some embodiments, the pH is adjusted by succinic acid. In some embodiments, the pharmaceutical composition comprises succinic acid, and the pharmaceutical composition has a pH in a range of 5.5 to 6.5 at room temperature. In some embodiments, the pharmaceutical composition comprises succinic acid, and the pharmaceutical composition has a pH in a range of 5.7 to 6.3 at room temperature. In some more specific embodiments, the pharmaceutical composition comprises succinic acid, and the pharmaceutical composition has a pH of about of 5.7 at room temperature. In some more specific embodiments, the pharmaceutical composition comprises succinic acid, and the pharmaceutical composition has a pH of about of 5.8 at room temperature. In some more specific embodiments, the pharmaceutical composition comprises succinic acid, and the pharmaceutical composition has a pH of about of 5.9 at room temperature. In some more specific embodiments, the pharmaceutical composition comprises succinic acid, and the pharmaceutical composition has a pH of about of 6.0 at room temperature. In some more specific embodiments, the pharmaceutical composition comprises succinic acid, and the pharmaceutical composition has a pH of about of 6.1 at room temperature. In some more specific embodiments, the pharmaceutical composition comprises succinic acid, and the pharmaceutical composition has a pH of about of 6.2 at room temperature. In some more specific embodiments, the pharmaceutical composition comprises succinic acid, and the pharmaceutical composition has a pH of about of 6.3 at room temperature.

In some embodiments, the pharmaceutical composition comprises succinic acid, and the pharmaceutical composition has a pH in a range of 5.5 to 6.5 at 15° C. to 27° C. In some embodiments, the pharmaceutical composition comprises succinic acid, and the pharmaceutical composition has a pH in a range of 5.7 to 6.3 at 15° C. to 27° C. In some more specific embodiments, the pharmaceutical composition comprises succinic acid, and the pharmaceutical composition has a pH of about of 5.7 at 15° C. to 27° C. In some more specific embodiments, the pharmaceutical composition comprises succinic acid, and the pharmaceutical composition has a pH of about of 5.8 at 15° C. to 27° C. In some more specific embodiments, the pharmaceutical composition comprises succinic acid, and the pharmaceutical composition has a pH of about of 5.9 at 15° C. to 27° C. In some more specific embodiments, the pharmaceutical composition comprises succinic acid, and the pharmaceutical composition has a pH of about of 6.0 at 15° C. to 27° C. In some more specific embodiments, the pharmaceutical composition comprises succinic acid, and the pharmaceutical composition has a pH of about of 6.1 at 15° C. to 27° C. In some more specific embodiments, the pharmaceutical composition comprises succinic acid, and the pharmaceutical composition has a pH of about of 6.2 at 15° C. to 27° C. In some more specific embodiments, the pharmaceutical composition comprises succinic acid, and the pharmaceutical composition has a pH of about of 6.3 at 15° C. to 27° C.

In some specific embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5.5% (w/v) trehalose dihydrate and HCl. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and HCl. In some embodiments, the pH is about 6.0 at room temperature. In some embodiments, the pH is about 6.0 at 25° C.

In other specific embodiments, the pharmaceutical composition provided herein comprises about 20 mM L-histidine, about 0.02% (w/v) TWEEN-20, about 5.5% (w/v) trehalose dihydrate and succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and succinic acid. In some embodiments, the pH is about 6.0 at room temperature. In some embodiments, the pH is about 6.0 at 25° C.

In a specific embodiment, provided herein comprises
(a) an antibody drug conjugate comprising the following structure:

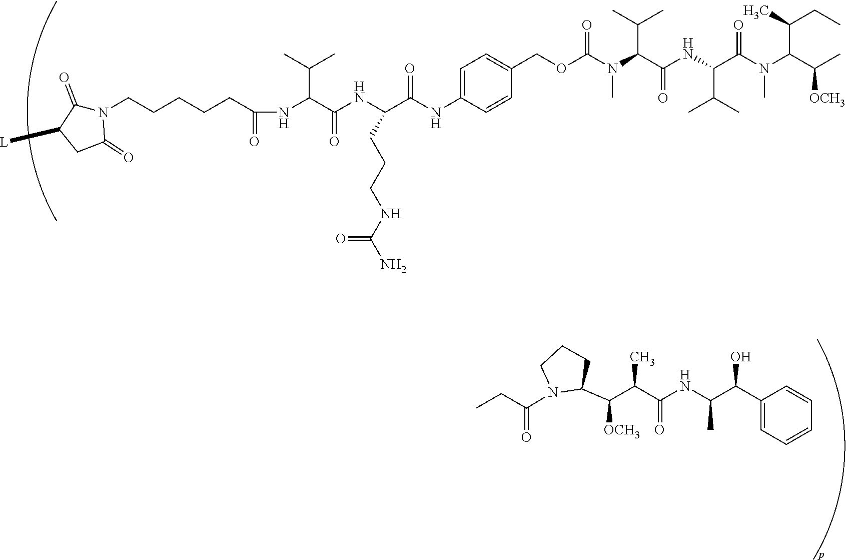

wherein L- represents the antibody or antigen binding fragment (e.g. anti-nectin-4 antibody or antigen binding fragment thereof) thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and HCl, wherein the antibody drug conjugate is at the concentration of about 10 mg/mL, and wherein the pH is about 6.0 at 25° C.

In another specific embodiment, the pharmaceutical composition provided herein comprises:
(a) an antibody drug conjugate comprising the following structure:

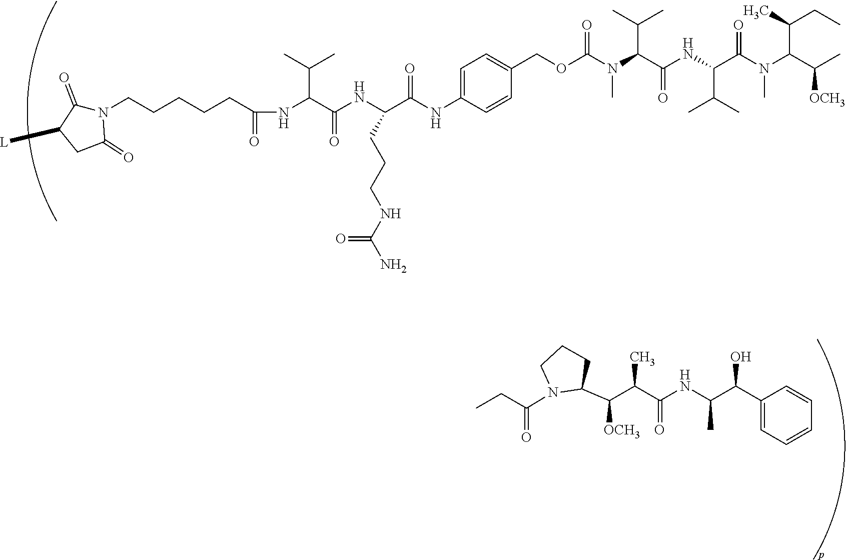

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.5% (w/v) trehalose dihydrate, and succinic acid, wherein the antibody drug conjugate is at the concentration of about 10 mg/mL, and wherein the pH is about 6.0 at 25° C.

In yet another specific embodiment, the pharmaceutical composition provided herein comprises:
(a) an antibody drug conjugate comprising the following structure:

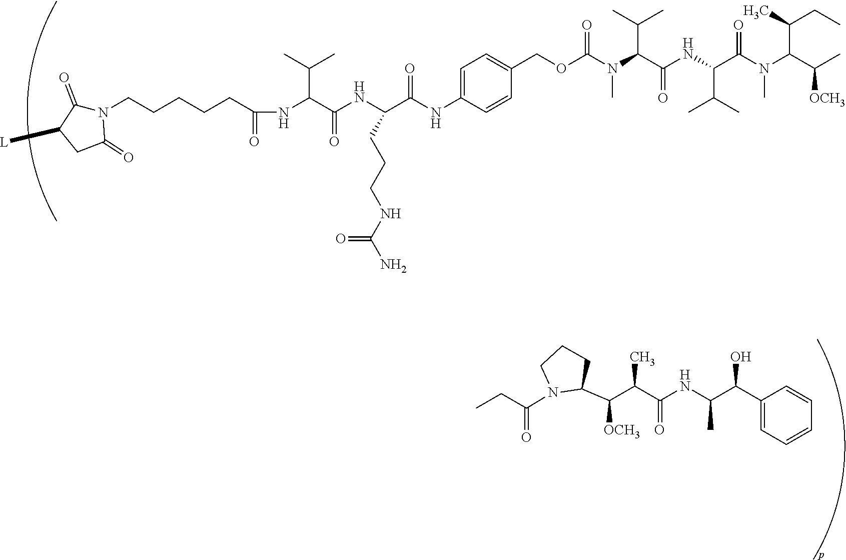

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.0% (w/v) sucrose, and HCl, wherein the antibody drug conjugate is at the concentration of about 10 mg/mL, and wherein the pH is about 6.0 at 25° C.

Although certain numbers (and numerical ranges thereof) are provided, it is understood that, in certain embodiments, numerical values within, e.g., 2%, 5%, 10%, 15% or 20% of said numbers (or numerical ranges) are also contemplated.

A primary solvent in a vehicle can be either aqueous or non-aqueous in nature. In addition, the vehicle can contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein can contain still other pharmaceutically acceptable formulation agents for modifying or maintaining the rate of release of an antibody drug conjugate and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, NJ); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

In some embodiments, the pharmaceutical composition provided herein is in a liquid form. In other embodiments, the pharmaceutical composition provided herein is lyophilized.

A pharmaceutical composition can be formulated to be compatible with its intended route of administration. Thus, pharmaceutical compositions include excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical). Other exemplary routes of administration are set forth herein.

Pharmaceutical compositions can be in the form of a sterile injectable aqueous or oleagenous suspension. This suspension can be formulated using suitable dispersing or wetting agents and suspending agents disclosed herein or known to the skilled artisan. The sterile injectable preparation can also be a sterile injectable solution or suspension in a nontoxic parenterally-acceptable diluent or solvent, for example, as a solution in 1,3-butane diol. Acceptable diluents, solvents and dispersion media that can be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil can be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

In one embodiment, the pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In one embodiment, the pharmaceutical compositions provided herein can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, e.g., Remington, The Science and Practice of Pharmacy, supra).

In one embodiment, the pharmaceutical compositions intended for parenteral administration can include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

In one embodiment, suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

In one embodiment, suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, KS).

In one embodiment, the pharmaceutical compositions provided herein can be formulated for single or multiple dosage administration. The single dosage formulations are packaged in an ampoule, a vial, or a syringe. The multiple dosage parenteral formulations can contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

In one embodiment, the pharmaceutical compositions provided herein can be formulated as immediate or modified release dosage forms, including delayed-, sustained, pulsed-, controlled, targeted-, and programmed-release forms.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions can also include excipients to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The pharmaceutical composition provided herein can be stored at −80° C., 4° C., 25° C. or 37° C.

A lyophilized composition can be made by freeze-drying the liquid pharmaceutical composition provided herein. In a specific embodiment, the pharmaceutical composition provided here is a lyophilized pharmaceutical composition. In some embodiments, the pharmaceutical formulations are lyophilized powders, which can be reconstituted for administration as solutions, emulsions and other mixtures. They can also be reconstituted and formulated as solids or gels.

In some embodiments, preparation of the lyophilized formulation provided herein involves batching of the formulated bulk solution for lyophilization, aseptic filtration, filling in vials, freezing vials in a freeze-dryer chamber, followed by lyophilization, stoppering and capping.

A lyophilizer can be used in preparing the lyophilized formulation. For example, a VirTis Genesis Model EL pilot unit can be employed. The unit incorporates a chamber with three working shelves (to a total usable shelf area of ca 0.4 square meters), an external condenser, and a mechanical vacuum pumping system. Cascaded mechanical refrigeration allows the shelves to be cooled to −70° C. or lower, and the external condenser to −90° C. or lower. Shelf temperature and chamber pressure were controlled automatically to +/−0.5° C. and +/−2 microns (milliTorr), respectively. The unit was equipped with a capacitance manometer vacuum gauge, a Pirani vacuum gauge, a pressure transducer (to measure from 0 to 1 atmosphere), and a relative humidity sensor.

The lyophilized powder can be prepared by dissolving an antibody drug conjugate provided herein, or a pharmaceutically acceptable derivative thereof, in a suitable solvent. In some embodiments, the lyophilized powder is sterile. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the antibody drug conjugate. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable excipient. Such amount can be empirically determined and adjusted according to specific needs.

An exemplary reconstitution procedure is illustrated as follows: (1) fit the 5 mL or 3 mL syringe with a with a 18 or 20 Gauge needle and filled the syringe with water of the grade Water for Injection (WFI); (2) measure appropriate amount of WFI using the syringe graduations, ensuring that the syringe was free of air bubbles; (3) inserted the needle through the rubber stopper; (4) dispense the entire contents of the syringe into the container down the vial wall, removed the syringe and needle and put into the sharp container; (4) swirl the vial continuously to carefully solubilize the entire vial contents until fully reconstituted (e.g., about 20-40 seconds) and minimize excessive agitation of the protein solution that could result in foaming.

In some embodiments, the pharmaceutical composition provided herein is supplied as a dry sterilized lyophilized powder or water free concentrate in a hermetically sealed container and can be reconstituted, e.g., with water or saline to the appropriate concentration for administration to a subject. In certain embodiments, the antibody drug conjugate is supplied as a dry sterile lyophilized powder in a hermetically sealed container at a unit dosage of at least 0.1 mg, at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody drug conjugate can be stored at between 2 and 8° C. in its original container and the antibody drug conjugate can be administered within 12 hours, such as within 6 hours, within 5 hours, within 3 hours, or within 1 hour after being reconstituted. In an alternative embodiment, the pharmaceutical composition comprising the antibody drug conjugate provided herein is supplied in liquid form in a hermetically sealed container indicating the quantity and concentration of the antibody drug conjugate. In certain embodiments, the liquid form of the antibody drug conjugate is supplied in a hermetically sealed container at least 0.1 mg/ml, at least 0.5 mg/ml, at least 1 mg/ml, at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

Additional embodiments for the pharmaceutical compositions have been described in U.S. Pat. No. 8,637,642 and International Application No. PCT/US2019/056214 (Publication No. WO2020/117373), both of which are hereby incorporated in their entireties by reference.

### 5.5 Methods for a Combination Therapy

The method for inhibiting growth of tumor cells using the pharmaceutical composition provided herein in combination with chemotherapy or radiation or both comprises administering the present pharmaceutical composition before, during, or after commencing chemotherapy or radiation therapy, as well as any combination thereof (i.e. before and during, before and after, during and after, or before, during, and after commencing the chemotherapy and/or radiation therapy). Depending on the treatment protocol and the specific patient needs, the method is performed in a manner that will provide the most efficacious treatment and ultimately prolong the life of the patient. Additional embodiments for such combination therapy have been described in U.S. Pat. No. 8,637,642 and International Application No. PCT/US2019/056214 (Publication No. WO2020/117373), both of which are hereby incorporated in their entireties by reference.

### 5.6 Doses for the Immune Checkpoint Inhibitors

In some embodiments, the amount of the checkpoint inhibitor for the various methods provided herein be determined by standard clinical techniques.

A dosage of the checkpoint inhibitor results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose of the checkpoint inhibitor to be employed will also depend on the route of administration, and the seriousness of a cancer in a subject, and should be decided according to the judgment of the practitioner and each patient's circumstances.

In some embodiments, the dosage of the checkpoint inhibitor (e.g., a PD-1 inhibitor or a PD-L1 inhibitor) administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 1 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight. In some embodiments, dosage administered to a patient is about 9.0 mg/kg of the subject's body weight. In some embodiments, dosage administered to a patient is about 10.0 mg/kg of the subject's body weight. In some embodiments, dosage administered to a patient is about 15.0 mg/kg of the subject's body weight. In some embodiments, dosage administered to a patient is about 20.0 mg/kg of the subject's body weight.

### 5.7 Dosage of the ADCs for the Methods

In some embodiments, the amount of a prophylactic or therapeutic agent (e.g., an antibody drug conjugate provided herein), or a pharmaceutical composition provided herein that will be effective in the prevention and/or treatment of a cancer can be determined by standard clinical techniques.

In some embodiments, the ADC of the methods for which the various dosages are described in this Section (Section 5.7) is enfortumab vedotin (EV).

Accordingly, a dosage of an antibody drug conjugate in the pharmaceutical composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose to be employed in the formulation will also depend on the route of administration, and the seriousness of a cancer in a subject, and should be decided according to the judgment of the practitioner and each patient's circumstances.

Effective doses can be extrapolated from dose-response curves derived from in vitro or animal model test systems.

For the pharmaceutical composition comprising the antibody drug conjugate provided herein, the dosage of the antibody drug conjugate administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 0.5 mg/kg of the subject's body weight. In some embodiments, dosage administered to a patient is about 0.75 mg/kg of the subject's body weight. In some embodiments, dosage administered to a patient is about 1 mg/kg of the subject's body weight. In some embodiments, dosage administered to a patient is about 1.25 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight.

In some embodiments, the antibody drug conjugate formulated in the pharmaceutical composition provided herein is administered based on the patient's actual body weight at baseline and doses will not change unless the patient's weight changes by ≥10% from baseline of the previous cycle, or the dose adjustment criteria is met. In some embodiments, actual weight will be used except for patients weighing greater than 100 kg, in such cases, the dose will be calculated based on a weight of 100 kg. In some embodiments, the maximum doses are 100 mg for patients receiving the 1.00 mg/kg dose level and 125 mg for patients receiving the 1.25 mg/kg dose level.

In one embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1.5 mg/kg or less, approximately 1.25 mg/kg or less, approximately 1 mg/kg or less, approximately 0.75 mg/kg or less, approximately 0.5 mg/kg or less, or approximately 0.1 mg/kg or less of an antibody drug conjugate formulated in the present pharmaceutical composition is administered 5 times, 4 times, 3 times, 2 times or 1 time to treat a cancer. In some embodiments, the pharmaceutical composition comprising the antibody drug conjugate provided herein is administered about 1-12 times, wherein the doses can be administered as necessary, e.g., weekly, biweekly, monthly, bimonthly, trimonthly, etc., as determined by a physician. In some embodiments, a lower dose (e.g., 0.1-15 mg/kg) can be administered more frequently (e.g., 3-6 times). In other embodiments, a higher dose (e.g., 25-100 mg/kg) can be administered less frequently (e.g., 1-3 times).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every two-week cycle (e.g., about 14 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every three-week cycle (e.g., about 21 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every four-week cycle (e.g., about 28 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, 6, 7, 8, 9, 10, 11, or 12 times at about monthly (e.g., about 30 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, or 6 times at about bi-monthly (e.g., about 60 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In yet another embodiment, a single dose of an antibody drug conjugate formulated in the pharmaceutical composition provided herein is administered to patient to prevent and/or treat a cancer 1, 2, 3 or 4 times at about tri-monthly (e.g., about 120 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In certain embodiments, the route of administration for a dose of an antibody drug conjugate formulated in the pharmaceutical composition provided herein to a patient is intranasal, intramuscular, intravenous, or a combination thereof, but other routes described herein are also acceptable. Each dose may or may not be administered by an identical route of administration. In some embodiments, an antibody drug conjugate formulated in the pharmaceutical composition provided herein can be administered via multiple routes of administration simultaneously or subsequently to other doses of one or more additional therapeutic agents.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion over about 30 minutes twice every three-week cycle. In some embodiments, the antibody drug conjugate formulated in the pharmaceutical composition is administered by an intravenous (IV) injection or infusion over about 30 minutes on Days 1 and 8 of every three-week cycle. In some embodiments, the method further comprises administering an immune checkpoint inhibitor by an intravenous (IV) injection or infusion one or more times in each three-week cycle. In some embodiments, the method further comprises administering an immune checkpoint inhibitor by an intravenous (IV) injection or infusion on Day 1 of every three-week cycle. In some embodiments, the immune checkpoint inhibitor is pembrolizumab, and wherein pembrolizumab is administered at amount of about 200 mg over about 30 minutes. In other embodiments, the immune checkpoint inhibitor is atezolizumab, and wherein atezolizumab is administered at amount of about 1200 mg over about 60 minutes or 30 minutes. In some embodiments, the antibody drug conjugate is administered to patients with urothelial or bladder cancer who have shown disease progression or relapse during or after treatment with another cancer treatment. In some embodiments, the antibody drug conjugate is administered to patients with metastatic urothelial or bladder cancer who have shown disease progression or relapse during or after treatment with another cancer treatment. In some embodiments, the antibody drug conjugate is administered to patients with locally advanced urothelial or bladder cancer who have shown disease progression or relapse during or after treatment with another cancer treatment. In some embodiments, the ADC of the methods for which the various dosages are described in this paragraph is enfortumab vedotin (EV).

In other more specific embodiments, the antibody drug conjugate formulated in the pharmaceutical composition provided herein is administered at a dose of about 0.5 mg/kg, about 0.75 mg/kg, 1 mg/kg, about 1.25 mg/kg, or about 1.5 mg/kg of the subject's body weight by an intravenous (IV) injection or infusion over about 30 minutes three times every four-week cycle. In some embodiments, the antibody drug conjugate formulated in the pharmaceutical composition is administered on Days 1, 8 and 15 of every 28-day (four-week) cycle. In some embodiments, the antibody drug conjugate formulated in the pharmaceutical composition is administered by an intravenous (IV) injection or infusion over about 30 minutes on Days 1, 8 and 15 of every 28-day (four-week) cycle. In some embodiments, the method further comprises administering an immune checkpoint inhibitor by an intravenous (IV) injection or infusion one or more times in each four-week cycle. In some embodiments, the immune checkpoint inhibitor is pembrolizumab. In other embodiments, the immune checkpoint inhibitor is atezolizumab. In some embodiments, the antibody drug conjugate is administered to patients with urothelial or bladder cancer who have shown disease progression or relapse during or after treatment with another cancer treatment. In some embodiments, the antibody drug conjugate is administered to patients with metastatic urothelial or bladder cancer who have shown disease progression or relapse during or after treatment with another cancer treatment. In some embodiments, the antibody drug conjugate is administered to patients with locally advanced urothelial or bladder cancer who have shown disease progression or relapse during or after treatment with another cancer treatment. In some embodiments, the ADC of the methods for which the various dosages are described in this paragraph is enfortumab vedotin (EV).

In some embodiments of the various methods provided herein, the ADC is administered at a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments, the ADC is administered at a dose of about 1 to about 10 mg/kg of the subject's body weight. In certain embodiments, the ADC is administered at a dose of about 1 to about 5 mg/kg of the subject's body weight. In other embodiments, the ADC is administered at a dose of about 1 to about 2.5 mg/kg of the subject's body weight. In further embodiments, the ADC is administered at a dose of about 1 to about 1.25 mg/kg of the subject's body weight. In some embodiments, the ADC is administered at a dose of about 0.25 mg/kg of the subject's body weight. In some embodiments, the ADC is administered at a dose of about 0.5 mg/kg of the subject's body weight. In some embodiments, the ADC is administered at a dose of about 0.75 mg/kg of the subject's body weight. In some embodiments, the ADC is administered at a dose of about 1.0 mg/kg of the subject's body weight. In some embodiments, the ADC is administered at a dose of about 1.25 mg/kg of the subject's body weight. In some embodiments, the ADC is administered at a dose of about 1.5 mg/kg of the subject's body weight. In some embodiments, the ADC is administered at a dose of about 1.75 mg/kg of the subject's body weight. In some embodiments, the ADC is administered at a dose of about 2.0 mg/kg of the subject's body weight. In some embodiments, the ADC is administered at a dose of about 2.25 mg/kg of the subject's body weight. In some embodiments, the ADC is administered at a dose of about 2.5 mg/kg of the subject's body weight.

In certain embodiments of the various methods provided herein, the ADC is administered at a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In some embodiments, the ADC is administered at a dose of 1 to 10 mg/kg of the subject's body weight. In certain embodiments, the ADC is administered at a dose of 1 to 5 mg/kg of the subject's body weight. In other embodiments, the ADC is administered at a dose of 1 to 2.5 mg/kg of the subject's body weight. In further embodiments, the ADC is administered at a dose of 1 to 1.25 mg/kg of the subject's body weight. In some embodiments, the ADC is administered at a dose of 0.25 mg/kg of the subject's body weight. In some embodiments, the ADC is administered at a dose of 0.5 mg/kg of the subject's body weight. In some embodiments, the ADC is administered at a dose of 0.75 mg/kg of the subject's body weight. In some embodiments, the ADC is administered at a dose of 1.0 mg/kg of the subject's body weight. In some embodiments, the ADC is administered at a dose of 1.25 mg/kg of the subject's body weight. In some embodiments, the ADC is administered at a dose of 1.5 mg/kg of the subject's body weight. In some embodiments, the ADC is administered at a dose of 1.75 mg/kg of the subject's body weight. In some embodiments, the ADC is administered at a dose of 2.0 mg/kg of the subject's body weight. In some embodiments, the ADC is administered at a dose of 2.25 mg/kg of the subject's body weight. In some embodiments, the ADC is administered at a dose of 2.5 mg/kg of the subject's body weight.

In some embodiments of the various methods provided herein, including those methods requiring a first and a second dose, the first dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the first dose of the ADC is a dose of about 1 to about 10 mg/kg of the subject's body weight. In some embodiments of the various methods provided herein, including those methods requiring a first and a second dose, the first dose of the ADC is a dose of about 1 to about 5 mg/kg of the subject's body weight. In some embodiments of the various methods provided herein, including those methods requiring a first and a second dose, the first dose of the ADC is a dose of about 1 to about 2.5 mg/kg of the subject's body weight. In some embodiments of the various methods provided herein, including those methods requiring a first and a second dose, the first dose of the ADC is a dose of about 1 to about 1.25 mg/kg of the subject's body weight. In some embodiments of the various methods provided herein, including those methods requiring a first and a second dose, the first dose of the ADC is a dose of about 0.25 mg/kg of the subject's body weight. In some embodiments of the various methods provided herein, including those methods requiring a first and a second dose, the first dose of the ADC is a dose of about 0.5 mg/kg of the subject's body weight. In some embodiments, the first dose of the ADC is a dose of about 0.75 mg/kg of the subject's body weight. In some embodiments, the first dose of the ADC is a dose of about 1.0 mg/kg of the subject's body weight. In some embodiments, the first dose of the ADC is a dose of about 1.25 mg/kg of the subject's body weight. In some embodiments, the first dose of the ADC is a dose of about 1.5 mg/kg of the subject's body weight. In some embodiments, the first dose of the ADC is a dose of about 1.75 mg/kg of the subject's body weight. In some embodiments, the first dose of the ADC is a dose of about 2.0 mg/kg of the subject's body weight. In some embodiments, the first dose of the ADC is a dose of about 2.25 mg/kg of the subject's body weight. In some embodiments, the first dose of the ADC is a dose of or about 2.5 mg/kg of the subject's body weight.

In certain embodiments of the various methods provided herein, including those methods requiring a first and a second dose, the first dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the first dose of the ADC is a dose of 1 to 10 mg/kg of the subject's body weight. In some embodiments of the various methods provided herein, including those methods requiring a first and a second dose, the first dose of the ADC is a dose of 1 to 5 mg/kg of the subject's body weight. In some embodiments of the various methods provided herein, including those methods requiring a first and a second dose, the first dose of the ADC is a dose of 1 to 2.5 mg/kg of the subject's body weight. In some embodiments of the various methods provided herein, including those methods requiring a first and a second dose, the first dose of the ADC is a dose of 1 to 1.25 mg/kg of the subject's body weight. In some embodiments of the various methods provided herein, including those methods requiring a first and a second dose, the first dose of the ADC is a dose of 0.25 mg/kg of the subject's body weight. In some embodiments of the various methods provided herein, including those methods requiring a first and a second dose, the first dose of the ADC is a dose of 0.5 mg/kg of the subject's body weight. In some embodiments, the first dose of the ADC is a dose of 0.75 mg/kg of the subject's body weight. In some embodiments, the first dose of the ADC is a dose of 1.0 mg/kg of the subject's body weight. In some embodiments, the first dose of the ADC is a dose of 1.25 mg/kg of the subject's body weight. In some embodiments, the first dose of the ADC is a dose of 1.5 mg/kg of the subject's body weight. In some embodiments, the first dose of the ADC is a dose of 1.75 mg/kg of the subject's body weight. In some embodiments, the first dose of the ADC is a dose of 2.0 mg/kg of the subject's body weight. In some embodiments, the first dose of the ADC is a dose of 2.25 mg/kg of the subject's body weight. In some embodiments, the first dose of the ADC is a dose of 2.5 mg/kg of the subject's body weight.

In some embodiments of the various methods provided herein, including those methods requiring a first and a second dose, the second dose of the ADC is lower than the first dose by about 0.1 mg/kg to about 2 mg/kg of the subject's body weight. In some embodiments of the various methods provided herein, including those methods requiring a first and a second dose, the second dose of the ADC is lower than the first dose by about 0.1 mg/kg of the subject's body weight. In some embodiments of the various methods provided herein, including those methods requiring a first and a second dose, the second dose of the ADC is lower than the first dose by about 0.2 mg/kg of the subject's body weight. In some embodiments of the various methods provided herein, including those methods requiring a first and a second dose, the second dose of the ADC is lower than the first dose by about 0.25 mg/kg of the subject's body weight. In some embodiments of the various methods provided herein, including those methods requiring a first and a second dose, the second dose of the ADC is lower than the first dose by about 0.3 mg/kg of the subject's body weight. In some embodiments of the various methods provided herein, including those methods requiring a first and a second dose, the second dose of the ADC is lower than the first dose by about 0.4 mg/kg of the subject's body weight. In some embodiments of the various methods provided herein, including those methods requiring a first and a second dose, the second dose of the ADC is lower than the first dose by about 0.5 mg/kg of the subject's body weight. In some embodiments of the various methods provided herein, including those methods requiring a first and a second dose, the second dose of the ADC is lower than the first dose by about 0.6 mg/kg of the subject's body weight. In some embodiments of the various methods provided herein, including those methods requiring a first and a second dose, the second dose of the ADC is lower than the first dose by about 0.7 mg/kg of the subject's body weight. In some embodiments of the various methods provided herein, including those methods requiring a first and a second dose, the second dose of the ADC is lower than the first dose by about 0.75 mg/kg of the subject's body weight. In some embodiments of the various methods provided herein, including those methods requiring a first and a second dose, the second dose of the ADC is lower than the first dose by about 0.8 mg/kg of the subject's body weight. In some embodiments of the various methods provided herein, including those methods requiring a first and a second dose, the second dose of the ADC is lower than the first dose by about 0.9 mg/kg of the subject's body weight. In some embodiments of the various methods provided herein, including those methods requiring a first and a second dose, the second dose of the ADC is lower than the first dose by about 1 mg/kg of the subject's body weight. In some embodiments of the various methods provided herein, including those methods requiring a first and a second dose, the second dose of the ADC is lower than the first dose by about 1.1 mg/kg of the subject's body weight. In some embodiments of the various methods provided herein, including those methods requiring a first and a second dose, the second dose of the ADC is lower than the first dose by about 1.2 mg/kg of the subject's body weight. In some embodiments of the various methods provided herein, including those methods requiring a first and a second dose, the second dose of the ADC is lower than the first dose by about 1.25 mg/kg of the subject's body weight. In some embodiments of the various methods provided herein, including those methods requiring a first and a second dose, the second dose of the ADC is lower than the first dose by about 1.3 mg/kg of the subject's body weight. In some embodiments of the various methods provided herein, including those methods requiring a first and a second dose, the second dose of the ADC is lower than the first dose by about 1.4 mg/kg of the subject's body weight. In some embodiments of the various methods provided herein, including those methods requiring a first and a second dose, the second dose of the ADC is lower than the first dose by about 1.5 mg/kg of the subject's body weight. In some embodiments of the various methods provided herein, including those methods requiring a first and a second dose, the second dose of the ADC is lower than the first dose by about 1.6 mg/kg of the subject's body weight. In some embodiments of the various methods provided herein, including those methods requiring a first and a second dose, the second dose of the ADC is lower than the first dose by about 1.7 mg/kg of the subject's body weight. In some embodiments of the various methods provided herein, including those methods requiring a first and a second dose, the second dose of the ADC is lower than the first dose by about 1.75 mg/kg of the subject's body weight. In some embodiments of the various methods provided herein, including those methods requiring a first and a second dose, the second dose of the ADC is lower than the first dose by about 1.8 mg/kg of the subject's body weight. In some embodiments of the various methods provided herein, including those methods requiring a first and a second dose, the second dose of the ADC is lower than the first dose by about 1.9 mg/kg of the subject's body weight. In some embodiments of the various methods provided herein, including those methods requiring a first and a second dose, the second dose of the ADC is lower than the first dose by about 2 mg/kg of the subject's body weight.

In certain embodiments of the various methods provided herein, including those methods requiring a first and a second dose, the second dose of the ADC is lower than the first dose by 0.1 mg/kg to 2 mg/kg of the subject's body weight. In some embodiments of the various methods provided herein, including those methods requiring a first and a second dose, the second dose of the ADC is lower than the first dose by 0.1 mg/kg of the subject's body weight. In some embodiments of the various methods provided herein, including those methods requiring a first and a second dose, the second dose of the ADC is lower than the first dose by 0.2 mg/kg of the subject's body weight. In some embodiments of the various methods provided herein, including those methods requiring a first and a second dose, the second dose of the ADC is lower than the first dose by 0.25 mg/kg of the subject's body weight. In some embodiments of the various methods provided herein, including those methods requiring a first and a second dose, the second dose of the ADC is lower than the first dose by 0.3 mg/kg of the subject's body weight. In some embodiments of the various methods provided herein, including those methods requiring a first and a second dose, the second dose of the ADC is lower than the first dose by 0.4 mg/kg of the subject's body weight. In some embodiments of the various methods provided herein, including those methods requiring a first and a second dose, the second dose of the ADC is lower than the first dose by 0.5 mg/kg of the subject's body weight. In some embodiments of the various methods provided herein, including those methods requiring a first and a second dose, the second dose of the ADC is lower than the first dose by 0.6 mg/kg of the subject's body weight. In some embodiments of the various methods provided herein, including those methods requiring a first and a second dose, the second dose of the ADC is lower than the first dose by 0.7 mg/kg of the subject's body weight. In some embodiments of the various methods provided herein, including those methods requiring a first and a second dose, the second dose of the ADC is lower than the first dose by 0.75 mg/kg of the subject's body weight. In some embodiments of the various methods provided herein, including those methods requiring a first and a second dose, the second dose of the ADC is lower than the first dose by 0.8 mg/kg of the subject's body weight. In some embodiments of the various methods provided herein, including those methods requiring a first and a second dose, the second dose of the ADC is lower than the first dose by 0.9 mg/kg of the subject's body weight. In some embodiments of the various methods provided herein, including those methods requiring a first and a second dose, the second dose of the ADC is lower than the first dose by 1 mg/kg of the subject's body weight. In some embodiments of the various methods provided herein, including those methods requiring a first and a second dose, the second dose of the ADC is lower than the first dose by 1.1 mg/kg of the subject's body weight. In some embodiments of the various methods provided herein, including those methods requiring a first and a second dose, the second dose of the ADC is lower than the first dose by 1.2 mg/kg of the subject's body weight. In some embodiments of the various methods provided herein, including those methods requiring a first and a second dose, the second dose of the ADC is lower than the first dose by 1.25 mg/kg of the subject's body weight. In some embodiments of the various methods provided herein, including those methods requiring a first and a second dose, the second dose of the ADC is lower than the first dose by 1.3 mg/kg of the subject's body weight. In some embodiments of the various methods provided herein, including those methods requiring a first and a second dose, the second dose of the ADC is lower than the first dose by 1.4 mg/kg of the subject's body weight. In some embodiments of the various methods provided herein, including those methods requiring a first and a second dose, the second dose of the ADC is lower than the first dose by 1.5 mg/kg of the subject's body weight. In some embodiments of the various methods provided herein, including those methods requiring a first and a second dose, the second dose of the ADC is lower than the first dose by 1.6 mg/kg of the subject's body weight. In some embodiments of the various methods provided herein, including those methods requiring a first and a second dose, the second dose of the ADC is lower than the first dose by 1.7 mg/kg of the subject's body weight. In some embodiments of the various methods provided herein, including those methods requiring a first and a second dose, the second dose of the ADC is lower than the first dose by 1.75 mg/kg of the subject's body weight. In some embodiments of the various methods provided herein, including those methods requiring a first and a second dose, the second dose of the ADC is lower than the first dose by 1.8 mg/kg of the subject's body weight. In some embodiments of the various methods provided herein, including those methods requiring a first and a second dose, the second dose of the ADC is lower than the first dose by 1.9 mg/kg of the subject's body weight. In some embodiments of the various methods provided herein, including those methods requiring a first and a second dose, the second dose of the ADC is lower than the first dose by 2 mg/kg of the subject's body weight.

In some embodiments of the various methods provided herein, including those methods requiring a first and a second dose, the second dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the second dose of the ADC is a dose of about 1 to about 10 mg/kg of the subject's body weight. In some embodiments of the various methods provided herein, including those methods requiring a first and a second dose, the second dose of the ADC is a dose of about 1 to about 5 mg/kg of the subject's body weight. In some embodiments of the various methods provided herein, including those methods requiring a first and a second dose, the second dose of the ADC is a dose of about 1 to about 2.5 mg/kg of the subject's body weight. In some embodiments of the various methods provided herein, including those methods requiring a first and a second dose, the second dose of the ADC is a dose of about 1 to about 1.25 mg/kg of the subject's body weight. In some embodiments of the various methods provided herein, including those methods requiring a first and a second dose, the second dose of the ADC is a dose of about 0.25 mg/kg of the subject's body weight. In some embodiments of the various methods provided herein, including those methods requiring a first and a second dose, the second dose of the ADC is a dose of about 0.5 mg/kg of the subject's body weight. In some embodiments of the various methods provided herein, including those methods requiring a first and a second dose, the second dose of the ADC is a dose of about 0.75 mg/kg of the subject's body weight. In some embodiments of the various methods provided herein, including those methods requiring a first and a second dose, the second dose of the ADC is a dose of about 1.0 mg/kg of the subject's body weight. In some embodiments of the various methods provided herein, including those methods requiring a first and a second dose, the second dose of the ADC is a dose of about 1.25 mg/kg of the subject's body weight. In some embodiments of the various methods provided herein, including those methods requiring a first and a second dose, the second dose of the ADC is a dose of about 1.5 mg/kg of the subject's body weight. In some embodiments of the various methods provided herein, including those methods requiring a first and a second dose, the second dose of the ADC is a dose of about 1.75 mg/kg of the subject's body weight. In some embodiments of the various methods provided herein, including those methods requiring a first and a second dose, the second dose of the ADC is a dose of about 2.0 mg/kg of the subject's body weight. In some embodiments of the various methods provided herein, including those methods requiring a first and a second dose, the second dose of the ADC is a dose of about 2.25 mg/kg of the subject's body weight. In some embodiments of the various methods provided herein, including those methods requiring a first and a second dose, the second dose of the ADC is a dose of about 2.5 mg/kg of the subject's body weight.

In certain embodiments of the various methods provided herein, including those methods requiring a first and a second dose, the second dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the second dose of the ADC is a dose of 1 to 10 mg/kg of the subject's body weight. In some embodiments of the various methods provided herein, including those methods requiring a first and a second dose, the second dose of the ADC is a dose of 1 to 5 mg/kg of the subject's body weight. In some embodiments of the various methods provided herein, including those methods requiring a first and a second dose, the second dose of the ADC is a dose of 1 to 2.5 mg/kg of the subject's body weight. In some embodiments of the various methods provided herein, including those methods requiring a first and a second dose, the second dose of the ADC is a dose of 1 to 1.25 mg/kg of the subject's body weight. In some embodiments of the various methods provided herein, including those methods requiring a first and a second dose, the second dose of the ADC is a dose of 0.25 mg/kg of the subject's body weight. In some embodiments of the various methods provided herein, including those methods requiring a first and a second dose, the second dose of the ADC is a dose of 0.5 mg/kg of the subject's body weight. In some embodiments of the various methods provided herein, including those methods requiring a first and a second dose, the second dose of the ADC is a dose of 0.75 mg/kg of the subject's body weight. In some embodiments of the various methods provided herein, including those methods requiring a first and a second dose, the second dose of the ADC is a dose of 1.0 mg/kg of the subject's body weight. In some embodiments of the various methods provided herein, including those methods requiring a first and a second dose, the second dose of the ADC is a dose of 1.25 mg/kg of the subject's body weight. In some embodiments of the various methods provided herein, including those methods requiring a first and a second dose, the second dose of the ADC is a dose of 1.5 mg/kg of the subject's body weight. In some embodiments of the various methods provided herein, including those methods requiring a first and a second dose, the second dose of the ADC is a dose of 1.75 mg/kg of the subject's body weight. In some embodiments of the various methods provided herein, including those methods requiring a first and a second dose, the second dose of the ADC is a dose of 2.0 mg/kg of the subject's body weight. In some embodiments of the various methods provided herein, including those methods requiring a first and a second dose, the second dose of the ADC is a dose of 2.25 mg/kg of the subject's body weight. In some embodiments of the various methods provided herein, including those methods requiring a first and a second dose, the second dose of the ADC is a dose of 2.5 mg/kg of the subject's body weight.

In certain embodiments of the various methods provided herein, including those methods requiring a first and a second dose, the second dose of the ADC is identical to the first dose of the ADC.

In some embodiments of the methods provided herein, the ADC is administered by an intravenous (IV) injection or infusion. In one embodiment, the first dose of the ADC is administered by an IV injection. In another embodiment, the first dose of the ADC is administered by an IV infusion. In yet another embodiment, the second dose of the ADC is administered by an IV injection. In yet another embodiment, the second dose of the ADC is administered by an IV injection infusion. In one embodiment, the first dose of the ADC is administered by an IV injection and the second dose of the ADC is administered by an IV injection. In another embodiment, the first dose of the ADC is administered by an IV infusion and the second dose of the ADC is administered by an IV injection. In yet another embodiment, the second dose of the ADC is administered by an IV injection and the second dose of the ADC is administered by an IV injection infusion. In yet another embodiment, the second dose of the ADC is administered by an IV injection infusion and the second dose of the ADC is administered by an IV injection infusion. In some embodiments, the ADC of the methods for which the various dosages are described in this paragraph is enfortumab vedotin (EV).

In certain embodiments of the methods provided herein, the ADC is administered by an IV injection or infusion three times every four-week cycle. In some embodiments of the methods provided herein, the first dose of the ADC is administered by an IV injection or infusion three times every four-week cycle. In some embodiments of the methods provided herein, the second dose of the ADC is administered by an IV injection or infusion three times every four-week cycle. In some embodiments of the methods provided herein, the first dose of the ADC is administered by an IV injection or infusion three times every four-week cycle and the second dose of the ADC is administered by an IV injection or infusion three times every four-week cycle. In some embodiments, the ADC of the methods for which the various dosages are described in this paragraph is enfortumab vedotin (EV).

In some embodiments of the methods provided herein, the ADC is administered by an IV injection or infusion on Days 1, 8 and 15 of every four-week cycle. In some embodiments, the first dose of ADC is administered by an IV injection or infusion on Days 1, 8 and 15 of every four-week cycle. In some embodiments, the second dose of ADC is administered by an IV injection or infusion on Days 1, 8 and 15 of every four-week cycle. In some embodiments, the first dose of ADC is administered by an IV injection or infusion on Days 1, 8 and 15 of every four-week cycle and the second dose of ADC is administered by an IV injection or infusion on Days 1, 8 and 15 of every four-week cycle. In some embodiments, the ADC of the methods for which the various dosages are described in this paragraph is enfortumab vedotin (EV).

In certain embodiments of the methods provided herein, the ADC is administered by an IV injection or infusion over about 30 minutes three times every four-week cycle. In some embodiments, the first dose of the ADC is administered by an IV injection or infusion over about 30 minutes three times every four-week cycle. In some embodiments, the second dose of the ADC is administered by an IV injection or infusion over about 30 minutes three times every four-week cycle. In some embodiments, the first dose of the ADC is administered by an IV injection or infusion over about 30 minutes three times every four-week cycle and the second dose of the ADC is administered by an IV injection or infusion over about 30 minutes three times every four-week cycle. In some embodiments, the ADC of the methods for which the various dosages are described in this paragraph is enfortumab vedotin (EV).

In some embodiments of the methods provided herein, the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle. In some embodiments of the methods provided herein, the second dose of the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle and the second dose of the ADC is administered by an IV injection or infusion over about 30 minutes on Days 1, 8 and 15 of every four-week cycle. In some embodiments, the ADC of the methods for which the various dosages are described in this paragraph is enfortumab vedotin (EV).

In other more specific embodiments, the antibody drug conjugate formulated in the pharmaceutical composition provided herein is administered at a dose of about 1 mg/kg, 1.25 mg/kg, or about 1.5 mg/kg of the subject's body weight by an intravenous (IV) injection or infusion over about 30 minutes three times every 28-day cycle. In some embodiments, the antibody drug conjugate formulated in the pharmaceutical composition is administered by an intravenous (IV) injection or infusion over about 30 minutes on Days 1, 8 and 15 of every 28-day cycle. In some embodiments, the method further comprises administering an immune checkpoint inhibitor by an intravenous (IV) injection or infusion one or more times in each four-week cycle. In some embodiments of the methods provided herein, the ADC is administered three times within a 28 day cycle. In some embodiments of the methods provided herein, the ADC is administered on Days 1, 8 and 15 of a 28 day cycle. In some embodiments, the ADC of the methods for which the various dosages are described in this paragraph is enfortumab vedotin (EV).

### 5.8 Methods for Determining the Biomarkers

The disclosure provides that the expression of any of the markers provided herein can be determined by various methods known in the field. In some embodiments, the expression of the markers can be determined by the amount or relative amount of mRNA transcribed from the marker genes. In one embodiment, the expression of the marker genes can be determined by the amount or relative amount of the protein products encoded by the marker genes. In another embodiment, the expression of the marker genes can be determined by the level of biological or chemical response induced by the protein products encoded by the marker genes. Additionally, in certain embodiments, the expression of the marker genes can be determined by the expression of one or more genes that correlates with the expression of the marker genes.

As described above, levels or amounts of gene transcripts (e.g. mRNA) of the marker genes can be used as a proxy for the expression levels of markers genes. Numerous different PCR or qPCR protocols are known in the art including those exemplified herein. In some embodiments, the various PCR or qPCR methods are applied or adapted for determining the mRNA level of the various marker genes. Quantitative PCR (qPCR) (also referred as real-time PCR) is applied and adapted in some embodiments as it provides not only a quantitative measurement, but also reduced time and contamination. As used herein, “quantitative PCR (or “qPCR”) refers to the direct monitoring of the progress of PCR amplification as it is occurring without the need for repeated sampling of the reaction products. In quantitative PCR, the reaction products can be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. When qPCR is applied to determine mRNA expression level, an extra step of reverse-transcription of mRNA to DNA is performed before the qPCR analysis. Examples of PCR methods can be found in the literature (Wong et al., BioTechniques 39:75-85 (2005); D'haene et al., Methods 50:262-270 (2010)), which is incorporated by reference herein in its entirety. Examples of PCR assays can also be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In one specific embodiment, qPCR can be performed to determine or measure the mRNA levels of the marker genes as follows. Briefly, mean Ct (cycle threshold) values (or referred to herein interchangeably as Cq (quantification cycle)) of replicate qPCR reactions for the marker genes and one or more housekeeping genes are determined. Mean Ct values for the marker genes can be then normalized to the Ct values of the housekeeping genes using the following exemplary formula: marker-gene-ΔCt=(mean Ct of marker gene−mean Ct of housekeeping gene A). The relative marker-gene-ΔCt can then be used to determine relative level of marker gene mRNA, for example by using the formula of mRNA expression=2^{-ΔCt}. For a summary of Ct and Cq values, see MIQE guideline (Bustin et al., The MIQE Guidelines: Minimum Information for Publication of Quantitative Real-Time PCR Experiments, Clinical Chemistry 55:4 (2009)).

Other commonly used methods known in the art can also be used for the quantification of RNA transcripts of the marker genes in a sample as the proxy for the expression of the marker genes, including northern blotting and in situ hybridization (Parker & Barnes, Methods in Molecular Biology 106:247-283 (1999)); RNAse protection assays (Hod, Biotechniques 13:852-854 (1992)); microarrays (Hoheisel et al., Nature Reviews Genetics 7:200-210 (2006); Jaluria et al., Microbial Cell Factories 6:4 (2007)); and polymerase chain reaction (PCR) (Weis et al, Trends in Genetics 8:263-264 (1992)). RNA in situ hybridization (ISH) is a molecular biology technique widely used to measure and localize specific RNA sequences, for example, messenger RNAs (mRNAs), long non-coding RNAs (lncRNAs), and microRNAs (miRNAs) within cells, such as circulating tumor cells (CTCs) or tissue sections, while preserving the cellular and tissue context. ISH is a type of hybridization that uses a directly or indirectly labeled complementary DNA or RNA strand, such as a probe, to bind to and localize a specific nucleic acid, such as DNA or RNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)). RNA ISH therefore provides for spatial-temporal visualization as well as quantification of gene expression within cells and tissues. It has wide applications in research and in diagnostics (Hu et al., Biomark. Res. 2(1):1-13, doi: 10.1186/2050-7771-2-3 (2014); Ratan et al., Cureus 9(6):e1325. doi: 10.7759/cureus.1325 (2017); Weier et al., Expert Rev. Mol. Diagn. 2(2):109-119 (2002)). Fluorescent RNA ISH utilizes fluorescent dyes and fluorescent microscopes for RNA labeling and detection, respectively. Fluorescent RNA ISH can provides for multiplexing of four to five target sequences.

Alternatively, RNA transcripts of the marker genes in a sample as the proxy for the expression of the marker genes can be determined by sequencing techniques. Representative methods for sequencing-based gene expression analysis include Serial Analysis of Gene Expression (SAGE), and gene expression analysis by massively parallel signature sequencing (MPSS).

In some embodiments, expression of the marker genes can be determined by the relative abundance of the RNA transcripts (including for example mRNA) of the marker genes in a pool of total transcribed RNA. Such relative abundance of the RNA transcripts of the marker genes can be determined by next generation sequencing, which is known as RNA-seq. In one example of the RNA-seq procedure, RNAs from different sources (blood, tissue, cells) are purified, optionally enriched (e.g. with oligo (dT) primers), converted to cDNA, and fragmented. Millions or even billions of short sequence reads are generated from the randomly fragmented cDNA library. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference. The expression level of each mRNA transcript of the marker genes is determined by the total number of mapped fragments upon normalization, which is directly proportional to its abundance level. A few normalization schemes are known and used to facilitate the use of the abundance of the RNA transcripts as the parameter for determining gene expression, including RPKM (Reads Per Kilobase Million), FPKM (Fragments Per Kilobase Million), and/or TPM (Transcripts Per Kilobase Million). Briefly, RPKM can be calculated as follows: count up the total reads in a sample and divide that number by 1,000,000—which is the “per million” scaling factor; divide the read counts by the “per million” scaling factor, which normalizes for sequencing depth, giving the reads per million (RPM); and divide the RPM values by the length of the gene, in kilobases, which gives RPKM. FPKM is closely related to RPKM except with fragment replacing read. RPKM was made for single-end RNA-seq, where every read corresponded to a single fragment that was sequenced. FPKM was made for paired-end RNA-seq, in which two reads can correspond to a single fragment, or, if one read in the pair did not map, one read can correspond to a single fragment. TPM is very similar to RPKM and FPKM and is calculated as follows: divide the read counts by the length of each gene in kilobases, which gives the reads per kilobase (RPK); count up all the RPK values in a sample and divide this number by 1,000,000, which gives the “per million” scaling factor; divide the RPK values by the “per million” scaling factor, which gives TPM. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference.

In one embodiment, the expression of the marker genes is determined by RNA-seq, for example by TPM, RPKM, and/or FPKM. In some embodiments, the expression of the marker genes is determined by TPM. In some embodiments, the expression of the marker genes is determined by RPKM. In some embodiments, the expression of the marker genes is determined by FPKM.

As described earlier, the expression of the marker genes can be determined in a sample from a subject. In some embodiments, the sample is a blood sample, a serum sample, a plasma sample, bodily fluid (e.g. tissue fluid including cancer tissue fluid), or a tissue (e.g. cancer tissue or the tissue surrounding the cancer). In some embodiments, the sample is a tissue sample. In some embodiments, the tissue sample is tissue fractions isolated or extracted from a mammal, in particular a human. In some embodiments, the tissue sample is a population of cells isolated or extracted from a mammal, in particular a human. In some embodiments, the tissue sample is a sample obtained from a biopsy. In certain embodiments, the samples can be obtained from a variety of organs of a subject, including a human subject. In some embodiments, the samples are obtained from organs of a subject having a cancer. In some embodiments, the samples are obtained from organs having a cancer in a subject having a cancer. In other embodiments, the samples, for example reference samples, are obtained from normal organs from the patient or from a second human subject.

In certain embodiments of the methods provided herein, the tissue includes a tissue from bladder, ureter, breast, lung, colon, rectum, ovary, Fallopian tube, esophagus, cervix, uterine endometrium, skin, larynx, bone marrow, salivary gland, kidney, prostate, brain, spinal cord, placenta, adrenal, pancreas, parathyroid, hypophysis, testis, thyroid, spleen, tonsil, thymus, heart, stomach, small intestine, liver, skeletal muscle, peripheral nerve, mesothelium, or eye.

In further embodiments of the methods provided herein, the expression of the various marker genes can be detected by a variety of immunoassays known in the art, including an immunohistochemistry (IHC) assay, an immunoblotting assay, a FACS assay, and an ELISA.

The expression of the various marker genes can be detected by antibodies against the protein products encoded by the marker genes in a variety of IHC assays. IHC staining of tissue sections has been shown to be a reliable method of assessing or detecting the presence of proteins in a sample. IHC techniques utilize an antibody to probe and visualize cellular antigens in situ, generally by chromogenic or fluorescent methods. Primary antibodies or antisera, such as polyclonal antisera and monoclonal antibodies that specifically target the protein products encoded by the marker genes, can be used to detect expression of the marker genes in an IHC assay. In some embodiments, the tissue sample is contacted with a primary antibody for a specific target for a period of time sufficient for the antibody-target binding to occur. As discussed in detail earlier, the antibodies can be detected by direct labels on the antibodies themselves, for example, radioactive labels, fluorescent labels, hapten labels such as biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Leica BOND Autostainer and Leica Bond Refine Detection system is an example of such an automated system.

In some embodiments, an IHC assay is performed with an unlabeled primary antibody in conjunction with a labeled secondary antibody in an indirect assay. The indirect assay utilizes two antibodies for the detection of the protein products encoded by the marker genes in a tissue sample. First, an unconjugated primary antibody was applied to the tissue (first layer), which reacts with the target antigen in the tissue sample. Next, an enzyme-labeled secondary antibody is applied, which specifically recognize the antibody isotype of the primary antibody (second layer). The secondary antibody reacts with the primary antibody, followed by substrate-chromogen application. The second-layer antibody can be labeled with an enzyme such as a peroxidase, which reacts with the chromogen 3,3′-diaminobenzidine (DAB) to produce brown precipitate at the reaction site. This method is sensitive and versatile due to the potential signal amplification through a signal amplification system.

In certain embodiments to increase the sensitivity of the detection, a signal amplification system can be used. “A signal amplification system”, as used herein, means a system of reagents and methods that can be used to increase the signal from detecting the bound primary or the secondary antibody. A signal amplification system increases the sensitivity of the target protein detection, increases the detected signal, and decreases the lower boundary of the detection limits. There are several types of signal amplification systems including an enzyme labeling system and macrolabeling system. These systems/approaches are not mutually exclusive and can be used in combination for additive effect.

Macrolabels or macrolabeling system are collections of labels numbering in the tens (e.g. phycobiliproteins) to millions (e.g. fluorescent microspheres) attached to or incorporated in a common scaffold. The scaffold can be coupled to a target-specific affinity reagent such as an antibody, and the incorporated labels are thereby collectively associated with the target upon binding. The labels in the macrolabels can be any of the labels described herein such as fluorophores, haptens, enzymes, and/or radioisotopes. In one embodiment of the signal amplification system, a labeled chain polymer-conjugated secondary antibody was used. The polymer technology utilized an HRP enzyme-labeled inert “spine” molecule of dextran to which 1, 2, 3, 4, 5, 6, 7, 8, 9 10, 15, 20, 25, 30, 50 or more molecules of secondary antibodies can be attached, making the system even more sensitive.

Signal amplification system based on an enzyme labeling system utilizes the catalytic activity of enzymes, such as horseradish peroxidase (HRP) or alkaline phosphatase to generate high-density labeling of a target protein or nucleic acid sequence in situ. In one embodiment, tyramide can be used to increase the signal of HRP. In such a system, HRP enzymatically converts the labeled tyramide derivative into highly reactive, short-lived tyramide radicals. The labeled active tyramide radicals then covalently couple to residues (principally the phenol moiety of protein tyrosine residues) in the vicinity of the HRP-antibody-target interaction site, resulting amplification of the number of labels at the site with minimal diffusion-related loss of signal localization. Consequently, the signal can be amplified 1, 2, 3, 4, 5, 6, 7, 8, 9 10, 15, 20, 25, 30, 50, 75, or 100 folds. As known to a person skilled in the art, the labels on the tyramide can be any labels described herein, including fluorophores, enzymes, haptens, radioisotopes, and/or photophoresis. Other enzyme-based reactions can be utilized to create signal amplification as well. For example, Enzyme-Labeled Fluorescence (ELF) signal amplification is available for alkaline phosphatase, wherein the alkaline phosphatase enzymatically cleaves a weakly blue-fluorescent substrate (ELF 97 phosphate) and converts it into a bright yellow-green-fluorescent precipitate that exhibits an unusually large Stokes shift and excellent photostability. Both tyramide-based signal amplification system and ELF signal amplification are available commercially, for example from ThermoFisher Scientific (Waltham, MA USA 02451).

Thus in some embodiments of the methods provided herein, the expression level of the marker genes is detected with IHC using a signal amplification system. In some embodiments, the specimen is then counterstained to identify cellular and subcellular elements.

In some embodiments, the expression level of the protein products encoded by the marker genes can also be detected with antibodies against the protein products encoded by the marker genes using an immunoblotting assay. In some embodiments of an immunoblotting assay, proteins are often (but do not have to be) separated by electrophoresis and transferred onto membranes (usually nitrocellulose or PVDF membrane). Similar to the IHC assays, primary antibodies or antisera, such as polyclonal antisera and monoclonal antibodies that specifically target the protein products encoded by the marker genes, can be used to detect expression of the marker genes. In some embodiments, the membrane is contacted with a primary antibody for a specific target for a period of time sufficient for the antibody-antigen binding to occur and the bound antibodies can be detected by direct labels on the primary antibodies themselves, e.g. with radioactive labels, fluorescent labels, hapten labels such as biotin, or enzymes such as horseradish peroxidase or alkaline phosphatase. In other embodiments, unlabeled primary antibody is used in an indirect assay as described above in conjunction with a labeled secondary antibody specific for the primary antibody. As described herein, the secondary antibodies can be labeled, for example, with enzymes or other detectable labels such as fluorescent labels, luminescent labels, colorimetric labels, or radioisotopes. Immunoblotting protocols and kits are well known in the art and are commercially available. Automated systems for immunoblotting, e.g. iBind Western Systems for Western blotting (ThermoFisher, Waltham, MA USA 02451), are available commercially. Immunoblotting includes, but is not limited to, Western blot, in-cell Western blot, and dot blot. Dot blot is a simplified procedure in which protein samples are not separated by electrophoresis but are spotted directly onto a membrane. In cell Western blot involves seeding cells in microtiter plates, fixing/permeabilizing the cells, and subsequent detection with a primary labeled primary antibody or unlabelled primary antibody followed by labeled secondary antibody as described herein.

In other embodiments, the expression levels of the protein products encoded by the marker genes can also be detected with the antibodies described herein in a flow cytometry assay, including a fluorescence-activated cell sorting (FACS) assay. Similar to the IHC or immunoblotting assays, primary antibodies or antisera, such as polyclonal antisera and monoclonal antibodies that specifically target the protein products encoded by the marker genes, can be used to detect protein expression in a FACS assay. In some embodiments, cells are stained with primary antibodies against specific target protein for a period of time sufficient for the antibody-antigen binding to occur and the bound antibodies can be detected by direct labels on the primary antibodies, for example, fluorescent labels or hapten labels such as biotin on the primary antibodies. In other embodiments, unlabeled primary antibody is used in an indirect assay as described above in conjunction with a fluorescently labeled secondary antibody specific for the primary antibody. FACS provides a method for sorting or analyzing a mixture of fluorescently labeled biological cells, one cell at a time, based upon the specific light scattering and fluorescent characteristics of each cell. The flow cytometer thus detects and reports the intensity of the fluorochrome-tagged antibody, which indicates the expression level of the target protein. Therefore, the expression level of the protein products encoded by the marker genes can be detected using antibodies against such protein products. Non-fluorescent cytoplasmic proteins can also be observed by staining permeabalized cells. Methods for performing FACS staining and analyses are well known to a person skilled in the art and are described by Teresa S. Hawley and Robert G. Hawley in Flow Cytometry Protocols, Humana Press, 2011 (ISBN 1617379506, 9781617379505).

In other embodiments, the expression levels of the protein products encoded by the marker genes can also be detected using immunoassays such as an Enzyme Immune Assay (EIA) or an ELISA. Both EIA and ELISA assays are known in the art, e.g. for assaying a wide variety of tissues and samples, including blood, plasma, serum or bone marrow. A wide range of ELISA assay formats are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target protein. Sandwich assays are commonly used assay format. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results can either be qualitative, by simple observation of the visible signal, or can be quantitated by comparing with a control sample containing known amounts of target protein.

In some embodiments of the EIA or ELISA assays, an enzyme is conjugated to the second antibody. In other embodiments, fluorescently labeled secondary antibodies can be used in lieu of the enzyme-labeled secondary antibody to produce a detectable signal an ELISA assay format. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA and ELISA, the fluorescent labeled antibody is allowed to bind to the first antibody-target protein complex. After washing off the unbound reagent, the remaining tertiary complex is then exposed to the light of the appropriate wavelength; the fluorescence observed indicates the presence of the target protein of interest. Immunofluorescence and EIA techniques are both very well established in the art and are disclosed herein.

For the immunoassays described herein, any of a number of enzymes or non-enzyme labels can be utilized so long as the enzymatic activity or non-enzyme label, respectively, can be detected. The enzyme thereby produces a detectable signal, which can be utilized to detect a target protein. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present disclosure.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis (2-amino-2-methyl-1,3-propanediol), 4-Methylumbelliferyl phosphate bis (cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β- D-glucopyranoside for β-glucosidase; and 4-Methylumbelliferyl-α- D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

In some embodiments of the immunoassays, a detectable label can be directly coupled to either the primary antibody or the secondary antibody that detects the unlabeled primary antibody can have. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene Oreg.); pyrenes and sulfonated pyrenes such as Cascade Blue′ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

Methods well known to a person skilled in the art such as microscopy or spectroscopy can be utilized to visualize chromogenic or fluorescent detectable signals associated with the bound primary or secondary antibodies.

The methods provided in this Section (Section 5.8) can be used with various cancer models known in the art. In one embodiment, mouse xenograft cancer models are used. Briefly, T-24 and UM-UC-3 cells are purchased from ATCC and cultured using the recommended media conditions. The T-24 hNectin-4 (human nectin-4) and the UM-UC-3 Nectin-4 cells are generated by transducing parental cells with lentivirus containing the human Nectin-4 using the pRCDCMEP-CMV-hNectin-4 EF1-Puro construct and selected using puromycin. The T-24 Nectin-4 (clone 1A9) cells are implanted into nude mice and passaged via trocar, allowed to reach approximately 200 mm³ tumor volume, and subsequently treated with a single intraperitoneal (IP) dose of enfortumab vedotin (3 mg/kg) or non-binding ADC (3 mg/kg) with 7 animals per treatment group. Follow-up ICD studies with this model involve collecting tumors 5 days post treatment for downstream analysis by RNA-seq, flow, immunohistochemistry (IHC), and Luminex. Tumors are fixed in formalin and prepared as FFPE tissue blocks. Blocks are cut at 4 μm and immunohistochemistry is performed using F4/80, CD11c. The immunohistochemically stained slides sections are scanned with a Leica AT2 digital whole slide scanner, and the images are analyzed with Visiopharm software by use of custom-made algorithms for Nectin 4, CD11c and F4/80 staining. The algorithms are optimized on the basis of staining intensity and background staining. Percent positive staining is calculated for Nectin 4 and positive cells per mm² is calculated for F480 and CD11c.

Sections of tumor are lysed in Cell Lysis Buffer 2 (R&D Systems®, Catalog #895347). The cytokines and chemokines from the tumor samples are measured using the MILLIPLEX MAP mouse cytokine/chemokine magnetic bead panel (Millipore) and read on the LUMINEX MAGPIX system.

For the RNA-seq analysis RNA from flash frozen tumors is isolated using the TRIZOL Plus RNA Purification Kit (Life Technologies) according to the manufacturer's protocol yielding high quality RNA (average RNA integrity number >8). RNA selection method is using Poly(A) selection and the mRNA Library Prep Kit from Illumina and read on the Hi-Seq 2×150 bp, single index (Illumina). The sequence reads are mapped to the human and mouse transcriptome and total reads per million were determined.

The disclosure is generally provided using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

Particular embodiments of this disclosure are described herein, including the best mode known to the inventors for carrying out the disclosure. Upon reading the foregoing description, variations of the disclosed embodiments can become apparent to individuals working in the art, and it is expected that those skilled artisans can employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference in their entireties as if each individual publication or patent application were specifically and individually indicated to be incorporated by reference.

A number of embodiments of the disclosure have been described. Nevertheless, it will be understood that various modifications can be made without departing from the spirit and scope of the disclosure.

### 6. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

### 6.1 Example 1—An Open-Label, Randomized Phase 3 Study to Evaluate Enfortumab Vedotin vs Chemotherapy in Subjects with Previously Treated Locally Advanced or Metastatic Urothelial Cancer (EV-301)

### 6.1.1 Drug Used in the Clinical Study

In one embodiment, the ADC provided herein is enfortumab vedotin, also known as PADCEV. In one specific embodiment tested in this example (6.1), the enfortumab vedotin-ejfv included an anti-191P4D12 antibody, wherein the antibody or antigen binding fragment thereof comprises a heavy chain comprising amino acid residue 20 to amino acid residue 466 of SEQ ID NO: 7 and a light chain comprising amino acid residue 23 to amino acid residue 236 of SEQ ID NO:8.

Enfortumab vedotin-ejfv is a Nectin-4 directed antibody-drug conjugate (ADC) comprised of a fully human anti-nectin-4 IgG1 kappa monoclonal antibody (AGS-22C3) conjugated to the small molecule microtubule disrupting agent, monomethyl auristatin E (MMAE) via a protease-cleavable maleimidocaproyl valine-citrulline (vc) linker (SGD-1006). Conjugation takes place on cysteine residues that comprise the interchain disulfide bonds of the antibody to yield a product with a drug-to-antibody ratio of approximately 3.8:1. The molecular weight is approximately 152 kDa.

Enfortumab vedotin-ejfv has the following structural formula:

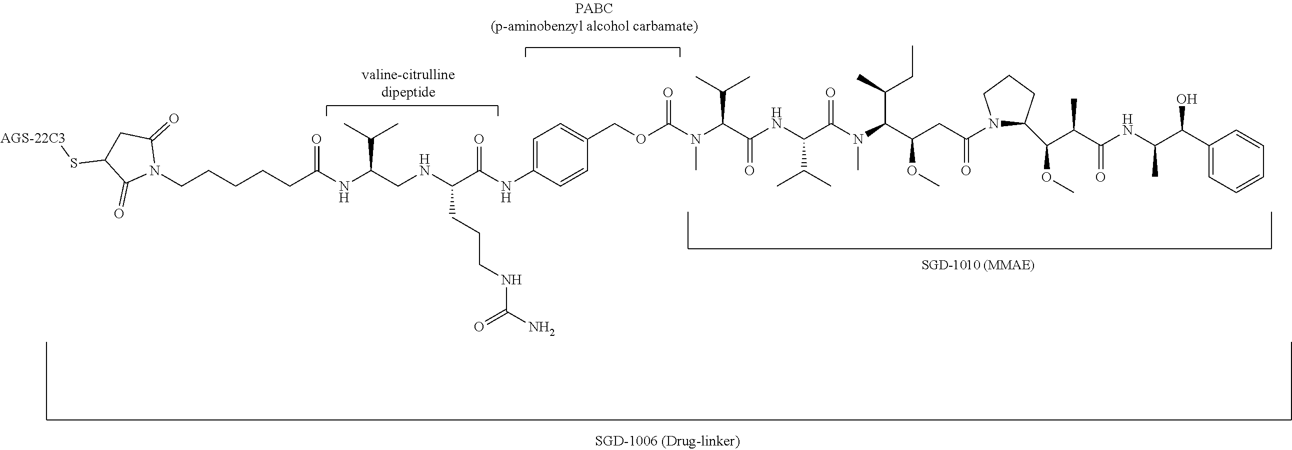

Approximately 4 molecules of MMAE are attached to each antibody molecule. Enfortumab vedotin-ejfv is produced by chemical conjugation of the antibody and small molecule components. The antibody is produced by mammalian (Chinese hamster ovary) cells and the small molecule components are produced by chemical synthesis.

PADCEV (enfortumab vedotin-ejfv) for injection was provided as a sterile, preservative-free, white to off-white lyophilized powder in single-dose vials for intravenous use. PADCEV was supplied as a 20 mg per vial and a 30 mg per vial and requires reconstitution with Sterile Water for Injection, USP, (2.3 mL and 3.3 mL, respectively) resulting in a clear to slightly opalescent, colorless to slightly yellow solution with a final concentration of 10 mg/mL (see Dosage and Administration (6.1.6.10). After reconstitution, each vial allows the withdrawal of 2 mL (20 mg) and 3 mL (30 mg). Each mL of reconstituted solution contained 10 mg of enfortumab vedotin-ejfv, histidine (1.4 mg), histidine hydrochloride monohydrate (2.31 mg), polysorbate 20 (0.2 mg) and trehalose dihydrate (55 mg) with a pH of 6.0.

### 6.1.2 Summary of the Study

### 6.1.2.1 Synopsis

(i) Name of Study Drug

Enfortumab Vedotin (ASG-22CE)

(ii) Phase of Development

Phase 3

(iii) Title of Study

An Open-Label, Randomized Phase 3 Study to Evaluate Enfortumab Vedotin vs Chemotherapy in Subjects with Previously Treated Locally Advanced or Metastatic Urothelial Cancer (EV-301)

(iv) Planned Study Period

2Q2018 to 2Q2021. The planned study enrollment is approximately 24 months from first subject enrolled with an additional 12 months anticipated for overall survival (OS) follow-up after the last subject enrolled. The total study duration will be approximately 36 months.

(v) Study Objective(s)

(a) Primary Objective

To compare the OS of subjects with locally advanced or metastatic urothelial cancer treated with EV to the OS of patients treated with chemotherapy.

(b) Secondary Objectives

To compare progression free survival on study therapy (PFS1) per Response Evaluation Criteria in Solid Tumors (RECIST) V1.1 of subjects treated with EV to patients treated with chemotherapy

To compare the overall response rate (ORR) per RECIST V1.1 of EV to chemotherapy

To evaluate the duration of response (DOR) per RECIST V1.1 of EV and chemotherapy

To compare the disease control rate (DCR) per RECIST V1.1 of EV to chemotherapy

To assess the safety and tolerability of EV

To assess quality of life (QOL) and Patient Reported Outcomes (PRO) parameters

(c) Exploratory Objectives

Exploratory genomic and/or other biomarkers in tumor tissue and in peripheral blood that may correlate with treatment outcome, including Nectin-4 expression

To assess the pharmacokinetics of EV (total antibody (Tab), antibody-drug conjugate (ADC) and monomethyl auristatin E (MMAE))

To assess the incidence of antitherapeutic antibodies (ATA)

To evaluate PFS in the next line of therapy (PFS2) of EV compared to chemotherapy

Healthcare resources utilization (HRU)

(vi) Planned Total Number of Study Centers and Locations

Approximately 185 study centers in North America, Europe, Asia Pacific and Latin America

(vii) Study Population

Subjects with locally advanced or metastatic urothelial cancer previously treated with platinum- based chemotherapy and an immune checkpoint inhibitor (CPI)

(viii) Number of Subjects to be Enrolled/Randomized

Approximately 600 subjects

(ix) Study Design Overview

This is a global, open-label, randomized Phase 3 study in adult subjects with locally advanced or metastatic urothelial cancer who have received a platinum-containing chemotherapy and have experienced disease progression or relapse during or following treatment with an immune checkpoint inhibitor. Approximately 600 subjects will be randomized to EV (Arm A) or chemotherapy (Arm B) in a 1:1 ratio. Subjects will be stratified according to the following: Eastern Cooperative Oncology Group Performance Status (ECOG PS), regions of the world and liver metastasis.

OS is the primary endpoint. OS is defined as the time from randomization to the date of death. Secondary endpoints include PFS1, ORR, DOR, DCR, safety and QOL/PRO.

Subjects in Arm A will receive EV on Days 1, 8 and 15 of each 28-day cycle. Subjects in arm B will receive either docetaxel, paclitaxel or vinflunine (as decided by the investigator prior to randomization: vinflunine is a choice of comparator only in countries where it is approved for urothelial cancer) on Day 1 of every 21-day cycle. Within the control arm, the overall proportion of subjects receiving vinflunine will be capped at approximately 35%. Subjects will continue to receive study treatment until radiological disease progression as determined per investigator assessment or other discontinuation criteria are met or upon study termination, or study completion, whichever occurs first. No on-study crossover will be allowed. This study will consist of 3 phases: screening, treatment and follow-up.

Screening will take place up to 28 days prior to randomization. Subjects will start with cycle 1 and continue on to subsequent 21-day or 28-day cycles until one of the discontinuation criteria are met. A treatment cycle is defined as 28 days for Arm A and 21 days for Arm B. Subjects randomized to Arm A (EV) will receive treatment and evaluation on Days 1, 8 and 15 of all treatment cycles. Subjects randomized to Arm B (docetaxel, paclitaxel or vinflunine) will receive treatment and evaluation on Day 1 of all treatment cycles.

Subjects will be evaluated for response according to the RECIST V1.1. Imaging for both arms will be performed at baseline and every 56 days (±7 days) from the first dose of study treatment throughout the study until PFS1 is documented by radiological disease progression or the subject is lost to follow-up, death, withdraws study consent or starts a subsequent anti-cancer therapy.

Baseline imaging performed prior to informed consent as standard of care may be used so long as it is performed within 28 days prior to randomization. All subjects will have a bone scan (scintigraphy) performed at screening/baseline. Subjects with positive bone scans at baseline will have a bone scan performed every 56 days (±7 days) throughout the study or more frequently if clinically indicated. Subjects should have a follow-up bone scan performed if clinically indicated regardless of baseline status. Brain scans (computed tomography with contrast/magnetic resonance imaging (MRI)) will only be performed if clinically indicated at screening/baseline and repeated as clinically indicated or per standard of care throughout the study.

QOL assessments and PRO will be collected at protocol-specified time points from all randomized subjects. The following validated tools will be used: European Organisation for Research and Treatment of Cancer (EORTC) Quality of Life Questionnaire (QLQ-C30) and EuroQOL 5-dimensions (EQ-5D-5L). Healthcare Resource Utilization (HRU) information will be collected at protocol-specified time points with particular focus on the number of subjects who have an unplanned use of healthcare resources related to clinical or AEs from subjects assigned to treatment arms A and B.

Blood samples for pharmacokinetics and ATA will be collected throughout the study for subjects randomized into Arm A. Validated assays will be used to measure the concentrations of EV ADC and MMAE in serum or plasma and to assess ATA. Pharmacokinetic samples will not be collected from subjects randomized into Arm B. Samples for exploratory biomarkers will be collected at protocol-specified timepoints. Biomarker assessments will not be used for subject selection.

Following discontinuation from study drug, subjects will have a follow-up visit 30 days (+7 days) after their last dose of drug for safety assessments. If a subject discontinues study drug prior to radiographic disease progression (i.e., PFS1), the subject should enter the post treatment follow-up period and continue to undergo imaging assessments every 56 days (±7 days) until PFS1 is documented or the subject starts another anticancer treatment, whichever occurs earlier.

Following PFS1, subjects will enter the long-term follow-up period and be followed per institutional guidelines, but not less than every 3 months from the date of the follow-up visit for survival status and progression status on subsequent therapy (i.e., PFS2).

Subjects will be followed until PFS2 is documented or the subject starts another anticancer treatment, whichever occurs earlier. All subsequent anticancer therapy including date and site of progression for PFS2 will be recorded on the case report form.

Following PFS2, subjects will enter the survival follow-up period and be followed every 3 months for survival status until death, lost to follow-up, withdrawal of study consent, or study termination by sponsor. This study is expected to end once final survival analysis is complete.

An Independent Data Monitoring Committee (IDMC) will be chartered to oversee safety and the planned interim efficacy analysis, which will occur after at least 285 OS events (about 65% of the total planned events) are observed. The primary analysis will occur at 439 OS events. The IDMC may recommend to the sponsor whether the trial should be terminated, modified or continue unchanged based on ongoing reviews of safety data and interim efficacy analysis. Further details will be outlined in the IDMC charter.

(x) Inclusion/Exclusion Criteria

Inclusion: Subject is eligible for the study if all of the following apply:
1. Institutional Review Board (IRB)/Independent Ethics Committee (IEC) approved written informed consent and privacy language as per national regulations (e.g., Health Insurance Portability and Accountability Act (HIPAA) Authorization for US sites) must be obtained from the subject prior to any study-related procedures (including withdrawal of prohibited medication, if applicable).
2. Subject is legally an adult according to local regulation at the time of signing informed consent.
3. Subject has histologically or cytologically confirmed urothelial carcinoma (i.e., cancer of the bladder, renal pelvis, ureter or urethra). Subjects with urothelial carcinoma (transitional cell) with squamous differentiation or mixed cell types are eligible.
4. Subject must have experienced radiographic progression or relapse during or after a CPI (anti-programmed cell death-1 (PD-1) or anti-programmed cell death-ligand 1 (PD-L1)) for locally advanced or metastatic disease. Subjects who discontinued CPI treatment due to toxicity are eligible provided that they have evidence of disease progression following discontinuation. The CPI need not be the most recent therapy. Subjects for whom the most recent therapy has been a non-CPI based regimen are eligible if they have progressed/relapsed during or after their most recent therapy. Locally advanced disease must not be amenable to resection with curative intent per the treating physician.
5. Subject must have received a platinum containing regimen (cisplatin or carboplatin) in the metastatic/locally advanced, neoadjuvant or adjuvant setting. If platinum was administered in the adjuvant/neoadjuvant setting subject must have progressed within 12 months of completion.
6. Subject has radiologically documented metastatic or locally advanced disease at baseline.
7. An archival tumor tissue sample should be available for submission to central laboratory prior to study treatment. If an archival tumor tissue sample is not available, a fresh tissue sample should be provided. If a fresh tissue sample cannot be provided due to safety concerns, enrollment into the study must be discussed with the medical monitor.
8. Subject has ECOG PS of 0 or 1
9. The subject has the following baseline laboratory data:
   absolute neutrophil count (ANC) ≥1500/mm³
   platelet count ≥100×10⁹/L
   hemoglobin ≥9 g/dL
   serum total bilirubin ≤1.5×upper limit of normal (ULN)* or ≤3×ULN for subjects with Gilbert's disease
   creatinine clearance (CrCl) ≥30 mL/min as estimated per institutional standards or as measured by 24 hour urine collection (glomerular filtration rate (GFR) can also be used instead of CrCl)
   alanine aminotransferase (ALT) and aspartate aminotransferase (AST) ≤2.5×ULN or
   ≤3×ULN for subjects with liver metastases*
      Docetaxel should not be chosen as a comparator for subjects if total bilirubin >ULN, or if AST and/or ALT >1.5×ULN concomitant with alkaline phosphatase >2.5×ULN.
10. Female subject must either:
   Be of nonchildbearing potential:
      Postmenopausal (defined as at least 1 year without any menses for which there is no other obvious pathological or physiological cause) prior to screening, or
      Documented surgically sterile (e.g., hysterectomy, bilateral salpingectomy, bilateral oophorectomy).
   Note: Those who are amenorrheic due to an alternative medical cause are not considered postmenopausal and must follow the criteria for childbearing potential subjects.
   Or, if of childbearing potential:
      Agree not to try to become pregnant during the study and for at least 6 months after the final study drug administration,
      And have a negative urine or serum pregnancy test within 7 days prior to Day 1 (Females with false positive results and documented verification of negative pregnancy status are eligible for participation),
      And if heterosexually active, agree to consistently use a condom plus 1 form of highly effective birth control * per locally accepted standards starting at screening and throughout the study period and for at least 6 months after the final study drug administration.
11. Female subject must agree not to breastfeed or donate ova starting at screening and throughout the study period, and for at least 6 months after the final study drug administration.
12. A sexually active male subject with female partner(s) who is of childbearing potential is eligible if:
   Agrees to use a male condom starting at screening and continue throughout the study treatment and for at least 6 months after final study drug administration. If the male subject has not had a vasectomy or is not sterile as defined below their female partner(s) is utilizing 1 form of highly effective birth control* per locally accepted standards starting at screening and continue throughout study treatment and for at least 6 months after the male subject receives his final study drug administration.
   Highly effective forms of birth control include:
      Consistent and correct usage of established hormonal contraceptives that inhibit ovulation,
      Established intrauterine device (IUD) or intrauterine hormone releasing system (IUS).
      Bilateral tubal occlusion
      Vasectomy (A vasectomy is a highly effective contraception method provided the absence of sperm has been confirmed. If not, an additional highly effective method of contraception should be used)
      Male is sterile due to a bilateral orchiectomy or radical cystoprostatectomy/removal of seminal vesicles
      Sexual Abstinence is considered a highly effective method only if defined as refraining from heterosexual activity during the entire period of risk associated with the study drug. The reliability of sexual abstinence needs to be evaluated in relation to the duration of the study and the preferred and usual lifestyle of the participant.
      Note: Sexual abstinence is not sufficient as contraception method in Switzerland.
13. Male subject must not donate sperm starting at screening and throughout the study period, and for at least 6 months after the final study drug administration.
14. Male subject with a pregnant or breastfeeding partner(s) must agree to abstinence or use a condom for the duration of the pregnancy or time partner is breastfeeding throughout the study period and for at least 6 months after the final study drug administration.
15. Subject agrees not to participate in another interventional study while on treatment in present study.
   Waivers to the inclusion criteria will NOT be allowed.

Exclusion: Subject will be excluded from participation if any of the following apply:
1. Subject has preexisting sensory or motor neuropathy Grade ≥2.
2. Subject has active central nervous system (CNS) metastases. Subjects with treated CNS metastases are permitted on study if all the following are true:
   CNS metastases have been clinically stable for at least 6 weeks prior to screening
   If requiring steroid treatment for CNS metastases, the subject is on a stable dose ≤20 mg/day of prednisone or equivalent for at least 2 weeks
   Baseline scans show no evidence of new or enlarged brain metastasis
   Subject does not have leptomeningeal disease
3. Subject has ongoing clinically significant toxicity (Grade 2 or higher with the exception of alopecia) associated with prior treatment (including systemic therapy, radiotherapy or surgery). Subject with ≤Grade 2 immunotherapy-related hypothyroidism or panhypopituitarism may be enrolled when well-maintained/controlled on a stable dose of hormone replacement therapy (if indicated). Patients with ongoing ≥Grade 3 immunotherapy-related hypothyroidism or panhypopituitarism are excluded. Subjects with ongoing immunotherapy related colitis, uveitis, myocarditis, or pneumonitis or subjects with other immunotherapy related AEs requiring high doses of steroids (>20 mg/day of prednisone or equivalent) are excluded.
4. Subject has prior treatment with EV or other MMAE-based ADCs.
5. Subject has received prior chemotherapy for urothelial cancer with all available study therapies in the control arm (i.e., both prior paclitaxel and docetaxel in regions where vinflunine is not an approved therapy, or prior paclitaxel, docetaxel and vinflunine in regions where vinflunine is an approved therapy).
   Note: after vinflunine cap is reached subjects who have received both docetaxel and paclitaxel will be excluded.
6. Subject has received more than 1 prior chemotherapy regimen for locally advanced or metastatic urothelial cancer, including chemotherapy for adjuvant or neo-adjuvant disease if recurrence occurred within 12 months of completing therapy. The substitution of carboplatin for cisplatin does not constitute a new regimen provided no new chemotherapeutic agents were added to the regimen.
7. Subject has history of another malignancy within 3 years before the first dose of study drug, or any evidence of residual disease from a previously diagnosed malignancy. Subjects with nonmelanoma skin cancer, localized prostate cancer treated with curative intent with no evidence of progression, low-risk or very low-risk (per standard guidelines) localized prostate cancer under active surveillance/watchful waiting without intent to treat, or carcinoma in situ of any type (if complete resection was performed) are allowed.
8. Subject is currently receiving systemic antimicrobial treatment for viral, bacterial, or fungal infection at the time of first dose of EV. Routine antimicrobial prophylaxis is permitted.
9. Subject has known active Hepatitis B (e.g., HBsAg reactive) or active hepatitis C (e.g., HCV RNA (qualitative) is detected).
10. Subject has known history of human immunodeficiency virus (HIV) infection (HIV 1 or 2).
11. Subject has documented history of a cerebral vascular event (stroke or transient ischemic attack), unstable angina, myocardial infarction, or cardiac symptoms (including congestive heart failure) consistent with New York Heart Association Class III-IV within 6 months prior to the first dose of study drug.
12. Subject has radiotherapy or major surgery within 4 weeks prior to first dose of study drug.
13. Subject has had chemotherapy, biologics, investigational agents, and/or antitumor treatment with immunotherapy that is not completed 2 weeks prior to first dose of study drug.
14. Subject has known hypersensitivity to EV or to any excipient contained in the drug formulation of EV (including histidine, trehalose dihydrate, and polysorbate 20); OR subject has known hypersensitivity to biopharmaceuticals produced in Chinese hamster ovary (CHO) cells.
15. Subject has known hypersensitivity to:
   docetaxel or to any of the other excipients listed in product label, including polysorbate 80;
   paclitaxel or to any of the other excipients listed in product label, including macrogolglycerol ricinoleate 35 (Ph.Eur.); and
   vinflunine or to any of the other excipients listed in product label, including other vinca alkaloids (vinblastine, vincristine, vindesine, vinorelbine).
16. Subject has known active keratitis or corneal ulcerations. Subject with superficial punctate keratitis is allowed if the disorder is being adequately treated in the opinion of the investigator.
17. Subject has other underlying medical condition that, in the opinion of the investigator, would impair the ability of the subject to receive or tolerate the planned treatment and follow-up.
18. History of uncontrolled diabetes mellitus within 3 months of the first dose of study drug. Uncontrolled diabetes is defined as hemoglobin A1C (HbA1c) ≥8% or HbA1c between 7 and <8% with associated diabetes symptoms (polyuria or polydipsia) that are not otherwise explained.
   Waivers to the exclusion criteria will NOT be allowed.

(xi) Drug Product: Enfortumab Vedotin: Dose, Mode of Administration and Dose Modification

EV 1.25 mg/kg will be administered on Days 1, 8, and 15 of every 28-day cycle. The drug product will be administered intravenously over a 30-minute period.

EV will be administered based on the subject's actual body weight on Day 1 of every cycle except for subjects weighing greater than 100 kg; in such cases, the dose will be calculated based on a maximum weight of 100 kg. The dose does not need to be re-calculated based on actual weight on Day 8 and 15 of each cycle for Arm A unless it is required by institutional standards.

Dose reduction to 1 mg/kg (dose level—1) and to 0.75 mg/kg (dose level—2) will be allowed depending on the type and severity of toxicity. Subjects requiring a dose reduction may be re- escalated by 1 dose level (i.e., subjects reduced to 0.75 mg/kg may only be re-escalated to 1 mg/kg) provided the toxicity does not require study drug discontinuation and has returned to baseline or ≤Grade 1. If the toxicity recurs, re-escalation will not be permitted. Subjects with ≥Grade 2 corneal AEs will not be permitted to dose re-escalate. EV should not be administered to subjects with CrCl<30 mL/min. Dose modification recommendations for EV associated toxicity are presented in Table 6 and Table 7.

Dose interruptions for other EV associated toxicity is permitted at the discretion of the site investigator. Dose interruptions may last up to 8 weeks (2 cycles). Dose interruptions for subjects who are deriving clinical benefit from treatment may be extended beyond 8 weeks, if the subject's toxicity does not otherwise require permanent discontinuation. If there is a dose interruption, the schedule for response assessments will not be adjusted.

TABLE 6
Recommended dose modifications for enfortumab
vedotin associated hematologic toxicity*

| Grade 1 | Grade 2 | Grade 3 | Grade 4 |
|---|---|---|---|
| Continue at same | Continue at | Withhold dose until | Withhold dose until |
| dose level. | same dose level. | toxicity is ≤ Grade 1 | toxicity is ≤ Grade 1 |
| | For Grade 2 | or has returned to | or has returned to |
| | thrombocytopenia, | baseline, then | baseline, then |
| | withhold dose | resume treatment at | reduce dose by 1 |
| | until toxicity | the same dose level | dose level and |
| | is ≤ Grade 1 or has | or consider dose | resume treatment, or |
| | returned to | reduction by 1 dose | discontinue at the |
| | baseline, then | level. | discretion of the |
| | resume treatment at | Transfusions or | investigator. |
| | the same dose | growth factors | Transfusions or |
| | level. | may be used as | growth factors |
| | | indicated per | may be used as |
| | | institutional | indicated per |
| | | guidelines. | institutional |
| | | | guidelines. |
| | | | For anemia, |
| | | | treatment |
| | | | discontinuation |
| | | | should be strongly |
| | | | considered. |

*Note:
hematological toxicity refers to anemia, thrombocytopenia, neutropenia and febrile neutropenia.

TABLE 7
Recommended dose modifications for enfortumab
vedotin associated nonhematologic toxicity

| Grade 1 | Grade 2 | Grade 3 | Grade 4 |
|---|---|---|---|
| Continue at | Continue at same | Withhold dose until | For Grade 4 AEs, |
| same dose level. | dose level, except | toxicity is ≤ Grade | discontinue |
| If ocular symptoms | in the event of | 1 or has returned to | treatment.* |
| and/or changes in | Grade 2 | baseline, then | Grade 4 vomiting |
| vision are | neuropathy or | resume treatment at | and/or diarrhea that |
| identified, the | corneal AEs. | the same dose level | improves to ≤ |
| subject should be | For Grade 2 | or consider dose | Grade 2 within |
| evaluated with an | neuropathy or | reduction by 1 dose | 72 hours with |
| ophthalmologic | corneal AE's, | level.* | supportive |
| exam.** | withhold dose until | For Grade 3 | management does |
| | toxicity is ≤ | neuropathy or | not require |
| | Grade 1 or has | corneal AEs, | discontinuation. |
| | returned to | discontinue | |
| | baseline, and then | treatment | |
| | resume treatment | For Grade 3 | |
| | at the same dose | hyperglycemia/elevated | |
| | level. For the | blood glucose, | |
| | second occurrence | withhold EV | |
| | of Grade 2 | treatment. Resume | |
| | neuropathy or | treatment once | |
| | corneal AE's | hyperglycemia/elevated | |
| | withhold dose until | blood glucose | |
| | toxicity is ≤ Grade | has improved to ≤ | |
| | 1, and then reduce | Grade 2 and subject | |
| | the dose by 1 dose | is clinically and | |
| | level and resume | metabolically | |
| | treatment. | stable. | |
| | If ocular symptoms | If ocular symptoms | |
| | and/or changes in | and/or changes in | |
| | vision are | vision are | |
| | identified, the | identified, the | |
| | subject should be | subject should be | |
| | evaluated with an | evaluated with an | |
| | ophthalmologic | ophthalmologic | |
| | exam.** | exam.** | |

AE: adverse events;
EV: enfortumab vedotin
*Grade 3/4 electrolyte imbalances/laboratory abnormalities that are not associated with clinical sequelae and/or are corrected with supplementation/appropriate management within 72 hours of their onset do not require discontinuation (e.g., Grade 4 hyperuricemia). Grade 3 rash that is not limiting self-care activities of daily living or associated with infection requiring systemic antibiotics does not require treatment interruption, provided symptoms are not severe and can be managed with supportive treatment.

(xii) Comparative Drug(s)

In general, treatment with the chemotherapy comparators (docetaxel, paclitaxel, or vinflunine) should be withheld for drug related Grade 4 hematologic toxicities and for non-hematologic toxicities ≥Grade 3, and subsequent doses modified as per Table 15. Recommended dose modification guidelines specific for subjects receiving docetaxel, paclitaxel, or vinflunine are detailed below. Dose modifications should also be considered according to local product labels or summary of product characteristics (SmPC) and institutional guidelines. For docetaxel, paclitaxel, or vinflunine associated hematologic toxicities ≥Grade 3, transfusions or growth factors may be used as indicated per institutional guidelines.

(a) Docetaxel: Dose, Mode of Administration and Dose Modification

Docetaxel will be administered intravenously on Day 1 of every 21-day cycle. The starting dose of docetaxel 75 mg/m² will be administered over 60-minute period or per local requirement. Refer to local product label or SmPC and institution guidelines for docetaxel for further guidance on docetaxel dosing.

Docetaxel should not be given to subjects with total bilirubin >ULN, or to subjects with AST and/or ALT >1.5×ULN with concomitant alkaline phosphatase >2.5×ULN. Subjects with elevations of bilirubin or abnormalities of transaminase concurrent with alkaline phosphatase are at increased risk for the development of Grade 4 neutropenia, febrile neutropenia, infections, severe thrombocytopenia, severe stomatitis, severe skin toxicity, and toxic death. Docetaxel should also not be given to subjects with a neutrophil count of <1500 cells/mm³. Severe fluid retention has been reported following docetaxel therapy.

Subjects should be premedicated with corticosteroids per institutional guidelines prior to each docetaxel administration. Subjects with pre-existing effusions should be closely monitored from the first dose for the possible exacerbation of the effusions. Subjects developing peripheral edema may be treated with standard measures, e.g., salt restriction, oral diuretic(s). Dose interruptions may last up to 6 weeks (2 cycles). Dose interruptions for subjects who are deriving clinical benefit from treatment may be extended beyond 6 weeks, if the subject's toxicity does not otherwise require permanent discontinuation.

Dose modifications not specified in Table 8 (e.g., severe or cumulative cutaneous reactions) should also be considered according to local product label or SmPC and institutional guidelines.

TABLE 8
Recommended dose modifications for subjects receiving docetaxel

| | | | Hold | Dose | Discontinue |
|---|---|---|---|---|---|
| Toxicity | Grade | Occurrence | Treatment | Modification | Treatment |
| Peripheral | Grade 1, 2 | | No | 60 mg/m² | N/A |
| Neuropathy | Grade 3, 4 | | Yes | N/A | Discontinue |
| | | | | | upon onset |
| Neutropenic | | 1 | Hold | 60 mg/m² | |
| fever (defined as | | | treatment | | |
| T ≥ 100.5° F. | | | until | | |
| (38.1° C.) and | | | ANC ≥ 1,500/L | | |
| ANC ≤ 1,000/L) | | 2 | Hold | 50 mg/m² | |
| | | | treatment | | |
| | | | until | | |
| | | | ANC ≥ 1,500/L | | |
| | | 3 | Yes | N/A | Yes |

ANC: absolute neutrophil count;
N/A: not applicable;
T: temperature

(b) Vinflunine: Dose, Mode of Administration and Dose Modification

Vinflunine will be administered intravenously on Day 1 of every 21-day cycle. The starting dose of vinflunine 320 mg/m² will be administered over a 20-minute period (or per local requirement) unless otherwise specified below. In case of WHO/ECOG PS of >1 or ECOG PS of 0 and prior pelvic irradiation, vinflunine treatment should be started at the dose of 280 mg/m². In the absence of any hematological toxicity during the first cycle causing treatment delay or dose reduction, the dose may be increased to 320 mg/m² every 21-days for the subsequent cycles.

In subjects with moderate renal impairment (40 mL/min≤CrCl≤60 mL/min), the recommended dose is 280 mg/m² given once every 21-day cycle. In subjects with renal impairment (30 mL/min≤CrCl<40 mL/min), the recommended dose is 250 mg/m² given once every 21-day cycle. The recommended dose of vinflunine is 250 mg/m² given once every 21-day cycle in subjects with mild liver impairment (Child-Pugh grade A).

The doses recommended in subjects >75 years old are as follows:
in subjects at least 75 years old but less than 80 years, the dose of vinflunine to be given is 280 mg/m² every 21-day cycle.
in subjects 80 years old and beyond, the dose of vinflunine to be given is 250 mg/m² every 21-day cycle.

Refer to local product label or SmPC and institution guidelines for vinflunine for further guidance on vinflunine dosing.

Dose interruptions may last up to 6 weeks (2 cycles). Dose interruptions for subjects who are deriving clinical benefit from treatment may be extended beyond 6 weeks, if the subject's toxicity does not otherwise require permanent discontinuation. Refer to the approved product label for specific dose modifications for subjects receiving vinflunine.

(c) Paclitaxel: Dose, Mode of Administration and Dose Modification

Study treatment of paclitaxel should be administered intravenously on Day 1 of every 21-day cycle after all procedures/assessments have been completed. The starting dose of paclitaxel 175 mg/m² will be administered as an intravenous infusion administered over 3 hours or per local requirement See guidelines on adjustment of initial dose. Refer to local product label or SmPC and institution guidelines for paclitaxel for further guidance on paclitaxel dosing.

All subjects should be premedicated prior to paclitaxel administration per institutional guidelines in order to prevent severe hypersensitivity reactions. Such premedication may consist of dexamethasone 20 mg orally administered approximately 12 and 6 hours before paclitaxel, diphenhydramine (or its equivalent) 50 mg IV 30 to 60 minutes prior to paclitaxel, and cimetidine (300 mg) or ranitidine (50 mg) IV 30 to 60 minutes before paclitaxel. The appropriate premedication regimen may be determined by the investigator.

Paclitaxel should not be administered to subjects with baseline neutrophil counts of less than 1500 cells/mm³. Subjects should not be re-treated with subsequent cycles of paclitaxel until neutrophils recover to a level >1500 cells/mm³ and platelets recover to a level >100000/mm³. Severe conduction abnormalities have been documented in <1% of subjects during paclitaxel therapy and in some cases requiring pacemaker placement. If subjects develop significant conduction abnormalities during paclitaxel infusion, appropriate therapy should be administered and continuous cardiac monitoring should be performed during subsequent therapy with paclitaxel.

In case of mild hepatic impairment (total bilirubin ≥1.25 ULN), paclitaxel should be started at a dose of 135 mg/m².

Recommended dose modification guidelines specific for subjects receiving paclitaxel are detailed in Table 9 below. Dose modifications should also be considered according to local product label or SmPC and institutional guidelines.

Dose interruptions may last up to 6 weeks (2 cycles). Dose interruptions for subjects who are deriving clinical benefit from treatment may be extended beyond 6 weeks, if the subject's toxicity does not otherwise require permanent discontinuation.

TABLE 9
Recommended dose modifications for subjects receiving paclitaxel

| | | | Hold | Dose | Treatment |
|---|---|---|---|---|---|
| Toxicity | Grade | Occurrence | Treatment | Modification | Discontinuation |
| Peripheral | Grade 1, 2 | | No | 135 mg/m² | N/A |
| Neuropathy | Grade 3, 4 | | Yes | N/A | Hold treatment or |
| | | | | | Discontinue upon |
| | | | | | onset |
| Neutropenic | | 1 | Hold until | 135 mg/m² | |
| fever (defined | | | ANC ≥ | | |
| as T ≥ | | | 1,500/L | | |
| 100.5° F. | | 2 | Hold until | 100 mg/m² | |
| (38.1° C.) and | | | ANC ≥ | | |
| ANC ≤ | | | 1,500/L | | |
| 1,000/L) | | 3 | yes | N/A | Yes |

N/A: not applicable;
T: temperature

(xiii) Discontinuation Criteria

A discontinuation from treatment applies to a subject who enrolled in the study and for whom study treatment is permanently discontinued for any reason.

The subject is free to withdraw from the study treatment and/or study for any reason and at any time without giving reason for doing so and without penalty or prejudice. The investigator is also free to discontinue the subject from study treatment or to terminate a subject's involvement in the study at any time if the subject's clinical condition warrants it. If a subject is discontinued from the study with an ongoing adverse event (AE) or an unresolved laboratory result that is significantly outside of the reference range, the investigator will attempt to provide follow-up until the condition stabilizes or no longer is clinically significant.

The following are discontinuation criteria from treatment for individual subjects:
Subject develops radiological disease progression.
Subject is required to receive another systemic anti-cancer treatment for underlying or new cancer.
Subject develops unacceptable toxicity.
Female subject becomes pregnant.
Investigator decides it is in the subject's best interest to discontinue.
Subject declines further treatment.
Subject is noncompliant with the protocol based on the investigator or medical monitor assessment.
Subject is lost to follow-up despite reasonable efforts by the investigator to locate the subject.
Death.

Subjects who discontinue treatment prior to radiological disease progression will enter the post treatment follow-up period and continue to undergo imaging assessments every 56 days (±7 days) until PFS1 is documented by radiological disease progression or the subject starts another anticancer treatment, whichever occurs earlier.

Following PFS1, subjects will enter the long-term follow up period and be followed per institutional guidelines but not less than every 3 months from the date of the follow-up visit for survival status and progression on next line therapy until PFS2 is documented or the subject starts another anticancer treatment, whichever occurs earlier.

Subjects will then enter the survival follow-up period. Subjects will be followed every 3 months for survival status until any of the discontinuation criteria for OS are met. The subject will be discontinued from the OS post treatment follow-up period if any of the following occur:
Subject declines further study participation (i.e., withdraws consent).
Subject is lost to follow-up despite reasonable efforts by the investigator to locate the subject.
Death.
Study termination.

(xiv) Concomitant Medication Restrictions or Requirements:

If the investigator determines that any of the following medications are deemed necessary to provide adequate medical support to the subject, the subject must be withdrawn from further administration of the study treatment:
Other investigational drugs
Chemotherapy or other medications intended for antitumor activity. This does not apply to subjects with a history of breast cancer on adjuvant endocrine therapy, or to subjects on agents intended for the treatment of bone metastasis (e.g., bisphosphonates, or RANK ligand inhibitors).
Radiation therapy
Note: Radiation therapy to a symptomatic solitary lesion or to the bone may be considered on an exceptional case-by-case basis after consultation with Sponsor. The radiated lesion must be a non-target lesion per RECIST V1.1 and the subject must have clear measurable disease outside the radiated field.

Arm a (Enfortumab Vedotin)
Subjects who are receiving strong cytochrome P450 (CYP)3A4 inhibitors or P-pg inhibitors concomitantly with EV should be closely monitored for adverse reactions.

Arm B (Docetaxel)
Concomitant use of drugs that strongly inhibit or induce CYP3A4 may affect exposure to docetaxel and should be avoided.

Arm B (Vinflunine)
Strong inhibitors or inducers of the CYP3A4 enzymes for subjects receiving vinflunine should be avoided.
QT/QTc interval prolonging medicinal products should be avoided.

Arm B (Paclitaxel)
Caution should be exercised when paclitaxel is administered with strong inhibitors or inducers of CYP3A4 and CYP2C8.

Refer to the local package insert for concomitant medication restrictions or requirements for docetaxel, paclitaxel and vinflunine.

(xv) Duration of Treatment

Subjects will be allowed to receive EV or comparator until discontinuation criteria are met or upon study termination, or study completion, whichever occurs first.

(xvi) Endpoints for Evaluation

Primary
OS

Secondary
PFS1 per RECIST V1.1
ORR (complete response (CR)+PR) per RECIST V1.1
DCR (CR+PR+stable disease (SD)) per RECIST V1.1
DOR per RECIST V1.1
Safety variables (e.g., AEs, laboratory tests, vital sign measurements, 12-lead electrocardiogram and ECOG PS)
QOL and PRO parameters (QLQ-C30 and EQ-5D-5L)

Exploratory
Exploratory genomic and/or other biomarkers in tumor tissue and in peripheral blood that may correlate with treatment outcome, including Nectin-4 expression
Selected plasma or serum concentrations of TAb, ADC and MMAE
Incidence of ATA to EV
PFS2 per RECIST V1.1
HRU

(xvii) Statistical Methods:

Approximately 600 subjects will be randomized in a 1:1 ratio to 2 treatment arms: Arm A (EV) and Arm B (docetaxel, paclitaxel or vinflunine). Randomization will be stratified by:
Liver Metastasis (yes/no)
ECOG PS (0 vs 1)
Regions of the world (US, Western Europe and Rest of World)

(a) Sample Size Justification

Approximately 600 subjects (with 10% dropout rate and 1 interim analysis) will be randomized in a 1:1 ratio to receive EV or chemotherapy.
Primary endpoint: OS
One-sided 2.5% Type I error; 85% Power
OS Assumption: hazard ratio (HR)=0.75 (median OS of 10.7 m vs 8 m, for EV vs Chemotherapy arm)
A formal interim efficacy analysis will be done when approximately 65% of deaths have occurred.
Primary analysis will occur at about 439 OS events

For planned interim efficacy analysis, a group sequential design using the O'Brien-Fleming boundaries as implemented by Lan-DeMets method will be used to control the overall 1-sided 0.025 type I error. If the interim analysis demonstrates a statistically significant outcome for EV in efficacy, the study may be stopped and concluded due to efficacy.

The Full Analysis Set (FAS) will be used for the efficacy analysis on OS and PFS1. All subjects who are randomized will be included in the FAS. For time to event endpoints including OS and PFS1 log-rank test stratified by randomization stratification factors including liver metastasis, baseline ECOG PS and regions of the world at baseline will be used to compare the 2 treatment arms. The hazard ratio and corresponding 95% confidence interval from the stratified Cox proportional hazards regression model will also be presented. The median OS, PFS1 and DOR will be estimated using the Kaplan-Meier method and will be reported along with the corresponding 95% confidence interval by treatment arm.

ORR and DCR will be compared between treatment arms using Cochran-Mantel-Haenszel test, stratified by the same stratification factors used in time to event analyses. The difference in response rates between the treatment arms will be estimated along with the corresponding 95% confidence interval.

(b) Safety

The Safety Analysis Set (SAF) will be used for the safety analysis. All subjects who are randomized and received study drug will be included in the SAF. The frequency of AEs and the serious AEs will be summarized by MedDRA system organ class and preferred term. In addition, summary statistics will be provided for the following safety parameters:
Laboratory values
Vital sign measurements
ECOG PS

(c) Pharmacokinetics

Descriptive statistics (e.g., number, mean, standard deviation, minimum, median, maximum, coefficient of variation and geometric mean) will be provided for plasma or serum concentrations of TAb, ADC and MMAE. The incidence of ATA to EV will be summarized by cycle and overall, and possible relationship to pharmacokinetics explored. Additional model-based analyses and exposure response may be performed and reported separately.

### 6.1.2.2 Study Scheme and Schedule of Assessments

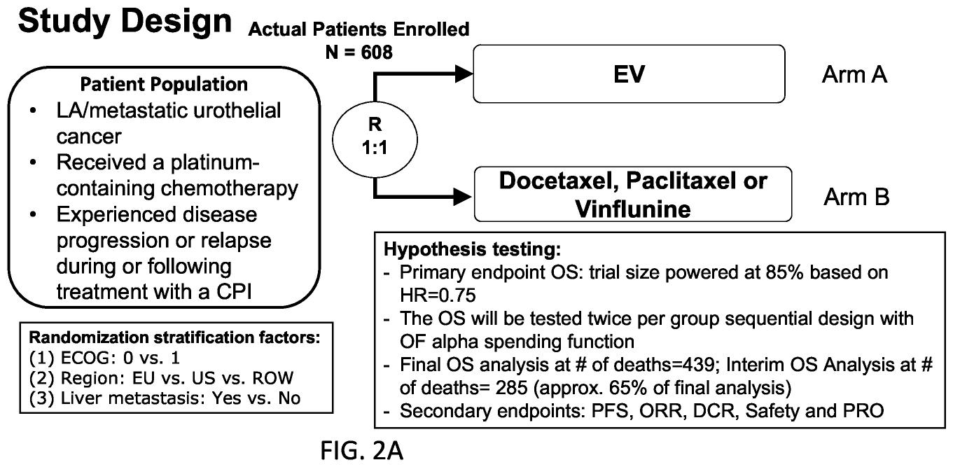

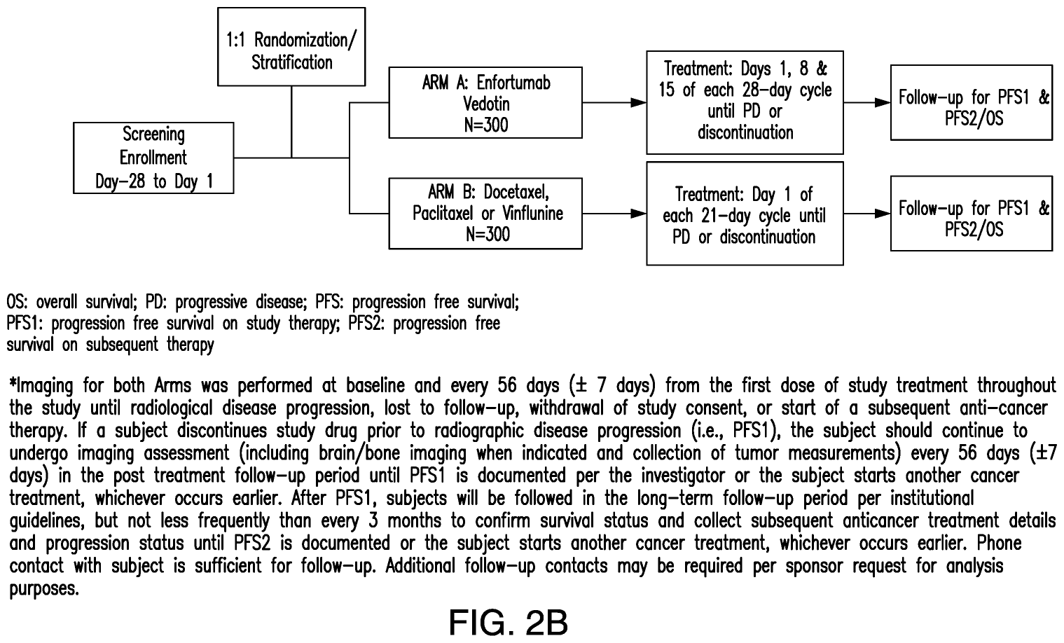

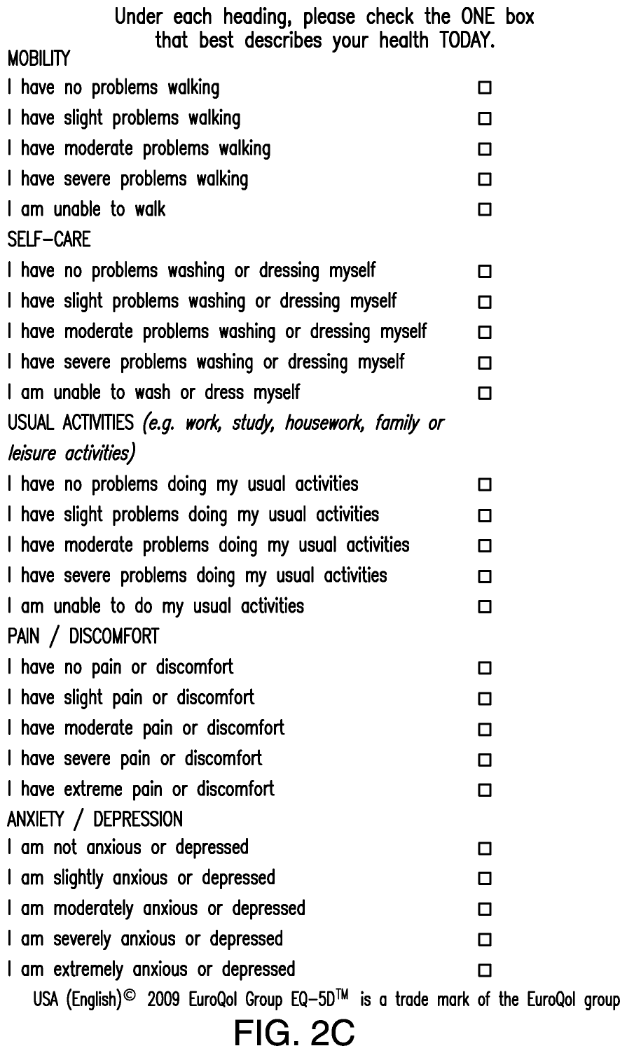

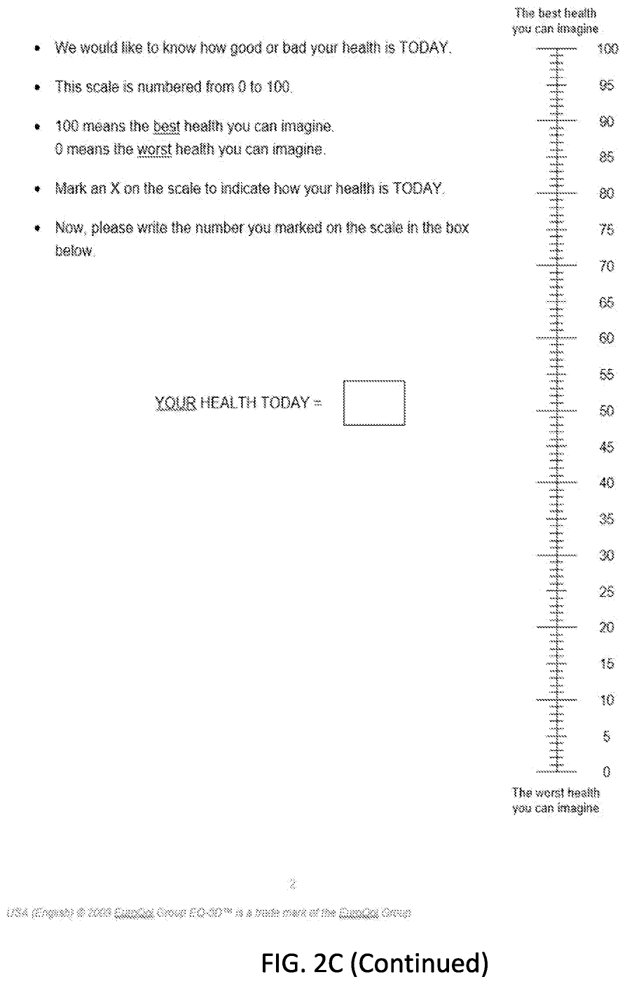

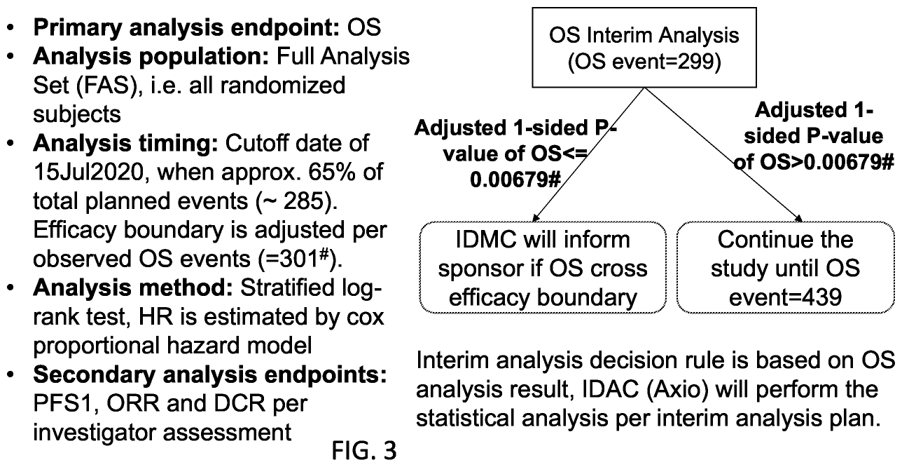

The study scheme and schedule of assessments are shown in FIG. 2B, Table 10, Table 11, and Table 12.

TABLE 10
Schedule of Assessments for Arms A and B

| | Visit | | | | | | | | | | |
|---|---|---|---|---|---|---|---|---|---|---|---|
| | | | | | | | | | Post | Long Term | Survival |
| | | | | | | | End of | Follow- | treatment | Treatment | Follow- |
| | Screening/Baseline¹ | | Every Cycle | | | | Treatment²¹ | up¹⁸ | Follow-Up | Follow-up | up |
| | Base Date | | | | | | | | | | |
| | | | | Day 8 | Day 15 | | | Date of | | | |
| | | | | Arm | Arm | Every | Date of | last | Every | | |
| | Day −28 | Day −7 | Day | A | A | 56 | Last | Dose + | 56 | Every 3 | Every 3 |
| | to −1 | to −1 | 1 | (only) | (only) | Days | Dose | 30 days | days | months | months |
| | Visit Window | | | | | | | | | | |
| | | | ±3 | ±3 | ±3 | ±7 | ±7 | ±7 | ±7 | ±7 | ±7 |
| | NA | NA | days | day | days | days | days | days | days | days | days |
| Informed Consent | X | | | | | | | | | | |
| Medical and Disease | X | | | | | | | | | | |
| History | | | | | | | | | | | |
| Confirmation of | | | X | | | | | | | | |
| Eligibility | | | | | | | | | | | |
| Tumor Tissue Sample² | X | | | | | | | | | | |
| Brain Scan³ | X | | | | | X | | | X | | |
| Bone Scan⁴ | X | | | | | X | | | X | | |
| PGx blood sample | | X | | | | | | | | | |
| (optional) | | | | | | | | | | | |
| Serum/Urine Pregnancy | | X | X | | | | X | X | X⁵ | X⁵ | X⁵ |
| Test⁵ | | | | | | | | | | | |
| Physical Examination⁶ | | X | X | | | | X | | | | |
| Weight | | X | X | X⁶ | X⁶ | | X | | | | |
| Vital Signs | | X | X | X⁶ | X⁶ | | X | X | | | |
| Biochemistry⁷ | | X | X | X⁷ | X⁷ | | X | X | | | |
| Hemoglobin A1C⁸ | | X | | | | | X | | | | |
| Hematology⁹ | | X | X | X⁹ | X⁹ | | X | X | | | |
| ECOG PS | | X | X | | | | X | X | | | |
| 12-lead ECG¹⁰ | | X | | | | | X | | | | |
| Ophthalmology | X | | | | | | X | | | | |
| Assessment¹¹ | | | | | | | | | | | |
| Randomization¹² | | X | | | | | | | | | |
| Arm A: EV | | | X | X | X | | | | | | |
| Administration −28 | | | | | | | | | | | |
| Day Cycle¹³ | | | | | | | | | | | |
| Arm B: | | | X | | | | | | | | |
| Docetaxel/Paclitaxel/ | | | | | | | | | | | |
| Vinflunine | | | | | | | | | | | |
| Administration −21 | | | | | | | | | | | |
| Day Cycle¹⁴ | | | | | | | | | | | |
| Image Assessment¹⁵ | X | | | | | X¹⁵ | | | X ¹⁹ | | |
| Subsequent Therapy | | | | | | | X | X | X | X | |
| Assessment²⁰ | | | | | | | | | | | |
| Concomitant Medication | X | X | X | X | X | X | X | X | | | |
| AE | X | X | X | X | X | X | X | X | | | |
| Quality of Life (QOL) ¹⁶ | | X | X | | | | X | X | | | |
| Healthcare Resource | | | X²² | | | | X | X | | | |
| Utilization (HRU)²² | | | | | | | | | | | |
| Overall Survival¹⁷ | | | | | | | | X | X | X | X |

AE: adverse event;
ALT: alanine transaminase;
AST: aspartate transaminase;
CR: complete response;
CT: computed tomography;
ECG: electrocardiogram;
ECOG PS: Eastern Cooperative Oncology Group Performance Status;
EORTC-QLQ-C30: European Organisation for Research and Treatment of Cancer Core Quality of Life Questionnaire;
EOT: end of treatment;
EQ-5D-5L: EuroQOL 5-Dimension 5-Level Questionnaire;
EV: enfortumab vedotin;
HRU: health resource utilization;
MRI: magnetic resonance imaging;
NA: Not Applicable;
PD: progressive disease;
PFS1: progression free survival on study therapy;
PGx: Pharmacogenetic analyses;
PR: partial response;
QOL: quality of life;
RECIST: Response Evaluation Criteria in Solid Tumors
¹Screening period is 28 days. Subjects may have screening assessments repeated once. If more than 1 assessment is taken during the screening, the assessment closest to enrollment date should be used for eligibility.
²Archival tumor tissue (from primary or metastatic site) for biomarker studies should be available for submission to the Sponsor prior to study treatment. If an archival tumor tissue sample is not available, a fresh tissue sample should be provided. A tissue block or a minimum of 10 and up to 15 freshly sectioned, unstained charged slides should be provided.
³Only if clinically indicated at baseline. Repeat as clinically indicated or per standard of care throughout the study.
⁴All subjects will have a baseline bone scan (scintigraphy) performed at screening. Subjects with positive bone scans at baseline will have a bone scan performed every 56 days (±7 days) throughout the study or more frequently if clinically indicated. Subjects should have a follow-up bone scan performed if clinically indicated regardless of baseline status.
⁵For all female subjects of child bearing potential only. A urine or serum pregnancy test will be performed at baseline. A urine or serum pregnancy test will then be repeated on day 1 of each cycle prior to EV or chemotherapy administration, at EOT and Follow-up visits. After EOT, a monthly (±7 days) pregnancy test will be maintained until 6 months after the last dose of study treatment.
⁶Full Physical examination and other evaluations including height (at screening only); weight, ECOG PS and vital signs (pulse, temperature and blood pressure) will be performed at screening. The physical examination will also be performed on day 1 of each cycle and EOT visit. Physical examination is only repeated on Cycle 1 Day 1 if clinically significant changes from Screening (in the opinion of the investigator) are observed. For subsequent and EOT visits physical examinations maybe more directed but should include examination of lungs, abdomen, skin and cardiovascular systems. Vital signs and weight will be completed on days 1, 8 and 15 of each cycle for Arm A and on day 1 of each cycle for Arm B, and at EOT visit. Vital signs will also be performed at follow up visit.
⁷Biochemistry: See Section 6.1.6.5(iii) Laboratory Assessments. Amylase and lipase will only be collected at screening and Day 1 of each Cycle. All biochemistry laboratory tests should be collected at the start of the following timepoints: Screening, Cycle 1 Day 1, Cycle 1 Day 8 (Arm A only), Cycle 1 Day 15 (Arm A only) and Day 1 of each subsequent cycle. If all biochemistry laboratory tests were performed within 7 days prior to the first day of dosing, they do not need to be repeated on Cycle 1 Day 1. Biochemistry tests will be sent to a central laboratory for analysis. Local laboratory results may be used to determine eligibility if the screening results from the central laboratory are not available in time for planned randomization. In the event that the central laboratory results received after randomization are not within eligibility parameters, the subject will still be considered eligible, if local labs met the eligibility criteria, and will not be considered a protocol deviation. Local laboratory results that support eligibility and dosing decisions must be entered into the clinical database. If local laboratory is to be used to support dosing decisions, local laboratory tests will include complete blood count (CBC) with differential, glucose, serum creatinine, ALT and AST. Additional assessments may be done centrally or locally to monitor AEs or as required by dose modification requirements.
⁸If HbA1c is elevated (≥6.5%), refer subject to appropriate provider during Cycle 1 for glucose management.
⁹Hematology: See Section 6.1.6.5(iii) Laboratory Assessments. Hematology tests should be collected at the following timepoints: Screening, Cycle 1 Day 1, Cycle 1 Day 8 (Arm A only), Cycle 1 Day 15 (Arm A only) and Day 1 of each subsequent cycle. If hematology tests were performed within 7 days prior to the first day of dosing, they do not need to be repeated on Cycle 1 Day 1. Hematology tests will be sent to a central laboratory for analysis. Local laboratory results may be used to determine eligibility if the screening results from the central laboratory are not available in time for planned randomization. In the event that the central laboratory results received after randomization are not within eligibility parameters, the subject will still be considered eligible if local laboratory results met the eligibility criteria; such evens will not be considered protocol deviations. Local laboratory results that support eligibility and dosing decisions must be entered into the clinical database. If local laboratory is to be used to support dosing decisions, local laboratory tests will include CBC with differential, glucose, serum creatinine, ALT and AST. Additional assessments may be done centrally or locally to monitor AEs or as required by dose modification requirements.
¹⁰ECGs will be read locally.
¹¹Ophthalmologic assessment for subjects with recent ocular complaints (within 3 months of screening) are required. Assessments should include the following: visual acuity, slit lamp, tonometry examination and dilated fundus examination. Prior ophthalmologic exam done within 3 months of screening is acceptable provided symptoms are not new since the exam. Ophthalmology assessments should be performed per standard of care or if clinically indicated (e.g., subject develops new or worsening ocular symptoms). EOT slit lamp examinations are required for subjects who experience corneal adverse events during the study. EOT slit lamp examinations must be performed ≥4 weeks from last dose. Additional eye examinations are to be conducted as clinically indicated.
¹²Randomization will be allowed starting at Day −3 to allow for premedication in Arm B. Cycle 1 Day 1 treatment should occur within 3 days of randomization.
¹³EV will be administered on Days 1, 8 and 15 of every 28-day cycle. Weight-based dosing is calculated using the subject's actual body weight on Day 1 of each cycle. The dose does not need to be recalculated based on actual weight on Day 8 and 15 of each cycle for Arm A unless it is required by institutional standards. At least 1 week must elapse between doses of EV. Subjects receiving enfortumab vedotin should be observed during enfortumab vedotin administration and for at least 60 minutes following the infusion for the first 3 cycles.
¹⁴Docetaxel, paclitaxel or vinflunine will be administered on Day 1 of every 21-day cycle. Subjects receiving docetaxel, paclitaxel or vinflunine should be observed during study drug administration and for at least 30 minutes following the infusion during the first 3 cycles.
¹⁵Imaging for Arms A & B will be evaluated at Baseline and every 56 days (±7 days) throughout the study. CT scan with contrast (chest, abdomen and pelvis) is the preferred modality for tumor assessment. MRI is acceptable if local standard practice or if CT scans are contraindicated in a subject (e.g., subject is allergic to contrast media). All other RECIST approved scanning methods such as x-ray are optional. To ensure comparability, the screening and subsequent assessment of response should be performed using identical techniques. The same method should be employed and assessed by the same individual on each occasion if possible. Imaging assessments methods used at Baseline are to be used throughout the study.
¹⁶ QOL questionnaires (EORTC-QLQ-C30 and EQ-5D-5L) will be completed at Baseline (Day −7 to −1) and on Day 1 of each week (+7 days) for the first 12 weeks and then every 12 weeks afterward, EOT visit and at the follow-up visit. QOL questionnaire completion timing should be calculated based on Cycle 1 Day 1 dosing. If a visit occurs out of the assessment window, QOL questionnaires should still be completed. QOL questionnaires will be completed by the subject at home on hand-held devices prior to coming to the clinic visit with the exception of Baseline Day 1 of the first week. the EOT and the follow-up visits at which the QOL questionnaires will be completed by the subject at the clinic.
¹⁷Contact subjects in the survival follow-up period approximately every 3 months to collect survival status until subject death or study closure. Additional follow-up contacts may be required per sponsor request for analysis purposes.
¹⁸Follow up assessments should be completed prior to the initiation of the next therapy.
¹⁹ If a subject discontinues study drug prior to radiographic disease progression (i.e., PFS1), the subject should continue to undergo imaging assessment (including brain/bone imaging when indicated and collection of tumor measurements) every 56 days (±7 days) in the post-treatment follow-up period until PFS1 is documented per the investigator, or the subject starts another cancer treatment, whichever occurs earlier.
²⁰After PFS1, subjects will be followed in the long-term follow-up period per institutional guidelines, but not less frequently than every 3 months to confirm survival status and collect subsequent anticancer treatment details and progression status until PFS2 is documented or the subject starts another cancer treatment, whichever occurs earlier. Phone contact with subject is sufficient for follow-up. Additional follow-up contacts may be required per sponsor request for analysis purposes.
²¹EOT visit will occur within 7 days after the last dose or when the decision is made by the investigator to discontinue subject from treatment.
²²HRU questionnaires will be completed monthly (Day 1 of every 4 weeks (+7 days)), starting on Week 5 Day 1 (timing calculated based on Cycle 1 Day 1 dosing), and at the EOT and follow-up visit. HRU questionnaires will be completed by the subject at home on hand-held devices prior to coming to the clinic visit with the exception of the EOT and the follow-up visits at which the HRU questionnaires will be completed by the subject at the clinic.

TABLE 11
Pharmacokinetic, ATA, and biomarker blood sample collection timepoints - Arm A

| | | | | | Blood | | | | |
|---|---|---|---|---|---|---|---|---|---|
| | | | | | | | Biomarkers | | |
| | | | | | | | Plasma | | PBMC |
| | | | | | Pharmaco- | | | cf | Immuno- |
| | Study Day | Time | Window | Relative Time | kinetics | ATA | Cytokines | DNA | phenotyping |
| Cycles 1 | Day 1 | Pre-dose | within 24 hr | START of infusion | X | X | X | X | X |
| | | End of infusion | Within 15 min | END of infusion | X | | | | |
| | Day 8 | Pre-dose | Within 24 hrs | START of infusion | X | | X | | X |
| | Day 15 | Pre-dose | Within 24 hrs | START of infusion | X | | X | | X |
| | | End of infusion | Within 15 min | END of infusion | X | | | | |
| Cycles 2 | Day 1 | Pre-dose | Within 24 hrs | START of infusion | X | X | X | X | X |
| Subsequent Dosing Cycles | Day 1 | Pre-dose | Within 24 hrs | START of infusion | X^{A} | X^{A} | X^{B} | X^{C} | X^{B} |

| | | | | | |
|---|---|---|---|---|---|
| End of Treatment (Date of last dose + 7 days) | X | X | X | X | X |
| Follow-up (Date of last dose + 30 days) | X | X | | | |

ATA: antitherapeutic antibodies;
PBMC: peripheral blood mononuclear cells;
cfDNA: circulating free deoxyribonucleic acid
^{A}Pharmacokinetics and ATA: Pre-dose of cycle 3, 4 and every even numbered cycle there after
^{B}Cytokines and Immuno-phenotyping: Pre-dose cycles 3 and 4 only
^{C}cfDNA: Pre-dose every even numbered cycle (e.g., cycle 4, 6, etc.) only

TABLE 12
Biomarker blood sample collection timepoints - Arm B

| | | | | | Biomarkers | | |
|---|---|---|---|---|---|---|---|
| | | | | | Plasma | | PBMC |
| | | | | | | cf | Immuno- |
| | Study Day | Time | Window | Relative Time | Cytokines | DNA | phenotyping |
| Cycle 1 | Day 1 | Pre-dose | Within 24 hr | START of infusion | X | X | X |
| Cycle 2 | Day 1 | Pre-dose | Within 24 hr | START of infusion | X | X | X |
| Subsequent Dosing Cycles | Day 1 | Pre-dose | Within 24 hr | START of infusion | X^{B} | X^{A} | X^{B} |

| | | | |
|---|---|---|---|
| End of Treatment (Date of last dose + 7 days) | X | X | X |

cfDNA: circulating free deoxyribonucleic acid;
PBMC: peripheral blood mononuclear cells
^{A}cfDNA: Pre-dose every even numbered cycle (e.g., cycle 4, 6, etc.) only
^{B}Cytokines and immune-phenotyping: pre-dose cycles 3 and 4 only

### 6.1.3 Study Objective(S), Design and Endpoints

### 6.1.3.1 Study Objective(s)

(i) Primary Objective

To compare the OS of subjects with locally advanced or metastatic urothelial cancer treated with EV to the OS of patients treated with chemotherapy.

(ii) Secondary Objectives

To compare progression free survival on study therapy (PFS1) per Response Evaluation Criteria in Solid Tumors (RECIST) V1.1 of subjects treated with EV to patients treated with chemotherapy

To compare the overall response rate (ORR) per RECIST V1.1 of EV to chemotherapy

To evaluate the duration of response (DOR) per RECIST V1.1 of EV and chemotherapy

To compare the disease control rate (DCR) per RECIST V1.1 of EV to chemotherapy

To assess the safety and tolerability of EV

To assess quality of life (QOL) and Patient Reported Outcomes (PRO) parameters

(iii) Exploratory Objectives

Exploratory genomic and/or other biomarkers in tumor tissue and in peripheral blood that may correlate with treatment outcome, including Nectin-4 expression

To assess the pharmacokinetics of EV (TAb, ADC and MMAE)

To assess the incidence of ATA

To evaluate PFS as assessed by RECIST V1.1 by investigator review in the next line of therapy (PFS2) in subjects treated with EV compared to docetaxel, paclitaxel or vinflunine.

Healthcare resources utilization (HRU)

### 6.1.3.2 Study Design and Dose Rationale

(i) Study Design

This is a global, open-label, randomized Phase 3 study in adult subjects with locally advanced or metastatic urothelial cancer who have received a platinum-containing chemotherapy and have experienced disease progression or relapse during or following treatment with an immune checkpoint inhibitor. Subjects who discontinued CPI treatment due to toxicity are eligible provided that they have evidence of disease progression following discontinuation.

Approximately 600 subjects will be randomized to EV (Arm A) or chemotherapy (Arm B) in a 1:1 ratio. Subjects will be stratified according to the following: Eastern Cooperative Oncology Group Performance Status (ECOG PS), regions of the world and liver metastasis.

OS is the primary endpoint. OS is defined as the time from randomization to the date of death. Secondary endpoints include PFS1, ORR, DOR, DCR, safety and QOL/PRO.

Subjects in Arm A will receive EV on Days 1, 8 and 15 of each 28-day cycle. Subjects in arm B will receive either docetaxel, paclitaxel or vinflunine as decided by the investigator prior to randomization, (vinflunine is a choice of comparator only in countries where it is approved for urothelial cancer) on Day 1 of every 21-day cycle. Within the control arm, the overall proportion of subjects receiving vinflunine will be capped at approximately 35%. Subjects will continue to receive study treatment until radiological disease progression as determined per investigator assessment or other discontinuation criteria are met or upon study termination, or study completion, whichever occurs first. No on-study crossover will be allowed. Subjects assigned to the chemotherapy arm will not be allowed to switch to a different chemotherapy treatment during study treatment. This study will consist of three phases: screening, treatment and follow-up.

Screening will take place up to 28 days prior to randomization. Screening assessments may be repeated within the 28-day screening period.

Subjects do not need to be “screen failed” in IRT and re-entered in screening with a new subject ID as long as the subject is enrolled within the 28-day window from signing of the informed consent. If more than 28 days elapses from the date of signing the informed consent, the subject must be screen failed in IRT. A new consent must be signed and the subject entered into screening with a new subject ID. Subjects may only be rescreened once.

Subjects will start with cycle 1 and continue on to subsequent 21-day or 28-day cycles until one of the discontinuation criteria are met or upon study termination, or study completion, whichever occurs first. A treatment cycle is defined as 28 days for Arm A and 21 days for Arm B.

Subjects will be evaluated for response according to RECIST V1.1. Imaging for both arms will be performed at baseline and every 56 days (±7 days) from the first dose of study treatment throughout the study until PFS1 is documented by radiological disease progression or the subject is lost to follow-up, death, withdraws study consent or starts a subsequent anti-cancer therapy. Baseline imaging performed prior to informed consent as standard of care may be used so long as it is performed within 28 days prior to randomization. All subjects will have a bone scan (scintigraphy) performed at screening/baseline. Subjects with positive bone scans at baseline will have a bone scan performed every 56 days (±7 days) throughout the study or more frequently if clinically indicated. Subjects should have a follow-up bone scan performed if clinically indicated regardless of baseline status. Brain scans (computed tomography (CT) with contrast/magnetic resonance imaging (MRI)) will only be performed if clinically indicated at screening/baseline and repeated as clinically indicated or per standard of care throughout the study.

QOL assessments and PRO will be collected at protocol-specified time points from all randomized subjects. The following validated tools will be used: European Organisation for Research and Treatment of Cancer (EORTC) Quality of Life Questionnaire (QLQ-C30) and EuroQOL 5-dimension 5-Level Questionnaire (EQ-5D-5L). HRU information will be collected at protocol-specified time points with particular focus on the number of subjects who have an unplanned use of healthcare resources related to clinical or AEs from subjects assigned to treatment arms A and B.

Blood samples for pharmacokinetics and ATA will be collected throughout the study for subjects randomized into Arm A. Validated assays will be used to measure the concentrations of EV ADC and monomethylauristatin E (MMAE) in serum or plasma and to assess ATA. Pharmacokinetic samples will not be collected from subjects randomized into Arm B. Samples for exploratory biomarkers will be collected at protocol-specified timepoints.

Following discontinuation from study drug, subjects will have a follow-up visit 30 days (+7 days) after their last dose of drug for safety assessments. If a subject discontinues study drug prior to radiographic disease progression (i.e., PFS1), the subject should enter the post- treatment follow-up period and continue to undergo imaging assessments every 56 days (±7 days) until PFS1 is documented, or the subject starts another anticancer treatment, whichever occurs earlier.

Following PFS1, subjects will enter the long-term follow-up period and be followed per institutional guidelines but not less than every 3 months from the date of the follow-up visit for survival status and progression status on subsequent therapy (i.e., PFS2).

Subjects will be followed until PFS2 is documented or the subject starts another anticancer treatment, whichever occurs earlier. All subsequent anticancer therapy including date and site of progression for PFS2 will be recorded on the case report form.

Following PFS2, subjects will enter the survival follow-up period and be followed every 3 months for survival status until death, lost to follow-up, withdrawal of study consent, or study termination by sponsor. This study is expected to end once final survival analysis is complete. Subjects will be eligible to continue receiving treatment in this study until they meet a discontinuation criterion as outlined in Section 6.1.7 Discontinuation or upon study termination, or study completion, whichever occurs first.

An IDMC will be chartered to oversee safety and the planned interim efficacy analysis, which will occur after at least 285 OS events (about 65% of the total planned events) are observed. The primary analysis will occur at 439 OS events. The IDMC may recommend to the sponsor whether the trial should be terminated, modified or continue unchanged based on ongoing reviews of safety data and interim efficacy analysis. Further details will be outlined in the IDMC charter.

(ii) Dose Rationale

(a) Enfortumab Vedotin

EV will be administered at a dose of 1.25 mg/kg as an intravenous infusion over approximately 30 minutes on Days 1, 8, and 15 of each 28-day cycle. This dose and regimen has demonstrated an acceptable safety profile and encouraging clinical activity in the phase 1 study, which evaluated escalating dose levels of 0.5, 0.75, 1 and 1.25 mg/kg, with expansion cohorts at the 0.75, 1, and 1.25 mg/kg dose levels. A maximum tolerated dose (MTD) was not reached in this study. At the 1 mg/kg dose level, 2 dose- limiting toxicities (DLTs) were observed: Grade 3 proctalgia (later changed to grade 2 by the investigator) thought related to radiation recall and Grade 4 hyperuricemia without clinical sequelae. No DLT was observed at 1.25 mg/kg and doses above 1.25 mg/kg were not tested.

Incidence of some of the most frequent drug-related AEs, such as diarrhea and rash, although primarily Grades 1-2 and clinically manageable, increased with increasing dose levels.

Moreover, dose reductions due to AEs were more frequent for the 1.25 mg/kg vs lower dose levels. Safety assessments of both metastatic urothelial cancer patients and non-metastatic urothelial cancer patients showed that frequency of all TEAEs, AEs leading to withdrawal, and Grade 3-4 TEAEs were comparable across all dose levels.

While all doses of EV demonstrated activity, the 1.25 mg/kg dose was associated with the highest activity and had an acceptable safety profile.

Based on pharmacokinetic data from the phase 1 study, the half-life of EV is ˜1-2 days. No notable (<30%) intra-cycle accumulation of ADC was observed with the current dosing regimen (on Days 1, 8 and 15 of every 28-day cycle) at any dose level. Minimal (<50%) intra-cycle accumulation of MMAE was observed. It is anticipated that the dosing schedule in the study will maintain ADC exposures over each 28-day cycle, contributing to a favorable balance of activity and safety, as observed in the phase 1 trial.

In summary, the dose regimen of 1.25 mg/kg on Days 1, 8 and 15 of each 28-day cycle proposed for this study has demonstrated an acceptable safety profile and encouraging clinical activity that was higher than at lower dose levels.

(b) Comparators

There is no accepted standard of care following CPI treatment for locally advanced or mUC; however, prior to CPI approvals, taxanes were commonly used following platinum-based therapy as recommended in the treatment guidelines. In addition, vinflunine is approved in Europe for treatment of mUC after failure of a prior platinum prior platinum-based regimen.

KEYNOTE-045, a multicenter, randomized, active-controlled trial in patients with locally advanced or mUC with disease progression on or after platinum-containing chemotherapy, compared pembrolizumab to investigator's choice of paclitaxel, docetaxel, or vinflunine every 3 weeks. Bellmunt J, et al., N Engl J Med. 2017; 376:1015-26. DOI: 10.1056/NEJMoa1613683.

Although no labeled dosing guidelines for taxanes are available in this setting, this is the only large randomized trial that has reported combined survival data for these agents, as such the current phase 3 study of enfortumab vs chemotherapy will use the same choice of comparators and their corresponding doses. Patients randomized to the chemotherapy arm in the KEYNOTE-045 study were treated with paclitaxel (175 mg/m²), docetaxel (75 mg/m²) or vinflunine (320 mg/m²), administered intravenously every 3 weeks. The overall proportion of subjects receiving vinflunine in the control arm was capped at approximately 35% (KEYNOTE protocol).

### 6.1.3.3 Endpoints

(i) Primary Endpoints

OS

(ii) Secondary Endpoints

PFS1 by RECIST V1.1

ORR (CR+PR) by RECIST V1.1

DCR (CR+PR+SD) by RECIST V1.1

DOR by RECIST V1.1

Safety variables (e.g., AEs, laboratory tests, vital sign measurements, 12-lead ECG and ECOG PS)

QOL and PRO parameters (QLQ-C30 and EQ-5D-5L)

(iii) Exploratory Endpoints

Exploratory genomic and/or other biomarkers in tumor tissue and in peripheral blood that may correlate with treatment outcome, including Nectin-4 expression

Plasma or serum concentrations of TAb, ADC and MMAE

Incidence of ATA to EV

PFS2

HRU

### 6.1.4 Study Population

### 6.1.4.1 Selection of Study Population

Patients with locally advanced or metastatic urothelial cancer previously treated with platinum-based chemotherapy and an immune checkpoint inhibitor (CPI).

### 6.1.4.2 Inclusion Criteria

Subject is eligible for the study if all of the following apply:

Institutional Review Board (IRB)/Independent Ethics Committee (IEC) approved written informed consent and privacy language as per national regulations (e.g., Health Insurance Portability and Accountability Act (HIPAA) Authorization for US sites) must be obtained from the subject prior to any study-related procedures (including withdrawal of prohibited medication, if applicable).

Subject is legally an adult according to local regulation at the time of signing informed consent.

Subject has histologically or cytologically confirmed urothelial carcinoma (i.e., cancer of the bladder, renal pelvis, ureter, or urethra). Subjects with urothelial carcinoma (transitional cell) with squamous differentiation or mixed cell types are eligible.

Subject must have experienced radiographic progression or relapse during or after a CPI (anti-programmed cell death protein-1 (PD-1) or anti-programmed cell death-ligand 1 (PD-L1)) for locally advanced or metastatic disease. Subjects who discontinued CPI treatment due to toxicity are eligible provided that they have evidence of disease progression following discontinuation. The CPI need not be the most recent therapy. Subjects for whom the most recent therapy has been a non-CPI based regimen are eligible if they have progressed/relapsed during or after their most recent therapy. Locally advanced disease must not be amenable to resection with curative intent per the treating physician.

Subject must have received a platinum containing regimen (cisplatin or carboplatin) in the metastatic/locally advanced, neoadjuvant or adjuvant setting. If platinum was administered in the adjuvant/neoadjuvant setting subject must have progressed within 12 months of completion.

Subject has radiologically documented metastatic or locally advanced disease at baseline.

An archival tumor tissue sample should be available for submission to central laboratory prior to study treatment. If an archival tumor tissue sample is not available, a fresh tissue sample should be provided. If a fresh tissue sample cannot be provided due to safety concerns, enrollment into the study must be discussed with the medical monitor.

Subject has ECOG PS of 0 or 1.

The subject has the following baseline laboratory data:
absolute neutrophil count (ANC) ≥1500/mm³
platelet count ≥100×10⁹/L
hemoglobin ≥9 g/dL
serum total bilirubin ≤1.5×upper limit of normal (ULN)* or ≤3×ULN for subjects with Gilbert's disease
creatinine clearance (CrCl) ≥30 mL/min as estimated per institutional standards or as measured by 24 hour urine collection (glomerular filtration rate (GFR) can also be used instead of CrCl)
alanine aminotransferase (ALT) and aspartate aminotransferase (AST) ≤2.5×ULN or ≤3×ULN for subjects with liver metastases*
   Docetaxel should not be chosen as a comparator for subjects if total bilirubin >ULN, or if AST and/or ALT >1.5×ULN concomitant with alkaline phosphatase >2.5×ULN.

Female subject must either:
Be of nonchildbearing potential:
   Postmenopausal (defined as at least 1 year without any menses for which there is no other obvious pathological or physiological cause) prior to screening, or
   Documented surgically sterile (e.g., hysterectomy, bilateral salpingectomy, bilateral oophorectomy).
   Note: Those who are amenorrheic due to an alternative medical cause are not considered postmenopausal and must follow the criteria for childbearing potential subjects.
Or, if of childbearing potential:
   Agree not to try to become pregnant during the study and for at least 6 months after the final study drug administration,
   And have a negative urine or serum pregnancy test within 7 days prior to Day 1 (Females with false positive results and documented verification of negative pregnancy status are eligible for participation),
   And if heterosexually active, agree to consistently use a condom plus 1 form of highly effective birth control * per locally accepted standards starting at screening and throughout the study period and for at least 6 months after the final study drug administration.

Female subject must agree not to breastfeed or donate ova starting at screening and throughout the study period, and for at least 6 months after the final study drug administration.

A sexually active male subject with female partner(s) who is of childbearing potential is eligible if:
Agrees to use a male condom starting at screening and continue throughout the study treatment and for at least 6 months after final study drug administration. If the male subject has not had a vasectomy or is not sterile as defined below his female partner(s) is utilizing 1 form of highly effective birth control* per locally accepted standards starting at screening and continue throughout study treatment and for at least 6 months after the male subject receives his final study drug administration.
Highly effective forms of birth control include:
Consistent and correct usage of established hormonal contraceptives that inhibit ovulation,
Established intrauterine device (IUD) or intrauterine hormone releasing system (IUS).
Bilateral tubal occlusion
Vasectomy (A vasectomy is a highly effective contraception method provided the absence of sperm has been confirmed. If not, an additional highly effective method of contraception should be used)
Male is sterile due to a bilateral orchiectomy or radical cystoprostatectomy/removal of seminal vesicles
Sexual Abstinence is considered a highly effective method only if defined as refraining from heterosexual activity during the entire period of risk associated with the study drug. The reliability of sexual abstinence needs to be evaluated in relation to the duration of the study and the preferred and usual lifestyle of the participant
Note: Sexual abstinence is not sufficient as contraception method in Switzerland

Male subject must not donate sperm starting at screening and throughout the study period, and for at least 6 months after the final study drug administration.

Male subject with a pregnant or breastfeeding partner(s) must agree to abstinence or use a condom for the duration of the pregnancy or time partner is breastfeeding throughout the study period and for at least 6 months after the final study drug administration.

Subject agrees not to participate in another interventional study while on treatment in present study.

Waivers to the inclusion criteria will not be allowed.

### 6.1.4.3 Exclusion Criteria

Subject will be excluded from participation if any of the following apply:

Subject has preexisting sensory or motor neuropathy Grade ≥2.

Subject has active central nervous system (CNS) metastases. Subjects with treated CNS metastases are permitted on study if all the following are true:
CNS metastases have been clinically stable for at least 6 weeks prior to screening
If requiring steroid treatment for CNS metastases, the subject is on a stable dose ≤20 mg/day of prednisone or equivalent for at least 2 weeks
Baseline scans show no evidence of new or enlarged brain metastasis
Subject does not have leptomeningeal disease

Subject has ongoing clinically significant toxicity (Grade 2 or higher with the exception of alopecia) associated with prior treatment (including systemic therapy, radiotherapy or surgery). Subject with ≤Grade 2 immunotherapy-related hypothyroidism or panhypopituitarism may be enrolled when well-maintained/controlled on a stable dose of hormone replacement therapy (if indicated). Patients with ongoing ≥Grade 3 immunotherapy-related hypothyroidism or panhypopituitarism are excluded. Subjects with ongoing immunotherapy related colitis, uveitis, myocarditis, or pneumonitis or subjects with other immunotherapy related AEs requiring high doses of steroids (>20 mg/day of prednisone or equivalent) are excluded.

Subject has prior treatment with EV or other MMAE-based ADCs.

Subject has received prior chemotherapy for urothelial cancer with all available study therapies in the control arm (i.e., both prior paclitaxel and docetaxel in regions where vinflunine is not an approved therapy, or prior paclitaxel, docetaxel and vinflunine in regions where vinflunine is an approved therapy). Note: after vinflunine cap is reached, subjects who have received both docetaxel and paclitaxel will be excluded.

Subject has received more than 1 prior chemotherapy regimen for locally advanced or metastatic urothelial cancer, including chemotherapy for adjuvant or neo-adjuvant disease if recurrence occurred within 12 months of completing therapy. The substitution of carboplatin for cisplatin does not constitute a new regimen provided no new chemotherapeutic agents were added to the regimen.

Subject has history of another malignancy within 3 years before the first dose of study drug, or any evidence of residual disease from a previously diagnosed malignancy. Subjects with nonmelanoma skin cancer, localized prostate cancer treated with curative intent with no evidence of progression, low-risk or very low-risk (per standard guidelines) localized prostate cancer under active surveillance/watchful waiting without intent to treat, or carcinoma in situ of any type (if complete resection was performed) are allowed.

Subject is currently receiving systemic antimicrobial treatment for viral, bacterial, or fungal infection at the time of first dose of EV. Routine antimicrobial prophylaxis is permitted.

Subject has known active Hepatitis B (e.g., HBsAg reactive) or active hepatitis C (e.g., HCV RNA (qualitative) is detected).

Subject has known history of human immunodeficiency virus (HIV) infection (HIV 1 or 2).

Subject has documented history of a cerebral vascular event (stroke or transient ischemic attack), unstable angina, myocardial infarction, or cardiac symptoms (including congestive heart failure) consistent with New York Heart Association Class III-IV within 6 months prior to the first dose of study drug.

Subject has radiotherapy or major surgery within 4 weeks prior to first dose of study drug.

Subject has had chemotherapy, biologics, investigational agents, and/or antitumor treatment with immunotherapy that is not completed 2 weeks prior to first dose of study drug.

Subject has known hypersensitivity to EV or to any excipient contained in the drug formulation of EV (including histidine, trehalose dihydrate, and polysorbate 20); OR subject has known hypersensitivity to biopharmaceuticals produced in Chinese hamster ovary (CHO) cells.

Subject has known hypersensitivity to the following:
docetaxel or to any of the other excipients listed in product label, including polysorbate 80;
Paclitaxel or to any of the other excipients listed in product label, including macrogolglycerol ricinoleate 35 (Ph.Eur.); and
vinflunine or to any of the other excipient listed in product label, including other vinca alkaloids (vinblastine, vincristine, vindesine, vinorelbine).

Subject has known active keratitis or corneal ulcerations. Subject with superficial punctate keratitis is allowed if the disorder is being adequately treated in the opinion of the investigator.

Subject has other underlying medical condition that, in the opinion of the investigator, would impair the ability of the subject to receive or tolerate the planned treatment and follow-up.

History of uncontrolled diabetes mellitus within 3 months of the first dose of study drug. Uncontrolled diabetes is defined as hemoglobin A1C (HbA1c) ≥8% or HbA1c between 7 and <8% with associated diabetes symptoms (polyuria or polydipsia) that are not otherwise explained.

Waivers to the exclusion criteria will not be allowed.

### 6.1.5 Treatment(s)

### 6.1.5.1 Identification of Drug Product(s)

(i) Study Drug(s)

The drug product, EV (ASG-22CE), is a sterile, preservative-free, white to off-white lyophilized powder to be reconstituted for intravenous administration. The drug product is supplied by sponsor in single-use glass vials containing 30 mg EV (ASG-22CE) in each vial. The drug product should be stored at 2-8° C. Details of drug product receipt, labeling, storage and preparation are provided in a supplemental pharmacy guide.

(ii) Comparative Drug(s)

Comparative drugs will be supplied by the responsible site pharmacy of each clinical trial site. If the site is unable to procure or utilize local supplies for the comparative drug, supplies may be provided centrally by the sponsor as applicable. Sites are permitted to utilize generic docetaxel and paclitaxel that is approved by the respective regulatory authority. Refer to product labels for docetaxel, paclitaxel and vinflunine for storage & handling conditions and caution statements.

The study refers to Section 6.1.6.1 Dosing and Administration of Study Drug(s) and Other Medication(s) for specific dosing instructions.

### 6.1.5.2 Packaging and Labeling

EV (ASG-22CE) used in this study will be prepared, packaged, and labeled under the responsibility of qualified staff in accordance with Standard Operating Procedures (SOPs), Good Manufacturing Practice (GMP) guidelines, ICH GCP guidelines, and applicable local laws/regulations.

Each carton and vial will bear a label conforming to regulatory guidelines, GMP and local laws and regulations that identifies the contents as drug product.

Where supplied by the Sponsor, the comparative drug(s) used in this study will be labeled in accordance with SOPs, GMP guidelines, ICH GCP guidelines, and applicable local laws/regulations.

A qualified person will perform the final release of the medication according to the requirements of the EUDirective 2003/94/EC annex 13.

### 6.1.5.3 Study Drug Handling

Current ICH GCP Guidelines require the investigator to ensure that study drug deliveries from the sponsor are received by the investigator/or designee and that:
such deliveries are recorded,
study drug is handled and stored according to labeled storage conditions,
study drug with appropriate expiry/retest and is only dispensed to study subjects in accordance with the protocol, and
any unused study drug is returned to the sponsor or standard procedures for the alternative disposition of unused study drug are followed.

The head of the study site or the study drug storage manager should take accountability of the study drugs as follows:
The study drug storage manager should store and take accountability of the study drugs in conforming to the procedures for handling the study drugs written by the sponsor.
The study drug storage manager should prepare and retain records of the study drug's receipt, the inventory at the study site, the use by each subject, and the return to the sponsor or alternative disposal of unused study drugs. These records should include dates, quantities, batch/serial numbers, expiration dates (if applicable), and the unique code numbers assigned to the study drugs and subjects.
The study drug storage manager should prepare and retain records that document adequately that the subjects were provided the doses specified by the protocol, and reconcile all the study drugs supplied from the sponsor.

### 6.1.5.4 Blinding

This is an open label study.

Although the study is an open label study, to maintain trial integrity, analyses or summaries by randomized treatment assignment or actual treatment assignment will be limited and documented while the study is ongoing and before the primary hard lock. Interim analysis will be conducted externally by independent data analysis center (IDAC). Details will be included in SAP.

### 6.1.5.5 Assignment and Allocation

Subjects will be randomized in a 1:1 ratio to a treatment arm according to the randomization schedules through Interactive Response Technology (IRT). All subjects who meet the eligibility criteria will be randomized. The site personnel will dispense the treatment according to the IRT system's assignment. Specific procedures for randomization through the IRT are contained in the study procedures manual. Investigators must select one treatment among the Arm B options before randomization occurs to be used in the event that the subject is randomized to the Arm B. Within the control arm, the overall proportion of subjects receiving vinflunine will be capped at approximately 35%.

### 6.1.6 Treatments and Evaluation

### 6.1.6.1 Dosing and Administration of Study Drug(s) and Other Medication(s)

(i) Dose/Dose Regimen and Administration Period

(a) Enfortumab Vedotin

EV at a dose of 1.25 mg/kg will be administered as an intravenous infusion over approximately 30 minutes on Days 1, 8, and 15 of every 28-day cycle. In the absence of IRRs, the infusion rate for all subjects should be calculated in order to achieve an approximate 30-minute infusion period. EV must not be administered as an intravenous push or bolus. EV should not be mixed with other medications. At least 1 week must elapse between doses of EV.

Weight-based dosing is calculated using the subject's actual body weight on Day 1 of each cycle. The dose does not need to be re-calculated based on actual weight on Day 8 and 15 of each cycle for Arm A unless it is required by institutional standards. An exception to weight-based dosing is made for subjects weighing greater than 100 kg; doses will be based on 100 kg for these individuals. The maximum dose permitted on this study is 125 mg.

Subject weight must be measured during all relevant assessment timepoints as described in the schedule of events.

The subject should be observed during enfortumab vedotin administration and for at least 60 minutes following the infusion during the first 3 cycles. All supportive measures consistent with optimal patient care should be given throughout the study according to institutional standards.

The injection site should be monitored closely for redness, swelling, pain, and infection during and at any time after administration. Subjects should be advised to report redness or discomfort promptly at the time of administration or after infusion.

(b) Comparative Drug(s)

Local product labels or summary of product characteristics (SmPC) and institutional guidelines will be followed for the administration of chemotherapy agents and precautions taken to prevent extravasation per institutional standards and as described in “Chemotherapy and Biotherapy Guidelines and Recommendations for Practice” (Polovich M, et al., Chemotherapy and biotherapy guidelines and recommendations for practice. 4th ed. Pittsburgh: Oncology Nursing Society; 2014. 473 p.) and “Management of Chemotherapy Extravasation: ESMO-EONS Clinical Practice Guidelines.” Fidalgo J A, et al., Ann Oncol. 2012; 23(7): 167-73.

Male subjects randomized to receive docetaxel, vinflunine or paclitaxel should seek medical advice regarding cryopreservation of sperm prior to receiving treatment due to the possibility of infertility.

Docetaxel

Study treatment of docetaxel should be administered as an intravenous infusion on Day 1 of every 21-day cycle after all procedures/assessments have been completed, including the required premedication per local standard of care prior to Day 1. Docetaxel will be administered at 75 mg/m² unless specified otherwise in Sections 6.1.6.1(i)(b) and 6.1.6.1(ii)(c). Refer to local product label or SmPC where supplied centrally and institution guidelines for docetaxel for further guidance on docetaxel dosing.

Docetaxel will be administered over 1 hour or per local guidelines. The subject should be observed during docetaxel administration and for at least 30 minutes following the infusion during the first 3 cycles. All supportive measures consistent with optimal patient care should be given throughout the study according to institutional standards.

All subjects should be premedicated per local standard of care with corticosteroids, such as dexamethasone 16 mg/day orally (e.g., 8 mg twice daily) for 3 days starting 1 day prior to docetaxel administration in order to reduce the incidence and severity of fluid retention as well as the severity of hypersensitivity reactions. The appropriate premedication regimen may be determined by the investigator.

Vinflunine

Study treatment of vinflunine should be administered as an intravenous infusion on Day 1 of every 21-day cycle after all procedures/assessments have been completed. Vinflunine will be administered at 320 mg/m² unless otherwise specified in Section 6.1.6.1(ii)(d). See 6.1.6.1(ii)(d) Vinflunine Dose Modifications for guidelines on adjustment of initial dose. The subject should be observed during vinflunine administration and for at least 30 minutes following the infusion during the first 3 cycles. All supportive measures consistent with optimal patient care should be given throughout the study according to institutional standards. Refer to local product label or SmPC and institution guidelines for vinflunine for further guidance on vinflunine dosing.

Paclitaxel

Study treatment of paclitaxel should be administered as an intravenous infusion on Day 1 of every 21-day cycle after all procedures/assessments have been completed. Paclitaxel will be administered at 175 mg/m² unless specified otherwise in Section 6.1.6.1(ii)(e). See Section 6.1.6.1(ii)(e) Paclitaxel Dose Modifications on adjustment of initial dose. Paclitaxel should be administered over 3 hours or per local guidelines. The subject should be observed during paclitaxel administration and for at least 30 minutes following the infusion during the first 3 cycles. All supportive measures consistent with optimal patient care should be given throughout the study according to institutional standards. Refer to local product label or SmPC and institution guidelines for paclitaxel for further guidance on paclitaxel dosing.

All subjects should be premedicated prior to paclitaxel administration in order to prevent severe hypersensitivity reactions. Such premedication may consist of dexamethasone 20 mg orally administered approximately 12 and 6 hours before paclitaxel, diphenhydramine (or its equivalent) 50 mg intravenous 30 to 60 minutes prior to paclitaxel, and cimetidine (300 mg) or ranitidine (50 mg) intravenous 30 to 60 minutes before paclitaxel. The appropriate premedication regimen may be determined by the investigator.

(ii) Increase or Reduction in Dose of the Study Drug(s)

(a) Enfortumab Vedotin

Dose reduction to 1 mg/kg (dose level—1) and to 0.75 mg/kg (dose level—2) will be allowed depending on the type and severity of toxicity. Subjects requiring a dose reduction may be re-escalated by 1 dose level (i.e., subjects reduced to 0.75 mg/kg may only be re-escalated to 1 mg/kg) provided the toxicity does not require study drug discontinuation and has returned to baseline or ≤Grade 1. If the toxicity recurs, re-escalation will not be permitted. Subjects with ≥Grade 2 corneal AEs will not be permitted to dose re-escalate.

EV should not be administered to subjects with CrCl<30 mL/min. Dose modification recommendations for EV associated toxicity are presented in Table 13 and Table 14. Dose interruptions for other EV associated toxicity is permitted at the discretion of the site investigator. Dose interruptions may last up to 8 weeks (2 cycles). Dose interruptions for subjects who are deriving clinical benefit from treatment may be extended beyond 8 weeks, if the subject's toxicity does not otherwise require permanent discontinuation. If there is a dose interruption, the schedule for response assessments will not be adjusted.

TABLE 13
Recommended dose modifications for enfortumab
vedotin associated hematologic toxicity*

| Grade 1 | Grade 2 | Grade 3 | Grade 4 |
|---|---|---|---|
| Continue at | Continue at | Withhold dose until | Withhold dose until |
| same dose | same dose | toxicity is ≤ Grade 1 | toxicity is ≤ Grade 1 or |
| level. | level. | or has returned to | has returned to |
| | For Grade 2 | baseline, then resume | baseline, then reduce |
| | thrombocytopenia, | treatment at the same | dose by 1 dose level |
| | withhold dose | dose level or consider | and resume treatment, |
| | until toxicity is ≤ | dose reduction by 1 | or discontinue at the |
| | Grade 1 or has | dose level. | discretion of the |
| | returned to | Transfusions or growth | investigator. |
| | baseline, then | factors may be used as | Transfusions or growth |
| | resume treatment | indicated per | factors may be used as |
| | at the same dose | institutional guidelines. | indicated per |
| | level. | | institutional guidelines. |
| | | | For anemia, treatment |
| | | | discontinuation should |
| | | | be strongly considered. |

*Note:
hematological toxicity refers to anemia, thrombocytopenia, neutropenia and febrile neutropenia.

TABLE 14
Recommended dose modifications for enfortumab
vedotin associated nonhematologic toxicity

| Grade 1 | Grade 2 | Grade 3 | Grade 4 |
|---|---|---|---|
| Continue at same | Continue at same | Withhold dose until | For Grade 4 AEs, |
| dose level. | dose level, except in | toxicity is ≤ Grade | discontinue |
| If ocular symptoms | the event of Grade 2 | 1 or has returned to | treatment.* |
| and/or changes in | neuropathy or | baseline, then | Grade 4 vomiting |
| vision are | corneal AEs. | resume treatment at | and/or diarrhea that |
| identified, the | For Grade 2 | the same dose level | improves to ≤ Grade |
| subject should be | neuropathy or | or consider dose | 2 or less within 72 |
| evaluated with an | corneal AE's, | reduction by 1 dose | hours with |
| ophthalmologic | withhold dose until | level.* | supportive |
| exam.** | toxicity is ≤ Grade | For Grade 3 | management does |
| | 1 or has returned to | neuropathy or | not require |
| | baseline, and then | corneal AEs, | discontinuation. |
| | resume treatment at | discontinue | |
| | the same dose level. | treatment. | |
| | For the second | For Grade 3 | |
| | occurrence of | hyperglycemia/ | |
| | Grade 2 neuropathy | elevated blood | |
| | or corneal AE's, | glucose, withhold | |
| | withhold dose until | EV treatment. | |
| | toxicity is ≤ Grade | Resume treatment | |
| | 1, and then reduce | once hyperglycemia/ | |
| | the dose by 1 dose | elevated blood | |
| | level and resume | glucose has | |
| | treatment. | improved to ≤ Grade | |
| | If ocular symptoms | 2 and subject is | |
| | and/or changes in | clinically and | |
| | vision are | metabolically stable. | |
| | identified, the | If ocular symptoms | |
| | subject should be | and/or changes in | |
| | evaluated with an | vision are | |
| | ophthalmologic | identified, the | |
| | exam.** | subject should be | |
| | | evaluated with an | |
| | | ophthalmologic | |
| | | exam.** | |

AE: adverse event;
EV: enfortumab vedotin
*Grade 3/4 electrolyte imbalances/laboratory abnormalities, except hyperglycemia, that are not associated with clinical sequelae and/or are corrected with supplementation/appropriate management within 72 hours of their onset do not require discontinuation (e.g., Grade 4 hyperuricemia). Grade 3 rash that is not limiting self-care activities of daily living or associated with infection requiring systemic antibiotics does not require treatment interruption, provided symptoms are not severe and can be managed with supportive treatment.
**Ophthalmologic exam should be performed by an ophthalmologist. In countries where optometrists can perform exams and prescribe medications, an optometrist may be used instead.

Enfortumab Vedotin Related Rash

In the phase 1 study, rash and similar dermatologic AE's were common among patients treated with EV, and were seen more frequently at the highest dose. Although the exact etiology of dermatologic toxicities associated with EV is unclear at this time, due to the expression of nectin-4 in the skin, rash maybe an on target toxicity. The most common type of dermatological AE reported in ASG-22CE-13-2 was maculo-papular rash, rash, skin exfoliation, and skin pigmentation disorder. Most occurred early on (during cycle 1), and some were associated with pruritus. Mild rash due to EV should be treated using local supportive care as needed. Topical corticosteroids have been used along with antihistamines for pruritus as needed. Grade 3 rash that is not limiting self-care activities of daily living or associated with infection requiring systemic antibiotics does not require treatment interruption, provided symptoms are not severe and can be managed with supportive treatment.

Management of Hyperglycemia

Investigators should monitor blood glucose levels and are advised to perform additional assessments if any symptoms of hyperglycemia are observed, including a thorough evaluation for infection. In addition, if steroids are used to treat any other condition, blood glucose levels may require additional monitoring. If elevated blood glucose levels are observed, subjects should be treated according to local standard of care and referral to endocrinology may be considered.

Subjects, especially those with a history of or ongoing diabetes mellitus or hyperglycemia, should be advised to immediately notify their physician if their glucose level becomes difficult to control or if they experience symptoms suggestive of hyperglycemia such as frequent urination, increased thirst, blurred vision, fatigue, and headache.

Subjects who enter the study with an elevated HbA1c (≥6.5%) at baseline should be referred to an appropriate provider during Cycle 1 for glucose management. Blood glucose should be checked prior to each dosing and dose should be withheld for blood glucose >250 mg/dL (13.9 mmol/L) (Grade 3 or higher). Dosing may continue once the subject's blood glucose has improved to ≤Grade 2 and subject is clinically and metabolically stable. Blood glucose >500 mg/dL (27.8 mmol/L) (Grade 4) considered related to EV requires treatment discontinuation. If a subject experiences new onset diabetes mellitus, evaluate subjects with a metabolic panel, urine ketones, glycosylated hemoglobin, and C-peptide to assess for new onset type 1 diabetes in the setting of prior CPI.

Management of Enfortumab Vedotin Infusion Related Reactions (IRR)

An IRR may occur during the infusion of study treatment. The infusion should be administered at a site properly equipped and staffed to manage anaphylaxis should it occur. All supportive measures consistent with optimal patient care should be given throughout the study according to institutional standards. Supportive measures may include administering medications for IRRs.

Subjects who have experienced an IRR may be premedicated for subsequent infusions. Premedication may include pain medication (e.g., acetaminophen or equivalent), an antihistamine (e.g., diphenhydramine hydrochloride), and a corticosteroid administered approximately 30-60 minutes prior to each infusion or according to institutional standards. Should a subject experience IRRs in the setting of premedication, continued treatment with enfortumab vedotin must be discussed with the medical monitor prior to the next planned dose.

If anaphylaxis occurs, study treatment administration should be immediately and permanently discontinued.

(b) Dose Modification for treatment with Docetaxel, Paclitaxel, or Vinflunine

In general, treatment with the chemotherapy comparators (docetaxel, paclitaxel, or vinflunine) should be withheld for drug-related Grade 4 hematologic toxicities and for non-hematologic toxicity ≥Grade 3, and subsequent doses modified as per Table 15 below. Dose modifications will be applied for all subsequent doses. Specific dose modification guidance for docetaxel, paclitaxel, and vinflunine is found below in Sections 6.1.6.1(ii)(b), 6.1.6.1(ii)(c), and 6.1.6.1(ii)(d). Dose modifications should also be considered according to local product labels or SmPC and institutional guidelines. For docetaxel, paclitaxel, or vinflunine associated hematologic toxicities ≥3, transfusions or growth factors may be used as indicated per institutional guidelines.

TABLE 15
Dose Modification Guidelines for Drug-Related Adverse Events on the Active Comparator Arm

| | | | | | Treatment |
|---|---|---|---|---|---|
| Toxicity* | Grade | Occurrence | Hold Treatment | Dose Modification | Discontinuation |
| Neutropenia | Grade 1, 2, 3 or | All | Hold treatment until | NA | NA |
| | Grade 4 | | neutrophils recover | | |
| | lasting ≤7 days | | to >1500 cells/mm³ | | |
| | Grade 4 | 1^{st} | Hold treatment until | Restart treatment at: | Treatment Discontinuation |
| | lasting >7 days | | neutrophils recover | Paclitaxel: 135 mg/m² | should be considered |
| | | | to >1500 cells/mm³ | Docetaxel: 60 mg/m² | |
| | | | | Vinflunine: 280 mg/m² | |
| | | 2^{nd} | Hold treatment until | Restart treatment at: | Treatment Discontinuation |
| | | | neutrophils recover | Paclitaxel: 100 mg/m² | should be considered |
| | | | to >1500 cells/mm³ | Docetaxel: 50 mg/m² | |
| | | | | Vinflunine: 250 mg/m² | |
| | | 3^{rd} | Yes | NA | Yes |
| Thrombocytopenia | Grade 1, 2, 3 | All | Hold treatment until | NA | NA |
| | | | platelets recover | | |
| | | | to >100000 cells/mm³ | | |
| | Grade 4 | 1^{st} | Hold treatment until | Restart treatment at: | Treatment discontinuation |
| | | | platelets recover | Paclitaxel: 135 mg/m² | should be considered |
| | | | to >100000 cells/mm³ | Docetaxel: 60 mg/m² | |
| | | | | Vinflunine: 280 mg/m² | |
| | | 2^{nd} | Hold treatment until | Restart treatment at: | Treatment Discontinuation |
| | | | platelets recover | Paclitaxel: 100 mg/m² | should be considered |
| | | | to >100000 cells/mm³ | Docetaxel: 50 mg/m² | |
| | | | | Vinflunine: 250 mg/m² | |
| | | 3^{rd} | Yes | NA | Yes |
| Anemia | Grade 1, 2, 3 | All | Until anemia resolves to | NA | NA |
| | | | Grade 1 or baseline | | |
| | Grade 4 | 1^{st} | Until anemia resolves to | Restart treatment at: | Treatment discontinuation |
| | | | Grade 1 or baseline | Paclitaxel: 135 mg/m² | should be considered |
| | | | | Docetaxel: 60 mg/m² | |
| | | | | Vinflunine: 280 mg/m² | |
| | | 2^{nd} | Until anemia resolves to | Restart treatment at: | Treatment Discontinuation |
| | | | Grade 1 or baseline | Paclitaxel: 100 mg/m² | should be considered |
| | | | | Docetaxel: 50 mg/m² | |
| | | | | Vinflunine: 250 mg/m² | |
| | | 3^{rd} | Yes | NA | Yes |
| Nonhematological toxicity | Grade 1, 2 | All | No | None | NA |
| and other hematological | Grade 3, 4 | 1^{st} | Yes, until toxicity | Restart treatment at: | Treatment Discontinuation |
| toxicity not described | | | resolves to Grade 0-1 or | Paclitaxel: 135 mg/m² | should be considered |
| above.** | | | baseline | Docetaxel: 60 mg/m² | |
| | | | | Vinflunine: 280 mg/m² | |
| | | 2^{nd} | Yes, until toxicity | Restart treatment at: | Treatment Discontinuation |
| | | | resolves to Grade 0-1 or | Paclitaxel: 100 mg/m² | should be considered |
| | | | baseline | Docetaxel: 50 mg/m² | |
| | | | | Vinflunine: 250 mg/m² | |
| | | 3^{rd} | Yes | NA | Yes |

*See Table 16, Table 17, and Table 18 for additional dose modifications for drug-related adverse events specific to docetaxel, vinflunine and paclitaxel,
respectively.
**Subjects who experience suspected severe hypersensitivity reaction to paclitaxel or vinflunine (e.g., generalized rash/erythema, hypotension and/or bronchospasm, angioedema or anaphylaxis) should be discontinued from trial treatment.
*** For docetaxel, paclitaxel or vinflunine associated hematologic toxicities ≥ Grade 3, transfusions or growth factors may be used as indicated per institutional guidelines.
See Table 16 and Table 18 for guidelines on management of peripheral neuropathy specific to docetaxel and paclitaxel, respectively.
NA: not applicable

(c) Docetaxel Dose Modifications

Docetaxel should not be given to subjects with total bilirubin >ULN, or to subjects with AST and/or ALT >1.5×ULN with concomitant alkaline phosphatase >2.5×ULN. Subjects with elevations of bilirubin or abnormalities of transaminase concurrent with alkaline phosphatase are at increased risk for the development of Grade 4 neutropenia, febrile neutropenia, infections, severe thrombocytopenia, severe stomatitis, severe skin toxicity, and toxic death. Docetaxel should also not be given to subjects with a neutrophil count of <1500 cells/mm³.

Severe fluid retention has been reported following docetaxel therapy. Subjects should be premedicated with corticosteroids prior to each docetaxel injection administration to reduce the incidence and severity of fluid retention. Subjects with pre-existing effusions should be closely monitored from the first dose for the possible exacerbation of the effusions. Subjects developing peripheral edema may be treated with standard measures, e.g., salt restriction, oral diuretic(s). Dose interruptions may last up to 6 weeks (2 cycles). Dose interruptions for subjects who are responding to treatment may be extended beyond 6 weeks, if the subject's toxicity does not otherwise require permanent discontinuation. Recommended dose modification guidelines for subjects receiving docetaxel are detailed below in Table 16. Dose modifications not specified below (e.g., severe or cumulative cutaneous reactions) and starting doses should also consider local product label or SmPC and institutional guidelines.

TABLE 16
Recommended Docetaxel Dose Modification Guidelines

| | | | Hold | Dose | Discontinue |
|---|---|---|---|---|---|
| Toxicity | Grade | Occurrence | Treatment | Modification | Treatment |
| Peripheral | Grade | | No | 60 mg/m² | N/A |
| Neuropathy | 1, 2 | | | | |
| | Grade | | Yes | N/A | Discontinue |
| | 3, 4 | | | | upon onset |
| Neutropenic | | 1 | Hold treatment | 60 mg/m² | |
| fever (defined | | | until ANC ≥ | | |
| as T ≥ 100.5° F. | | | 1500/L | | |
| (38.1° C.) and | | 2 | Hold treatment | 50 mg/m² | |
| ANC ≤ 1000/L) | | | until ANC ≥ | | |
| | | | 1500/L | | |
| | | 3 | Yes | N/A | Yes |

ANC: absolute neutrophil count;
N/A: not applicable;
T: temperature

(d) Vinflunine Dose Modifications

In case of WHO/ECOG PS of 1 or ECOG PS of 0 and prior pelvic irradiation, vinflunine treatment should be started at the dose of 280 mg/m². In the absence of any hematological toxicity during the first cycle causing treatment delay or dose reduction, the dose may be increased to 320 mg/m² every 21 days for the subsequent cycles. In subjects with moderate renal impairment (40 mL/min≤CrCl≤60 mL/min), the recommended dose is 280 mg/m² given once every 21 day cycle. In subjects with renal impairment (30 mL/min≤CrCl<40 mL/min), the recommended dose is 250 mg/m² given once every 21 day cycle.

The recommended dose of vinflunine is 250 mg/m² given once every 21-day cycle in subjects with mild liver impairment (Child-Pugh grade A). Refer to local product label or SmPC and institutional guidelines for further dose modifications.

The doses recommended in subjects ≥75 years old are as follows:
in subjects at least 75 years old but less than 80 years, the dose of vinflunine to be given is 280 mg/m² every 21 day cycle.
in subjects 80 years old and beyond, the dose of vinflunine to be given is 250 mg/m² every 21 day cycle.

In subjects who initiate vinflunine at 280 mg/m² and who experience an AE requiring dose modification, the dose should be reduced to 250 mg/m² following the 1st occurrence and resolution, and discontinued following a 2nd occurrence. In subjects who initiate vinflunine at 250 mg/m² and who experience an AE requiring dose modification, vinflunine should be discontinued.

Cases of Posterior Reversible Encephalopathy Syndrome (PRES) have been observed after administration of vinflunine. The typical clinical symptoms are, with various degrees: neurological (headache, confusion, seizure, visual disorders), systemic (hypertension), and gastrointestinal (nausea, vomiting). Radiological signs are white matter abnormalities in the posterior regions of the brain. Vinflunine must be discontinued in subjects who develop neurological signs of PRES. Dose modifications for subjects receiving vinflunine are detailed below in Table 17. Dose interruptions may last up to 6 weeks (2 cycles). Dose interruptions for subjects who are deriving clinical benefit from treatment may be extended beyond 6 weeks, if the subject's toxicity does not otherwise require permanent discontinuation. Refer to the vineflunine (Javlor®) SmPC for additional information.

TABLE 17
Vinflunine Dose Modifications

| | Dose Adjustments | | | | |
|---|---|---|---|---|---|
| | Initial Dose: Vinflunine 320 mg/m² | | | Initial Dose: Vinflunine 280 mg/m² | |
| | | 2nd | 3rd | | 2nd |
| | | Consecutive | Consecutive | | Consecutive |
| Toxicity | 1st Event | Event | Event | 1st Event | Event |
| Neutropenic | Vinflunine | Vinflunine | Discontinue | Vinflunine | Discontinue |
| fever (defined as | 280 | 250 | treatment | 250 mg/m² | treatment |
| T ≥ 100.5° F. | mg/m² | mg/m² | | | |
| (38.1° C.) and | | | | | |
| ANC ≤ 1000/L) | | | | | |
| Mucositis or | | | | | |
| Constipation | | | | | |
| Grade 2 ≥ 5 days | | | | | |
| or Grade 3 ≥ any | | | | | |
| duration¹ | | | | | |
| Cardiac | Discontinue | N/A | N/A | Discontinue | N/A |
| ischemia in | treatment | | | treatment | |
| patients with | | | | | |
| prior history of | | | | | |
| myocardial | | | | | |
| infarction or | | | | | |
| angina pectoris | | | | | |

ANC: absolute neutrophil count;
N/A: not applicable;
T: temperature
¹National Cancer Institute Common Terminology Criteria for Adverse Events Grade 2 constipation is defined as requiring laxatives, Grade 3 as an obstipation requiring manual evacuation or enema, Grade 4 as an obstruction or toxic megacolon. Mucositis Grade 2 is defined as “moderate,” Grade 3 as “severe” and Grade 4 as “life-threatening.”

(e) Paclitaxel Dose Modifications

Paclitaxel should not be administered to subjects with baseline neutrophil counts of less than 1500 cells/mm³. Subjects should not be re-treated with subsequent cycles of paclitaxel until neutrophils recover to a level >1500 cells/mm³ and platelets recover to a level >100000/mm³. Severe conduction abnormalities have been documented in <1% of subjects during paclitaxel therapy and in some cases requiring pacemaker placement. If subjects develop significant conduction abnormalities during paclitaxel infusion, appropriate therapy should be administered and continuous cardiac monitoring should be performed during subsequent therapy with paclitaxel provided the subject does not require discontinuation.

In case of mild hepatic impairment (total bilirubin ≥1.25 ULN), paclitaxel should be started at a dose of 135 mg/m². Dose modifications for subjects receiving paclitaxel are detailed below. Dose interruptions may last up to 6 weeks (2 cycles). Dose interruptions for subjects who are responding to treatment may be extended beyond 6 weeks if the subject's toxicity does not otherwise require permanent discontinuation. Recommended dose modification guidelines for subjects receiving paclitaxel are detailed below in Table 18. Refer to local product label or SmPC and institutional guidelines for further dose modifications.

TABLE 18
Recommended Dose Modification Guidelines Paclitaxel

| | | | Hold | Dose | Treatment |
|---|---|---|---|---|---|
| Toxicity | Grade | Occurrence | Treatment | Modification | Discontinuation |
| Peripheral | Grade 1, | | No | 135 mg/m² | N/A |
| Neuropathy | 2 | | | | |
| | Grade 3, | | Yes | N/A | Discontinue |
| | 4 | | | | upon onset |
| Neutropenic fever | | 1 | Hold until | 135 mg/m² | |
| (defined as T ≥ | | | ANC ≥ | | |
| 100.5° F. (38.1° C.) | | | 1,500/L | | |
| and ANC ≤ | | 2 | Hold until | 100 mg/m² | |
| 1,000/L) | | | ANC ≥ | | |
| | | | 1 | | |
| | | | 1,500/L | | |
| | | 3 | yes | N/A | Yes |

ANC: absolute neutrophil count;
N/A: not applicable;
T: temperature

(iii) Previous and Concomitant Treatment (Medication and Nonmedication Therapy)

If the investigator determines that any of the following medications are deemed necessary to provide adequate medical support to the subject, the subject must be withdrawn from further administration of the study treatment:
Other investigational drugs
Chemotherapy or other medications intended for antitumor activity. This does not apply to subjects with a history of breast cancer on adjuvant endocrine therapy, or for subjects on agents intended for the treatment of bone metastasis (e.g., bisphosphonates, or RANK ligand inhibitors).
Radiation therapy
   Note: Radiation therapy to a symptomatic solitary lesion or to the bone may be considered on an exceptional case-by-case basis after consultation with Sponsor. The radiated lesion must be a non-target lesion per RECIST V1.1 and the subject must have clear measurable disease outside the radiated field.

(a) Arm A (enfortumab vedotin)

Subjects who are receiving strong cytochrome P450 (CYP)3A4 inhibitors or P-pg inhibitors concomitantly with EV should be closely monitored for adverse reactions.

(b) Arm B (Docetaxel)

Concomitant use of drugs that strongly inhibit or induce CYP3A4 may affect exposure to docetaxel and should be avoided.

(c) Arm B (Vinflunine)

Strong inhibitors of the CYP3A4 enzymes for subjects receiving vinflunine should be avoided.

QT/QTc interval prolonging medicinal products should be avoided.

(d) Arm B (Paclitaxel)

Caution should be exercised when paclitaxel is administered with strong inhibitors or inducers of CYP3A4 and CYP2C8.

Refer to the local package inserts for concomitant medication restrictions or requirements for docetaxel, paclitaxel and vinflunine. All concomitant treatments will be recorded in the eCRF or electronic data source unless otherwise specified.

(iv) Treatment Compliance

The dose and schedule of EV, paclitaxel, docetaxel and vinflunine administered to each subject will be recorded on the appropriate electronic case report form (eCRF) at every cycle. Reasons for dose delay, reduction or omission will also be recorded.

If toxicities or adverse events occur on Day 1 of any cycle and EV cannot be administered, then the start of the cycle may be delayed. If toxicities occur on Days 8 or 15 of any cycle and require the dose to be held >3 days, then the dose(s) must be eliminated, rather than delayed. If a subject only receives day 1 and needs to skip days 8 and 15, the subject could resume the next cycle as early as day 22 (new day 1), if the toxicity has resolved by then.

### 6.1.6.2 Demographics and Baseline Characteristics

(i) Demographics

Demographic information will be collected for all subjects as allowed per local regulation and will include date of birth, sex, race, ethnicity and tobacco use history (pack years).

(ii) Medical History

Medical history will include all significant medical conditions other than urothelial cancer that have resolved prior to informed consent or are ongoing at the time of consent. Details that will be collected include the onset date and recovery date and Common Terminology Criteria for Adverse Events (CTCAE) grade, if applicable for ongoing conditions.

(iii) Diagnosis of the Target Disease, Severity, and Duration of Disease

For urothelial carcinoma, the following information including but not limited to will be collected during the screening period, and be entered in the eCRF:
Date of initial diagnosis of the primary carcinoma, histological type, date of histopathological or cytopathological diagnosis
Date of diagnosis for the locally advanced or metastatic or recurrent disease
TNM classification and disease stage at screening
Sampling method and the type of the tumor tissues for Nectin-4 mutation analysis
Previous treatment (including medication, radiotherapy and surgery) for underlying disease

(iv) Performance Status

The ECOG PS Scale will be used to assess performance status. Refer to Section 6.1.9.9. Oken et al., Am J Clin Oncol. 1982; 5:649-55.

### 6.1.6.3 Efficacy Assessments

Response and progression will be evaluated using RECIST V1.1 6.1.9.8.

Imaging for both Arms will be performed at screening/baseline and every 56 days (±7 days) from the first dose of study treatment throughout the study. Baseline imaging performed prior to informed consent as standard of care may be used so long as it is performed within 28 days prior to randomization. All subjects will have a bone scan (scintigraphy) performed at screening/baseline. Subjects with positive bone scans at baseline will have a bone scan performed every 56 days (±7 days) throughout the study or more frequently if clinically indicated. Subjects with negative bone scans at baseline should have a bone scan performed if clinically indicated throughout the study even if not positive at baseline. Brain scans

(CT with contrast/MRI) will only be performed if clinically indicated at screening/baseline and repeated as clinically indicated or per standard of care throughout the study. If a subject discontinues study drug prior to radiological disease progression, the subject should continue to undergo imaging assessments every 56 days (±7 days) until disease progression is documented, or the subject starts another anticancer treatment whichever occurs earlier.

A CT scan with contrast (chest and abdomen) is the preferred modality for tumor assessment. Magnetic resonance imaging is acceptable if local standard practice or if CT scans are contraindicated in a subject (e.g., subject is allergic to contrast media). All other RECIST- approved scanning methods such as x-ray are optional. Additional instructions for imaging assessments can be found in the study procedures manual.

The assessment will include tumor measurements for target lesions, nontarget lesions and any new lesions. An overall assessment will be characterized for a given time point evaluation.

At the end of study for that subject, the best overall response to the study regimen will be characterized. To ensure comparability, the screening and subsequent assessment of response should be performed using identical techniques. The same individual should assess images for any 1 subject for the duration of the study if possible. For subjects with known brain metastases at study entry, it is recommended that repeat imaging also include the brain and the same methods used to detect brain lesions at Baseline are to be used to follow the lesions throughout the study.

The site of disease progression including target, non-target and/or new lesions should be documented in the eCRF. Additional imaging may be performed at any time to confirm suspected progression of disease.

This study will be analyzed based on the results of local (investigator site) radiologic assessments, including dates of progression and death. Since imaging scans may be needed for future regulatory purposes or an independent review of all or a representative sample of scans may be considered following the completion of PFS1 analysis, copies of all scans will be collected throughout the study and stored centrally by a coordinating vendor. Images from all randomized subjects will be sent to the imaging vendor according to the frequency of Table 10.

(i) Evaluation of Target Lesions

(a) Complete Response

CR is defined as disappearance of all target and nontarget lesions. Any pathological lymph nodes (whether target or nontarget) must have reduction in short axis to <10 mm from baseline measurement.

(b) Partial Response

PR is defined as at least a 30% decrease in the sum of diameters (longest for nonnodal lesions, short axis for nodal lesions) of target lesions taking as reference to the baseline sum of diameters.

(c) Stable Disease

SD is defined as neither sufficient decrease to qualify for PR nor sufficient increase to qualify for progressive disease taking as reference the smallest sum of diameters while on study drug.

(d) Progressive Disease

PD is defined as at least a 20% increase in the sum of diameters (longest for nonnodal lesions, short axis for nodal lesions) of the target lesions taking as reference the smallest sum on study (this includes the baseline sum if that is the smallest on study). In addition to the relative increase of 20%, the sum must also demonstrate an absolute increase of at least 5 mm. The appearance of 1 or more new lesions is also considered progression.

(ii) Evaluation of Nontarget Lesions

To achieve unequivocal progression on the basis of nontarget lesions, there must be an overall level of substantial worsening in nontarget disease such that, even in presence of SD or PR of target lesions, the overall tumor burden has increased sufficiently to merit discontinuation of therapy. A modest increase in the size of 1 or more nontarget lesions is usually not sufficient to qualify for unequivocal progression.

(a) Complete Response

For CR of nontarget lesions, subjects must have disappearance of all nontarget lesions and all lymph nodes must be nonpathological in size (<10 mm short axis).

(b) NonCR/NonPD

NonCR/NonPD of nontarget lesions is defined as persistence of 1 or more nontarget lesions.

(c) Progressive Disease

PD of nontarget lesions is defined as unequivocal progression of existing nontarget lesions or the appearance of 1 or more new lesions.

(iii) Evaluation of Time Point Response

The overall response status at each time point for subjects with measurable disease at baseline will be reported according to the table in Sections 6.1.9.6 and 6.1.9.7.

### 6.1.6.4 Pharmacokinetic Assessment

PK samples will be collected during the treatment period and at the post-treatment follow-up visit for subjects who receive EV for determination of ADC and MMAE concentrations. If a subject presents to clinic but does not get dosed, a predose PK sample collection should be performed.

Blood samples (7 mL/sample) for pharmacokinetic analyses should be collected at the time points indicated in the Schedule of Assessments (Table 10). Blood samples should be collected via a peripherally placed intravenous cannula or by direct venipuncture.

Blood should not be drawn from the arm or port used for study drug infusion.

Bioanalysis of TAb, ADC and MMAE in serum or plasma will be performed using validated methods at bioanalytical laboratories specified by the sponsor.

### 6.1.6.5 Safety Assessment

(i) Vital Signs

Vital signs, including systolic and diastolic blood pressures (mmHg), radial pulse rate (beats/minute) and temperature will be obtained according to the Schedule of Assessments (Table 10) and recorded. All vital sign measures will be obtained with the subject in the sitting or supine position.

If clinically significant vital sign changes from Baseline (pretreatment) are noted, the changes will be documented as AEs on the AE page of the eCRF. Clinical significance will be defined as a variation in vital signs that has medical relevance as deemed by the investigator that could result in an alteration in medical care. The investigator will continue to monitor the subject until the parameter returns to Grade ≤1, or to the Baseline (pretreatment) value, or until the investigator determines that follow up is no longer medically necessary.

(ii) Adverse Events

See Section 6.1.6.6 (Adverse Events and Other Safety Aspects) for information regarding AE collection and data handling.

(a) Adverse Events of Possible Hepatic Origin

See Section 6.1.9.2 Liver Safety Monitoring and Assessment for detailed information on liver abnormalities, monitoring and assessment, if the AE for a subject enrolled in a study and receiving study drug is accompanied by increases in Liver Function Tests value ((LFT), e.g., AST, ALT, bilirubin, etc.) or is suspected to be due to hepatic dysfunction.

Subjects with AEs of hepatic origin accompanied by LFT abnormalities should be carefully monitored.

(iii) Laboratory Assessments

Table 19 below is a table of the laboratory tests that will be performed during the conduct of the study. See Schedule of Assessments for study visit collection dates.

TABLE 19

| | |
|---|---|
| Biochemistry | Sodium (Na), Magnesium (Mg), Potassium (K), Calcium (Ca), Chloride |
| | (Cl), Phosphate (P), Serum Creatinine, Glucose (Gl), Blood Urea |
| | Nitrogen (BUN), ALP, AST, ALT, Lactate Dehydrogenase (LDH), |
| | Bilirubin (total and direct), Total Protein (TP), Albumin (Alb), |
| | Bicarbonate (HCO3), Amylase, Lipase, Uric Acid, Hemoglobin A1c |
| | (screening only), Serum HCG for female subjects of childbearing |
| | potential |
| Hematology | Red Blood Cell Count (RBC), Hematocrit (Hct), Hemoglobin (Hgb), |
| | Platelets, white blood cell count (WBC)/differential (Neutrophils, |
| | Eosinophils, Basophils, Lymphocytes, Monocytes), |
| Urinalysis | Pregnancy |
| HbA1c | |

Laboratory tests will be performed predose according to the Schedule of Assessments and sent to a central laboratory for analysis. All screening labs must be sent to the central laboratory, but local laboratory results may be used to determine eligibility if the screening results from the central laboratory are not available in time for planned randomization. In the event that the central laboratory results received after randomization are not within eligibility parameters, the subject will still be considered eligible if local labs met the eligibility criteria and will not be considered a protocol deviation. Local laboratory results that support eligibility and dosing decisions must be entered into the clinical database. If local laboratory is to be used to support dosing decisions, local laboratory tests will include complete blood count with differential, glucose, serum creatinine, ALT and AST. In case of multiple laboratory data within this period, the most recent data should be used. Laboratory assessments collected outside of −7 days of randomization should be repeated. Additional assessments may be done centrally or locally to monitor AEs or as required by dose modification requirements.

Assessment of creatinine clearance or estimation of GFR per institutional guidelines should be performed prior to administration of vinflunine and EV as recommended in Section 6.1.6.1(ii) (Increase or Reduction in Dose of the Study Drug(s)).

Additional laboratory tests should be performed according to institutional standard of care. Clinical significance of out-of-range laboratory findings is to be determined and documented by the investigator/sub-investigator who is a qualified physician.

(iv) Physical Examination

Standard, full physical examinations will be performed at screening to assess general appearance, skin, eyes, ears, nose, throat, neck, cardiovascular, chest and lungs, abdomen, musculoskeletal, neurologic status, mental status and lymphatic systems. For subsequent and end of treatment (EOT) visits, physical examinations maybe more directed but should include examination of lungs, abdomen, skin, and cardiovascular system. Physical examinations will be conducted at visits as outlined in the Schedule of Assessments (Table 10). Each physical examination will include weight; height is only required at Screening. If clinically significant worsening of findings from Baseline is noted at any study visit, the changes will be documented as AEs on the AE eCRF. Clinical significance is defined as any variation in physical findings that has medical relevance that could result in an alteration in medical care. The investigator will continue to monitor the subject until the parameter returns to Grade ≤1, or to the Baseline condition, or until the investigator determines that follow up is no longer medically necessary.

(v) Ophthalmology Examination

Ophthalmologic assessment for subjects with recent ocular complaints (within 3 months of screening) are required. Assessments should include the following: visual acuity, slit lamp, tonometry examination, and dilated fundus examination. Prior ophthalmologic exam done within 3 months of screening is acceptable provided symptoms are not new since the exam. Ophthalmology assessments during treatment should be performed per standard of care or if clinically indicated (e.g., subject develops new or worsening ocular symptoms). EOT slit lamp examinations are required for subjects who experience corneal adverse events during the study. EOT slit lamp examinations must be performed ≥4 weeks from last dose.

Additional eye examinations are to be conducted as clinically indicated.

(vi) Electrocardiogram

A standard 12-lead ECG will be performed and assessed using local standard procedures according to the schedule of assessments in Table 10. Clinically significant abnormal findings at screening should be recorded as medical history.

### 6.1.6.6 Adverse Events and Other Safety Aspects

(i) Definition of Adverse Events

An AE is any untoward medical occurrence in a subject, temporally associated with the use of a medicinal product, whether or not considered related to the medicinal product. An AE can therefore be any unfavorable and unintended sign (including an abnormal laboratory finding), symptom, or disease (new or exacerbated) temporally associated with the use of a medicinal product.

In order to identify any events that may be associated with study procedures and could lead to a change in the conduct of the study, sponsor collects AEs even if the subject has not received study drug treatment. AE collection begins after the signing of the informed consent and will be collected until 30 days after the last dose of study drug.

(a) Abnormal Laboratory Findings

Any abnormal laboratory test result (e.g., hematology, clinical chemistry, or urinalysis) or other safety assessment (e.g., ECGs, radiographic scans, vital signs measurements, physical examination), including those that worsen from baseline, that is considered to be clinically significant in the medical and scientific judgment of the investigator and not related to underlying disease, is to be reported as an (S)AE.

Any clinically significant abnormal laboratory finding or other abnormal safety assessment which is associated with the underlying disease does not require reporting as an (S)AE, unless judged by the investigator to be more severe than expected for the subject's condition.

Repeating an abnormal laboratory test or other safety assessment, in the absence of any of the above criteria, does not constitute an AE. Any abnormal test result that is determined to be an error does not require reporting as an AE.

(b) Potential Cases of Drug-Induced Liver Injury

Refer to Section 6.1.9.2 Liver Safety Monitoring and Assessment for detailed instructions on Drug Induced Liver Injury (DILI). Abnormal values in aspartate transaminase (AST) and/or alanine transaminase (ALT) concurrent or with abnormal elevations in total bilirubin that meet the criteria outlined in Section 6.1.9.2 Liver Safety Monitoring and Assessment in the absence of other causes of liver injury, are considered potential cases of drug-induced liver injury (potential Hy's Law cases) and are always to be considered important medical events and reported per Section 6.1.6.6(v) (Reporting of Serious Adverse Events).

(c) Disease Progression and Study Endpoints

Under this protocol, the following event(s) will not be considered as an(S)AE:
Disease Progression: events including defined study endpoints that are clearly consistent with the expected pattern of progression of the underlying disease are not to be recorded as AEs unless resulting in death. These data will be captured as efficacy assessment data as outlined in Section 6.1.6.3 Efficacy Assessments. If there is any uncertainty as to whether an event is due to anticipated disease progression and/or if there is evidence suggesting a causal relationship between the study drug and the event, it should be reported as an (S)AE. All deaths within 30 days of the last dose of study drug must be reported as SAEs.
Pre-planned and elective hospitalizations or procedures for diagnostic, therapeutic, or surgical procedures for a pre-existing condition that did not worsen during the course of the clinical trial. These procedures are collected per the eCRFs Completion Guidelines.

(ii) Definition of Serious Adverse Events (SAEs)

An AE is considered “serious” if, in the view of either the investigator or sponsor, it results in any of the following outcomes:
Results in death
Is life-threatening (an AE is considered “life-threatening” if, in the view of either the investigator or sponsor, its occurrence places the subject at immediate risk of death. It does not include an AE that, had it occurred in a more severe form, might have caused death)
Results in persistent or significant disability/incapacity or substantial disruption of the ability to conduct normal life functions
Results in congenital anomaly, or birth defect
Requires inpatient hospitalization (except for planned procedures as allowed per study) or leads to prolongation of hospitalization (except if prolongation of planned hospitalization is not caused by an AE). Hospitalization for treatment/observation/examination caused by AE is to be considered as serious.)
Other medically important events (defined in paragraph below)

Medical and scientific judgment should be exercised in deciding whether expedited reporting is appropriate in other situations, such as important medical events that may not be immediately life-threatening or result in death or hospitalization but may jeopardize the subject or may require intervention to prevent one of the other outcomes listed in the definition above. These events, including those that may result in disability/incapacity, usually are considered serious. Examples of such events are intensive treatment in an emergency room or at home for allergic bronchospasm; blood dyscrasias or convulsions that do not result in hospitalization; or development of drug dependency or drug abuse.

The sponsor has a list of events that they classify as “always serious” events. If an AE is reported that is considered to be an event per this classification as “always serious”, additional information on the event may be requested.

(iii) Criteria for Causal Relationship to Study Drug

The investigator is obligated to assess the relationship between the study drug and each occurrence of each (S)AE. The investigator will use clinical judgment to determine the relationship. The investigator should also use the information provided herein and/or Product Information, for marketed products. The investigator is requested to provide an explanation for the causality assessment for each SAE and must document this on the SAE worksheet.

The causality assessment is one of the criteria used when determining regulatory reporting requirements. The investigator may revise his/her assessment of causality in light of new information regarding the SAE and shall send an SAE follow-up report and update the eCRF with the new information and updated causality assessment.

Following a review of the relevant data, the causal relationship between the study drug and each (S)AE will be assessed by answering ‘yes’ or ‘no’ to the question “Do you consider that there is a reasonable possibility that the event may have been caused by the study drug”.

When making an assessment of causality, the following factors are to be considered when deciding if there is evidence and/or arguments to suggest there is a ‘reasonable possibility’ that an (S)AE may have been caused by the study drug (rather than a relationship cannot be ruled out) or if there is evidence to reasonably deny a causal relationship:
Plausible temporal relationship between exposure to the study drug and (S)AE onset and/or resolution. Has the subject actually received the study drug? Did the (S)AE occur in a reasonable temporal relationship to the administration of the study drug?
Plausibility; i.e., could the event been caused by the study drug? Consider biologic and/or pharmacologic mechanism, half-life, literature evidence, drug class, preclinical and clinical study data, etc.
Dechallenge/Dose reduction/Rechallenge:
   Did the (S)AE resolve or improve after stopping or reducing the dose of the suspect drug? Also consider the impact of treatment for the event when evaluating a dechallenge experience.
   Did the (S)AE reoccur if the suspected drug was reintroduced after having been stopped?
Laboratory or other test results; a specific lab investigation supports the assessment of the relationship between the (S)AE and the study drug (e.g., based on values pre-, during and post-treatment)
Available alternative explanations independent of study drug exposure; such as other concomitant drugs, past medical history, concurrent or underlying disease, risk factors including medical and family history, season, location, etc. and strength of the alternative explanation

There may be situations in which an SAE has occurred and the investigator has minimal information to include in the initial report to the sponsor. However, it is very important that the investigator always make an assessment of causality for every event before the initial transmission of the SAE data to the sponsor. With limited or insufficient information about the event to make an informed judgment and in absence of any indication or evidence to establish a causal relationship, a causality assessment of ‘no’ is to be considered. In such instance, the investigator is expected to obtain additional information regarding the event as soon as possible and to re-evaluate the causality upon receipt of additional information.

(iv) Criteria for Defining the Severity of an Adverse Event

AEs, including abnormal clinical laboratory values, will be graded using the NCI-CTCAE guidelines (Version 4.03). The items that are not stipulated in the NCI-CTCAE Version 4.03 will be assessed according to the criteria in Table 20 below and entered into the eCRF.

TABLE 20

| Grade | Assessment Standard |
|---|---|
| 1-Mild | Asymptomatic or mild symptoms, clinical or diagnostic |
| | observations noted; intervention not indicated. |
| 2-Moderate | Local or noninvasive intervention indicated. |
| 3-Severe | Medically significant but not immediately life threatening, |
| | hospitalization or prolonged hospitalization. |
| 4-Life | Life threatening consequences, urgent intervention indicated |
| Threatening | |
| 5-Death | Death related to AE |

The investigator will use the following definitions to rate the severity of each adverse event
Mild: No disruption of normal daily activities
Moderate: Affect normal daily activities
Severe: Inability to perform daily activities

(v) Reporting of Serious Adverse Events

The collection of AEs and the expedited reporting of SAEs will start following receipt of the informed consent and will continue to 30 days after last administration of study drug.

In the case of a SAE, the investigator must contact the sponsor by fax or email immediately (within 24 hours of awareness).

The investigator must complete and submit a SAE worksheet containing all information that is required by local and/or regional regulations to the sponsor by email or fax immediately (within 24 hours of awareness). If the faxing of an SAE Worksheet is not possible or is not possible within 24 hours, the local drug safety contact should be informed by phone.

The following minimum information is required:
International Study Number (ISN)/Study number,
Subject number, sex and age,
The date of report,
A description of the SAE (event, seriousness criteria),
Causal relationship to the study drug (including reason), and
The drug provided (if any)

(vi) Follow-up of Adverse Events

All AEs occurring during or after the subject has discontinued the study are to be followed up until resolved or judged to be no longer clinically significant, or until they become chronic to the extent that they can be fully characterized by the investigator.

If after the protocol defined AE collection period (see Section 6.1.6.6(i) Definition of Adverse Event), an AE progresses to a SAE, or the investigator learns of any (S)AE including death, where he/she considers there is reasonable possibility it is related to the study drug treatment or study participation, the investigator must promptly notify the sponsor.

(vii) Monitoring of Common Serious Adverse Events

Common SAEs are SAEs commonly anticipated to occur in the study population independent of drug exposure. SAEs classified as “common” are provided in Section 6.1.9.3 Common Serious Adverse Events for reference. The list does NOT change the investigator's reporting obligations, nor his obligations to perform a causality assessment, or prevent the need to report an AE meeting the definition of an SAE as detailed above. The purpose of this list is to alert the investigator that some events reported as SAEs may not require expedited reporting to the regulatory authorities based on the classification of “common SAEs” as specified in Section 6.1.9.3 Common Serious Adverse Events. The sponsor will monitor these events throughout the course of the study for any change in frequency. Any changes to this list will be communicated to the participating clinical trial sites. Investigators must report individual occurrences of these events as stated in Section 6.1.6.6(v) Reporting of Serious Adverse Events.

(viii) Special Situations

Certain Special Situations observed in association with the study drug(s), such as incorrect administration (e.g., wrong dose of study drug, comparator, or background therapy) are collected in the eCRF, as Protocol Deviation per Section 6.1.8.8 Major Protocol Deviations or may require special reporting, as described below. These Special Situations are not considered AEs, but do require to be communicated to sponsor as per the timelines defined below.

If a Special Situation is associated with, or results in, an AE, the AE is to be assessed separately from the Special Situation and captured as an AE in the eCRF. If the AE meets the definition of a SAE, the SAE is to be reported as described in Section 6.1.6.6(v) Reporting of Serious Adverse Events and the details of the associated Special Situation are to be included in the clinical description on the SAE worksheet.

The Special Situations are:
Pregnancy
Medication Error and Overdose
Misuse/abuse
Occupational exposure
Suspected Drug-Drug interaction

(a) Pregnancy

If a female subject becomes pregnant during the study dosing period or within 6 months from the discontinuation of dosing, the investigator is to report the information to the sponsor according to the timelines in Section 6.1.6.6(v) Reporting of Serious Adverse Events using the Pregnancy Reporting Form and in the eCRF.

The investigator will attempt to collect pregnancy information on any female partner of a male subject who becomes pregnant during the study dosing period or within 6 months from the discontinuation of dosing and report the information to the sponsor according to the timelines in Section 6.1.6.6(v) Reporting of Serious Adverse Events using the Pregnancy Reporting Form.

The expected date of delivery or expected date of the end of the pregnancy, last menstruation, estimated conception date, pregnancy result and neonatal data etc., should be included in this information.

While pregnancy itself is not considered to be an AE or SAE, any pregnancy complication or termination (including elective termination) of a pregnancy is to be reported for a female study subject as an AE in the eCRF or SAE per Section 6.1.6.6(v) Reporting of Serious Adverse Events. For (S)AEs experienced by a female partner of a male subject, (S)AEs are to be reported via the Pregnancy Reporting Form.

Additional information regarding the outcome of a pregnancy when also categorized as an SAE is mentioned below:
“Spontaneous abortion” includes miscarriage, abortion and missed abortion.
Death of a newborn or infant within 1 month after birth is to be reported as an SAE regardless of its relationship with the study drug.
If an infant dies more than 1 month after the birth, is to be reported if a relationship between the death and intrauterine exposure to the study drug is judged as “possible” by the investigator.
Congenital anomaly (including anomaly in miscarried fetus)

Unless a congenital anomaly is identified prior to spontaneous abortion or miscarriage, the embryo or fetus should be assessed for congenital defects by visual examination. (S)AEs experienced by the newborn/infant should be reported via the Pregnancy Reporting Form. Generally, follow-up will be no longer than 6 to 8 weeks following the estimated delivery date.

(b) Medication Error, Overdose and “Off-Label Use”

If a Medication Error, Overdose or “Off label Use” (i.e., use outside of what is stated in the protocol) is suspected, refer to Section 6.1.8.8 Major Protocol Deviations. Any associated (S)AEs are to be reported in the eCRF. If the AE meets the definition of a SAE, the SAE is also to be reported as described in Section 6.1.6.6(v) Reporting of Serious Adverse Events together with the details of the medication error, overdose or “Off-Label Use”.

In the event of suspected EV overdose or accidental infusion as a bolus, the subject should receive supportive care and monitoring. The Medical Monitor/Expert should be contacted as applicable.

No specific procedures are available to treat overdose of EV or accidental infusion as a bolus, and only supportive treatment can be given. If EV is accidentally overdosed or accidentally infused as a bolus, the investigator/sub-investigator will provide emergency procedures and/or general maintenance therapy according to the symptoms to assure the subject's safety.

In the event of suspected overdose of paclitaxel, docetaxel or vinflunine, refer to the approved Package Insert, SmPC, or local product information supplied by the manufacturer for each agent.

Events of overdose should be recorded in the eCRF with the dosages actually administered.

(c) Misuse/Abuse

If misuse or abuse of the study drug(s) is suspected, the investigator must forward the Special Situation worksheet to the sponsor by fax or email immediately (within 24 hours of awareness). Any associated (S)AEs are to be reported in the eCRF. If the AE meets the definition of a SAE, the SAE is also to be reported as described in Section 6.1.6.6(v) Reporting of Serious Adverse Events together with details of the misuse or abuse of the study drug(s).

(d) Occupational Exposure

If occupational exposure (e.g., inadvertent exposure to the study drug(s) of site staff whilst preparing it for administration to the patient) to the study drug(s) occurs, the investigator must forward the Special Situation worksheet to the sponsor by fax or email immediately (within 24 hours of awareness). Any associated (S)AEs occurring to the individual associated with or resulting from the Special Situation are to be reported on the Special Situations worksheet.

(e) Suspected Drug-Drug Interaction

If a drug-drug interaction associated with the study drug(s) is suspected, the investigator must forward the Special Situation worksheet to the sponsor by fax or email immediately (within 24 hours of awareness). Any associated (S)AEs are to be reported in the eCRF. If the AE meets the definition of a SAE, the SAE is also to be reported as described in Section 6.1.6.6(v) Reporting of Serious Adverse Events together with details of the suspected drug-drug interaction.

(ix) Supply of New Information Affecting the Conduct of the Study

When new information necessary for conducting the clinical study properly becomes available, the sponsor will inform all investigators involved in the clinical study as well as the regulatory authorities. Investigators should inform the IRB/IEC of such information when needed.

The investigator will also inform the subjects, who will be required to sign an updated informed consent form in order to continue in the clinical study.

(x) Deviations from the Protocol and Other Actions Taken to Avoid Life-Threatening Risks to Subjects

The investigator must not deviate from the protocol, excluding an emergency case for avoiding risks to the subjects. When the investigator does not follow the protocol in order to avoid urgent risks for subjects, the investigator should take the following actions.
1. Describe the contents of the deviation and the reasons for it in a written notice, and immediately send the document stating the deviation or amendment and the reasons to the sponsor and the head of the study site. Keep a copy of the notice.
2. Consult with the sponsor at the earliest possibility for cases in which it is necessary to amend the protocol. Obtain approval for a draft of the amended protocol from the IRB and the head of the study site as well as written approval from the sponsor.

### 6.1.6.7 Test Drug Concentration

Blood samples for PK and ATA will be collected throughout the study per the sample collection schedule provided in Table 11. Validated assays will be used to measure the concentrations of ADC, total antibody (TAb) and MMAE in serum or plasma. PK samples will be collected and archived for possible analysis of other EV-related species. A validated assay will also be used to determine the levels of ATA in serum.

Refer to the Laboratory Manual for information on collection, processing, storage, and shipment of sample.

### 6.1.6.8 Other Measurements, Assessments or Methods

(i) Exploratory Biomarker

The procedures for the collection, handling, and shipping of samples will be specified in the laboratory manual.

The samples described in Sections 6.1.6.8(ii) Biomarkers in Blood and 6.1.6.8(iii) Biomarkers in Pre-Treatment Tumor Tissue may be analyzed for other biomarkers including DNA, RNA and protein, to investigate possible associations with mechanisms of resistance or sensitivity to study treatment, dynamic changes associated with study treatment and method development or validation of diagnostic assays related to study treatment.

The samples may be stored at the study Sponsor's facility or a contract laboratory facility for up to 15 years after database closure, at which time the samples will be destroyed.

(ii) Biomarkers in Blood

The plasma and peripheral blood mononuclear cells (PBMC) samples collected at baseline and post-baseline time points may be analyzed for markers of immune function, immune cell subsets and cytokines. The plasma and PBMC sample may be used for additional exploratory analyses as described in Section 6.1.6.8(i) Exploratory Biomarker.

(iii) Biomarkers in Pre-Treatment Tumor Tissue

Submission of a FFPE tumor block or freshly sectioned unstained charged slides (at least 10 and up to 15 slides) at screening should be provided (unless prior approval is obtained from the sponsor). Either archival tissue or pretreatment fresh tumor tissue (obtained from a fresh biopsy) is acceptable. See the Laboratory Manual for details. The tumor tissue samples may be analyzed for Nectin-4 expression, markers of disease subtype and markers related to the tumor immune microenvironment. The tumor tissue sample may be used for additional exploratory analyses as described in Section 6.1.6.8(i) Exploratory Biomarker.

(iv) Blood Sample for Future Pharmacogenomic Analysis (Retrospective)

Pharmacogenomics (PGx) research may be conducted in the future to analyze or determine genes of relevance to clinical response, pharmacokinetics, and toxicity/safety issues. After randomization (see schedule of assessments), a 4 mL sample of whole blood for possible retrospective PGx analysis will be collected. Samples will be shipped to a sponsor designated banking CRO.

Details on sample collection, labeling, storage and shipment procedures will be provided in a separate laboratory manual.

See Section 6.1.9.4 Retrospective PGx Sub-study for further details on the banking procedures.

(v) EORTC Core Quality of Life Questionnaire, QLQ-C30

The QLQ-C30 was developed to measure aspects of QOL pertinent to patients with a broad range of cancers who are participating in clinical trials. Sneeuw K C, et al., J Clin Epidemiol. 1998; 51:617-31; Aaronson N K, et al., J Natl Cancer Inst. 1993; 85:365-76. The current version of the core instrument (QLQ-C30, Version 3) is a 30-item questionnaire consisting of the following:
functional domains (physical, role, cognitive, emotional, social);
3 symptom scales (fatigue, pain, nausea & vomiting);
Single items for symptoms (shortness of breath, loss of appetite, sleep disturbance, constipation, diarrhea) and financial impact of the disease; and
2 global items (health, overall QOL).

(vi) EuroQOL-5 Dimensions

The EQ-5D-5L is a standardized instrument developed by the EuroQOL Group for use as a generic, preference-based measure of health outcomes. It is applicable to a wide range of health conditions and treatments and provides a simple descriptive profile and a single index value for health status. The EQ-5D-5L is a 5-item self-reported measure of functioning and well-being, which assesses 5 dimensions of health, including mobility, self-care, usual activities, pain/discomfort, and anxiety/depression. Each dimension comprises 5 levels (no problems, slight problems, moderate problems, severe problems, extreme problems). A unique EQ-5D-5L health state is defined by combining 1 level from each of the 5 dimensions. This questionnaire also records the respondent's self-rated health status on a vertical graduated (0 to 100) visual analogue scale. Responses to the 5 items will also be converted to a weighted health state index (utility score) based on values derived from general population samples. Herdman M, et al., Qual Life Res. 2011; 20(10):1727-36

(vii) Healthcare Resource Utilization (HRU)

HRU information will be collected with particular focus on the number of subjects who have an unplanned use of healthcare resources related to clinical events or AEs from all subjects (Section 6.1.9.7).

### 6.1.6.9 Total Amount of Blood

The total amount of blood collected for study assessments for each subject will vary depending on how long the subject stays on treatment.

At any time during the study, if any laboratory abnormalities are found for a subject or for disease assessment, additional blood may be drawn for monitoring.

Additional blood beyond standard monitoring that will be drawn for this study will include draws for eligibility assessment; hematology and chemistry evaluations at specific study defined time points; pharmacokinetics; and bioanalytical sampling.

The maximum amount of blood collected is approximately 130.0 mL in Cycle 1 for subjects randomized to Arm A and 37.0 mL in Cycle 1 for subjects randomized to Arm B. The maximum amount of blood collected for subjects that participate from Cycle 2 up to Cycle 6 and complete the EOT visit is 219.0 mL for subjects randomized to Arm A and 164.0 mL for subjects randomized to Arm B.

### 6.1.7 Discontinuation

### 6.1.7.1 Discontinuation of Individual Subject(s)

A discontinuation from treatment is a subject who enrolled in the study and for whom study treatment is permanently discontinued for any reason.

The subject is free to discontinue from study treatment and/or withdraw from the study for any reason and at any time without giving reason for doing so and without penalty or prejudice. The investigator is also free to discontinue the subject from study treatment or to terminate a subject's involvement in the study at any time if the subject's clinical condition warrants it.

All subjects who discontinue study treatment will remain in the study and must continue to be followed for protocol specific follow up procedures as outlined in the Schedule of Assessments (Table 10) until the subject specifically withdraws consent for any further contact with him/her or persons previously authorized by the participant to provide this information.

If a subject is discontinued from the study with an ongoing AE or an unresolved laboratory result that is significantly outside of the reference range, the investigator will attempt to provide follow-up until the condition stabilizes or no longer is clinically significant.

The following are discontinuation criteria from treatment for individual subjects:
Subject develops radiological disease progression.
Subject is required to receive another systemic anti-cancer treatment for underlying or new cancer.
Subject develops unacceptable toxicity.
Female subject becomes pregnant.
Investigator decides it is in the subject's best interest to discontinue.
Subject declines further treatment.
Subject is noncompliant with the protocol based on the investigator or medical monitor assessment.
Subject is lost to follow-up despite reasonable efforts by the investigator to locate the subject.
Death.

Subjects who discontinue study drug prior to reaching PFS1 and enter the post treatment follow-up period will be discontinued from the post treatment period if any of the following occur:
Subject develops radiological progressive disease (i.e., PFS1) based on investigator assessment
Subject initiates a new systemic anticancer treatment (first line of anticancer therapy after discontinuation of study drug)
Death
Subject declines further study participation (i.e., withdraws consent)
Subject is lost to follow up despite reasonable efforts by the investigator to locate the subject

The subject will be discontinued from the long-term follow-up period (for PFS2) if any of the following occur:
Subject initiates a new systemic anticancer treatment (second line of anticancer therapy after discontinuation of study drug)
Subject exhibits evidence of PD based on investigator assessment
Subject declines further study participation (i.e., withdraws consent)
Subject is lost to follow-up despite reasonable efforts by the investigator to locate the subject
Death
Sponsor ends long-term follow-up collection period

The subject will be discontinued from the survival follow-up period if any of the following occur:
Subject declines further study participation (i.e., withdraws consent)
Subject is lost to follow-up despite reasonable efforts by the investigator to locate the subject
Death
Sponsor ends survival follow-up collection period

### 6.1.8 Statistical Methodology

A Statistical Analysis Plan (SAP) will be written to provide details of the analysis, along with specifications for tables, listings and figures to be produced. The SAP will be finalized before the first subject is enrolled. Any changes from the analyses planned in SAP will be justified in the Clinical Study Report.

In general, continuous data will be summarized with descriptive statistics (number of subjects, mean, SD, minimum, median and maximum), and frequency and percentage for categorical data.

### 6.1.8.1 Sample Size

Approximately 600 subjects will be randomized in a 1:1 ratio to 2 treatment arms: EV

(Arm A) and chemotherapy (Arm B). Randomization will be stratified by baseline ECOG PS (0 vs 1), regions of the world (Western EU, US or the rest of world) and liver metastasis (yes or no). Assuming HR=0.75 (median OS in Arm A and Arm B are 10.7 months and months, respectively) drop-out rate of 10%, the final analysis at the planned 439 death events and 1 interim analysis at 65% of the total planned events (285 death events), this sample size will provide 85% power to detect a statistically significant difference at overall type I error rate of 1-sided 0.025.

Sample size is determined by primary endpoint OS; the 600 subjects will provide more than 90% power to detect statistically significant differences on selected secondary endpoints: PFS1 (assuming median PFS1 in Arm A and Arm B are 6 months and 4 months, respectively), ORR and DCR (assuming 15% treatment difference between Arm A and Arm B for both ORR and DCR).

### 6.1.8.2 Analysis Sets

Detailed criteria for analysis sets will be laid out in SAP or Classification Specifications and the allocation of subjects to analysis sets will be determined prior to database hard-lock.

(i) Full Analysis Set

The full analysis set (FAS) will consist of all subjects who are randomized. This analysis set is in compliance with the intent to treat (ITT) principle that includes all randomized subjects, the FAS is equivalent to the ITT population. This will be the primary analysis set for efficacy analyses except for response related efficacy endpoints.

(ii) Safety Analysis Set

The safety analysis set (SAF), which consists of all subjects who received any amount of study drug, will be used for safety analyses.

(iii) Response Evaluable Set

The response evaluable set (RES) is defined as all subjects in FAS and with measurable disease at baseline and had at least 6 months follow up since randomization. RES will be used for primary efficacy analysis of response related endpoints, e.g., ORR and DCR.

(iv) Pharmacokinetic Analysis Set

Pharmacokinetics Analysis Set (PKAS) includes subjects who received active drug for whom at least one blood sample was collected and assayed for measurement of the TAb, ADC and MMAE serum/plasma concentrations and for whom the time of sampling and the time of dosing on the day of sampling is known.

### 6.1.8.3 Demographics and Baseline Characteristics

Demographics and baseline characteristics will be summarized by treatment group and overall. Ethnic origin will only be summarized in demographic table. Descriptive statistics will include number of subjects, mean, standard deviation, minimum, median and maximum for continuous endpoints, and frequency and percentage for categorical endpoints.

(i) Subject Disposition

The number and percentage of subjects who completed and discontinued treatment and reasons for treatment discontinuation will be presented for all randomized subjects and for subjects in the SAF by treatment group and overall. Similar tables for screening disposition, investigational period disposition and follow-up disposition will also be presented for all randomized subjects by treatment group and overall. All disposition details and dates of first and last evaluations for each subject will be listed.

(ii) Previous and Concomitant Medications

All previous and concomitant medications will be presented in a listing. The frequency of concomitant medications (prescription, over-the-counter and nutritional supplements) will be summarized by treatment group and preferred term (PT) for SAF. Medications will be coded using the WHO drug dictionary. Medications will be counted by the number of subjects who took each medication. A subject taking the same medication multiple times will only be counted once for that medication. Medications will be presented in decreasing order of frequency based on the total number of subjects who took each medication.

(iii) Medical History

Medical history for each subject will be presented in a listing. A detailed medical history for each subject will be obtained during screening period and will be summarized by treatment group and overall.

### 6.1.8.4 Analysis of Efficacy

Efficacy analysis for OS and PFS was conducted on the FAS. The interpretation of results from statistical tests was based on the FAS. Efficacy analysis for response related endpoints, e.g., ORR and DCR was conducted on RES.

The family-wise type I error rate for this study is strongly controlled at 2.5% (one-sided) that allows the study to declare positive on primary endpoint OS on the FAS population. OS was formally tested at both interim analysis and final analysis. At either interim or final analysis, the formal hypothesis tests on the selected secondary endpoints including PFS1, ORR and DCR, were performed hierarchically (per the order of PFS1->ORR->DCR) only when the OS analysis is rejected. The details about the significance levels at interim analysis and final analysis for each efficacy endpoints (OS, PFS1, ORR and DCR) is specified in SAP.

(i) Analysis of Primary Endpoint

(a) Primary Analysis

The primary efficacy endpoint of OS was analyzed using the log-rank test stratified by ECOG PS (0 vs 1), regions of the world (Western EU, US or the rest of world) and liver metastasis (yes or no). Randomized treatment and the strata used for randomization was used for the analysis. In addition, the stratified Cox proportional hazard model was used to estimate the hazard ratio and the corresponding 95% confidence interval. The median OS was estimated using the Kaplan-Meier method and will be reported along with the corresponding 95% confidence interval by treatment arm.

The primary analysis was performed using the FAS.

(b) Sensitivity Analysis

Additional analyses such as unstratified log-rank test and OS analyses adjusting the crossover effect may be conducted, if appropriate. Details are specified in the SAP.

(ii) Analysis of Secondary Endpoints

(a) Progression Free Survival

For each subject, PFS1 is defined as the time from the date of randomization until the date of radiological disease progression (per RECIST V1.1), or until death due to any cause. If a subject has neither progressed nor died, the subject was censored at the date of last radiological assessment. Subjects who receive any further anticancer therapy for the disease before radiological progression were censored at the date of the last radiological assessment before the anticancer therapy started.

The efficacy endpoint of PFS1 was analyzed using the log-rank test stratified by ECOG PS (0 vs 1), regions of the world (Western EU, US or the rest of world) and liver metastasis (yes or no). Randomized treatment and the strata used for randomization was used for the analysis. In addition, stratified Cox proportional hazard model was used to estimate the hazard ratio and the corresponding 95% confidence interval. The median PFS1 was estimated using the Kaplan-Meier method and reported along with the corresponding 95% confidence interval by treatment arm.

The primary analysis was performed using the FAS. Additional sensitivity analyses for PFS1 was also be performed. Details are specified in the SAP.

(b) Overall Response Rate

The overall response rate (ORR) is defined as the proportion of subjects with complete or partial objective response based on the RECIST V1.1. The comparison of ORR between Arm A and Arm B was performed using a stratified CMH test. Same stratification factors used in time to event analyses will be used for the stratified CMH test. The difference in response rates between the treatment arms was estimated along with the corresponding 95% confidence interval. In addition, ORR for each arm was estimated and corresponding 95% confidence interval will be constructed. The primary analysis was performed using the RES.

(c) Duration of Response

Duration of Response (DOR) is defined as the time from the date of the first response CR/PR per RECIST V1.1 (whichever is first recorded) that is subsequently confirmed as assessed by investigator to the date of radiological progression or date of death for subjects who achieved CR or PR. If a subject has not progressed or died, the subject was censored at the date of last radiological assessment or at the date of first CR/PR if no other post-baseline radiological assessment is available after the first CR/PR. The median DOR was estimated using the Kaplan-Meier method and is reported along with the corresponding 95% confidence interval by treatment arm.

(d) Disease Control Rate (DCR)

The DCR is defined as the proportion of subjects with a complete or partial objective response or a stable disease based on RECIST V1.1. The comparison of DCR between Arm A and Arm B was performed using a stratified CMH test. Same stratification factors used in time to event analyses were used for the stratified CMH test. The difference in response rates between the treatment arms were estimated along with the corresponding 95% confidence interval. In addition, DCR for each arm was estimated and corresponding 95% confidence interval will be constructed. The primary analysis was performed using the RES.

(e) QOL and PRO Parameters

Descriptive QOL and PRO analyses were performed on the FAS. Completion rate for each questionnaire is summarized. Additional analyses are discussed in detail in the statistical analysis plan.

(iii) Subgroup Analysis

Using FAS, the analysis for OS, PFS1 and ORR were repeated by ECOG PS (0 vs 1), regions of the world, and liver metastasis respectively.

In addition, subgroup analyses were conducted for selected endpoints to determine whether the treatment effect is consistent. Subgroups may include but are not limited to the following:
Age category (<65 vs ≥65 years; <75 vs ≥75 years)
Sex (female vs male)
Prior platinum (cisplatin vs carboplatin vs both)
Prior lines of systemic therapy in locally advanced or metastatic setting (1 to 2 vs ≥3)
Best response to most recent CPI (responder vs non-responder) CPI most recent treatment (yes vs no)
Baseline hemoglobin (≥10 vs <10 g/dL)
Histology (Urothelial Carcinoma/transitional cell vs Urothelial Carcinoma mixed vs other)
Primary site of tumor (upper tract vs bladder/other)
Smoking status (never vs former vs current)
Brain metastasis status (prior brain metastasis vs no prior brain metastasis)
Investigators' choice of paclitaxel/docetaxel or vinflunine
Baseline Nectin-4 IHC score (<150 vs 150-225 vs >225)
Prior taxane (yes vs no)
PD-L1 CPS (<10 vs ≥10)

(iv) Analysis of Exploratory Endpoints

Exploratory analysis for efficacy endpoints are discussed in the statistical analysis plan.

Serum or plasma TAb, ADC and MMAE concentrations are summarized with descriptive statistics at each PK sampling time point using the PK analysis set. These data may be combined with data from previous studies for population PK and PK/PD analyses. The relationship between TAb, ADC, MMAE and PD endpoints, safety, or efficacy may be explored.

### 6.1.8.5 the Incidence of ATA was Summarized by Visit and Overall Using the Safety Analyses Set

### 6.1.8.6 Analysis of Safety

SAF was used to perform all safety analysis. All treated subjects were analyzed according to the treatment they received.

(i) Adverse Events

AEs are coded using the Medical Dictionary for Regulatory Activities (MedDRA).

A TEAE is defined as an AE observed or worsened after starting administration of the study drug.

The number and percentage of subjects with treatment-emergent AEs, SAES, AEs leading to withdrawal of treatment, and AEs related to study drug were summarized by system organ class, preferred term and treatment group. The number and percentage of AEs by severity were also summarized. All AEs were listed.

A study drug-related TEAE is defined as any TEAE with a causal relationship of YES by the investigator. AEs of interest as classified by customized MedDRA queries and/or standard MedDRA queries were also summarized.

(ii) Laboratory Assessments

For quantitative laboratory measurements descriptive statistics were used to summarize results and change from baseline for subjects in the SAF by treatment group and time point.

Shifts relative to normal ranges from baseline to each time point during treatment period in lab tests were also tabulated. Laboratory data is displayed in listings.

(iii) Vital Signs

Vital sign results and changes from baseline at scheduled visits were summarized with descriptive statistics for subjects in the SAF by treatment group. Vital signs data were displayed in listings.

(iv) Routine 12-lead Electrocardiograms

The 12-lead ECG results were summarized by treatment group and time point.

(v) ECOG Performance Status

Summary statistics (number and percent of subjects) for each category of the ECOG PS at each assessment are provided. The change from baseline to final visit or early termination is also summarized. Negative change scores indicate an improvement. Positive scores indicate a decline in performance.

(vi) Exposure-Response Relationship Analysis

Relationships between TAb, ADC and MMAE concentrations and certain efficacy or safety endpoints may be analyzed in an exploratory manner. Further details of these analyses are described in an exposure-response analysis plan.

### 6.1.8.7 Analysis of Pharmacokinetics

Individual and summary tables of serum TAb and ADC and plasma MMAE concentrations and a listing of blood collection times and concentrations are provided.

Summary statistics are provided including n, mean, SD, geometric mean, minimum, median, maximum, and % CV. Values below the lower limit of quantification (BLOQ) are set to not be calculated if all values are BLOQ. In cases where more than half of the individual data in a group are BLOQ, the geometric mean is not calculated. Additional model-based analyses may be performed and are described in a separate population PK analysis plan.

### 6.1.8.8 Major Protocol Deviations and Other Analyses

Major protocol deviations as defined Section 6.1.8.8 Major Protocol Deviations will be summarized for all randomized subjects by treatment group and total as well as by site. A data listing will be provided by site and subject.

The major protocol deviation criteria will be uniquely identified in the summary table and listing. The unique identifiers will be as follows:
PD1—Entered into the study even though they did not satisfy entry criteria,
PD2—Developed withdrawal criteria during the study and was not withdrawn,
PD3—Received wrong treatment or incorrect dose,
PD4—Received excluded concomitant treatment.

### 6.1.8.9 Interim Analysis (and Early Discontinuation of the Clinical Study)

An interim efficacy analysis is planned to occur after approximately 285 OS events (about 65% of the total planned events) are observed. OS will be tested at 1-sided 0.00541 significance level for efficacy according to the O'Brien-Fleming boundary as implemented by Lan-DeMets alpha spending function. Lan K K G, DeMets D L. Biometrika. 1983; 70:659-63. The IDMC may recommend terminating the trial at the interim analysis based on statistically significant OS results favoring EV. When total deaths reach 439, the final OS analysis will be conducted at the 1-sided 0.02332 significance level. If the exact number of events at interim and final analyses are different than planned, the significance level will be adjusted accordingly, based on the O'Brien-Fleming method with a Lan-DeMets alpha spending function.

The interim analysis will be conducted by IDAC and reviewed by the IDMC. In addition, safety data reviews during the trial will be conducted by the IDMC on a periodic basis. For example, the IDMC will review safety data after the first 50 subjects have been randomized and on study drug for approximately 3 months. The full procedures for IDMC safety review and interim analysis will be described in a separate IDMC charter and Interim Analysis Plan.

### 6.1.8.10 Handling of Missing Data, Outliers, Visit Windows, and Other Information

Imputation methods for missing data, if applicable, and the definitions for windows to be used for analyses will be outlined in the SAP.

### 6.1.9 Appendices for the Clinical Study

### 6.1.9.1 List of Cautionary Concomitant Medications

The following list describes medications and foods that are common strong inhibitors/inducers of CYP3A, CYP2C8 and p-glycoprotein (P-gp) inhibitors that should be avoided, used with caution, or closely monitored. This list should not be considered all inclusive; consult individual drug labels for specific information. If there are concerns or questions about concomitant use of any drugs listed below, discussion with the sponsor is encouraged.

TABLE 21
List of Cautionary Concomitant Medications

| | Strong CYP3A | Strong CYP3A4 | Strong CYP2C8 | Strong CYP2C8 |
|---|---|---|---|---|
| P-gp Inhibitors | Inhibitors | Inducers | Inhibitors | Inducers |
| amiodarone | boceprevir | carbamazepine | clopidogrel | rifampin |
| carvedilol | cobicistat | enzalutamide | gemfibrozil | |
| clarithromycin | conivaptan | mitotane | | |
| drenedarone | danoprevir/ | phenytoin | | |
| itraconazole | ritonavir | rifampin | | |
| lapatinib | elvitegravir/ | St John's wort | | |
| lopinavir/ritonavir | ritonavir | | | |
| propafenone | grapefruit juice | | | |
| quinidine | indinavir/ritonavir | | | |
| ranolazine | itraconazole | | | |
| telaprevir | ketoconazole | | | |
| tipranavir/ritonavir | lopinavir/ritonavir | | | |
| verapamil | paritaprevir/ | | | |
| saquinavir/ritonavir | ritonavir/ | | | |
| | (ombitasvir and/or | | | |
| | dasabuvir) | | | |
| | posaconazole | | | |
| | ritonavir | | | |
| | saquinavir/ | | | |
| | ritonavir | | | |
| | telaprevir | | | |
| | tipranavir/ritonavir | | | |
| | troleandomycin | | | |
| | voriconazole | | | |

Note:
Any additional strong inhibitors/inducers of CYP3A, CYP2C8, and p-glycoprotein (P-gp) inhibitors that are identified or become commercially available while the clinical trial in ongoing are also applicable. P-gp: p-glycoprotein

TABLE 22
Summary of Potential Drug Reactions

| | Arm A: | Arm B: | Arm B: | Arm B: |
|---|---|---|---|---|
| | EV | Docetaxel | Vinflunine | Paclitaxel |
| Strong CYP3A4 | Closely | Should | Should | Exercise |
| inhibitor | monitor | avoid | avoid | caution |
| Strong CYP3A4 | | Should | Should | Exercise |
| inducer | | avoid | avoid | caution |
| P-gp inhibitor | Closely | | | |
| Strong CYP2C8 | monitor | | | Exercise |
| inhibitor/inducer | | | | caution |

EV: enfortumab vedotin;
P-gp: p-glycoprotein

Additional information for inhibitors/inducers can be found in FDA's guidance (Drug Interaction and Labeling). fda.gov/Drugs/DevelopmentApprovalProcess/DevelopmentResources/DrugInteractionsLabeling/ucm093664.htm#table5-2

### 6.1.9.2 Liver Safety Monitoring and Assessment

Any subject enrolled in a clinical study with active drug therapy and reveals an increase of serum aminotransferases (AT) to >3×ULN (to >5×ULN in subjects with liver metastases) or bilirubin >2×ULN should undergo detailed testing for liver enzymes (including at least ALT, AST, ALP, and TBL). Testing should be repeated within 72 hours of notification of the test results. For studies for which a central laboratory is used, alerts are generated by the central laboratory regarding moderate and severe liver abnormality to inform the investigator, study monitor and study team. Subjects should be asked if they have any symptoms suggestive of hepatobiliary dysfunction.

(i) Definition of Liver Abnormalities

Confirmed abnormalities will be characterized as moderate and severe where ULN:

| | ALT or AST | Total Bilirubin |
|---|---|---|
| Moderate | >3 × ULN (in patients without | or >2 × ULN |
| | liver | |
| | metastases), >5 × ULN (in | |
| | patients with liver metastases) | |
| Severe | >3 × ULN | and >2 × ULN |

In addition, the subject should be considered to have severe hepatic abnormalities for any of the following:
ALT or AST >8×ULN.
ALT or AST >5×ULN for more than 2 weeks (in the absence of liver metastases).
ALT or AST >3×ULN and International Normalized Ratio (INR) >1.5 (If INR testing is applicable/evaluated).
ALT or AST >3×ULN with the appearance of fatigue, nausea, vomiting, right upper quadrant pain or tenderness, fever, rash and/or eosinophilia (>5%).

The investigator may determine that abnormal liver function results, other than as described above, may qualify as moderate or severe abnormalities and require additional monitoring and follow-up.

(ii) Follow-up Procedures

Confirmed moderate and severe abnormalities in hepatic functions should be thoroughly characterized by obtaining appropriate expert consultations, detailed pertinent history, physical examination and laboratory tests. The site should complete the liver abnormality case report form (LA-CRF) that has been developed globally and can be activated for any study or appropriate document. Subjects with confirmed abnormal liver function testing should be followed as described below.

Confirmed moderately abnormal LFTs should be repeated 2 to 3 times weekly then weekly or less if abnormalities stabilize or the study drug has been discontinued and the subject is asymptomatic.

Severe hepatic liver function abnormalities as defined above, in the absence of another etiology may be considered an important medical event and may be reported as a SAE. The sponsor should be contacted and informed of all subjects for whom severe hepatic liver function abnormalities possibly attributable to study drug are observed.

To further assess abnormal hepatic laboratory findings, the investigator is expected to:
Obtain a more detailed history of symptoms and prior or concurrent diseases. Symptoms and new-onset diseases is to be recorded as “AEs” in the (e)CRF. Illnesses and conditions such as hypotensive events, and decompensated cardiac disease that may lead to secondary liver abnormalities should be noted. Nonalcoholic steatohepatitis is seen in obese hyperlipoproteinemic, and/or diabetic patients and may be associated with fluctuating AT levels. The investigator should ensure that the medical history form captures any illness that predates study enrollment that may be relevant in assessing hepatic function.
Obtain a history of concomitant drug use (including nonprescription medication, complementary and alternative medications), alcohol use, recreational drug use and special diets. Medications, including dose, is to be entered in the (e)CRF. Information on alcohol, other substance use and diet should be entered on the LA-CRF or an appropriate document.
Obtain a history of exposure to environmental chemical agents.
Based on the subject's history, other testing may be appropriate including:
   Acute viral hepatitis (A, B, C, D, E or other infectious agents),
   Ultrasound or other imaging to assess biliary tract disease,
   Other laboratory tests including INR, direct bilirubin.
Consider gastroenterology or hepatology consultations.
Submit results for any additional testing and possible etiology on the LA-CRF or an appropriate document.

(iii) Study Discontinuation

In the absence of an explanation for increased LFTs, such as viral hepatitis, preexisting or acute liver disease, presence of liver metastases, or exposure to other agents associated with liver injury, the subject may be discontinued from the study. The investigator may determine that it is not in the subject's best interest to continue study enrollment. Discontinuation of treatment should be considered if:
ALT or AST >8×ULN.
ALT or AST >5×ULN for more than 2 weeks (in subjects without liver metastases).
ALT or AST >3×ULN and TBL >2×ULN or INR >1.5 (If INR testing is applicable/evaluated).
ALT or AST >5×ULN and (TBL >2×ULN in patients with liver metastases).
ALT or AST >3×ULN with the appearance of fatigue, nausea, vomiting, right upper quadrant pain or tenderness, fever, rash and/or eosinophilia (>5%).

In addition, if close monitoring for a subject with moderate or severe hepatic laboratory tests is not possible, drug should be discontinued.

Hy's Law Definition:
1. Evidence that a drug can cause hepatocellular-type injury, generally shown by a higher rate than control of people with 3× and greater transaminase elevations over the upper limit of normal (2× elevations are too common in treated and untreated patients to be discriminating).
2. Cases of increased bilirubin (to at least 2×ULN) in people with concomitant transaminase elevation to at least 3×ULN (but it is almost invariably higher) and no evidence of intra- or extra-hepatic bilirubin obstruction (elevated alkaline phosphatase) or Gilbert's syndrome. Temple R. Hy's law: predicting serious hepatotoxicity. Pharmacoepidemiol Drug Saf. 2006; 15(S4):241-3
3. The drug causes hepatocellular injury, generally shown by a higher incidence of 3-fold or greater elevations above the ULN of ALT or AST than the (nonhepatotoxic) control drug or placebo.
4. Among trial subjects showing such AT elevations, often with ATs much greater than 3×ULN, one or more also show elevation of serum TBL to >2×ULN, without initial findings of cholestasis (elevated serum ALP).
5. No other reason can be found to explain the combination of increased AT and TBL, such as viral hepatitis A, B, or C; preexisting or acute liver disease; or another drug capable of causing the observed injury. Guidance for Industry. “Drug-Induced Liver Injury: Premarketing Clinical Evaluation” issued by FDA on July 2009.

### 6.1.9.3 Common Serious Adverse Events

The following is a list of SAE that the sponsor considers to be associated with the disease state being studied. The list does NOT change your reporting obligations or prevent the need to report an AE meeting the definition of an SAE as detailed in Section 6.1.6.6(ii) Definition of Serious Adverse Event. The purpose of this list is to alert the investigator that some events reported as SAEs may not require expedited reporting to the regulatory authorities based on the classification of “common SAEs”. The investigator is required to follow the requirements detailed in Section 6.1.6.6(v) Reporting of Serious Adverse Events.
Urinary tract pain
Bladder disorder
Dysuria
Haemorrhage urinary tract (hematuria)
Urinary tract obstruction

### 6.1.9.4 Retrospective PGx Sub-Study

(i) Introduction

PGx research aims to provide information regarding how naturally occurring changes in a subject's gene and/or expression based on genetic variation may impact what treatment options are best suited for the subject. Through investigation of PGx by technologies such as genotyping, gene sequencing, statistical genetics and Genome-Wide Association Studies, the relationship between gene profiles and a drug's kinetics, efficacy or toxicity may be better understood. As many diseases may be influenced by 1 or more genetic variations, PGx research may identify which genes are involved in determining the way a subject may or may not respond to a drug.

(ii) Objectives

The PGx research that may be conducted in the future with acquired blood samples is exploratory. The objective of this research will be to analyze or determine genes of relevance to clinical response, pharmacokinetics and toxicity/safety issues.

By analyzing genetic variations, it may be possible to predict an individual subject's response to treatment in terms of efficacy and/or toxicity.

(iii) Subject Participation

Subjects who have consented to participate in this study may participate in this PGx sub-study. As part of this sub-study, subjects must provide written consent prior to providing any blood samples that may be used at a later time for genetic analysis.

(iv) Sample Collection and Storage

Subjects who consent to participate in this sub-study will provide one approximately 4-6 mL tube of whole blood per sponsor's instructions. Each sample will be identified by the unique subject number (first code).

(v) PGx Analysis

Sponsor will initiate the PGx analysis in case evidence suggests that genetic variants may be influencing the drug's kinetics, efficacy and/or safety.

(vi) Disposal of PGx Samples/Data

All PGx samples collected will be stored for a period of up to 15 years following study database hardlock. If there is no requirement for analysis, the whole blood sample will be destroyed after the planned storage period. The subject has the right to withdraw consent at any time. When a subject's withdraw notification is received, the PGx sample will be destroyed.

(vii) Information Disclosure to the Subjects

Exploratory PGx analysis may be conducted following the conclusion of the clinical study, if applicable. The results of the genetic analysis will not be provided to any investigators or subjects, nor can the results be requested at a later date.

### 6.1.9.5 EORTC-QLQ-C30 (Version 3)

We are interested in some things about you and your health. Please answer all of the questions yourself by circling the number that best applies to you. There are no “right” or “wrong” answers. The information that you provide will remain strictly confidential.

### 6.1.9.6 EQ-5D-5L

EQ-5D-5L is shown in FIG. 2C.

### 6.1.9.7 Health Resource Utilization

### 6.1.9.8 RECIST V1.1

TABLE A
Time point response: patients with target (+/− non-target) disease.

| | | | Overall |
|---|---|---|---|
| Target lesions | Non-target lesions | New lesions | response |
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or No | PD |
| Any | PD | Yes or No | PD |
| Any | Any | Yes | PD |

CR = complete response,
PR = partial response,
SD = stable disease
PD = progressive disease, and
NE = inevaluable.

TABLE B
Time point response: patients with non-target disease only.

| | | Overall |
|---|---|---|
| Non-target lesions | New lesions | response |
| CR | No | CR |
| Non-CR/non-PD | No | Non-CR/non-PD^{a} |
| Not all evaluated | No | NE |
| Unequivocal PD | Yes or No | PD |
| Any | Yes | PD |

CR = complete response,
PD = progressive disease, and
NE = inevaluable.
^{a}‘Non-CR/non-PD’ is preferred over ‘stable disease’ for non-target disease since SD is increasingly used as endpoint for assessment of efficacy in some trials so to assign this category when no lesions can be measured is not advised

TABLE C
Best overall response when confirmation of CR and PR required.

| Overall | Overall | |
|---|---|---|
| response | response | |
| First | Subsequent | |
| time point | time point | Best overall response |
| CR | CR | CR |
| CR | PR | SD, PD or PR^{a} |
| CR | SD | SD provided minimum criteria for SD |
| | | duration met, otherwise, PD |
| CR | PD | SD provided minimum criteria for SD |
| | | duration met, otherwise, PD |
| CR | NE | SD provided minimum criteria for SD |
| | | duration met, otherwise, NE |
| PR | CR | PR |
| PR | PR | PR |
| PR | SD | SD |
| PR | PD | SD provided minimum criteria for SD |
| | | duration met, otherwise, PD |
| PR | NE | SD provided minimum criteria for SD |
| | | duration met, otherwise, NE |
| NE | NE | NE |

CR = complete response,
PR = partial response,
SD = stable disease,
PD = progressive disease, and
NE = inevaluable.
^{a}If a CR is truly met at first time point, then any disease seen at a subsequent point, even disease meeting PR criteria relative to baseline, makes the disease PD at that point (since disease must have reappeared after CR). Best response would depend on whether minimum duration for SD was met. However, sometimes ‘CR’ may be claimed when subsequent scans suggest small lesions were likely still present and in fact the patient had PR, not CR at the first time point. Under these circumstances, the original CR should be changed to PR and the best response is PR.
Reproduced from: Eisenhauer EA, Therasse P, Bogaerts J, Schwartz LH, Sargent D, Ford R, et al.
New response evaluation criteria in solid tumors: revised RECIST guideline (version 1.1). Eur J Cancer. 2009; 45: 228-47.

### 6.1.9.9 ECOG Performance Status

| GRADE | ECOG PERFORMANCE STATUS |
|---|---|
| 0 | Fully active, able to carry on all pre-disease performance without restriction |
| 1 | Restricted in physically strenuous activity but ambulatory and able to carry out |
| | work of a light or sedentary nature, e.g., light house work, office work |
| 2 | Ambulatory and capable of all selfcare but unable to carry out any work activities; |
| | up and about more than 50% of waking hours |
| 3 | Capable of only limited selfcare; confined to bed or chair more than 50% of |
| | waking hours |
| 4 | Completely disabled; cannot carry on any selfcare; totally confined to bed or chair |
| 5 | Dead |

ECOG: Eastern Cooperative Oncology Group
Reproduced from: Oken MM, Creech RH, Tormey DC, Horton J, Davis TE, McFadden ET, et al.
Toxicity and response criteria of the Eastern Cooperative Oncology Group. Am J Clin Oncol. 1982; 5: 649-55.

### 6.1.10 Descriptions of Terms or Abbreviations Used in the Clinical Studies

| Abbreviations/ | |
|---|---|
| Terms | Description of abbreviations |
| ADC | Antibody drug conjugate |
| AE | adverse event |
| Alb | albumin |
| ALP | alkaline phosphatase |
| ALT | alanine aminotransferase |
| ANC | absolute neutrophil count |
| AST | aspartate aminotransferase |
| AT | aminotransferases |
| ATA | antitherapeutic antibodies |
| BLOQ | below the lower limit of quantification |
| BUN | blood urea nitrogen |
| CA | competent authority |
| cfDNA | circulating free deoxyribonucleic acid |
| CFR | Code of Federal Regulations |
| CHO | Chinese Hamster Ovary |
| CIOMS | council for international organizations |
| | of medical sciences |
| CNS | central nervous system |
| CPI | checkpoint inhibitor |
| CR | complete response |
| CrCl | creatinine clearance |
| CRF | case report form |
| CRO | contract research organization |
| CT | computed tomography |
| CTCAE | Common Terminology Criteria for Adverse Events |
| CV | coefficient of variation |
| CYP3A4 | cytochrome P450 3A4 |
| DCR | disease control rate |
| DILI | drug-induced liver injury |
| DLT | dose-limiting toxicity |
| DOR | duration of response |
| ECG | electrocardiogram |
| ECOG PS | Eastern Cooperative Oncology Group |
| | Performance Status |
| eCRF | electronic case report form |
| EDC | electronic data capture |
| EEA | European Economic Area |
| EORTC | European Organisation for Research |
| | and Treatment of Cancer |
| EOT | end of treatment |
| ePRO | electronic patient reported outcome |
| EQ-5D-5L | EuroQOL 5-dimensions |
| EV | enfortumab vedotin |
| FAS | full analysis set |
| FDA | Food and Drug Administration |
| GCP | good clinical practice |
| GFR | glomerular filtration rate |
| GGT | gamma-glutamyl transferase |
| GMP | good manufacturing practice |
| HbA1c | Hemoglobin A1c |
| HBsAg | hepatitis B surface antigen |
| Hct | hematocrit |
| HCV | hepatitis C virus |
| Hgb | hemoglobin |
| HIPAA | health insurance portability and |
| | accountability act |
| HIV | human immunodeficiency virus |
| HRU | healthcare resource utilization |
| IARC | International Agency for Research on Cancer |
| ICF | informed consent form |
| ICH | International Council for Harmonisation |
| IDAC | Independent data analysis center |
| IDMC | Independent data monitoring committee |
| IEC | independent ethics committee |
| IND | investigational new drug |
| INR | international normalized ratio |
| IRB | institutional review board |
| IRR | infusion-related reaction |
| IRT | interactive response technology |
| ITT | Intent to treat |
| ISN | international study number |
| IUD | intrauterine device |
| IUS | intrauterine hormone releasing system |
| LA-CRF | liver abnormality case report form |
| LFT | liver function test |
| MMAE | monomethyl auristatin E |
| MRI | magnetic resonance imaging |
| MTD | maximum tolerated dose |
| mUC | metastatic urothelial cancer |
| MVAC | methotrexate, vinblastine, doxorubicin, and cisplatin |
| NCI-CTCAE | National Cancer Institute's Common |
| | Terminology Criteria for Adverse Events |
| NOAEL | no observed adverse effect level |
| ORR | overall response rate |
| OS | overall survival |
| PBMC | peripheral blood mononuclear cells |
| PD | progressive disease |
| PDAS | pharmacodynamics analysis set |
| PD-1 | programmed cell death protein 1 |
| PD-L1 | programmed death-ligand 1 |
| PFS | progression free survival |
| P-gp | p-glycoprotein |
| PGx | pharmacogenomics analyses |
| PKAS | pharmacokinetic analysis set |
| PR | partial response |
| PRES | posterior reversible encephalopathy syndrome |
| PRO | patient reported outcome |
| QLQ-C30 | EORTC Quality of Life Questionnaire |
| QOL | quality of life |
| RBC | red blood cell |
| RECIST | Response Evaluation Criteria in Solid Tumors |
| RES | response evaluable set |
| ROW | rest of the world |
| SAE | serious adverse event |
| SAF | safety analysis set |
| SAP | statistical analysis plan |
| SD | stable disease |
| SmPC | Summary of product characteristics |
| SOP | standard operating Procedure |
| TAb | total antibody |
| TBL | total bilirubin |
| TEAE | treatment emergent adverse events |
| TMF | trial master file |
| TP | total protein |
| ULN | upper limit of normal |
| US | United States |
| WBC | white blood cell |
| Baseline | Assessments of subjects as they enter |
| | a trial before they receive any treatment. |
| Endpoint | Variable that pertains to the efficacy or |
| | safety evaluations of a trial. |
| Enroll | To register or enter a subject into a |
| | clinical trial. NOTE: Once a subject has |
| | received the study drug or placebo, the clinical |
| | trial protocol applies to the subject. |
| Investigational | Period of time where major interests of protocol |
| period | objectives are observed, and where the test |
| | drug or comparative drug (sometimes without |
| | randomization) is usually given to a subject, |
| | and continues until the last assessment after |
| | completing administration of the test drug or |
| | comparative drug. |
| Randomization | The process of assigning trial subjects to |
| | treatment or control groups using an element |
| | of chance to determine assignments |
| | in order to reduce bias. |
| Screening | A process of active consideration of potential |
| | subjects for enrollment in a trial. |
| Screen failure | Potential subject who did not meet 1 or more |
| | criteria required for participation in a trial. |
| Screening period | Period of time before entering the investigational |
| | period, usually from the time when a subject |
| | signs the consent until just before the test |
| | drug or comparative drug (sometimes without |
| | randomization) is given to a subject. |
| Study period | Period of time from the first site initiation |
| | date to the last site completing the study. |
| Variable | Any entity that varies; any attribute, phenomenon |
| | or event that can have different qualitative |
| | or quantitative values. |

### 6.1.11 Study Results

The treatment disposition of the clinical study described herein is shown in Table 23 below:

TABLE 23
Treatment Disposition

| | Enfortumab | | |
|---|---|---|---|
| Data cutoff 15 Jul. 2020 | Vedotin | Chemotherapy | Total |
| Full Analysis Set | 301 | 307 | 608 |
| Treated | 296 | 291 | 587 |
| Not Treated | 5 | 16 | 21 |
| # of Deaths | 134 | 167 | 301 |

The patient demographics of the clinical study described herein is shown in Table 24 below:

TABLE 24
Demographic and Baseline Characteristics, Stratification Factors and Disease History

| | | Enfortumab | | | | | |
|---|---|---|---|---|---|---|---|
| | | vedotin | | Chemotherapy | | Total | |
| Parameter | Category/Statistic | (N = 301) | | (N = 307) | | (N = 608) | |
| Sex | Male | 238 | (79.1%) | 232 | (75.6%) | 470 | (77.3%) |
| | Female | 63 | (20.9%) | 75 | (24.4%) | 138 | (22.7%) |
| Age (Years) | Median (Range) | 68.00 | (34.0, 85.0) | 68.00 | (30.0, 88.0) | 68.00 | (30.0, 88.0) |
| Age Group | >=75 | 52 | (17.3%) | 68 | (22.1%) | 120 | (19.7%) |
| (Years) | | | | | | | |
| BMI Category | <18.5 | 12 | (4.0%) | 15 | (4.9%) | 27 | (4.4%) |
| | >=18.5 to <25 | 123 | (40.9%) | 136 | (44.3%) | 259 | (42.6%) |
| | >=25 to <30 | 123 | (40.9%) | 107 | (34.9%) | 230 | (37.8%) |
| | >=30 | 43 | (14.3%) | 48 | (15.6%) | 91 | (15.0%) |
| | Not Reported | 0 | | 1 | (0.3%) | 1 | (0.2%) |
| Bellmunt Risk | 0-1 | 201 | (66.8%) | 208 | (67.8%) | 409 | (67.3%) |
| Score‡ | >=2 | 90 | (29.9%) | 96 | (31.3%) | 186 | (30.6%) |
| | Not Reported | 10 | (3.3%) | 3 | (1.0%) | 13 | (2.1%) |
| ECOG PS per | ECOG PS = 0 | 120 | (39.9%) | 124 | (40.4%) | 244 | (40.1%) |
| IRT† | ECOG PS = 1 | 181 | (60.1%) | 183 | (59.6%) | 364 | (59.9%) |
| Region | Region = Western | 126 | (41.9%) | 129 | (42.0%) | 255 | (41.9%) |
| | Europe | | | | | | |
| | Region = US | 43 | (14.3%) | 44 | (14.3%) | 87 | (14.3%) |
| | Region = Rest of the | 132 | (43.9%) | 134 | (43.6%) | 266 | (43.8%) |
| | World | | | | | | |
| Tobacco use | Former user | 167 | (55.5%) | 164 | (53.4%) | | |
| | Current user | 29 | (9.6%) | 31 | (10.1%) | | |
| | Never used | 91 | (30.2%) | 102 | (33.2%) | | |
| | Not reported or | 14 | (4.7%) | 10 | (3.3%) | | |
| | unknown | | | | | | |
| History of | | 56 | (18.6%) | 58 | (18.9%) | | |
| diabetes or | | | | | | | |
| hyperglycemia | | | | | | | |
| Liver Metastasis | Liver Metastasis = No | 208 | (69.1%) | 212 | (69.1%) | 420 | (69.1%) |
| per IRT | Liver Metastasis = Yes | 93 | (30.9%) | 95 | (30.9%) | 188 | (30.9%) |
| Primary Disease | Upper tract | 98 | (32.6%) | 107 | (34.9%) | 205 | (33.7%) |
| Site of Origin | Bladder/Other | 203 | (67.4%) | 200 | (65.1%) | 403 | (66.3%) |
| Visceral | No | 67 | (22.3%) | 56 | (18.3%) | 123 | (20.3%) |
| Metastasis | Yes | 234 | (77.7%) | 250 | (81.7%) | 484 | (79.7%) |
| Histologic type at | Urothelial or | 229/301 | (76.1%) | 230/305 | (75.4%) | | |
| initial diagnosis | transitional-cell | | | | | | |
| | carcinoma | | | | | | |
| - no./total no. | Urothelial carcinoma, | 45/301 | (15.0%) | 42/305 | (13.8%) | | |
| (%) | mixed types | | | | | | |
| | Other§ | 27/301 | (9.0%) | 33/305 | (10.8%) | | |
| Previous | 1-2 | 262 | (87.0%) | 270 | (87.9%) | | |
| therapies | | | | | | | |
| systemic | ≥3 | 39 | (13.0%) | 37 | (12.1%) | | |
| Best response | Response | 61 | (20.3%) | 50 | (16.3%) | | |
| among patients | No response | 207 | (68.8%) | 215 | (70.0%) | | |
| who previously | | | | | | | |
| received | | | | | | | |
| checkpoint | | | | | | | |
| inhibitor | | | | | | | |
| treatment | | | | | | | |
| Median time | | 14.8 | (0.2-114.1) | 13.2 | (0.3-118.4) | | |
| since diagnosis | | | | | | | |

of metastatic or
locally advanced
disease (range)
- mo

*Percentages may not total 100 because of rounding.
†Eastern Cooperative Oncology Group (ECOG) performance-status scores range from 0 to 4, with higher scores indicating greater disability.
‡Bellmunt risk scores range from 0 to 3 according to the presence of the following risk factors: a hemoglobin level of less than 10 g per deciliter, an ECOG performance-status score of greater than 0, and liver metastasis.
§Other histologic types include adenocarcinoma, squamous-cell carcinoma, and pseudosarcomatic differentiation.
The best response among patients who had a response was defined as a confirmed complete or partial response; among patients who did not have a response, the best response was defined as stable disease or progressive disease.

The study exposure of the clinical study described herein is shown in Table 25 below:

TABLE 25
Study Exposure

| | | Enfortumab | Chemotherapy | Chemotherapy | Chemotherapy | Chemotherapy |
|---|---|---|---|---|---|---|
| | | Vedotin | Docetaxel | Paclitaxel | Vinflunine | Combined |
| | Statistics | (N = 296) | (N = 109) | (N = 107) | (N = 75) | (N = 291) |
| Duration | n | 296 | 109 | 107 | 75 | 291 |
| of | Mean | 163.3 | 110.2 | 118.6 | 138.5 | 120.6 |
| Exposure | SD | 113.3 | 86.9 | 79.4 | 105.0 | 89.7 |
| (days) | Min | 16 | 8 | 8 | 6 | 6 |
| | Median | 152.0 | 68.0 | 112.0 | 119.0 | 105.0 |
| | Max | 589 | 456 | 374 | 423 | 456 |

The overall survival of the clinical study described herein OS was analyzed on the Full Analysis Set (FAS) population at the interim analysis which included a total of 301 deaths, as shown in Table 26 below:

TABLE 26
Summary of Overall Survival in FAS

| | Enfortumab vedotin | | Chemotherapy | |
|---|---|---|---|---|
| | (N = 301) | | (N = 307) | |
| Measure | | | | |
| Deaths, n (%) | 134 | (44.5%) | 167 | (54.4%) |
| Median OS in Months (95% CI) | 12.88 | (10.58, 15.21) | 8.97 | (8.05, 10.74) |
| Stratified Analysis | | | | |
| 1-sided P-value (from log-rank) | 0.00142* | | | |
| Hazard Ratio (95% CI) | 0.702 | (0.556, 0.886) | | |
| Median (95% CI) Follow-up of OS, | 11.10 | (10.35, 11.93) | 11.07 | (9.99, 12.12) |

| | | |
|---|---|---|
| Months [6] | | |
| Overall | 11.10 | (10.55, 11.60) |

*pre-determined efficacy boundary = 0.00679 (adjusted per observed deaths of 301)

After a median follow-up of 11.1 months, the risk of death was 30% lower with enfortumab vedotin than with chemotherapy (hazard ratio, 0.70; 95% confidence interval [CI], 0.56 to 0.89; P=0.001), indicating significantly longer overall survival with enfortumab vedotin.

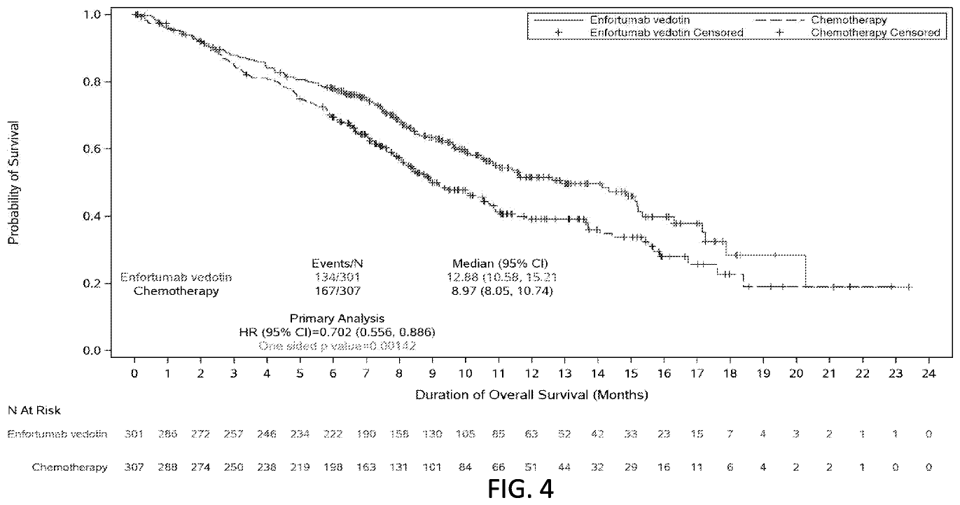

The overall survival in Kaplan Meier Plot—FAS is shown in FIG. 4.

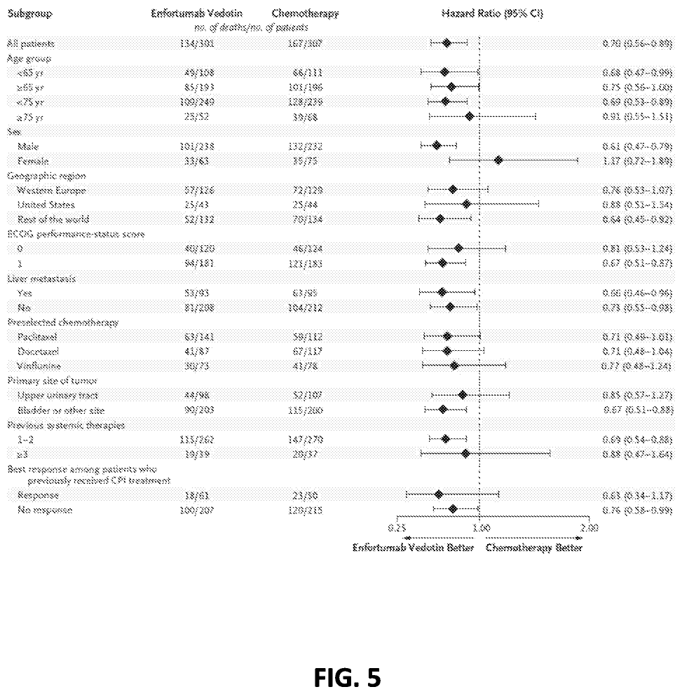

The overall survival for the sub-groups is shown in FIG. 5. The median overall survival was 12.88 months (95% CI, 10.58 to 15.21) in the enfortumab vedotin group and 8.97 months (95% CI, 8.05 to 10.74) in the chemotherapy group. The estimated percentage of patients alive at 12 months was 51.5% (95% CI, 44.6 to 58.0) in the enfortumab vedotin group and 39.2% (95% CI, 32.6 to 45.6) in the chemotherapy group.

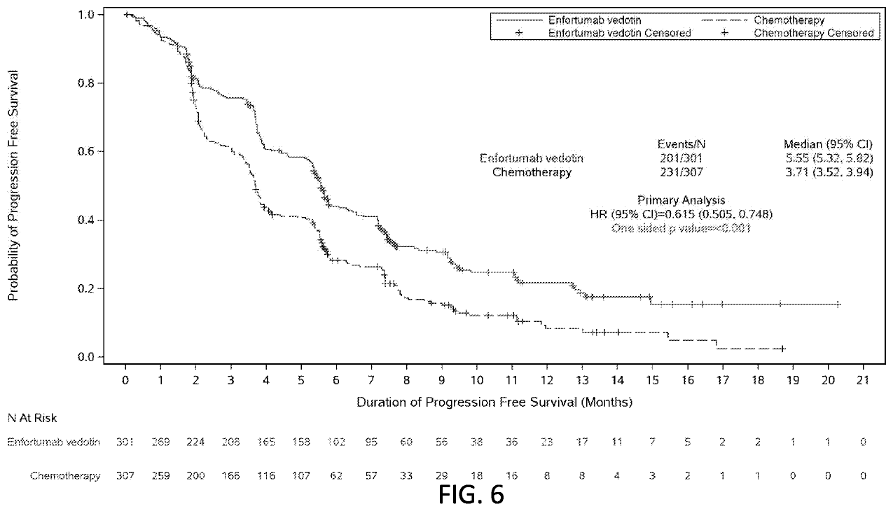

As an additional measure for the efficacy, the progression free survival (PFS) was analyzed on the FAS population at the interim analysis which included a total of 432 PFS events, as shown in Table 27. The PFS is additionally shown in FIG. 6.

TABLE 27
Summary of Progression-Free Survival, Investigator Assessment - FAS

| | Enfortumab vedotin | | Chemotherapy | |
|---|---|---|---|---|
| | (N = 301) | | (N = 307) | |
| Measure | | | | |
| PFS Events, n (%) | 201 | (66.8%) | 231 | (75.2%) |
| Median PFS in Months (95% CI) | 5.55 | (5.32, 5.82) | 3.71 | (3.52, 3.94) |

| Stratified Analysis | | |
|---|---|---|
| 1-sided P-value (from log-rank) | <0.00001 * | |
| Hazard Ratio (95% CI) | 0.615 | (0.505, 0.748) |

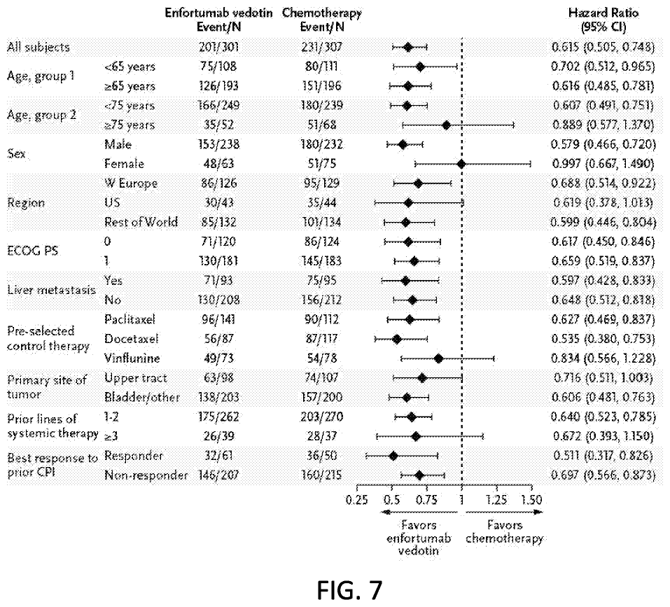

The results of subgroup analyses show that a progression-free survival benefit with enfortumab vedotin was present across multiple subgroups, as shown in FIG. 7. This forest plot shows investigator-assessed progression-free survival in prespecified key subgroups of the intention-to-treat population, consisting of all randomized patients. For “All Subjects,” results reported are based on stratified analysis with the following stratification factors: ECOG PS, region, and liver metastasis.

Results of sensitivity analyses were consistent with those of the primary analysis, as shown in Table 28.

TABLE 28
Sensitivity Analyses for Overall Survival

| Sensitivity Analysis | | 1-sided |
|---|---|---|
| Method/Description | HR (95% CI) | P-value |
| Unstratified analysis | 0.719 (0.572-0.903) | 0.002 |
| Adjusting in chemotherapy arm | 0.705 (0.516-0.853) | 0.001 |
| based on the rank preserving | | |
| structural failure time method* | | |
| Inverse probability of censoring weights | 0.630 (0.435-0.912) | 0.001 |
| method^{ł} | | |

*Stratified Cox proportional hazard model was analyzed on 1000 bootstrapping simulated datasets. Confidence interval was from 2.5 percentile and 97.5 percentile of 1000 simulations. P-value was calculated from 1000 simulations.
^{ł}HR (95% CI) was based on stratified Cox proportional hazard model with treatment as the explanatory variables. Stratification factors were ECOG PS, region, and liver metastasis. The weight was calculated from two logistic models. One model included only baseline covariates (age group, primary site of tumor, and prior lines of therapy in locally advanced or metastatic setting). The other model included both baseline covariates and time-dependent variables (sum of diameter and ECOG assessments). The 1-sided P-value was based on weight stratified logrank test.
CI denotes confidence interval, ECOG PS Eastern Cooperative Oncology Group Performance-Status Score, HR hazard ratio.

The ORR and DOR were analyzed and are shown in Table 29.

TABLE 29
ORR and DOR, Investigator Assessment - Responsive-Evaluable Population (RES)

| | Enfortumab vedotin | | Chemotherapy | |
|---|---|---|---|---|
| Parameter | (N = 288) | | (N = 296) | |
| Overall Response Rate (ORR), | 117 | (40.6%) | 53 | (17.9%) |
| Confirmed, n (%) | | | | |
| 95% CI for ORR (%) | | (34.90%, 46.54%) | | (13.71%, 22.76%) |
| Stratified 1-sided P-value | <0.001 * | | | |
| Disease control rate * | | | | |
| Patients, n (%) | 207 | (71.9%) | 158 | (53.4%) |
| 95% CI, % | 66.30, 76.99 | | 47.52, 59.17 | |
| Stratified 1-sided P-value | <0.001 | | | |
| Time to response, months | | | | |
| Median | 1.87 | | 1.91 | |
| Range | 1.1, 5.7 | | 1.2, 8.6 | |
| Duration of Response (DOR)^{ł} | | | | |
| Events, n (%) | 63/117 | (53.8%) | 29/53 | (54.7%) |
| Median of DOR in Months (95% CI) | 7.39 | (5.59, 9.46) | 8.11 | (5.65, 9.56) |
| At 6 months, % | 53.8 | | 56.0 | |
| At 12 months, % | 27.7 | | 19.8 | |
| Best overall response, n (%)‡ | | | | |
| Complete response | 14 | (4.9) | 8 | (2.7) |
| Partial response | 103 | (35.8) | 45 | (15.2) |
| Stable disease | 90 | (31.3) | 105 | (35.5) |
| Progressive disease | 44 | (15.3) | 83 | (28.0) |
| Not evaluable | 37 | (12.8) | 55 | (18.6) |

* Disease control rate is defined as the proportion of patients who had a best overall response of confirmed complete response, confirmed partial response, or stable disease (at least 7 weeks).
^{ł}In all patients with confirmed complete or partial response.
‡The definition of best overall response was according to RECIST v1.1. Complete or partial response was confirmed by two scans at least 4 weeks apart. The minimum duration for stable disease was 7 weeks. CI denotes confidence interval, and RECIST Response Evaluation Criteria in Solid Tumors.

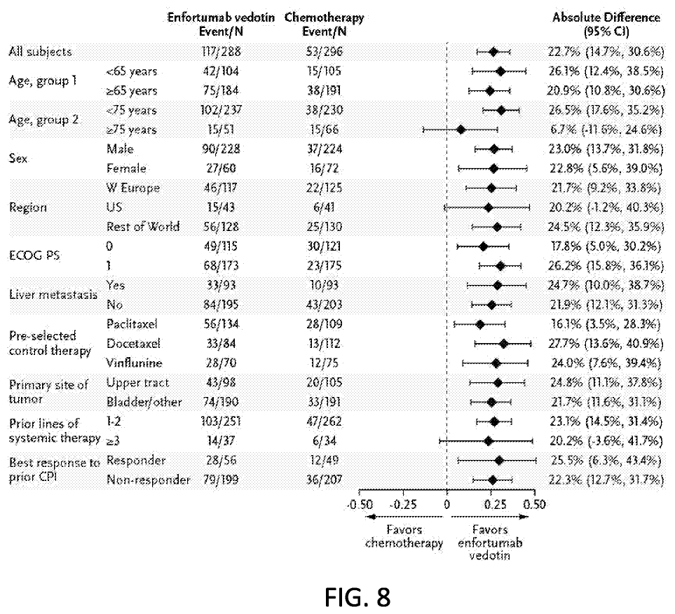

The results of subgroup analyses were consistent with those of the primary analysis, as shown in FIG. 8. This forest plot shows investigator-assessed overall response rate in prespecified key subgroups of the response-evaluable population, consisting of all randomized patients with measurable disease at baseline. For “All Subjects,” results reported are based on unstratified analysis. A complete response was observed in 4.9% of the patients (14 of 288) in the enfortumab vedotin group and in 2.7% of the patients (8 of 296) in the chemotherapy group. Disease control was observed in 71.9% (95% CI, 66.3 to 77.0) and 53.4% (95% CI, 47.5 to 59.2), respectively (P<0.001).

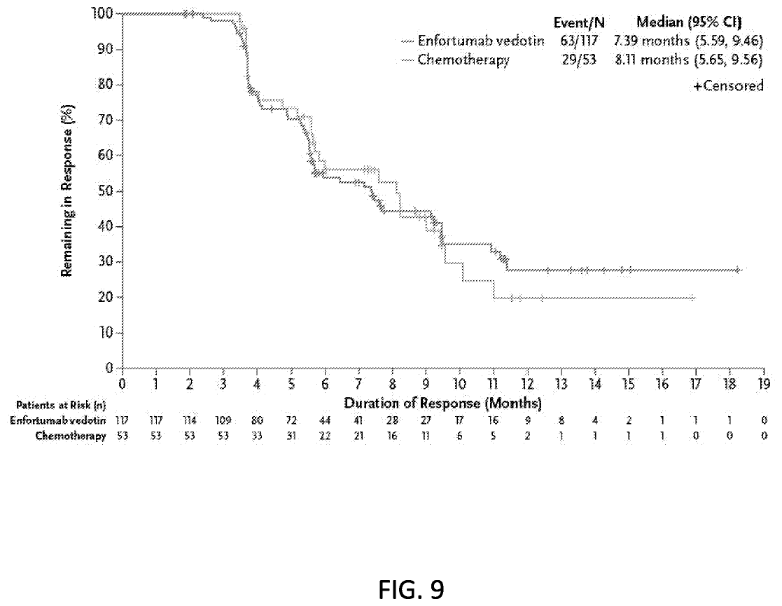

In patients who had a complete or partial response, the median duration of response was 7.39 months in the enfortumab vedotin group and 8.11 months in the chemotherapy group, as shown in FIG. 9.

Treatment-related adverse events of grade 3 or higher occurred in 51.4% of patients in the enfortumab vedotin group and in 49.8% in the chemotherapy group. After adjustment for treatment exposure, the rate was 2.4 and 4.3 events per patient-year in the enfortumab vedotin group and the chemotherapy group, respectively. An overview of adverse events adjusted by patient-year is shown in Table 30.

TABLE 30
Overview of Adverse Events Adjusted by Patient-Year

| | Enfortumab Vedotin | | Chemotherapy Group | |
|---|---|---|---|---|
| | Group (N = 296) | | (N = 291) | |
| Event, E (E/PY) | (PY = 132.3) | | (PY = 96.1) | |
| Overall | | | | |
| TEAE | 5051 | (38.2) | 3173 | (33.0) |
| TRAE* | 3033 | (22.9) | 1982 | (20.6) |
| Serious | | | | |
| TEAE | 314 | (2.4) | 269 | (2.8) |
| TRAE* | 114 | (0.9) | 120 | (1.2) |
| Grade ≥3 severity | | | | |
| TEAE | 617 | (4.7) | 594 | (6.2) |
| TRAE* | 320 | (2.4) | 416 | (4.3) |
| Leading to dose reduction | | | | |
| TEAE | 170 | (1.3) | 119 | (1.2) |
| TRAE* | 165 | (1.2) | 112 | (1.2) |
| Leading to dose interrup- | | | | |
| tion | | | | |
| TEAE | 536 | (4.1) | 140 | (1.5) |
| TRAE* | 384 | (2.9) | 90 | (0.9) |
| Leading to treatment | | | | |
| withdrawal | | | | |
| TEAE | 66 | (0.5) | 60 | (0.6) |
| TRAE* | 51 | (0.4) | 38 | (0.4) |
| Leading to death | | | | |
| TEAE | 21 | (0.2) | 18 | (0.2) |
| TRAE* | 7 | (0.1) | 3 | (0.0) |
| Leading to death, excluding | | | | |
| disease progression | | | | |
| TEAE | 11 | (0.1) | 12 | (0.1) |
| TRAE* | 7 | (0.1) | 3 | (0.0) |

*TRAE indicates reasonable possibility that the event may have been caused by the study treatment as assessed by the investigator. If the relationship is missing, then the adverse event is considered to be treatment-related.
E denotes number of events, PY patient-year, TEAE treatment-emergent adverse event, and TRAE treatment-related adverse event.

Treatment-Related Adverse Events Leading to Treatment Modification are shown in Table 31. In particular, grade 3 or higher treatment-related adverse events that occurred in at least 5% of patients included maculopapular rash (7.4%), fatigue (6.4%), and decreased neutrophil count (6.1%) in the enfortumab vedotin group and decreased neutrophil count (13.4%), anemia (7.6%), decreased white-cell count (6.9%), neutropenia (6.2%), and febrile neutropenia (5.5%) in the chemotherapy group. Treatment-related adverse events resulting in dose reduction, interruption of treatment, or withdrawal of treatment occurred in 32.4%, 51.0%, and 13.5% of patients in the enfortumab vedotin group, respectively, and in 27.5%, 18.9%, and 11.3% in the chemotherapy group, respectively.

TABLE 31
Treatment-Related Adverse Events
Leading to Treatment Modification

| | Enfortumab Vedotin | | Chemotherapy | |
|---|---|---|---|---|
| | Group | | Group | |
| Event, n (%) | (N = 296) | | (N = 291) | |
| Leading to dose reduction | 96 | (32.4) | 80 | (27.5) |
| Peripheral sensory neuropathy | 21 | (7.1) | 18 | (6.2) |
| Rash maculo-papular | 13 | (4.4) | 0 | |
| Decreased appetite | 10 | (3.4) | 3 | (1.0) |
| Fatigue | 8 | (2.7) | 11 | (3.8) |
| Neutrophil count decreased | 6 | (2.0) | 8 | (2.7) |
| Neutropenia | 5 | (1.7) | 6 | (2.1) |
| Febrile neutropenia | 0 | | 8 | (2.7) |
| Leading to dose interruption | 151 | (51.0) | 55 | (18.9) |
| Peripheral sensory neuropathy | 46 | (15.5) | 4 | (1.4) |
| Fatigue | 16 | (5.4) | 4 | (1.4) |
| Neutrophil count decreased | 15 | (5.1) | 10 | (3.4) |
| Rash maculo-papular | 13 | (4.4) | 0 | |
| Rash | 10 | (3.4) | 0 | |
| Neuropathy peripheral | 9 | (3.0) | 1 | (0.3) |
| Anemia | 8 | (2.7) | 6 | (2.1) |
| Drug eruption | 7 | (2.4) | 0 | |
| Neutropenia | 6 | (2.0) | 5 | (1.7) |
| Diarrhea | 6 | (2.0) | 0 | |
| Asthenia | 6 | (2.0) | 1 | (0.3) |
| Alanine aminotransferase increased | 6 | (2.0) | 1 | (0.3) |
| Leading to dose withdrawal | 40 | (13.5) | 33 | (11.3) |
| Peripheral sensory neuropathy | 7 | (2.4) | 6 | (2.1) |

Reporting events by Preferred Term with percentage ≥2% in either treatment arm (listed in order of highest frequency in the enfortumab vedotin arm).

The treatment-emergent adverse events and deaths were analyzed and are shown in Table 32. All adverse events that occurred during the treatment period are listed in Table 30.

TABLE 32
Overview of Treatment-Emergent Adverse Events and Death

| | Enfortumab vedotin | | Chemotherapy | | Total | |
|---|---|---|---|---|---|---|
| | (N = 296) | | (N = 291) | | (N = 587) | |
| Event, n (%) | n (%) | | n (%) | | n (%) | |
| TEAE | 290 | (98.0%) | 288 | (99.0%) | 578 | (98.5%) |
| Drug-Related TEAE | 278 | (93.9%) | 267 | (91.8%) | 545 | (92.8%) |
| Serious TEAE | 138 | (46.6%) | 128 | (44.0%) | 266 | (45.3%) |
| Drug-Related Serious TEAE | 67 | (22.6%) | 68 | (23.4%) | 135 | (23.0%) |
| TEAE Leading to Withdrawal of | 51 | (17.2%) | 51 | (17.5%) | 102 | (17.4%) |
| Treatment | | | | | | |
| Leading to dose reduction | 101 | (34.1) | 81 | (27.8) | | |
| Leading to dose interruption | 180 | (60.8) | 85 | (29.2) | | |
| Drug-Related TEAE Leading to | 40 | (13.5%) | 33 | (11.3%) | 73 | (12.4%) |
| Withdrawal of Treatment | | | | | | |
| TEAE with CTCAE Grade >=3 | 210 | (70.9%) | 193 | (66.3%) | 403 | (68.7%) |
| Drug-Related TEAE with CTCAE | 152 | (51.4%) | 145 | (49.8%) | 297 | (50.6%) |
| Grade >=3 | | | | | | |
| TEAE Leading to Death | 21 | (7.1%) | 16 | (5.5%) | 37 | (6.3%) |
| Drug-Related TEAE Leading to | 7 | (2.4%) | 3 | (1.0%) | 10 | (1.7%) |
| Death | | | | | | |
| TEAE Leading to Death, Exclusing | 11 | (3.7%) | 11 | (3.8%) | 22 | (3.7%) |
| Disease Progression | | | | | | |
| Drug-Related TEAE Leading to | 7 | (2.4%) | 3 | (1.0%) | 10 | (1.7%) |
| Death, Exclusing Disease Progression | | | | | | |
| Death | 130 | (43.9%) | 161 | (55.3%) | 291 | (49.6%) |

Adverse event occurring in ≥20% of patients in either treatment arm

| | | | | |
|---|---|---|---|---|
| Alopecia | 139 | (47.0) | 110 | (37.8) |

| | | | | |
|---|---|---|---|---|
| Decreased appetite | 121 | (40.9) | 78 | (26.8) |
| Fatigue | 107 | (36.1) | 78 | (26.8) |
| Diarrhea | 103 | (34.8) | 66 | (22.7) |
| Peripheral sensory neuropathy | 102 | (34.5) | 66 | (22.7) |
| Pruritis | 102 | (34.5) | 20 | (6.9) |
| Nausea | 89 | (30.1) | 74 | (25.4) |
| Constipation | 82 | (27.7) | 73 | (25.1) |
| Dysgeusia | 74 | (25.0) | 23 | (7.9) |
| Pyrexia | 65 | (22.0) | 41 | (14.1) |
| Anemia | 59 | (19.9) | 87 | (29.9) |

The adverse events of special interest were analyzed and are shown in Table 33.

TABLE 33
Treatment-Emergent Adverse Event with Special Safety Interest (MedDRA v23.0)

| | Enfortumab vedotin | | Chemotherapy | | Total | |
|---|---|---|---|---|---|---|
| | (N = 296) | | (N = 291) | | (N = 587) | |
| AEOI | n (%) | | n (%) | | n (%) | |
| Any infusion related reactions (SSQ/CMQ) | 27 | (9.1%) | 17 | (5.8%) | 44 | (7.5%) |
| Any corneal disorders (SMQ) | 3 | (1.0%) | 1 | (0.3%) | 4 | (0.7%) |
| Any rashes or severe cutaneous adverse | 159 | (53.7%) | 58 | (19.9%) | 217 | (37.0%) |
| reactions | | | | | | |
| Any hyperglycemia (SSQ/CMQ) | 35 | (11.8%) | 8 | (2.7%) | 43 | (7.3%) |
| Any peripheral neuropathy (SMQ) | 149 | (50.3%) | 100 | (34.4%) | 249 | (42.4%) |

The clinical study provided herein compared PADCEV to chemotherapy in adult patients with locally advanced or metastatic urothelial cancer who were previously treated with platinum-based chemotherapy and a PD-1/L1 inhibitor. The results from the clinical study shows that PADCEV® (enfortumab vedotin-ejfv) met its primary endpoint of overall survival compared to chemotherapy.

In the clinical study, PADCEV significantly improved overall survival (OS), with a 30 percent reduction in risk of death (Hazard Ratio (HR)=0.70; (95% Confidence Interval (CI): 0.56, 0.89); p=0.001). PADCEV also significantly improved progression-free survival (PFS), a secondary endpoint, with a 39 percent reduction in risk of disease progression or death (HR=0.61 (95% CI: 0.50, 0.75); p<0.00001).

For patients in the PADCEV arm of the trial, adverse events were consistent with those listed in the U.S. Prescribing Information, with rash, hyperglycemia, decreased neutrophil count, fatigue, anemia and decreased appetite as the most frequent Grade 3 or greater adverse event(s) occurring in more than 5 percent of patients.

Treatment-related adverse events in the safety population are shown in Table 34. The incidence of treatment-related adverse events was high overall but was similar in the two groups (93.9% in the enfortumab vedotin group and 91.8% in the chemotherapy group).

TABLE 34
Treatment-Related Adverse Events (Safety Population)

| | Enfortumab Vedotin | | | | | | | |
|---|---|---|---|---|---|---|---|---|
| | Group | | | | Chemotherapy Group | | | |
| | (N = 296) | | | | (N = 291) | | | |
| | Any Grade | | Grade ≥3 | | Any grade | | Grade ≥3 | |
| Adverse Event | Number of patients (percent) | | | | | | | |
| Any adverse event | 278 | (93.9) | 152 | (51.4) | 267 | (91.8) | 145 | (49.8) |
| Alopecia | 134 | (45.3) | 0 | | 106 | (36.4) | 0 | |
| Peripheral sensory neuropathy† | 100 | (33.8) | 9 | (3.0) | 62 | (21.3) | 6 | (2.1) |
| Pruritus | 95 | (32.1) | 4 | (1.4) | 13 | (4.5) | 0 | |
| Fatigue | 92 | (31.1) | 19 | (6.4) | 66 | (22.7) | 13 | (4.5) |
| Decreased appetite | 91 | (30.7) | 9 | (3.0) | 68 | (23.4) | 5 | (1.7) |
| Diarrhea | 72 | (24.3) | 10 | (3.4) | 48 | (16.5) | 5 | (1.7) |
| Dysgeusia | 72 | (24.3) | 0 | | 21 | (7.2) | 0 | |
| Nausea | 67 | (22.6) | 3 | (1.0) | 63 | (21.6) | 4 | (1.4) |
| Maculopapular rash | 48 | (16.2) | 22 | (7.4) | 5 | (1.7) | 0 | |
| Anemia | 34 | (11.5) | 8 | (2.7) | 59 | (20.3) | 22 | (7.6) |
| Decreased neutrophil count | 30 | (10.1) | 18 | (6.1) | 49 | (16.8) | 39 | (13.4) |
| Neutropenia | 20 | (6.8) | 14 | (4.7) | 24 | (8.2) | 18 | (6.2) |
| Decreased white-cell count | 16 | (5.4) | 4 | (1.4) | 31 | (10.7) | 20 | (6.9) |
| Febrile neutropenia | 2 | (0.7) | 2 | (0.7) | 16 | (5.5) | 16 | (5.5) |

* The safety population included all patients who received any amount of trial drug. Included are treatment-related adverse events that occurred in at least 20% of patients in either treatment group or treatment-related adverse events of grade 3 or higher that occurred in at least 5% of patients in either treatment group. Treatment-related adverse events are those for which there is a reasonable possibility that they were caused by the trial treatment, as assessed by the investigator. If data regarding the relationship to treatment were missing, the event was considered to be related to treatment.
†A total of 113 patients (55 in the enfortumab vedotin group and 58 in the chemotherapy group) had preexisting peripheral neuropathy.

Skin reactions and peripheral neuropathy were the most frequent treatment-related adverse events of special interest with enfortumab vedotin, as shown in Table 35.

TABLE 35
Treatment-Related Adverse Events of Special Interest* by Grade

| | Enfortumab Vedotin Group | | | | | | Chemotherapy Group | | | | | |
|---|---|---|---|---|---|---|---|---|---|---|---|---|
| | (N = 296) | | | | | | (N = 291) | | | | | |
| | Grade | | | | | | Grade | | | | | |
| Event, n (%) | Any | 1 | 2 | 3 | 4 | 5 | Any | 1 | 2 | 3 | 4 | 5 |
| Skin reactions^{ł} | 139 (47.0) | 41 (13.9) | 55 (18.6) | 42 (14.2) | 1 (0.3) | 0 | 46 (15.8) | 30 (10.3) | 14 (4.8) | 2 (0.7) | 0 | 0 |
| Rash | 130 (43.9) | 41 (13.9) | 46 (15.5) | 42 (14.2) | 1 (0.3) | 0 | 28 (9.6) | 21 (7.2) | 6 (2.1) | 1 (0.3) | 0 | 0 |
| Severe cutaneous | 60 (20.3) | 20 (6.8) | 25 (8.4) | 14 (4.7) | 1 (0.3) | 0 | 22 (7.6) | 12 (4.1) | 8 (2.7) | 2 (0.7) | 0 | 0 |
| adverse reaction^{‡} | | | | | | | | | | | | |
| Peripheral | 137 (46.3) | 44 (14.9) | 78 (26.4) | 15 (5.1) | 0 | 0 | 89 (30.6) | 45 (15.5) | 37 (12.7) | 7 (2.4) | 0 | 0 |
| neuropathy | | | | | | | | | | | | |
| Sensory events^{§} | 130 (43.9) | 43 (14.5) | 76 (25.7) | 11 (3.7) | 0 | 0 | 86 (29.6) | 44 (15.1) | 35 (12.0) | 7 (2.4) | 0 | 0 |
| Motor events | 22 (7.4) | 5 (1.7) | 12 (4.1) | 5 (1.7) | 0 | 0 | 7 (2.4) | 5 (1.7) | 2 (0.7) | 0 | 0 | 0 |
| Ocular disorders | 55 (18.6) | 40 (13.5) | 13 (4.4) | 2 (0.7) | 0 | 0 | 14 (4.8) | 11 (3.8) | 2 (0.7) | 1 (0.3) | 0 | 0 |
| Dry eye | 47 (15.9) | 34 (11.5) | 11 (3.7) | 2 (0.7) | 0 | 0 | 9 (3.1) | 6 (2.1) | 2 (0.7) | 1 (0.3) | 0 | 0 |
| Blurred vision | 12 (4.1) | 10 (3.4) | 2 (0.7) | 0 | 0 | 0 | 6 (2.1) | 5 (1.7) | 0 | 1 (0.3) | 0 | 0 |
| Corneal disorders | 2 (0.7) | 2 (0.7) | 0 | 0 | 0 | 0 | 0 | 0 | 0 | 0 | 0 | 0 |
| Infusion-related | 26 (8.8) | 11 (3.7) | 11 (3.7) | 4 (1.4) | 0 | 0 | 13 (4.5) | 6 (2.1) | 7 (2.4) | 0 | 0 | 0 |
| reactions | | | | | | | | | | | | |
| Systemic events | 23 (7.8) | 10 (3.4) | 9 (3.0) | 4 (1.4) | 0 | 0 | 9 (3.1) | 4 (1.4) | 5 (1.7) | 0 | 0 | 0 |
| Local events | 3 (1.0) | 1 (0.3) | 2 (0.7) | 0 | 0 | 0 | 6 (2.1) | 4 (1.4) | 2 (0.7) | 0 | 0 | 0 |
| Infusion site | 2 (0.7) | 0 | 2 (0.7) | 0 | 0 | 0 | 4 (1.3) | 3 (1.0) | 1 (0.3) | 0 | 0 | 0 |
| reactions | | | | | | | | | | | | |
| Extravasation site | 3 (1.0) | 1 (0.3) | 2 (0.7) | 0 | 0 | 0 | 4 (1.4) | 2 (0.7) | 2 (0.7) | 0 | 0 | 0 |
| reactions | | | | | | | | | | | | |
| Hyperglycemia | 19 (6.4) | 3 (1.0) | 4 (1.4) | 11 (3.7) | 0 | 1 (0.3) | 1 (0.3) | 0 | 1 (0.3) | 0 | 0 | 0 |

*Events represent listings by Preferred Term and may include Sponsor Specific Query/Customized Medical Queries (SSQ/CMQ) or Standard MedDRA Queries.
^{ł}Indicates rash or severe cutaneous adverse reactions.
^{‡}Composite Standard MedDRA Query High Level Term of severe cutaneous adverse reactions including: stomatitis, drug eruption, conjunctivitis, dermatitis bullous, skin exfoliation, blister, erythema multiforme, exfoliative rash, fixed eruption, mouth ulceration, pemphigus, and toxic skin eruption.
^{§}Represents “Any peripheral neuropathy sensory events (SSQ/CMQ)” including: peripheral sensory neuropathy, neuropathy peripheral, paraesthesia, polyneuropathy, hypoaesthesia, neurotoxicity, dysaesthesia, gait disturbance, burning sensation, neuralgia, and sensory loss.

Time to Onset of Treatment-Related Adverse Events of Special Interest is shown in Table 36.

TABLE 36
Time to Onset of Treatment-Related
Adverse Events of Special Interest

| | Enfortumab Vedotin Group | | | Chemotherapy Group | |
|---|---|---|---|---|---|
| | (N = 296) | | | (N = 291) | |
| | | Median (range), | | | Median (range), |
| Event | n | months | | n | months |
| Skin reactions | 139 | 0.427 | (0.03, 12.68) | 46 | 0.657 (0.07, 9.56) |
| Peripheral | 137 | 2.694 | (0.03, 11.99) | 89 | 0.821 (0.03, 9.07) |
| neuropathy* | | | | | |
| Corneal | 2 | 4.337 | (1.91, 6.77) | 0 | NA |
| disorders | | | | | |
| Dry eye | 47 | 1.906 | (0.30, 9.66) | 9 | 2.464 (0.03, 5.09) |
| Blurred vision | 12 | 2.448 | (0.07, 5.09) | 6 | 0.871 (0.03, 4.14) |
| Infusion-related | 26 | 0.509 | (0.03, 9.40) | 13 | 0.033 (0.03, 3.19) |
| reactions | | | | | |
| Hyperglycemia | 19 | 0.559 | (0.26, 5.78) | 1 | 1.413 (1.41, 1.41) |

*The time to first onset of grade ≥2 peripheral neuropathy was median (range) of 4.435 (0.36, 12.02) months and 1.725 (0.07, 9.89) months for enfortumab vedotin and chemotherapy groups, respectively.
NA denotes not applicable.

Treatment-Emergent Peripheral Neuropathy and Hyperglycemia by Baseline Status is shown in Table 37.

TABLE 37

| | Enfortumab Vedotin Group | | | Chemotherapy Group | | | |
|---|---|---|---|---|---|---|---|
| | (N = 296) | | | (N = 291) | | | |
| | Baseline PN | No baseline PN | | Baseline PN | | No baseline PN | |
| Event, n (%) | (n = 55) | (n = 241) | | (n = 58) | | (n = 233) | |
| Peripheral | 27 (49.1) | 122 | (50.6) | 17 | (29.3) | 83 | (35.6) |
| neuropathy | | | | | | | |
| Peripheral | 22 (40.0) | 117 | (48.5) | 16 | (27.6) | 81 | (34.8) |
| sensory | | | | | | | |
| neuropathy | | | | | | | |
| Peripheral | 11 (20.0) | 22 | (9.1) | 4 | (6.9) | 5 | (2.1) |
| motor | | | | | | | |
| neuropathy | | | | | | | |
| Hyperglycemia | 21 (38.2) | 14 | (5.8) | 4 | (7.1) | 4 | (1.7) |

| | Baseline BMI | Baseline BMI | Baseline BMI | Baseline BMI |
|---|---|---|---|---|
| | ≥30 kg/m² | <30 kg/m² | ≥30 kg/m2 | <30 kg/m² |
| | (n = 41) | (n = 255) | (n = 46) | (n = 244) |
| Hyperglycemia | 12 (29.3) | 23 (9.0) | 1 (2.2) | 7 (2.9) |

*Baseline status indicates the health condition (history or ongoing) of patients as they enter the trial before they receive any study treatment. Patients with preexisting grade ≥2 sensory or motor neuropathy or a history of uncontrolled diabetes mellitus within 3 months of the first dose of study drug were excluded from the trial. Uncontrolled diabetes was defined as a hemoglobin A1c ≥8% or between 7 and <8% with associated diabetes symptoms (polyuria or polydipsia) that were not otherwise explained. Aside from these exclusion criteria, patients with preexisting neuropathy and hyperglycemia were permitted to enter the trial.
BMI denotes body mass index and PN peripheral neuropathy.

Adverse events, regardless of relationship to treatment, that resulted in death (excluding disease progression) during the treatment period occurred in 11 patients in each group; the incidence remained the same after adjustment for treatment exposure. Investigator-assessed treatment-related adverse events that resulted in death occurred in 7 patients (2.4%) in the enfortumab vedotin group (multiorgan dysfunction syndrome [in 2 patients] and abnormal hepatic function, hyperglycemia, pelvic abscess, pneumonia, and septic shock [each in 1 patient]) and in 3 patients (1.0%) in the chemotherapy group (neutropenic sepsis, sepsis, and pancytopenia [each in 1 patient]). The demographic characteristics of the patients in the enfortumab vedotin group who died are provided in Table 38.

TABLE 38
Patient Details on Adverse Events Leading
to Death in the Enfortumab Vedotin Group

| | |
|---|---|
| Adverse Event | Brief Past Medical History |

Treatment-Related Adverse Events*

| | |
|---|---|
| Multi-organ dysfunction | Chronic kidney disease, stage II; atrial |
| | flutter; hypertension |
| Multi-organ dysfunction | Hypertension; diabetes mellitus (on metfor- |
| | min); chronic obstructive pulmonary disease; |
| | dyslipidemia; ischemic heart disease |
| Pneumonia | Hypertension; decreased appetite; anemia |
| Septic shock | Hypertension; dyslipidemia; myocardial |
| | infarction |
| Hepatic dysfunction | Hypertension; hyperuricemia; cancer pain |
| | (on oxycodone and acetaminophen) |
| Pelvic abscess | Angina pectoris; pelvic pain; colitis (while |
| | on pembrolizumab, requiring steroids) |
| Hyperglycemia | Hypertension; membranous glomerulonephritis; |
| | dyslipidemia; |
| | BMI: 33 kg/m² |

Treatment-Emergent Adverse Eventsł

| | |
|---|---|
| Pneumonia | Hypertension; hyperthyroidism |
| Cerebral edema | Urinary tract infections; ascites |
| Multi-organ dysfunction | Diabetes mellitus type 2; hypertension; |
| | peripheral edema; liver cirrhosis; anemia; |
| | enterococcal infection |
| Dyspnea | Anemia |

*“Treatment-related” adverse event indicates reasonable possibility that the event may have been caused by the study treatment as assessed by the investigator. If the relationship is missing, then the adverse event is considered to be treatment-related.
ł“Treatment-emergent” adverse events (n = 11) also include all events deemed treatment-related adverse events.
BMI denotes body mass index.

### 6.2 Example 2—Analysis of Hard-to-Treat Subgroups from the Clinical Study Described in Section 6.1

In the clinical study described in Section 6.1, Enfortumab Vedotin (EV) showed superior overall survival (OS) compared with standard chemotherapy (SC) in patients with previously treated locally advanced or metastatic urothelial carcinoma (la/mUC). This subgroup analysis evaluates EV efficacy/safety for those patients with poor prognostic factors, such as age ≥65 years, presence of liver metastasis, primary upper tract disease, and checkpoint inhibitor (CPI) nonresponse.

Methods: In this open-label trial, la/mUC patients who had received prior platinum-based chemotherapy and had disease progression during/after PD-1/L1 inhibitor were randomized 1:1 to EV or investigator's choice of SC (docetaxel, paclitaxel, vinflunine). Subgroup analyses were prespecified for the primary endpoint of OS and secondary endpoints of investigator-assessed progression-free survival (PFS) and overall response rate (ORR) per RECIST v1.1. Kaplan-Meier analyses compared OS and PFS between treatments for selected hard-to-treat subgroups as follows: age ≥65 years, presence of liver metastasis, primary upper tract disease, and checkpoint inhibitor (CPI) nonresponse. ORR and safety were also evaluated within subgroups. The Cox proportional hazards model was used to estimate hazard ratios. The Cochran-Mantel-Haenszel test was used to compare response and disease control rates between groups.

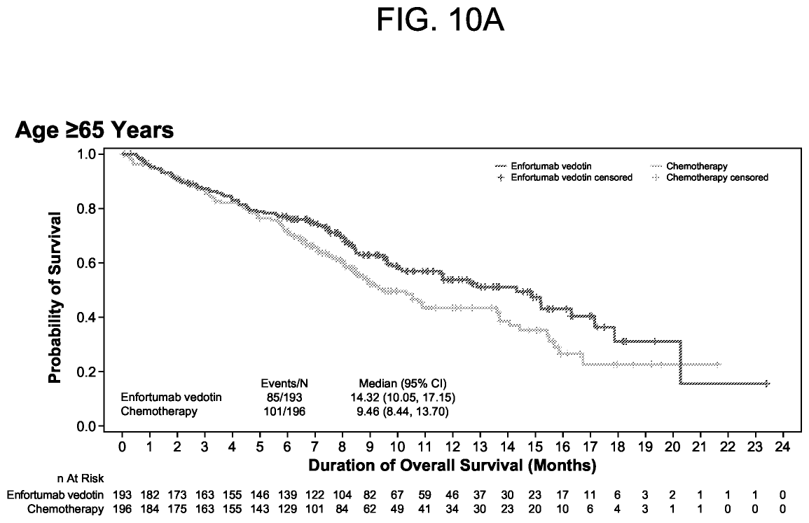

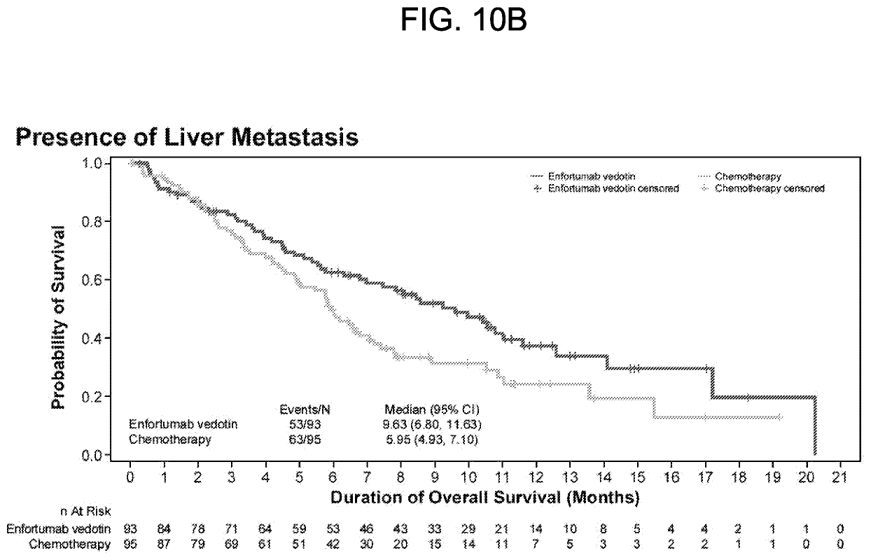

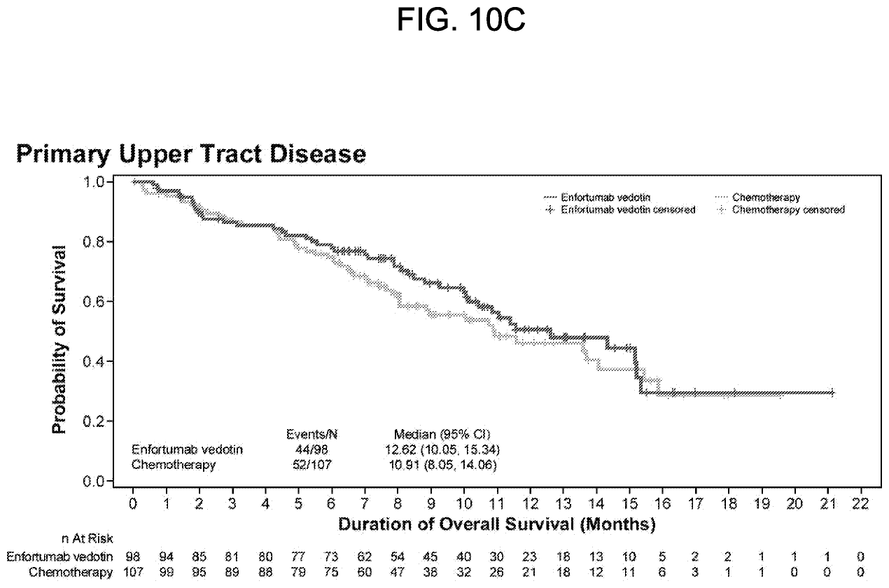

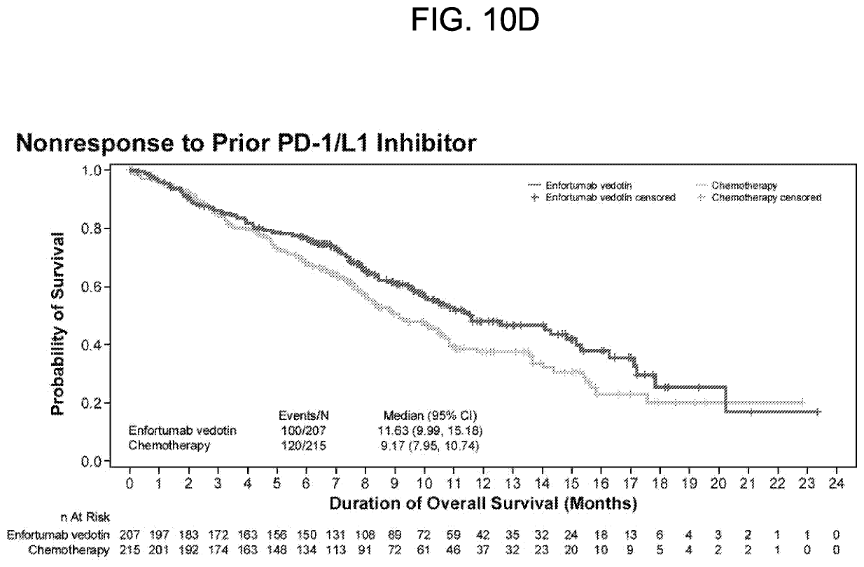

Results: 608 patients in total were randomized either to EV (n=301) or SC (n=307); median follow-up was 11.1 months. The OS benefit of EV was retained across the majority of subgroups (e.g., patients with liver metastasis and patients who are nonresponse to prior immune checkpoint inhibitor (i.e., PD-1/L1; “CPI nonresponsive” or “CPI non-responder”)), with a longer median OS observed in patients on EV vs. patients on SC, as shown in Table 39 and FIGS. 10A-10D. For the primary upper tract disease subgroup, the median OS was longer for EV versus SC and consistent with the median OS for the overall population as shown in Table 39 and FIGS. 10A-10D.

TABLE 39
Overall Survival, Progression-Free Survival, and Overall Response Rate in all patients, patients
with liver metastasis, patients with primary upper tract disease, and CPI nonresponsive patients

| | | | | | | | | | | | Overall response rate (ORR) | | | | |
|---|---|---|---|---|---|---|---|---|---|---|---|---|---|---|---|
| | Overall survival (OS) | | | | | Progression-free survival (PFS) | | | | | (Response-Evaluable Population) | | | | |
| | | | | | EV vs | | | | | EV vs | | | | | EV vs SC |
| | | | | | SC | | | | | SC | | | | | Absolute |
| | EV | | SC | | HR | EV | | SC | | HR | | | | | Difference |
| | | Os^{b} | | Os^{b} | (95% | | PFS^{b} | | PFS^{b} | (95% | EV | | SC | | %, (95% |
| | Events^{a} | (mo) | Events* | (mo) | CI) | Events^{a} | (mo) | Events^{a} | (mo) | CI) | n/N | % | n/N | % | CI) |
| All | 134/301 | 12.88 | 167/307 | 8.97 | 0.702 | 201/301 | 5.55 | 231/307 | 3.71 | 0.615 | 117/288 | 40.6 | 53/296 | 17.9 | 22.7 |
| | (44.5) | | (54.4) | | (0.556, | (66.8) | | (75.2) | | (0.505, | | (34.90, | | (13.71, | (14.70, |
| | | | | | 0.886) | | | | | 0.748) | | 46.54) | | 22.76) | 30.56) |
| Age ≥65 | 85/193 | 14.32 | 101/196 | 9.46 | 0.745 | 126/193 | 5.65 | 151/196 | 3.78 | 0.616 | 75/184 | 40.8 | 38/191 | 19.9 | 20.9 |
| yrs | (44) | | (52) | | (0.558, | (65) | | (77) | | (0.485, | | (33.59, | | (14.48, | (10.75, |
| | | | | | 0.995) | | | | | 0.781) | | 48.23) | | 26.27) | 30.61) |
| Presence | 53/93 | 9.63 | 63/95 | 5.95 | 0.660 | 71/93 | 4.14 | 75/95 | 2.63 | 0.597 | 33/93 | 35.5 | 10/93 | 10.8 | 24.7 |
| of Liver | (57) | | (66) | | (0.456, | (76) | | (79) | | (0.428, | | (25.83, | | (5.28, | (9.96, |
| metastasis | | | | | 0.957) | | | | | 0.833) | | 46.09) | | 18.89) | 38.70) |
| Primary | 44/98 | 12.62 | 52/107 | 10.91 | 0.848 | 63/98 | 5.62 | 74/107 | 3.78 | 0.716 | 43/98 | 43.9 | 20/105 | 19.0 | 24.8 |
| upper | (45) | | (49) | | (0.567, | (64) | | (69) | | (0.511, | | (33.87, | | (12.04, | (11.07, |
| tract | | | | | 1.269) | | | | | 1.003) | | 54.27) | | 27.87) | 37.80) |
| disease | | | | | | | | | | | | | | | |
| CPI non- | 100/207 | 11.63 | 120/215 | 9.17 | 0.757 | 146/207 | 5.42 | 160/215 | 3.65 | 0.697 | 79/199 | 39.7 | 36/207 | 17.4 | 22.3 |
| responder | (48) | | (56) | | (0.580, | (71) | | (74) | | (0.556, | | (32.85, | | (12.49, | (12.67, |
| | | | | | 0.988) | | | | | 0.873) | | 46.86) | | 23.25) | 31.68) |

^{a}n/N, (%).
^{b}Median months for OS, PFS.
SC, standard chemotherapy;
CI, Confidence Interval

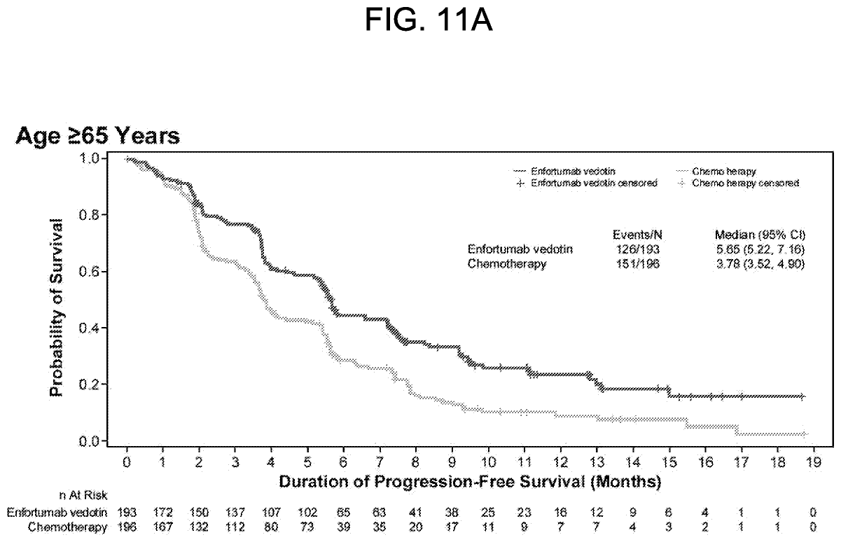

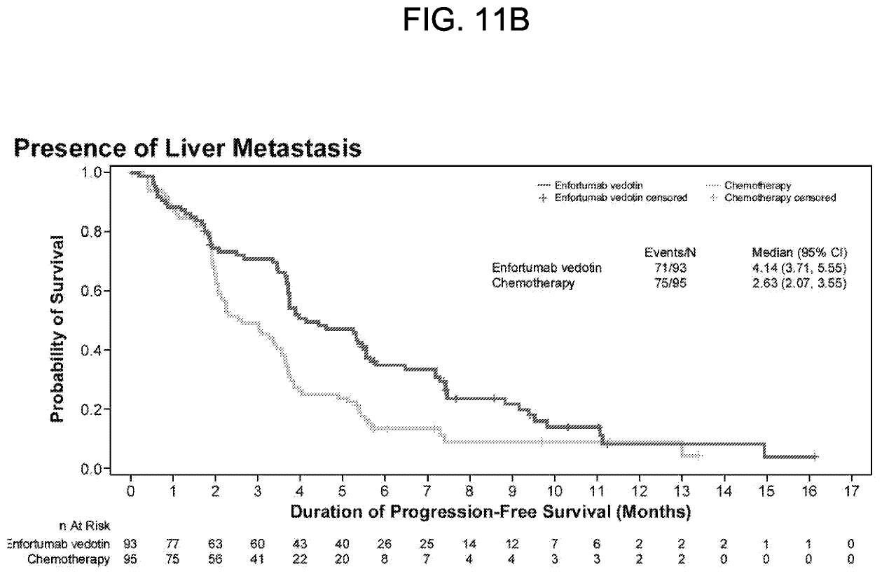

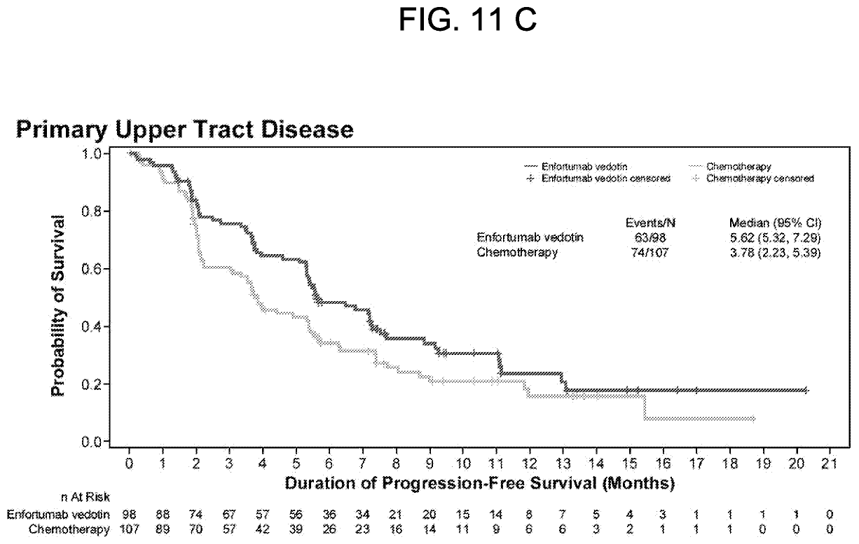

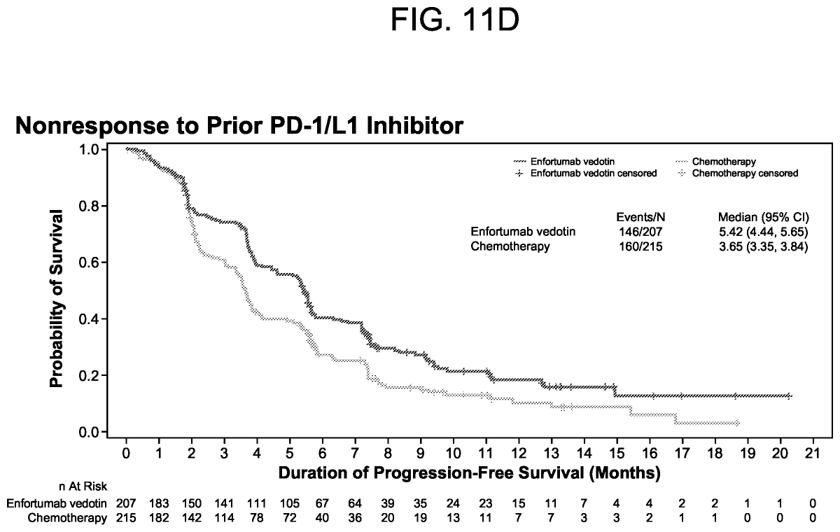

PFS was also longer and ORR was higher across all subgroups for EV compared to SC. In particular, a similar benefit was also observed for PFS across the subgroups for EV versus SC, as shown in Table 39 and FIGS. 11A-11D. For the primary upper tract disease subgroup, the median PFS was longer for EV versus SC and consistent with the median PFS for the overall population, as shown in Table 39 and FIGS. 11A-11D. In all randomized patients with measurable disease at baseline, the overall response rate (ORR) was higher in each subgroup for EV relative to SC and appeared consistent between treatments across all subgroups, as shown in Table 39.

Safety and Tolerability: Overall rates of treatment-emergent and treatment-related adverse events (AE's) were similar between treatments across subgroups. For example, the overall rate of AE's in patients ≥65 years of age treated with EV was 97.4% and the overall rate of AE's in patients ≥65 years of age treated with SC was 98.9%. The overall rate of AE's in patients with liver metastasis treated with EV was 97.8% and the overall rate of AE's in patients with liver metastasis treated with SC was 96.7%. The overall rate of AE's in patients with primary upper tract disease treated with EV was 99.0% and the overall rate of AE's in patients with primary upper tract disease treated with SC was 99.0%. The overall rate of AE's in patients who were non-responsive to prior treatment with PD-1/L1 inhibitors and treated with EV was 97.5% and the overall rate of AE's in patients who were non-responsive to prior treatment with PD-1/L1 inhibitors and treated with SC was 99.5%.

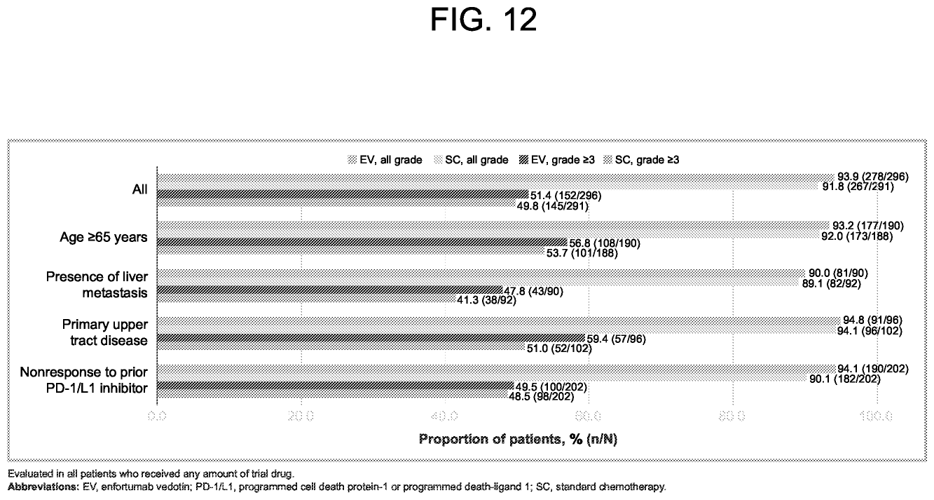

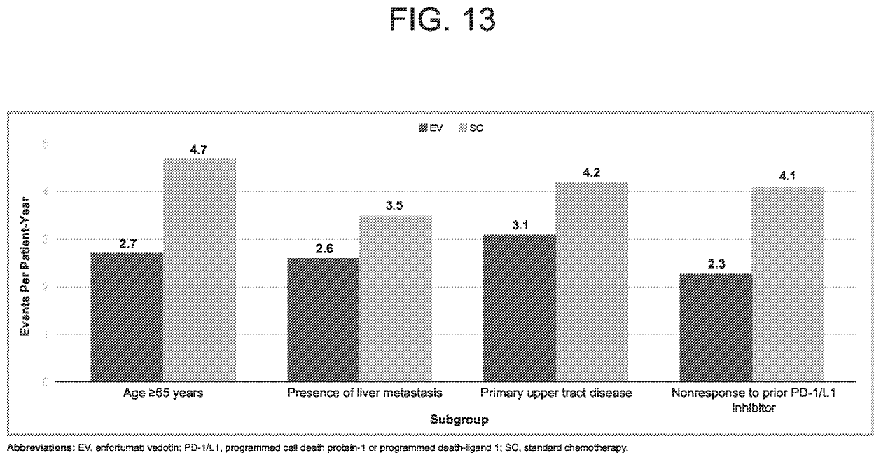

Treatment-related AE's were comparable between treatments across subgroups, as shown in FIG. 12. AE's were evaluated in all patients who received any amount of trial drug. When adjusted for treatment exposure, grade ≥3 treatment-related AEs occurred less frequently in the EV versus SC group across all four subgroups (i.e., in patients ≥65 years of age, in patients with liver metastasis, in patients with primary upper tract disease, and in patients who are non-responsive to prior treatment with PD-1/L1 inhibitors), as shown in FIG. 13. The incidence of grade ≥3 treatment-related AEs in the hard-to-treat subgroups generally aligned and was consistent with those observed in the overall EV-301 safety population, as shown in Table 40.

TABLE 40
Incidence of Grade ≥3 Treatment-Related Adverse Events (Safety Population)

| | | | | | | | | | Nonresponsive to | |
|---|---|---|---|---|---|---|---|---|---|---|
| | | | | | Presence of | | Primary Upper | | Prior | |
| | All | | Aged ≥65 Years | | Liver Metastasis | | Tract Disease | | PD-1/L1 Inhibitor | |
| Adverse | EV | SC | EV | SC | EV | SC | EV | SC | EV | SC |
| Event | N = 296 | N = 291 | N = 190 | N = 188 | N = 90 | N = 92 | N = 96 | N = 102 | N = 202 | N = 202 |
| Maculopapular | 22 (7.4) | 0 | 14 (7.4) | 0 | 8 (8.9) | 0 | 10 (10.4) | 0 | 19 (9.4) | 0 |
| rash | | | | | | | | | | |
| Fatigue | 19 (6.4) | 13 (4.5) | 15 (7.9) | 12 (6.4) | 5 (5.6) | 5 (5.4) | 9 (9.4) | 5 (4.9) | 10 (5.0) | 5 (2.5) |
| Decreased | 18 (6.1) | 39 (13.4) | 14 (7.4) | 26 (13.8) | 5 (5.6) | 7 (7.6) | 9 (9.4) | 18 (17.6) | 10 (5.0) | 27 (13.4) |
| neutrophil | | | | | | | | | | |
| count | | | | | | | | | | |
| Neutropenia | 14 (4.7) | 18 (6.2) | 7 (3.7) | 15 (8.0) | 5 (5.6) | 4 (4.3) | 6 (6.3) | 7 (6.9) | 9 (4.5) | 10 (5.0) |
| Anemia | 8 (2.7) | 22 (7.6) | 5 (2.6) | 15 (8.0) | 3 (3.3) | 3 (3.3) | 6 (6.3) | 5 (4.9) | 6 (3.0) | 12 (5.9) |
| Decreased | 4 (1.4) | 20 (6.9) | 4 (2.1) | 14 (7.4) | 0 | 3 (3.3) | 1 (1.0) | 9 (8.8) | 2 (1.0) | 15 (7.4) |
| white blood | | | | | | | | | | |
| cell count | | | | | | | | | | |
| Febrile | 2 (0.7) | 16 (5.5) | 2 (1.1) | 11 (5.9) | 2 (2.2) | 6 (6.5) | 2 (2.1) | 7 (6.9) | 2 (1.0) | 10 (5.0) |
| neutropenia | | | | | | | | | | |

^{a}Events occurring in at least 5% of patients in either treatment group from the total safety population.
Abbreviations: EV, enfortumab vedotin; PD-1/L1, programmed cell death protein-1 or programmed death-ligand 1; SC, standard chemotherapy.

Conclusions: Patients having locally advanced or metastatic urothelial carcinoma (La/mUC) with poor prognostic factors receiving EV had consistently longer OS, longer PFS, and higher ORR compared to patients receiving SC. Safety profiles for study treatments were consistent with those of prior studies and with the overall study population; no new safety signals were observed. These data support the use of EV in previously treated patients with la/mUC, including those with poor prognostic factors.

What is claimed is:

1. A method of treating urothelial or bladder cancer in a human subject having liver metastases, comprising administering to the subject having liver metastases an effective amount of an antibody drug conjugate,
wherein the subject has received an immune checkpoint inhibitor (CPI) therapy, and
wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

2. A method of treating urothelial or bladder cancer in a human subject having a primary site of tumor in the upper urinary tract, comprising administering to the subject having a primary site of tumor in the upper urinary tract an effective amount of an antibody drug conjugate,
wherein the subject has received an immune checkpoint inhibitor (CPI) therapy, and
wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

3. A method of treating urothelial or bladder cancer in a human subject, comprising administering to the subject an effective amount of an antibody drug conjugate,
wherein the subject has received an immune checkpoint inhibitor (CPI) therapy,
wherein the subject had progression or recurrence of the cancer during or following the CPI therapy, and
wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

4. The method of any one of claims 1 to 3, wherein the subject has a duration of response of at least or about 7 months following the treatment.

5. The method of any one of claims 1 to 3, wherein the subject has a duration of response ranging from 5 to 9 months following the treatment.

6. The method of claim 1, wherein the subject has a progression free survival of at least or about 4 months following the treatment.

7. The method of claim 2 or 3, wherein the subject has a progression free survival of at least or about 5 months following the treatment.

8. The method of claim 1, wherein the subject has a progression free survival ranging from 4 to 9 months following the treatment.

9. The method of claim 2 or 3, wherein the subject has a progression free survival ranging from 5 to 9 months following the treatment.

10. The method of claim 1, wherein the subject has an overall survival of at least or about 9 months following the treatment.

11. The method of claim 2, wherein the subject has an overall survival of at least or about 12 months following the treatment.

12. The method of claim 3, wherein the subject has an overall survival of at least or about 11 months following the treatment.

13. The method of any one of claims 1 to 3, wherein the subject has an overall survival ranging from 9 to 19 months following the treatment.

14. The method of any one of claims 1 to 3, wherein a population of the subjects is treated by the methods, and wherein the percentage of the subjects having complete response in the treated population is at least or about 4%.

15. The method of any one of claims 1 to 3, wherein a population of the subjects is treated by the methods, and wherein the percentage of the subjects having partial response in the treated population is at least or about 35%.

16. The method of claim 1, wherein a population of the subjects is treated by the methods, and wherein overall response rate in the treated population is at least or about 35%.

17. The method of claim 2, wherein a population of the subjects is treated by the methods, and wherein overall response rate in the treated population is at least or about 43%.

18. The method of claim 3, wherein a population of the subjects is treated by the methods, and wherein overall response rate in the treated population is at least or about 39%.

19. The method of any one of claims 1 to 3, wherein a population of the subjects is treated by the methods, and wherein the percentage of the subjects having stable disease in the treated population is at least or about 30%.

20. The method of any one of claims 1 to 3, wherein a population of the subjects is treated by the methods, and wherein median duration of response in the treated population is at least or about 7 months.

21. The method of any one of claims 1 to 3, wherein a population of the subjects is treated by the methods, and wherein duration of response in the treated population ranges from 5 to 9 months.

22. The method of claim 1, wherein a population of the subjects is treated by the methods, and wherein median progression free survival in the treated population is at least or about 4 months.

23. The method of claim 1, wherein a population of the subjects is treated by the methods, and wherein progression free survival in the treated population ranges from 4 to 9 months.

24. The method claim 2 or 3, wherein a population of the subjects is treated by the methods, and wherein median progression free survival in the treated population is at least or about 5 months.

25. The method of claim 2 or 3, wherein a population of the subjects is treated by the methods, and wherein progression free survival in the treated population ranges from 5 to 9 months.

26. The method of claim 1, wherein a population of the subjects is treated by the methods, and wherein median overall survival in the treated population is at least or about 9 months.

27. The method of claim 2, wherein a population of the subjects is treated by the methods, and wherein median overall survival in the treated population is at least or about 12 months.

28. The method of claim 3, wherein a population of the subjects is treated by the methods, and wherein median overall survival in the treated population is at least or about 11 months.

29. The method of any one of claims 1 to 3, wherein a population of the subjects is treated by the methods, and wherein overall survival in the treated population ranges from 9 to 19 months.

30. The method of any one of claims 1 to 3, wherein the complete response rate is at least or about 4% for a population of subjects treated with the method.

31. The method of any one of claims 1 to 3, wherein the partial response rate is at least or about 35% for a population of subjects treated with the method.

32. The method of claim 1, wherein overall response rate is at least or about 35% for a population of subjects treated with the method.

33. The method of claim 2, wherein overall response rate is at least or about 43% for a population of subjects treated with the method.

34. The method of claim 3, wherein overall response rate is at least or about 39% for a population of subjects treated with the method.

35. The method of any one of claims 1 to 3, wherein the median duration of response is at least or about 7 months for a population of subjects treated with the method.

36. The method of any one of claims 1 to 3, wherein the duration of response is from 5 to 9 months for a population of subjects treated with the method.

37. The method of claim 1, wherein the median progression free survival is at least or about 4 months for a population of subjects treated with the method.

38. The method of any one of claims 1 to 3, wherein the progression free survival is from 4 to 9 months for a population of subjects treated with the method.

39. The method of claim 2 or 3, wherein the median progression free survival is at least or about 5 months for a population of subjects treated with the method.

40. The method of claim 2 or 3, wherein the progression free survival is from 5 to 9 months for a population of subjects treated with the method.

41. The method of claim 1, wherein the median overall survival is at least or about 9 months for a population of subjects treated with the method.

42. The method of claim 2, wherein the median overall survival is at least or about 12 months for a population of subjects treated with the method.

43. The method of claim 3, wherein the median overall survival is at least or about 11 months for a population of subjects treated with the method.

44. The method of any one of claims 1 to 3, wherein the overall survival is from 9 to 19 months for a population of subjects treated with the method.

45. The method of any one of claims 1 to 44, wherein the subject is a subject that received platinum-based chemotherapy.

46. The method of any one of claims 1 to 45, wherein the cancer is urothelial cancer, and wherein the human subject has locally advanced or metastatic urothelial carcinoma.

47. The method of any one of claims 1 to 46, wherein the subject has one or more of the conditions selected from the group consisting of:
(i) absolute neutrophil count (ANC) no less than 1500/mm³;
(ii) platelet count no less than 100×10⁹/L;
(iii) hemoglobin no less than 9 g/dL;
(iv) serum bilirubin no more than either of 1.5 times of upper limit of normal (ULN) or 3 times ULN for patients with Gilbert's disease;
(v) CrCl no less than 30 mL/min, and
(vi) alanine aminotransferase and aspartate aminotransferase no more than 3 fold of ULN.

48. The method of claim 47, wherein the subject has all of conditions (i) to (vi) of claim 47.

49. The method of claim 47 or 48, wherein the CrCl is measured by 24 hour urine collection or estimated by the Cockcroft-Gault criteria.

50. The method of any one of claims 1 to 49, wherein the subject has no more than Grade 2 sensory or motor neuropathy.

51. The method of any one of claims 1 to 50, wherein the subject has no active central nervous system metastases.

52. The method of any one of claims 1 to 51, wherein the subject has no uncontrolled diabetes.

53. The method of claim 52, wherein the uncontrolled diabetes is determined by hemoglobin A1c (HbA1c) no less than 8% or HbA1c between 7 and 8% with associated diabetes symptoms that are not otherwise explained.

54. The method of claim 53, wherein the associated diabetes symptoms comprise or consist of polyuria, polydipsia, or both polyuria and polydipsia.

55. The method of any one of claims 1 to 54, wherein the CPI therapy is a therapy of programmed death receptor-1 (PD-1) inhibitor.

56. The method of any one of claims 1 to 54, wherein the CPI therapy is a therapy of programmed death-ligand 1 (PD-L1) inhibitor.

57. The method of claim 55, wherein PD-1 inhibitor is nivolumab or pembrolizumab.

58. The method of claim 56, wherein PD-L1 inhibitor is selected from a group consisting of atezolizumab, avelumab, and durvalumab.

59. The method of any one of claims 1 to 58, wherein the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or
wherein the antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

60. The method of any one of claims 1 to 58, wherein the antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
wherein the antibody or antigen binding fragment thereof comprises CDR-H1 consisting of the amino acid sequence of SEQ ID NO:16, CDR-H2 consisting of the amino acid sequence of SEQ ID NO:17, CDR-H3 consisting of the amino acid sequence of SEQ ID NO:18; CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.

61. The method of any one of claims 1 to 60, wherein the antibody or antigen binding fragment thereof comprises a heavy chain variable region comprising the amino acid sequence of SEQ ID NO:22 and a light chain variable region comprising the amino acid sequence of SEQ ID NO:23.

62. The method of any one of claims 1 to 61, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

63. The method of any one of claims 1 to 61, wherein the antigen binding fragment is an Fab, F(ab′)2, Fv or scFv.

64. The method of any one of claims 1 to 62, wherein the antibody is a fully human antibody.

65. The method of any one of claims 1 to 62 and 64, wherein the antibody is an IgG1 and light chain is a kappa light chain

66. The method of any one of claims 1 to 65, wherein the antibody or antigen binding fragment thereof is recombinantly produced.

67. The method of any one of claims 1 to 66, wherein the antibody or antigen binding fragment is conjugated to each unit of MMAE via a linker.

68. The method of claim 67, wherein the linker is an enzyme-cleavable linker, and wherein the linker forms a bond with a sulfur atom of the antibody or antigen binding fragment thereof.

69. The method of claim 67 or 68, wherein the linker has a formula of: -Aa-Ww-Yy-; wherein -A- is a stretcher unit, a is 0 or 1; -W- is an amino acid unit, w is an integer ranging from 0 to 12; and -Y- is a spacer unit, y is 0, 1, or 2.

70. The method of claim 69, wherein the stretcher unit has the structure of Formula (1) below; the amino acid unit is valine-citrulline; and the spacer unit is a PAB group comprising the structure of Formula (2) below:

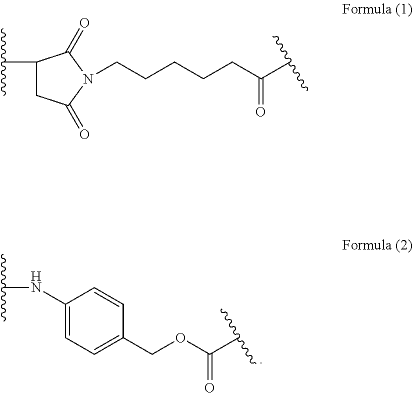 [SMILES: *C(=O)CCCCCN1C(=O)CC(*)C1=O ; *Nc1ccc(COC(*)=O)cc1]

71. The method of claim 69 or 70, wherein the stretcher unit forms a bond with a sulfur atom of the antibody or antigen binding fragment thereof; and wherein the spacer unit is linked to MMAE via a carbamate group.

72. The method of any one of claims 1 to 71, wherein the ADC comprises from 1 to 20 units of MMAE per antibody or antigen binding fragment thereof.

73. The method of any one of claims 1 to 72, wherein the ADC comprises from 1 to 10 units of MMAE per antibody or antigen binding fragment thereof.

74. The method of any one of claims 1 to 73, wherein the ADC comprises from 2 to 8 units of MMAE per antibody or antigen binding fragment thereof.

75. The method of any one of claims 1 to 74, wherein the ADC comprises from 3 to 5 units of MMAE per antibody or antigen binding fragment thereof.

76. The method of any one of claims 1 to 73, wherein the ADC has the following structure: 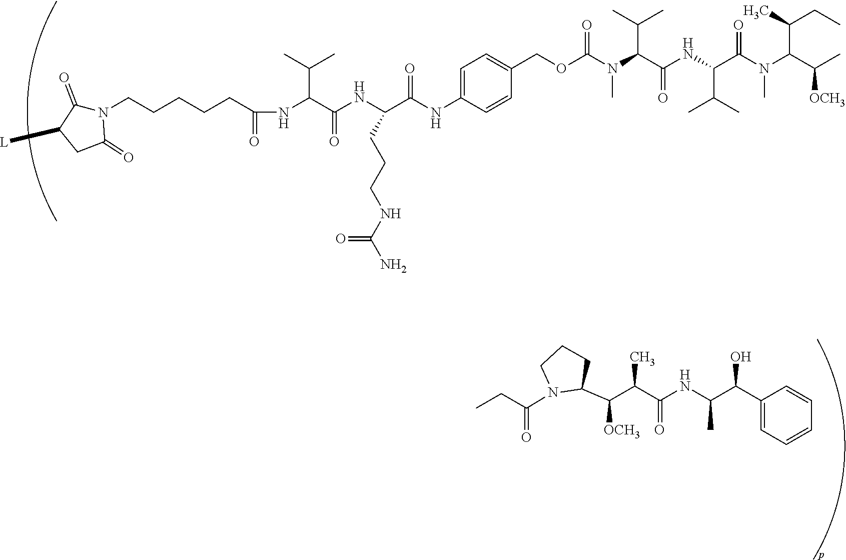
wherein L- represents the antibody or antigen binding fragment thereof and p is from 1 to 10.

77. The method of claim 76, wherein p is from 2 to 8.

78. The method of claim 76 or 77, wherein p is from 3 to 5.

79. The method of any one of claims 76 to 78, wherein p is from 3 to 4.

80. The method of any one of claims 77 to 79, wherein p is about 4.

81. The method of any one of claims 76 to 79, wherein the average p value of the effective amount of the antibody drug conjugates is about 3.8.

82. The method of any one of claims 1 to 81, wherein the ADC is administered at a dose of about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.

83. The method of any one of claims 1 to 82, wherein the ADC is administered at a dose of about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

84. The method of any one of claims 1 to 83, wherein the ADC is administered at a dose of about 1 mg/kg of the subject's body weight.

85. The method of any one of claims 1 to 83, wherein the ADC is administered at a dose of about 1.25 mg/kg of the subject's body weight.

86. The method of any one of claims 1 to 85, wherein the ADC is administered by an intravenous (IV) injection or infusion.

87. The method of any one of claims 1 to 86, wherein the ADC is administered by an IV injection or infusion three times every four-week cycle.

88. The method of any one of claims 1 to 87, wherein the ADC is administered by an IV injection or infusion on Days 1, 8 and 15 of every four-week cycle.

89. The method of any one of claims 1 to 88, wherein the ADC is administered by an IV injection or infusion over about 30 minutes three times every four-week cycle.

90. The method of any one of claims 1 to 89, wherein the ADC is administered by an IV injection or infusion over about 30 minutes on Days 1, 8 and 15 of every four-week cycle.

91. The method of any one of claims 1 to 90, wherein the ADC is formulated in a pharmaceutical composition comprising L-histidine, polysorbate-20 (TWEEN-20), and trehalose dehydrate.

92. The method of any one of claims 1 to 91, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

93. The method of any one of claims 1 to 91, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

94. The method of any one of claims 1 to 93, wherein the ADC is enfortumab vedotin (EV) or a biosimilar thereof, wherein the EV is administered at a dose of about 1.25 mg/kg of the subject's body weight, and wherein the dose is administered by an IV injection or infusion over about 30 minutes on Days 1, 8 and 15 of every four-week cycle.

95. The method of any one of claims 1 to 94, whereby a population of the subjects have a complete response following the treatment.

96. The method of any one of claims 1 to 94, wherein a population of the subjects have a partial response following the treatment.

97. The method of any one of claims 1 to 94, wherein a population of the subjects have a complete response or a partial response following the treatment.

98. The method of any one of claims 1 to 94, wherein a population of the subjects have a stable disease following the treatment.
